SEQUENCE LISTING (36 sequences)

SEQ ID NO: 1 - protein, 853 aa, Caenorhabditis elegans
MVTADETVLATTTNTTSMSVEPTDPRSAGESSSDSEPDTIEQLKAEQREVMADAANGSEVNGNQENGKEEAASADVEVIEIDDTEESTDPSPDGSDENGDAASTSVPIEEEARKKDEGASEVTVASSEIEQDDDGDVMEITEEPNGKSEDTANGTVTEEVLDEEEPEPSVNGTTEIATEKEPEDSSMPVEQNGKGVKRPVECIELDDDDDDEIQEISTPAPAKKAKIDDVKATSVPEEDNNEQAQKRLLDKLEEYVKEQKDQPSSKSRKVLDTLLGAINAQVQKEPLSVRKLILDKVLVLPNTISFPPSQVCDLLIEHDPEMPLTKVINRMFGEERPKLSDSEKRERAQLKQHNPVPNMTKLLVDIGQDLVQEATYCDIVHAKNLPEVPKNLETYKQVAAQLKPVWETLKRKNEPYKLKMHRCDVCGFQTESKLVMSTHKENLHFTGSKFQCTMCKETDTSEQRMKDHYFETHLVIAKSEEKESKYPCAICEEDFNFKGVREQHYKQCKKDYIRIRNIMMPKQDDHLYINRWLWERPQLDPSILQQQQQAALQQAQQKKQQQLLHQQQAAQAAAAAQLLRKQQLQQQQQQQQARLREQQQAAQFRQVAQLLQQQSAQAQRAQQNQGNVNHNTLIAAMQASLRRGGQQGNSLAVSQLLQKQMAALKSQQGAQQLQAAVNSMRSQNSQKTPTHRTPTFVCEICDASVQEKEKYLQHLQTTHKQMVGKVLQDMSQGAPLACSRCRDRFWTYEGLERHLVMSHGLVTADLLLKAQKKEDGGRCKTCGKNYAFNMLQHLVADHQVKLCSAEIMYSCDVCAFKCSSYQTLEAHLTSNHPKGDKKTSTPAKKDDCITLDD

SEQ ID NO: 2 - DNA, 4001 bp, Caenorhabditis elegans
tcacacactcatgacatacacacatcatttcgcctcacacaccgcgccgtcgccatccgcaccgcccgggtgggacgtgttcaaacttttcggttttcgtaattaatagtgagccccggtttattcgctttgagaatcagtataatggatatatcagattgtgtaattaggttgcgtgcttgaacttttaaaattaactgttttaaatttatctgcctttatcgttacagtaaatcattttgatgaacttttcggatgaatcataatgaagtacgcagcgctctaacaaaatgtgtttgtaaattccaattgctacaagttgcccggcttattttttggtgattgaagcatgattctgttgacgctcccgacgcggaataccaggacggaccgatgagagagtactgccagtgaagagacgcatgcgagcaggacgagtgctgctcacccttcttctcagcgtcggcggctgcgaccagcggccgaggaaggggaggagagaggccgatttggctgcgtaccacgtttgatactcagtcacttaccacagctggttctcttgtgcgttcaaatctggcttgccgcgcgcgcgcattttattcctaccagtttgaatctcccacctctccgactgtaactgtcctaatttgcttccttctcatcactctctctttgcctatttctcactatctagactctatttttccagaatggtcaccgccgacgagacggtactcgccacaacgaccaacaccacttccatgtctgtggaaccaacggatccgagaagcgctggtgaatcgtcctcagattcggagccagacacaattgaggtgaggaaaagttttgggaatttaaatctgaataaaacgttttcagcagctgaaggcagaacagcgcgaagtgatggccgacgcggcgaatggttccgaagtcaacggaaatcaagagaacggaaaagaggaagcggcatctgcagacgtggaagtgatcgagatagatgacaccgaagagtctacggatccctcacctgatggatctgatgaaaacggtgatgctgcatctacatcggttccaatcgaagaggaagcgcgtaaaaaggatgagggggcttccgaagtgactgtggcatcatctgagattgaacaagacgatgatggcgatgttatggaaatcactgaggagccgaacggaaagtcggaggatactgccaacggaacaggtgtgttttataattttaccaagtttaattttaactttctattttcagttactgaggaggtgctagatgaagaggagccagaaccttccgtaaacggaacaactgagatcgctacagagaaagagccagaagattcttcaatgcctgtcgaacagaatgggaagggtgtgaagcggcctgtcgaatgcatcgaactcgacgacgacgatgatgacgagattcaggaaatttctacccctgccccagctaaaaaagctaaaattgatgatgtcaaggcgacaagcgttccagaagaggacaacaatgagcaggcgcagaagagattgctcgacaagctggaagagtatgtgaaggagcagaaggatcaaccatccagcaaaagccgaaaagttctggacactcttctcggagcaatcaatgcgcaagttcaaaaggagcctctgtcggttcggaagctgatcctggacaaagttctcgttctcccaaacacaatatcattcccaccaagtcaagtttgcgacttattgattgagcacgatcccgaaatgcctttgacgaaggttatcaacaggatgtttggagaagaaagaccaaagttgagtgattccgagaaacgagagagagctcagctgaaacaacataatcctgttccaaatatgacaaaactgctcgtggacattggacaggatctcgttcaagaagctacctattgtgatatagttcacgcgaagaatcttccagaggtgccaaaaaatcttgaaacctataagcaagtcgctgcgcagttgaaaccagtttgggagacattgaaacgcaaaaatgagccgtacaagttgaaaatgcatcgatgcgacgtctgtggattccagacggaatcaaagctggttatgagcactcacaaggagaatttgcacttcacaggatccaaattccagtgcaccatgtgtaaagagacggacacgagtgagcaaagaatgaaggatcactacttgtaagttttttttttttcatctttcaatattcatttaattacagcgaaactcatcttgttattgcaaaatcggaagagaaggagtccaagtatccatgtgcaatctgcgaagaagacttcaatttcaaaggtgtccgtgagcagcattacaagcagtgcaagaaggactacattcgcattcgaaacatcatgatgccgaagcaagacgatcatctctatatcaacagatggctctgggagaggccccaattggatcccagcattcttcaacagcagcaacaagctgctcttcagcaagctcaacaaaagaagcaacagcaacttctgcatcaacagcaagcagcacaagctgcagccgctgcgcaactcttacggaagcaacaattacaacagcaacaacaacagcaacaggctcgtcttcgtgagcaacagcaagcggcccaattccggcaagtggctcaactgctgcaacaacaatcagcgcaggctcaacgtgcacagcagaatcaaggaaatgtgaatcataacactctgattgcaggtaatagctaaacatattttaaataagtattttgtataattatttatatttcagcaatgcaagcgtcgttgcgtagaggtggtcaacaaggaaattcgctggcagtttctcaacttctccaaaagcaaatggcagctttgaagtcgcaacaaggagctcaacaacttcaggctgcggtgaactccatgagaagccagaacagtcaaaagacgccaacacacagaagttcgaaacttgttactacgccgtctcatgctactgttggctcttcttcagctcccacgtttgtatgcgaaatttgtgatgcgtcagtgcaggaaaaggagaagtatctacagcatcttcaggtaattttaagaaacgtttctatttcaatttcaaaaccgattattaaatatcttaaacatcacattttcagactactcataagcagatggttggaaaagtgctgcaggacatgtcgcaaggagctccactggcatgttctcgatgccgtgacagattctggacttatgaagggttggagcggcacttggtgatgtcgcatggtctcgtcactgctgatctgctcctcaaagcgcaaaagaaggaagacggaggtcgatgcaagacatgcggcaagaactatgcgttcaacatgcttcaacacttggtagctgatcatcaagtgaagttgtgctcggctgaaatcatgtactcgtgcgatgtgtgcgcgttcaaatgctcgagttatcagactctggaagcccatctcacttcaaatcacccaaaaggagataagaagacatcaacaccagcaaaaaaagatgattgtattactctggatgattaataggaaaacgaatggcttatcccgttctacgaatgagtgctggaaacattcttcacaatgatctcaattatttctcttattctttacattcaatcattttaaatcaccagttctcccactttcattgatatacacattctattgcgggttccggaaccgaaatcaatcagtactttactttatttccccaatttttctcttcatgatatctggtttattctcgcatcttcccctaccttcaaaactccctatttttttttcaaaacctaactaccccacaattatcatgtaaaatcaaattgcaattccccataagacagatcagtatacactttcacttcatacgtctgttgttctcccccatctcatactttttttaccatttgtccagttaagatttttggaagatatctat

SEQ ID NO: 3 - DNA, 2562 bp, Caenorhabditis elegans
atggtcaccgccgacgagacggtactcgccacaacgaccaacaccacttccatgtctgtggaaccaacggatccgagaagcgctggtgaatcgtcctcagattcggagccagacacaattgagcagctgaaggcagaacagcgcgaagtgatggccgacgcggcgaatggttccgaagtcaacggaaatcaagagaacggaaaagaggaagcggcatctgcagacgtggaagtgatcgagatagatgacaccgaagagtctacggatccctcacctgatggatctgatgaaaacggtgatgctgcatctacatcggttccaatcgaagaggaagcgcgtaaaaaggatgagggggcttccgaagtgactgtggcatcatctgagattgaacaagacgatgatggcgatgttatggaaatcactgaggagccgaacggaaagtcggaggatactgccaacggaacagttactgaggaggtgctagatgaagaggagccagaaccttccgtaaacggaacaactgagatcgctacagagaaagagccagaagattcttcaatgcctgtcgaacagaatgggaagggtgtgaagcggcctgtcgaatgcatcgaactcgacgacgacgatgatgacgagattcaggaaatttctacccctgccccagctaaaaaagctaaaattgatgatgtcaaggcgacaagcgttccagaagaggacaacaatgagcaggcgcagaagagattgctcgacaagctggaagagtatgtgaaggagcagaaggatcaaccatccagcaaaagccgaaaagttctggacactcttctcggagcaatcaatgcgcaagttcaaaaggagcctctgtcggttcggaagctgatcctggacaaagttctcgttctcccaaacacaatatcattcccaccaagtcaagtttgcgacttattgattgagcacgatcccgaaatgcctttgacgaaggttatcaacaggatgtttggagaagaaagaccaaagttgagtgattccgagaaacgagagagagctcagctgaaacaacataatcctgttccaaatatgacaaaactgctcgtggacattggacaggatctcgttcaagaagctacctattgtgatatagttcacgcgaagaatcttccagaggtgccaaaaaatcttgaaacctataagcaagtcgctgcgcagttgaaaccagtttgggagacattgaaacgcaaaaatgagccgtacaagttgaaaatgcatcgatgcgacgtctgtggattccagacggaatcaaagctggttatgagcactcacaaggagaatttgcacttcacaggatccaaattccagtgcaccatgtgtaaagagacggacacgagtgagcaaagaatgaaggatcactacttcgaaactcatcttgttattgcaaaatcggaagagaaggagtccaagtatccatgtgcaatctgcgaagaagacttcaatttcaaaggtgtccgtgagcagcattacaagcagtgcaagaaggactacattcgcattcgaaacatcatgatgccgaagcaagacgatcatctctatatcaacagatggctctgggagaggccccaattggatcccagcattcttcaacagcagcaacaagctgctcttcagcaagctcaacaaaagaagcaacagcaacttctgcatcaacagcaagcagcacaagctgcagccgctgcgcaactcttacggaagcaacaattacaacagcaacaacaacagcaacaggctcgtcttcgtgagcaacagcaagcggcccaattccggcaagtggctcaactgctgcaacaacaatcagcgcaggctcaacgtgcacagcagaatcaaggaaatgtgaatcataacactctgattgcagcaatgcaagcgtcgttgcgtagaggtggtcaacaaggaaattcgctggcagtttctcaacttctccaaaagcaaatggcagctttgaagtcgcaacaaggagctcaacaacttcaggctgcggtgaactccatgagaagccagaacagtcaaaagacgccaacacacagaactcccacgtttgtatgcgaaatttgtgatgcgtcagtgcaggaaaaggagaagtatctacagcatcttcagactactcataagcagatggttggaaaagtgctgcaggacatgtcgcaaggagctccactggcatgttctcgatgccgtgacagattctggacttatgaagggttggagcggcacttggtgatgtcgcatggtctcgtcactgctgatctgctcctcaaagcgcaaaagaaggaagacggaggtcgatgcaagacatgcggcaagaactatgcgttcaacatgcttcaacacttggtagctgatcatcaagtgaagttgtgctcggctgaaatcatgtactcgtgcgatgtgtgcgcgttcaaatgctcgagttatcagactctggaagcccatctcacttcaaatcacccaaaaggagataagaagacatcaacaccagcaaaaaaagatgattgtattactctggatgattaa

SEQ ID NO: 4 - DNA, 10 bp, Caenorhabditis elegans
agtttcagac

SEQ ID NO: 5 - DNA, 10 bp, Caenorhabditis elegans
agtttcagac

SEQ ID NO: 6 - DNA, 13 bp, Caenorhabditis elegans
agaatcttcagtc

SEQ ID NO: 7 - DNA, 13 bp, Caenorhabditis elegans
agaatcttcagcc

SEQ ID NO: 8 - DNA, 13 bp, Caenorhabditis elegans
agaactttaagat

SEQ ID NO: 9 - DNA, 13 bp, Caenorhabditis elegans
agaactttaagat

SEQ ID NO: 10 - DNA, 10 bp, Caenorhabditis elegans
agttgcagaa

SEQ ID NO: 11 - DNA, 10 bp, Caenorhabditis elegans
agttgcagaa

SEQ ID NO: 12 - DNA, 16061 bp, Caenorhabditis elegans
gaggaagatgtagacgacgattcggtttccgtactctcatgacttttggcgaaaatcctcacgaattctttttccgtcatacgttgagttaaaaatctggcgatgtaacgaagaatgagaagagcgtttgatgtttgccataagtagattttactgaaataagaaaaagctttaattaaatataatgatgatttttttttccaactcacttttcgcattgttctgatgtttttagttctgtggctctgcgaaggaaaagtcgaataaatgcagcgaaatttcctgttgtttgtgtattgtacattagacattgaagatgatcatctaaagcagattccaaagcgattcgggtgtctctaaacgattataacatttttaaagcttttgcctaattttaatccttactcgtcgtcatcatcaaacttgagactgaaagagagaagtttgttccaaaatgggtcataatcgtcgacaggttccaaaccgctgagtttcttcagataaatattctcctgtaagaccgtttccttggttataactgatcccatgtgtctgaaatttgttattacactgttaataatcataaaaataaaagaaaaagtcaagaaagggtcaaatattaatcaggtcacatcttttttattcaataaaatctcctctctcgttcgtggcaatgcacgtgaaatgcgccaacaaccgcgagtgcgccaacacacacacatacgcgtcagcagacaattcgctctcgtttgaaatttagttgtttctttgtttctgctgaaataatgtcagttttccgataatttcagcgttttctgactgatttttcttgttgcattcacttcctaatagttcattctactccattcttcattttataatctgtttccttcgcaatttagtgaattaaacacgtaaatcttgtttcagataaattattcaaatagttgcacaaagctcaatagtttagaagtatcttcagtgctggtcactaatacaaaatggatccggctatggcttctccaggctatcggtctgtgcagtccgatcggagtaatcacctaacagagctggaaacgagaattcaaaatcttgccgataattcacaaagagatgatgtcaaattgaaaatgttacaagttagtttcaataattcgtgttaagtaatcaatttgttcggttgcaggagatttggagcacaatcgaaaatcatttcacactaagttcgcacgagaaagtcgtggagaggctcattctctcgttcctacaagttttctgcaacacaagtccacagttcattgctgaaaacaatacacaacagcttcgaaagttaatgcttgaaatcattcttcgactttcgaacgtagaagccatgaaacatcatagcaaagaaattatcaagcagatgatgaggctaatcaccgtggaaaatgaggagaatgccaatttggctatcaaaattgtcaccgatcaagggagaagtaccggcaaaatgcaatattgcggagaggtttcacagataatggtctccttcaaaacaatggtcattgatctgacggcgagtggtcgagctggtgatatgttcaacataaaagagcataaagctccaccgtcaactagctccgacgagcaagtcatcactgaatatttgaagacttgctactatcaacaaacggttcttctcaacggaacggaaggaaaaccgccattaaaatacaatatgattccatcagctcatcagtcaacgaaggtgctcctggaggttccgtatctcgtgattttcttctatcaacatttcaaaacagcgatccaaaccgaagcgcttgatttcatgaggcttggtcttgattttctaaatgtcagagttccagacgaggataaactcaaaacaaatcaaataataaccgatgattttgtcagtgcacagtcccgattcctgtcattcgtcaacattatggctaagattccagcggtaagtttcgttttttcaagttttttttctgtaatcctgatttttatttttcagtttatggatcttatcatgcaaaatggaccgcttctagtgtcgggaacaatgcagatgctcgagcggtgcccggctgatctgataagtgtccgacgagaagttctgatggctttgaagtatttcacatctggagaaatgaagtcgaaattctttccaatgctacctcgactcatcgctgaggaggttgttctgggaacaggattcactgcgattgagcatttgcgagttttcatgtatcaaatgctagcagatctgttgcatcacatgcgaaattctatagactatgaaatgatcacacagtaagtttgaataagactttctgatgaaaaatgttgaaatttcagcgtgattttcgtattctgtcgcactcttcacgatcctaacaactcttctcaagtccagattatgtctgctcggctgctcaactcactggccgaatctctgtgcaaaatggattcacatgataccgtaagacttattctatcaataatcgtatctcacttcgaaataagtttcagactcgtgatctgctcattgaaatcctggagtcgcacgtggccaagctcaaaactcttgcagtctatcacatgcctattctcttccaacaatacggaaccgaaatagactacgaatacaaaagttatgagagagacgccgagaaacctggaatgaatatcccaaaggacactatacgaggagtaccgaaacgaagaatccgtcggctctccattgattcagttgaagagctggaattcctggcatcagaaccatccacgtcggaagatgcagatgagagtggtggagatccgaacaagcttcctccgccaacaaaagagggaaagaaaacgtctcccgaagcgattttaaccgccatgtcaacgatgacacctcctccattggcaattgttgaagctcgaaatcttgtgaagtatataatgcatacgtgtaaattcgtgacaggacaattgagaatcgcccggccatcacaggatatgtatcattgttcgaaggagcgagatttattcgaacgtcttctacgatatggtgtaatgtgtatggatgtattcgtgcttccaacaactcgaaatcaaccacaaatgcattcttcaatgcggacaaaagatgagaaagatgctctggagtcgttggcaaacgtttttacaacaatcgaccatgcgatattccgggaaatcttcgaaaagtatatggatttcttgattgaaagaatttacaatcggaactatccattgcaattgatggtgaacaccttcttggttcgaaatgaagtgccattcttcgcatctacgatgctttcattcttgatgtctcgaatgaaattgctggaagttagcaatgacaagacgatgctatatgtgaagctcttcaaaattatcttctccgccatcggagccaatggctctgggcttcatggagataaaatgctcacttcatacctcccagagattctcaaacagtcaactgtcttggcattaacagctcgtgaacctctcaactatttccttttgcttcgtgcattgttccgcagtattggtggtggcgctcaggatattttgtatggaaagttcctgcagttactgccaaatcttcttcaattcttgaataaattgacggtgagtttcattttttgatatatcggtaatacactaaaaatccagaatcttcagtcatgtcaacatcggattcaaatgcgtgagctcttcgtcgagttgtgtttgactgtgccagttcgactcagttcccttctgccatacctaccgcttctgatggatccactggtgtgtgcgatgaatgggagtccgaacatagttacacaaggattgagaacattggaattatgtgtggataacttgcaacctgaatatcttctcgaaaatatgcttcctgtccgtggagctttgatgcaaggcctctggcgtgttgtatcgaaagctccagatacatcatcgatgacagcagcgttcaggatcctcggaaagttcggaggagccaatcgaaaacttctgaatcaaccgcaaattcttcaagtagccactttaggcgacgtaagtttatttagtttattctcttcctcgttttaagttctaacattgatcctattaacagactgttcagtcgtacatcaatatggaattctcgcggatgggactcgatggcaatcacagcattcacctgccactgtccgagttgatgagagtcgttgccgatcagatgagatatccagctgatatgatccttaatccaagtcctgcaatgatcccgtcaactcatatgaagaaatggtgtatggaattgtcgaaagccgtcttgttagccggacttggatcttcaggaagcccaattactccaagtgcaaatcttccgaagattatcaagaaacttcttgaagattttgatccaaacaatcgtaccactgaagtatacacatgtccgagggaaagtgatcgagagctttttgtgaatgcacttctcgcaatggcttgtaagttcttaagttcttttctctctaatcagatctatattttaaatttttcagacggaatatggaataaagacggtttccggcatgtctatagcaaattctttatcaaagttctccgccagtttgcgttgattggagtactcgaatacattggtggaaatggatggatgcgtcatgcagaagaggaaggtgttctaccattgtgccttgactcgtctgttatggttgatgctctgattatttgtctctctgaaacatcgtcaagcttcatcattgctggtgtcatgtctcttcgtcatatcaatgagactctctcgcttacacttcccgatattgatcaaatgtcgaaagttccaatgtgcaaatacttgatggagaaggtgttcaaattgtgtcacgggcctgcttggtatgcaagatctggtggaatcaatgcaattggatacatgatcgaatcgtttccacgaaaatttgttatggactttgtgatagatgttgttgattcgatcatggaagttattttgggaactgttgaagaaatatcaagtggatctgctgattctgcatacgattgtctcaagaaaatgatgcgagtctatttcatcaaagaagaaggccaagaagaggagaatctgacactcgcgactatttttgtgtctgcaatctctaagcattacttccacagtaatgaaagagtcagagaatttgcgattggtttaatggatcattgtatggttcactcaagacttgcaccatcccttgataagttctactatcgattcaaggagttctttgagccagaattaatgcgggtgctcacaacagttccaacaatgtcattggcagacgcaggaggaagtttggatggagttcaaaactatatgttcaactgtccggatggttttgatttcgaaaaagatatggacatgtacaagcgatatttgtcacatctgctggatattgcacaaaccgatacatttaccttaaaccaaaggaatgccttcaaaaaatgcgagacatgcccatcgcatttccttcctccattcccaatcactacacatattgattcaatgcgagccagtgctctacagtgtcttgtgatcgcgtatgatcgaatgaagaagcaatacatcgacaagggaatagagctgggtgatgagcataagatgatagagatcctcgcacttcgcagctccaagatcacagttgatcaagtctacgagagcgatgaatcttggagacgattgatgacagttctattgagagcagtcactgacagagaaactcctgaaattgcggagaagcttcatccttcacttttgaaggtctcaccaatatccacaatcatcatcgcaacatttggtgcttcttacataagaaatattagtggagcaggagatgacagtgattcagatcgtcatatttcgtacaacgatataatgaagttcaagtgtctcgtggagctcaatccaaagattctggtcacaaaaatggcagtgaatctcgcaaatcaaatggttaaatataagatgagtgacaagatctctaggattttgtcagttcccagtagcttcactgaagaggagctcgatgatttcgaagcggagaagatgaaaggaattcgagagttggatatgattggtcatacggttaaaatgcttgctggatgcccagtgaccacattcacggagcaaattattgtggatatcagtcgttttgctgctcattttgagtatgcttattcgcaagatgtacttgtaaattggattgatgatgtcacagtaatcctcaacaaaagtcccaaagatgtatggaagttcttcttgtctcgagaatcaattctagatcctgcacgcagatcctttattcgaagaatcatagtctatcaatcaagtggtccactgcgacaggaattcatggatactccggaatattttgagaaactcattgatcttgacgatgaggagaataaggatgaagatgagagaaaaatctgggatcgtgatatgtttgcattttcgattgtcgatcgtatctcgaagagctgccctgagtggcttatttctccgaattccccaattccaagaattaagaagttgttctccgaaacggaattcaatgagcgatatgtggttcgagcattgactgaggtgaagaaatttcaagaagagatcatagtgaaacggatgacagagcacaagtacaaggttccgaagctgattctgaataccttcctgagatatttgaggtaatttcaagatagtttgtaaaaattaattacaaagaaatataccaaaactgaaccccaaaaaaaaatttttgaatttcggatcaaaaaaatttaatattttctcgaaaaatccttcaaaataccaaaaaattcgaattctcacttctaaaattatttttgaatttttaaataatttttgaacatttctctatgaaattcatgttttgggcctatttcaggctataaaaattatttttctgattttaaataacttgcaaatttcaggctcaacatctatgactacgatctattcatcgttatcgcctcgtgtttcaatggcaatttcgtcaccgatctctcttttcttcgcgaatatcttgaaactgaagtcatcccgaaagtgccgttacaatggcggagagagctgtttcttcgaattatgcagaagtttgatacggatccacaaactgctggaacaagtatgcagcatgtgaaggcccttcaatatttggttattcccacgttgcattgggcgttcgagcgatatgatacggatgaaattgttggcaccgcaccaatagatgattcggattcttcgatggatgtagatccggcaggcagctcggataaccttgtggctcgtttaacatcagtcattgattctcatcgtaattatctgagcgatggaatggtcattgttttctatcaactttgcacattgttcgtacaaaacgcctccgaacatattcacaataataactgcaagaaacaaggtggacgcctacggatcctgatgctcttcgcctggccgtgcctgaccatgtacaatcatcaagatccaacaatgcggtacactggattcttcttcttggccaatattatagagcgtttcacaattaatcggaaaatcgtgcttcaagtgttccatcaacttatgactacttatcagcaggacactagagatcaaatccggaaagccattgatatattaactccagctttgaggacacgaatggaagatggacacttgcaaatattgagtcatgtgaagaaaattcttatcgaagaatgccataatttgcaacatgttcagcatgttttgtaagtttattatctaaaatgattttttttaatgttaaaaatttaattttaaaatgcgttcgtgctcctttaataattcctgaattttccagccaaatggtggttcgcaattatcgtgtctactatcatgttcgattggagcttctcacgcctcttctgaacggagttcaacgagcacttgtgatgccaaatagtgttctggaaaagtaagtttccagcccgttgttcgtaaactcaccccttgtaaatatttagctggcaaactcgacgtcatgcggtggagatctgcgagatggtcatcaagtgggaattgttcagaacgctgaaaacagatcatattatcagtgacgaagaagctctcgaagttgacaagcaattggataagctgcgaacagcttcatccacagatcgtttcgatttcgaggaggctcataacaagagagacatgcctgatgctcaacgcacgattatcaaagagcacgccgatgtgattgtcaatatgcttgtccgattctgtatgacgttccatcagaattcgggttcttcgtccacttctcaaagtgggaaccatggtgtcgagttgaccaaaaaatgtcagctgcttctacgtgcagccctacgaccaagcatgtggggagaatttgtcagcttccgattaacaatgatcgaaaagtttttgtcaattccgaatgataatgctctacgcaatgatataagttctacggcctacgctaatactatccaaaatgcacaacacactctggatatgctgtgtaatattattcctgttatgccaaaaactagcttgatgactatgatgagacaactccaacggccactcatacaatgtctcaataacggagctcaggtatgtgaagaacgatgaatagggggttataaatcactaatttctcttagaactttaagatgactcgtcttgtcactcaaattgtcagtcggttactcgaaaagacaaatgtttcggttaacgggcttgatgagctggagcaattgaatcaatacatttcccgattcctacatgaacattttggatctcttttgaagtaagttttatttttgaatttccatctttcaacccttcgccagttgcagaaacttgagtggaccagtgttgggagttctcggagcattttctcttttgcgaacaatttgtggacacgagccagcatacttggatcatttgatgccttcatttgtaaaagtgatggagagagctgcaaaagagcacttggcgtatgttgcgaactcgcaagatggaaatatggtgaagagtaagttctataaaaagattcagattttctaatccccttagatttctttccagatgttgctgaattgttgtgtgcatgcatggagctggtacgtcccagagtcgatcatatcagtatggagattaagagatcaattgttggtggtattatcgcggagctgattatcaaatcgaatcacgataagatcatccagacgtcagtgaagcttctcggagcaatgattagcacgcaggatatggaatttacaattctcactgttcttccgctacttgttcgtatccaatcaattattgtgaccaagttcaagaattgcaaggatctgatagcagactatcttgttgtggttattaccgtttttgagaacagcgaatatcggaactcggaagctggatctcgtctctgggaaggattcttctggggactcaagagtagcgatcctcaaacccgggagaaattctcgatagtttgggagaagacttggccacacatggcaacagtagatattgctcatcgaatgaaatatatcatgcaaaatcaagattggtccaagttcaaacacgcgttttggttgaaattcgcactttggggaatgctacgaacgattgccaaacggccaactgatccgaataataagagaaagaaagtgatactgttgaactgtgcaactccatggagaacaattgaatatgcagcgaaattgaaggatcagccaatggaagtggaaactgaaatgaaacgagaagagccagaaccgatggaagttgacgaaaaagactcgcaagatgattctaaggatgccggagagcccaaggagaaggaaaagctcacattggaattattgcttgctggacaacaagaacttttggatgaagcttccaattatgattttgcggatgctctagatacagtatcccagattacatttgcacttaatggtaaattgttcaaagtttatgaatatttttcttaaaaatcacaattttcagagaatcaagtgacaagcaagatgtgggtagtgttgttcaaatcattctggagttccttatcacaatccgaaatcgaagatttcacggcgctagtcgttccgtttatgagcagtggagtgcataataattatcagacgggtgtacaggatagtgtgcttgctgtttggcttgaagctgttggtgacgctgttcatttgccgtccagattgattgaggtacgttctgaaaatgaatgctggaaaaaattcgatttttctgtttaaaaaaagttaaaatttccgattttttgaatagcaaaaaaaaaagaaaacatttattttgaaaaaagagtcctcaccggaattttttaataaataaatttaaaaaaagaaaaaaaactaaaaacttcaatttttgaaaatcaaaaaaaaaattacagaaacagacgaggtaaaaaattttaaaaaagttctgtaaaaaaaatggagaatcacagttttcgttgtcttttctgaaaaaaatttgaaaaattaaaaattaacgattttttggtttttaatttaaaaaaatatacgaaaaaagactgaagaactttttttgtcaaaaaaacttgattttgatgagggaaaaagttcaaaaacttggagaaatcatcggaaattttagaagattcaataaaaatttccaaaaaaaaaaattgaacatttatgatttttgggtattttgaaaaattgaaaaattacgcttaatttttagattaaaaaaatcaaaaaaaaaccaacactccttttgaaacttgacacttttgaaacgttttttttttttgcaataataaatttctcatttcagtttatctcatcaaaacacgaatgctggcataccggaatcaggcttctcgagaatcatatatggacaattccaaagcaactcaacaacacgttactccgagaaatgaaagtggcaccaggtctcgctggagatattgagacactcgaatctcttggaacactctacaatgagatatcagagtttgatcagttcgctgcaatctgggaacgccgtgctgtatttcctgatacgatgagagcaatgtcagctatgcaattgggagatatggaattagctcaatcttatctggaaaaatcaatgagcagtacgtatgaaactcttgctccgacaatcaatcgtaagtttggatcaatcggttgtacttctcacacaaaatagtattcctttcagcaaacaacacttcaaattcggagaagcatgtttctccgattattgacaaagaatacgatcattggatggagatgtacatcacaaattgctcggagcttcttcagtggcaaaatgtggccgacgtatgcaatggcaaagacatgcaacatgttcgtggcctgatcaacgcagcatctcacattccggactggaatgtggtcgaggagtgtaaaagtcagatagctggatgtattccaccaagtttccatttagattacactcttttcaatttgatgagtactgttatggttagtttaagtcaaaaagtgatatataattattgtttaatttttcagcgaatgaatgaaaactcaagcccgacacatatgaaggaacgatgcaaaattgcaattcaagagtgcacagaagctcatattagtcgttggagagcacttccgtcagttgtttcatatggtcatgtcaagattcttcaggcaatgaacttggttcgagaaattgaagagtctacagatattcgcattgctctgctcgaggccccatcaaacaaagtggatcaggcgttgatgggcgatatgaagtcgttgatgaaagtattccgaaatagaacaccaaccacttcggatgatatgggattcgtttcgacttggtatgattggaggaatcagattcatggaatgatgcttcaaagattcgaatattgggataaagtaggactcaacgtcgctgcaactggaaaccagtcaattgttccgattcattcaatggctcaagcacagttggccgtagccaaacatgccaagaatcttggattccataatttaacgaaagatctactcaacaaattagctggattgacagccataccgatgatggatgctcaagataaagtttgcacttacggcaagacacttcgcgatatggcaaacagtgcggctgacgaaagagtgaaaaatgagctattgtgtgaagcgcttgaagttttggaagatgtgcgaattgatgatctacagaaggatcaggttgctgcattgctttatcatcgtgctaatattcattcagttcttgatcagtaagttttcaatgccgaaaaaaaattaaagttttacaaaaataaatttcagagctgaaaatgctgactacaccttctccgcagcctctcaacttgtcgacttgcaaaatagtgtgacaaccactggaatcaagctcatgaaaaattggggccaccatctttacaagagattcttctctacgacagtttgcaaggaaaccggaaacaacttcggacggcaggctctcgcttgttacttcattgcggctcgtgtggataacgatatcaaggcgagaaaaccgattgccaagattttgtggctctcgaagcacttgaatgcgtgtggatcacatgaagtgatgaatcgggttattaagaagcaacttcattcacttaatctcttcaattggctttactggcttccacaattggttactgatgttcgatataaaccaaattcgaactttgttctgattctctgcaaggtaagttttgaaatatttaaatattttcagaattttaaatgaaattcatttgcagatggctgctgctcatccacttcaagtattttaccacattcgggaggcagttagcgttgacgatattgactcggttctcgaagaagattacactgatgagcaaatgtcgatggatgtttcggatgaggattgttttgcagacgatccaccatttgatagaattctgaaaatatgtctgaaatatcgtccaactgatattcgagtcttccatcgtgtcctcaaagaacttgacgagatgaatgagacatgggttgaacgtcacttgcgtcatgcgatctgcctcaaggatcagatgttcaaagatttctcggaacaaatggacgcgacgttcaatgagatgcaatattcggaggatgtgactatgatgacgttgagatggaggaaacagctggaagaagacttggtgtatttccaacagaattataatcttgatttcctggagattcgtaacaagcgaaagatgatcgtgacgaagggatgtatgggagtcgagaaaagtcagataatgttcgaaaaagagctgagtcaagtgttcacagagccggccggcatgcaagatgaatttgattttgtcacaaatatgactaatatgatggtctcacagttggatattcatgcagtcgatgctccacgccctcagggatatattcgtattgttctcgactggattcgagcgattcgtcgtcgtttcgatcgacttccacgaagaatccctctggaatcgtcaagcccatatctcgccagattcagccatcgtacaggatgcatcgaaatgccatacgatttgctcaacgttttgcgcgccaagaatcatactctgatggcttccaatcaaacggggcaatacatatccatgctctctcgatttgagccaaactttgagattgtgatcaaaggtggtcaagtgataagaaagatctatattcgaggacaaaccggaaagagtgcggcgttttatctgaagaaatctgtgcaggatgagccaactaaccgagttccacaaatgttcaaacatcttgatcacgttctacaaaccgatagagagtcggcgagaagacatcttcatgctccaacagtgctgcagatgagagtcggacagaagacgacactctacgaagttgcatccgttcaaccatatgcaatgccaccggattgtaccagaaactatccagcatcacaaatcgacattgttcatccatatgatgtgctgactgccactttcaatggaagttattatccggatgatatggtattgcacttctttgagagattcgcccaaagttcttcatccatcggacaacctcttccaactccgacgaaccaagatggaacagttgctccgccacgactaacggaagctcaccacatcaagaatattatttatgagtacgtttgagaagctagtgtctaaaataataattaatgtaaaaaaattttcagagactttgcccgagatatgatcccattccgacttctctacgactacctcactgcacgatatcctgatccggttatgtactatgcaatgaagaagcaattgctgcacagtctcgccgtcctatccacaatcgaatatcattgcaatctgacaccaatgggacctgatcaaatgatgatgacaatgaatactggagtccttagcaatccttcatatagattcgaaatccgaggaggacgatcacttcatgatattcaacactttggacatgaagttccattccgattgactccaaatctatcgattttggttggtgttgcacaggatggtgacttgttatggagtatggctgctgcgtcaaaatgtttgatgaagaaggaacctgaagttatcatgagaccgttagtatgggatgaattcgccaacaatacagattgcgacaaatcggtaattttactttaatatgctaatagggaattgaactaatgttttccaagcgtttgcaggtattcgcgtgtcatgcatcgaattcttacatcaatggtgtcgcgagcaagcttcgaaacacgaatagcgccgacgccaaactcagaaaggacgattgtgtgtcgctgatcagtcgagccaaggattcggataatctggcccgaatgccacccacctaccacgcgtggttctagatctcataattaccgttctctattttgatcccgcctcccactctcacagatctctatacatttgtcaaatgtttccaaatcttttatctgcccatacattcgtttttattgttttgtttcttttctttctttatttcttttctaaactttaagatttatgtaaatatttaactgcgctggtatttatgaaaaattcagataaagttttcaagtttaaaaaatcgaaaattcgaagtcggaagttctcttacaggtgtagtaagtaggcacaatggcaataggtacatggaaggcttgcggaaggcacatgggtaggcataagatcgaaaaataagctgatatataaatatagataggtattggttaggcacaaattaggcacgtaggtgtgagctggcaaataggtaggcatgacgttcggcaaatcggcaaattgccgatttggcgaaaattttcaaatccggcgatttgccggaaatgtttagagaaattttttataagacagaaaaacttacaactgtgtctttttgaaattcttccggttttctttatacagtgcgtgcaacttctatagcgccccccccccccccccccccccctattttttcgcgtttcacgccattctgatttttatttttctgatttttttttttttgcactgaaacttggcattgaggatgcttggagagaaatatcagccagcaaaataaagaatctggtcaactcaatgtcgaatagattttttgaggttatcgttaagaagggaggtcccacgacgtattgatccttcatcgagttaacaaattatgatgttttaattgatttcattccacttctggacacagaaggacgaatagtgcaatctggtacaagtttatcaccacctacaacttcgtcgatttgtggaaaatctttcagacatgtctccatgagtgtctcagaacatcttggtcaggtttggagtcgatcccaccgctgggagccgagaatgggcctctaacac

SEQ ID NO: 13 - DNA, 12195 bp, Caenorhabditis elegans
atggatccggctatggcttctccaggctatcggtctgtgcagtccgatcggagtaatcacctaacagagctggaaacgagaattcaaaatcttgccgataattcacaaagagatgatgtcaaattgaaaatgttacaagagatttggagcacaatcgaaaatcatttcacactaagttcgcacgagaaagtcgtggagaggctcattctctcgttcctacaagttttctgcaacacaagtccacagttcattgctgaaaacaatacacaacagcttcgaaagttaatgcttgaaatcattcttcgactttcgaacgtagaagccatgaaacatcatagcaaagaaattatcaagcagatgatgaggctaatcaccgtggaaaatgaggagaatgccaatttggctatcaaaattgtcaccgatcaagggagaagtaccggcaaaatgcaatattgcggagaggtttcacagataatggtctccttcaaaacaatggtcattgatctgacggcgagtggtcgagctggtgatatgttcaacataaaagagcataaagctccaccgtcaactagctccgacgagcaagtcatcactgaatatttgaagacttgctactatcaacaaacggttcttctcaacggaacggaaggaaaaccgccattaaaatacaatatgattccatcagctcatcagtcaacgaaggtgctcctggaggttccgtatctcgtgattttcttctatcaacatttcaaaacagcgatccaaaccgaagcgcttgatttcatgaggcttggtcttgattttctaaatgtcagagttccagacgaggataaactcaaaacaaatcaaataataaccgatgattttgtcagtgcacagtcccgattcctgtcattcgtcaacattatggctaagattccagcgtttatggatcttatcatgcaaaatggaccgcttctagtgtcgggaacaatgcagatgctcgagcggtgcccggctgatctgataagtgtccgacgagaagttctgatggctttgaagtatttcacatctggagaaatgaagtcgaaattctttccaatgctacctcgactcatcgctgaggaggttgttctgggaacaggattcactgcgattgagcatttgcgagttttcatgtatcaaatgctagcagatctgttgcatcacatgcgaaattctatagactatgaaatgatcacacacgtgattttcgtattctgtcgcactcttcacgatcctaacaactcttctcaagtccagattatgtctgctcggctgctcaactcactggccgaatctctgtgcaaaatggattcacatgatacctttcagactcgtgatctgctcattgaaatcctggagtcgcacgtggccaagctcaaaactcttgcagtctatcacatgcctattctcttccaacaatacggaaccgaaatagactacgaatacaaaagttatgagagagacgccgagaaacctggaatgaatatcccaaaggacactatacgaggagtaccgaaacgaagaatccgtcggctctccattgattcagttgaagagctggaattcctggcatcagaaccatccacgtcggaagatgcagatgagagtggtggagatccgaacaagcttcctccgccaacaaaagagggaaagaaaacgtctcccgaagcgattttaaccgccatgtcaacgatgacacctcctccattggcaattgttgaagctcgaaatcttgtgaagtatataatgcatacgtgtaaattcgtgacaggacaattgagaatcgcccggccatcacaggatatgtatcattgttcgaaggagcgagatttattcgaacgtcttctacgatatggtgtaatgtgtatggatgtattcgtgcttccaacaactcgaaatcaaccacaaatgcattcttcaatgcggacaaaagatgagaaagatgctctggagtcgttggcaaacgtttttacaacaatcgaccatgcgatattccgggaaatcttcgaaaagtatatggatttcttgattgaaagaatttacaatcggaactatccattgcaattgatggtgaacaccttcttggttcgaaatgaagtgccattcttcgcatctacgatgctttcattcttgatgtctcgaatgaaattgctggaagttagcaatgacaagacgatgctatatgtgaagctcttcaaaattatcttctccgccatcggagccaatggctctgggcttcatggagataaaatgctcacttcatacctcccagagattctcaaacagtcaactgtcttggcattaacagctcgtgaacctctcaactatttccttttgcttcgtgcattgttccgcagtattggtggtggcgctcaggatattttgtatggaaagttcctgcagttactgccaaatcttcttcaattcttgaataaattgacgaatcttcagtcatgtcaacatcggattcaaatgcgtgagctcttcgtcgagttgtgtttgactgtgccagttcgactcagttcccttctgccatacctaccgcttctgatggatccactggtgtgtgcgatgaatgggagtccgaacatagttacacaaggattgagaacattggaattatgtgtggataacttgcaacctgaatatcttctcgaaaatatgcttcctgtccgtggagctttgatgcaaggcctctggcgtgttgtatcgaaagctccagatacatcatcgatgacagcagcgttcaggatcctcggaaagttcggaggagccaatcgaaaacttctgaatcaaccgcaaattcttcaagtagccactttaggcgacactgttcagtcgtacatcaatatggaattctcgcggatgggactcgatggcaatcacagcattcacctgccactgtccgagttgatgagagtcgttgccgatcagatgagatatccagctgatatgatccttaatccaagtcctgcaatgatcccgtcaactcatatgaagaaatggtgtatggaattgtcgaaagccgtcttgttagccggacttggatcttcaggaagcccaattactccaagtgcaaatcttccgaagattatcaagaaacttcttgaagattttgatccaaacaatcgtaccactgaagtatacacatgtccgagggaaagtgatcgagagctttttgtgaatgcacttctcgcaatggcttacggaatatggaataaagacggtttccggcatgtctatagcaaattctttatcaaagttctccgccagtttgcgttgattggagtactcgaatacattggtggaaatggatggatgcgtcatgcagaagaggaaggtgttctaccattgtgccttgactcgtctgttatggttgatgctctgattatttgtctctctgaaacatcgtcaagcttcatcattgctggtgtcatgtctcttcgtcatatcaatgagactctctcgcttacacttcccgatattgatcaaatgtcgaaagttccaatgtgcaaatacttgatggagaaggtgttcaaattgtgtcacgggcctgcttggtatgcaagatctggtggaatcaatgcaattggatacatgatcgaatcgtttccacgaaaatttgttatggactttgtgatagatgttgttgattcgatcatggaagttattttgggaactgttgaagaaatatcaagtggatctgctgattctgcatacgattgtctcaagaaaatgatgcgagtctatttcatcaaagaagaaggccaagaagaggagaatctgacactcgcgactatttttgtgtctgcaatctctaagcattacttccacagtaatgaaagagtcagagaatttgcgattggtttaatggatcattgtatggttcactcaagacttgcaccatcccttgataagttctactatcgattcaaggagttctttgagccagaattaatgcgggtgctcacaacagttccaacaatgtcattggcagacgcaggaggaagtttggatggagttcaaaactatatgttcaactgtccggatggttttgatttcgaaaaagatatggacatgtacaagcgatatttgtcacatctgctggatattgcacaaaccgatacatttaccttaaaccaaaggaatgccttcaaaaaatgcgagacatgcccatcgcatttccttcctccattcccaatcactacacatattgattcaatgcgagccagtgctctacagtgtcttgtgatcgcgtatgatcgaatgaagaagcaatacatcgacaagggaatagagctgggtgatgagcataagatgatagagatcctcgcacttcgcagctccaagatcacagttgatcaagtctacgagagcgatgaatcttggagacgattgatgacagttctattgagagcagtcactgacagagaaactcctgaaattgcggagaagcttcatccttcacttttgaaggtctcaccaatatccacaatcatcatcgcaacatttggtgcttcttacataagaaatattagtggagcaggagatgacagtgattcagatcgtcatatttcgtacaacgatataatgaagttcaagtgtctcgtggagctcaatccaaagattctggtcacaaaaatggcagtgaatctcgcaaatcaaatggttaaatataagatgagtgacaagatctctaggattttgtcagttcccagtagcttcactgaagaggagctcgatgatttcgaagcggagaagatgaaaggaattcgagagttggatatgattggtcatacggttaaaatgcttgctggatgcccagtgaccacattcacggagcaaattattgtggatatcagtcgttttgctgctcattttgagtatgcttattcgcaagatgtacttgtaaattggattgatgatgtcacagtaatcctcaacaaaagtcccaaagatgtatggaagttcttcttgtctcgagaatcaattctagatcctgcacgcagatcctttattcgaagaatcatagtctatcaatcaagtggtccactgcgacaggaattcatggatactccggaatattttgagaaactcattgatcttgacgatgaggagaataaggatgaagatgagagaaaaatctgggatcgtgatatgtttgcattttcgattgtcgatcgtatctcgaagagctgccctgagtggcttatttctccgaattccccaattccaagaattaagaagttgttctccgaaacggaattcaatgagcgatatgtggttcgagcattgactgaggtgaagaaatttcaagaagagatcatagtgaaacggatgacagagcacaagtacaaggttccgaagctgattctgaataccttcctgagatatttgaggctcaacatctatgactacgatctattcatcgttatcgcctcgtgtttcaatggcaatttcgtcaccgatctctcttttcttcgcgaatatcttgaaactgaagtcatcccgaaagtgccgttacaatggcggagagagctgtttcttcgaattatgcagaagtttgatacggatccacaaactgctggaacaagtatgcagcatgtgaaggcccttcaatatttggttattcccacgttgcattgggcgttcgagcgatatgatacggatgaaattgttggcaccgcaccaatagatgattcggattcttcgatggatgtagatccggcaggcagctcggataaccttgtggctcgtttaacatcagtcattgattctcatcgtaattatctgagcgatggaatggtcattgttttctatcaactttgcacattgttcgtacaaaacgcctccgaacatattcacaataataactgcaagaaacaaggtggacgcctacggatcctgatgctcttcgcctggccgtgcctgaccatgtacaatcatcaagatccaacaatgcggtacactggattcttcttcttggccaatattatagagcgtttcacaattaatcggaaaatcgtgcttcaagtgttccatcaacttatgactacttatcagcaggacactagagatcaaatccggaaagccattgatatattaactccagctttgaggacacgaatggaagatggacacttgcaaatattgagtcatgtgaagaaaattcttatcgaagaatgccataatttgcaacatgttcagcatgttttccaaatggtggttcgcaattatcgtgtctactatcatgttcgattggagcttctcacgcctcttctgaacggagttcaacgagcacttgtgatgccaaatagtgttctggaaaaatttagctggcaaactcgacgtcatgcggtggagatctgcgagatggtcatcaagtgggaattgttcagaacgctgaaaacagatcatattatcagtgacgaagaagctctcgaagttgacaagcaattggataagctgcgaacagcttcatccacagatcgtttcgatttcgaggaggctcataacaagagagacatgcctgatgctcaacgcacgattatcaaagagcacgccgatgtgattgtcaatatgcttgtccgattctgtatgacgttccatcagaattcgggttcttcgtccacttctcaaagtgggaaccatggtgtcgagttgaccaaaaaatgtcagctgcttctacgtgcagccctacgaccaagcatgtggggagaatttgtcagcttccgattaacaatgatcgaaaagtttttgtcaattccgaatgataatgctctacgcaatgatataagttctacggcctacgctaatactatccaaaatgcacaacacactctggatatgctgtgtaatattattcctgttatgccaaaaactagcttgatgactatgatgagacaactccaacggccactcatacaatgtctcaataacggagctcagaactttaagatgactcgtcttgtcactcaaattgtcagtcggttactcgaaaagacaaatgtttcggttaacgggcttgatgagctggagcaattgaatcaatacatttcccgattcctacatgaacattttggatctcttttgaattgcagaaacttgagtggaccagtgttgggagttctcggagcattttctcttttgcgaacaatttgtggacacgagccagcatacttggatcatttgatgccttcatttgtaaaagtgatggagagagctgcaaaagagcacttggcgtatgttgcgaactcgcaagatggaaatatggtgaagaatttctttccagatgttgctgaattgttgtgtgcatgcatggagctggtacgtcccagagtcgatcatatcagtatggagattaagagatcaattgttggtggtattatcgcggagctgattatcaaatcgaatcacgataagatcatccagacgtcagtgaagcttctcggagcaatgattagcacgcaggatatggaatttacaattctcactgttcttccgctacttgttcgtatccaatcaattattgtgaccaagttcaagaattgcaaggatctgatagcagactatcttgttgtggttattaccgtttttgagaacagcgaatatcggaactcggaagctggatctcgtctctgggaaggattcttctggggactcaagagtagcgatcctcaaacccgggagaaattctcgatagtttgggagaagacttggccacacatggcaacagtagatattgctcatcgaatgaaatatatcatgcaaaatcaagattggtccaagttcaaacacgcgttttggttgaaattcgcactttggggaatgctacgaacgattgccaaacggccaactgatccgaataataagagaaagaaagtgatactgttgaactgtgcaactccatggagaacaattgaatatgcagcgaaattgaaggatcagccaatggaagtggaaactgaaatgaaacgagaagagccagaaccgatggaagttgacgaaaaagactcgcaagatgattctaaggatgccggagagcccaaggagaaggaaaagctcacattggaattattgcttgctggacaacaagaacttttggatgaagcttccaattatgattttgcggatgctctagatacagtatcccagattacatttgcacttaatgagaatcaagtgacaagcaagatgtgggtagtgttgttcaaatcattctggagttccttatcacaatccgaaatcgaagatttcacggcgctagtcgttccgtttatgagcagtggagtgcataataattatcagacgggtgtacaggatagtgtgcttgctgtttggcttgaagctgttggtgacgctgttcatttgccgtccagattgattgagtttatctcatcaaaacacgaatgctggcataccggaatcaggcttctcgagaatcatatatggacaattccaaagcaactcaacaacacgttactccgagaaatgaaagtggcaccaggtctcgctggagatattgagacactcgaatctcttggaacactctacaatgagatatcagagtttgatcagttcgctgcaatctgggaacgccgtgctgtatttcctgatacgatgagagcaatgtcagctatgcaattgggagatatggaattagctcaatcttatctggaaaaatcaatgagcagtacgtatgaaactcttgctccgacaatcaatccaaacaacacttcaaattcggagaagcatgtttctccgattattgacaaagaatacgatcattggatggagatgtacatcacaaattgctcggagcttcttcagtggcaaaatgtggccgacgtatgcaatggcaaagacatgcaacatgttcgtggcctgatcaacgcagcatctcacattccggactggaatgtggtcgaggagtgtaaaagtcagatagctggatgtattccaccaagtttccatttagattacactcttttcaatttgatgagtactgttatgcgaatgaatgaaaactcaagcccgacacatatgaaggaacgatgcaaaattgcaattcaagagtgcacagaagctcatattagtcgttggagagcacttccgtcagttgtttcatatggtcatgtcaagattcttcaggcaatgaacttggttcgagaaattgaagagtctacagatattcgcattgctctgctcgaggccccatcaaacaaagtggatcaggcgttgatgggcgatatgaagtcgttgatgaaagtattccgaaatagaacaccaaccacttcggatgatatgggattcgtttcgacttggtatgattggaggaatcagattcatggaatgatgcttcaaagattcgaatattgggataaagtaggactcaacgtcgctgcaactggaaaccagtcaattgttccgattcattcaatggctcaagcacagttggccgtagccaaacatgccaagaatcttggattccataatttaacgaaagatctactcaacaaattagctggattgacagccataccgatgatggatgctcaagataaagtttgcacttacggcaagacacttcgcgatatggcaaacagtgcggctgacgaaagagtgaaaaatgagctattgtgtgaagcgcttgaagttttggaagatgtgcgaattgatgatctacagaaggatcaggttgctgcattgctttatcatcgtgctaatattcattcagttcttgatcaagctgaaaatgctgactacaccttctccgcagcctctcaacttgtcgacttgcaaaatagtgtgacaaccactggaatcaagctcatgaaaaattggggccaccatctttacaagagattcttctctacgacagtttgcaaggaaaccggaaacaacttcggacggcaggctctcgcttgttacttcattgcggctcgtgtggataacgatatcaaggcgagaaaaccgattgccaagattttgtggctctcgaagcacttgaatgcgtgtggatcacatgaagtgatgaatcgggttattaagaagcaacttcattcacttaatctcttcaattggctttactggcttccacaattggttactgatgttcgatataaaccaaattcgaactttgttctgattctctgcaagatggctgctgctcatccacttcaagtattttaccacattcgggaggcagttagcgttgacgatattgactcggttctcgaagaagattacactgatgagcaaatgtcgatggatgtttcggatgaggattgttttgcagacgatccaccatttgatagaattctgaaaatatgtctgaaatatcgtccaactgatattcgagtcttccatcgtgtcctcaaagaacttgacgagatgaatgagacatgggttgaacgtcacttgcgtcatgcgatctgcctcaaggatcagatgttcaaagatttctcggaacaaatggacgcgacgttcaatgagatgcaatattcggaggatgtgactatgatgacgttgagatggaggaaacagctggaagaagacttggtgtatttccaacagaattataatcttgatttcctggagattcgtaacaagcgaaagatgatcgtgacgaagggatgtatgggagtcgagaaaagtcagataatgttcgaaaaagagctgagtcaagtgttcacagagccggccggcatgcaagatgaatttgattttgtcacaaatatgactaatatgatggtctcacagttggatattcatgcagtcgatgctccacgccctcagggatatattcgtattgttctcgactggattcgagcgattcgtcgtcgtttcgatcgacttccacgaagaatccctctggaatcgtcaagcccatatctcgccagattcagccatcgtacaggatgcatcgaaatgccatacgatttgctcaacgttttgcgcgccaagaatcatactctgatggcttccaatcaaacggggcaatacatatccatgctctctcgatttgagccaaactttgagattgtgatcaaaggtggtcaagtgataagaaagatctatattcgaggacaaaccggaaagagtgcggcgttttatctgaagaaatctgtgcaggatgagccaactaaccgagttccacaaatgttcaaacatcttgatcacgttctacaaaccgatagagagtcggcgagaagacatcttcatgctccaacagtgctgcagatgagagtcggacagaagacgacactctacgaagttgcatccgttcaaccatatgcaatgccaccggattgtaccagaaactatccagcatcacaaatcgacattgttcatccatatgatgtgctgactgccactttcaatggaagttattatccggatgatatggtattgcacttctttgagagattcgcccaaagttcttcatccatcggacaacctcttccaactccgacgaaccaagatggaacagttgctccgccacgactaacggaagctcaccacatcaagaatattatttatgaagactttgcccgagatatgatcccattccgacttctctacgactacctcactgcacgatatcctgatccggttatgtactatgcaatgaagaagcaattgctgcacagtctcgccgtcctatccacaatcgaatatcattgcaatctgacaccaatgggacctgatcaaatgatgatgacaatgaatactggagtccttagcaatccttcatatagattcgaaatccgaggaggacgatcacttcatgatattcaacactttggacatgaagttccattccgattgactccaaatctatcgattttggttggtgttgcacaggatggtgacttgttatggagtatggctgctgcgtcaaaatgtttgatgaagaaggaacctgaagttatcatgagaccgttagtatgggatgaattcgccaacaatacagattgcgacaaatcgcgtttgcaggtattcgcgtgtcatgcatcgaattcttacatcaatggtgtcgcgagcaagcttcgaaacacgaatagcgccgacgccaaactcagaaaggacgattgtgtgtcgctgatcagtcgagccaaggattcggataatctggcccgaatgccacccacctaccacgcgtggttctag

SEQ ID NO: 14 - protein, 4064 aa, Caenorhabditis elegans
MDPAMASPGYRSVQSDRSNHLTELETRIQNLADNSQRDDVKLKMLQEIWSTIENHFTLSSHEKVVERLILSFLQVFCNTSPQFIAENNTQQLRKLMLEIILRLSNVEAMKHHSKEIIKQMMRLITVENEENANLAIKIVTDQGRSTGKMQYCGEVSQIMVSFKTMVIDLTASGRAGDMFNIKEHKAPPSTSSDEQVITEYLKTCYYQQTVLLNGTEGKPPLKYNMIPSAHQSTKVLLEVPYLVIFFYQHFKTAIQTEALDFMRLGLDFLNVRVPDEDKLKTNQIITDDFVSAQSRFLSFVNIMAKIPAFMDLIMQNGPLLVSGTMQMLERCPADLISVRREVLMALKYFTSGEMKSKFFPMLPRLIAEEVVLGTGFTAIEHLRVFMYQMLADLLHHMRNSIDYEMITHVIFVFCRTLHDPNNSSQVQIMSARLLNSLAESLCKMDSHDTFQTRDLLIEILESHVAKLKTLAVYHMPILFQQYGTEIDYEYKSYERDAEKPGMNIPKDTIRGVPKRRIRRLSIDSVEELEFLASEPSTSEDADESGGDPNKLPPPTKEGKKTSPEAILTAMSTMTPPPLAIVEARNLVKYIMHTCKFVTGQLRIARPSQDMYHCSKERDLFERLLRYGVMCMDVFVLPTTRNQPQMHSSMRTKDEKDALESLANVFTTIDHAIFREIFEKYMDFLIERIYNRNYPLQLMVNTFLVRNEVPFFASTMLSFLMSRMKLLEVSNDKTMLYVKLFKIIFSAIGANGSGLHGDKMLTSYLPEILKQSTVLALTAREPLNYFLLLRALFRSIGGGAQDILYGKFLQLLPNLLQFLNKLTNLQSCQHRIQMRELFVELCLTVPVRLSSLLPYLPLLMDPLVCAMNGSPNIVTQGLRTLELCVDNLQPEYLLENMLPVRGALMQGLWRVVSKAPDTSSMTAAFRILGKFGGANRKLLNQPQILQVATLGDTVQSYINMEFSRMGLDGNHSIHLPLSELMRVVADQMRYPADMILNPSPAMIPSTHMKKWCMELSKAVLLAGLGSSGSPITPSANLPKIIKKLLEDFDPNNRTTEVYTCPRESDRELFVNALLAMAYGIWNKDGFRHVYSKFFIKVLRQFALIGVLEYIGGNGWMRHAEEEGVLPLCLDSSVMVDALIICLSETSSSFIIAGVMSLRHINETLSLTLPDIDQMSKVPMCKYLMEKVFKLCHGPAWYARSGGINAIGYMIESFPRKFVMDFVIDVVDSIMEVILGTVEEISSGSADSAYDCLKKMMRVYFIKEEGQEEENLTLATIFVSAISKHYFHSNERVREFAIGLMDHCMVHSRLAPSLDKFYYRFKEFFEPELMRVLTTVPTMSLADAGGSLDGVQNYMFNCPDGFDFEKDMDMYKRYLSHLLDIAQTDTFTLNQRNAFKKCETCPSHFLPPFPITTHIDSMRASALQCLVIAYDRMKKQYIDKGIELGDEHKMIEILALRSSKITVDQVYESDESWRRLMTVLLRAVTDRETPEIAEKLHPSLLKVSPISTIIIATFGASYIRNISGAGDDSDSDRHISYNDIMKFKCLVELNPKILVTKMAVNLANQMVKYKMSDKISRILSVPSSFTEEELDDFEAEKMKGIRELDMIGHTVKMLAGCPVTTFTEQIIVDISRFAAHFEYAYSQDVLVNWIDDVTVILNKSPKDVWKFFLSRESILDPARRSFIRRIIVYQSSGPLRQEFMDTPEYFEKLIDLDDEENKDEDERKIWDRDMFAFSIVDRISKSCPEWLISPNSPIPRIKKLFSETEFNERYVVRALTEVKKFQEEIIVKRMTEHKYKVPKLILNTFLRYLRLNIYDYDLFIVIASCFNGNFVTDLSFLREYLETEVIPKVPLQWRRELFLRIMQKFDTDPQTAGTSMQHVKALQYLVIPTLHWAFERYDTDEIVGTAPIDDSDSSMDVDPAGSSDNLVARLTSVIDSHRNYLSDGMVIVFYQLCTLFVQNASEHIHNNNCKKQGGRLRILMLFAWPCLTMYNHQDPTMRYTGFFFLANIIERFTINRKIVLQVFHQLMTTYQQDTRDQIRKAIDILTPALRTRMEDGHLQILSHVKKILIEECHNLQHVQHVFQMVVRNYRVYYHVRLELLTPLLNGVQRALVMPNSVLEKFSWQTRRHAVEICEMVIKWELFRTLKTDHIISDEEALEVDKQLDKLRTASSTDRFDFEEAHNKRDMPDAQRTIIKEHADVIVNMLVRFCMTFHQNSGSSSTSQSGNHGVELTKKCQLLLRAALRPSMWGEFVSFRLTMIEKFLSIPNDNALRNDISSTAYANTIQNAQHTLDMLCNIIPVMPKTSLMTMMRQLQRPLIQCLNNGAQNFKMTRLVTQIVSRLLEKTNVSVNGLDELEQLNQYISRFLHEHFGSLLNCRNLSGPVLGVLGAFSLLRTICGHEPAYLDHLMPSFVKVMERAAKEHLAYVANSQDGNMVKNFFPDVAELLCACMELVRPRVDHISMEIKRSIVGGIIAELIIKSNHDKIIQTSVKLLGAMISTQDMEFTILTVLPLLVRIQSIIVTKFKNCKDLIADYLVVVITVFENSEYRNSEAGSRLWEGFFWGLKSSDPQTREKFSIVWEKTWPHMATVDIAHRMKYIMQNQDWSKFKHAFWLKFALWGMLRTIAKRPTDPNNKRKKVILLNCATPWRTIEYAAKLKDQPMEVETEMKREEPEPMEVDEKDSQDDSKDAGEPKEKEKLTLELLLAGQQELLDEASNYDFADALDTVSQITFALNENQVTSKMWVVLFKSFWSSLSQSEIEDFTALVVPFMSSGVHNNYQTGVQDSVLAVWLEAVGDAVHLPSRLIEFISSKHECWHTGIRLLENHIWTIPKQLNNTLLREMKVAPGLAGDIETLESLGTLYNEISEFDQFAAIWERRAVFPDTMRAMSAMQLGDMELAQSYLEKSMSSTYETLAPTINPNNTSNSEKHVSPIIDKEYDHWMEMYITNCSELLQWQNVADVCNGKDMQHVRGLINAASHIPDWNVVEECKSQIAGCIPPSFHLDYTLFNLMSTVMRMNENSSPTHMKERCKIAIQECTEAHISRWRALPSVVSYGHVKILQAMNLVREIEESTDIRIALLEAPSNKVDQALMGDMKSLMKVFRNRTPTTSDDMGFVSTWYDWRNQIHGMMLQRFEYWDKVGLNVAATGNQSIVPIHSMAQAQLAVAKHAKNLGFHNLTKDLLNKLAGLTAIPMMDAQDKVCTYGKTLRDMANSAADERVKNELLCEALEVLEDVRIDDLQKDQVAALLYHRANIHSVLDQAENADYTFSAASQLVDLQNSVTTTGIKLMKNWGHHLYKRFFSTTVCKETGNNFGRQALACYFIAARVDNDIKARKPIAKILWLSKHLNACGSHEVMNRVIKKQLHSLNLFNWLYWLPQLVTDVRYKPNSNFVLILCKMAAAHPLQVFYHIREAVSVDDIDSVLEEDYTDEQMSMDVSDEDCFADDPPFDRILKICLKYRPTDIRVFHRVLKELDEMNETWVERHLRHAICLKDQMFKDFSEQMDATFNEMQYSEDVTMMTLRWRKQLEEDLVYFQQNYNLDFLEIRNKRKMIVTKGCMGVEKSQIMFEKELSQVFTEPAGMQDEFDFVTNMTNMMVSQLDIHAVDAPRPQGYIRIVLDWIRAIRRRFDRLPRRIPLESSSPYLARFSHRTGCIEMPYDLLNVLRAKNHTLMASNQTGQYISMLSRFEPNFEIVIKGGQVIRKIYIRGQTGKSAAFYLKKSVQDEPTNRVPQMFKHLDHVLQTDRESARRHLHAPTVLQMRVGQKTTLYEVASVQPYAMPPDCTRNYPASQIDIVHPYDVLTATFNGSYYPDDMVLHFFERFAQSSSSIGQPLPTPTNQDGTVAPPRLTEAHHIKNIIYEDFARDMIPFRLLYDYLTARYPDPVMYYAMKKQLLHSLAVLSTIEYHCNLTPMGPDQMMMTMNTGVLSNPSYRFEIRGGRSLHDIQHFGHEVPFRLTPNLSILVGVAQDGDLLWSMAAASKCLMKKEPEVIMRPLVWDEFANNTDCDKSRLQVFACHASNSYINGVASKLRNTNSADAKLRKDDCVSLISRAKDSDNLARMPPTYHAWF

SEQ ID NO: 15 - DNA, 4896 bp, Caenorhabditis elegans
ttgttttcggattttttgtgtgcttcgtagttgctccgatgatgccggattcaacatttgaatgtaacatttgaattttgaaattgaaggaattcatttgaatctaaagcttgcagggtcaagaccgatacattcttgcaacacatgactcgaaagtatgtaggaaaaattgaagttggaaacttggaatttgatgaaaaagtacagtaatccattctctcttatttcgcaactttcttcgatttttgatttttcctagattttttaagctaaaattttgctgttttattttcatttttcatgcttttcaatttcggttttcaacaaaattatgtttttcagagaaaatctcgtgaacaataactcggctactgtaccatttaaaggcgcacaccttttcgcgcagcattgatttaaatttttttgttcgtggctcaacagtgcaatggacatctagatatctgaaattttaccactgaattcagttcattttttaagcatcttcaaaaatttgcgttttcctaattttcttgtgatcgttttttttttgaaagtacaatcgtacattataaataactatttttcaattcgaataatttaattcaagatcatttcgcaaaataattgccttgaaacgttatgccgcggtcaattttcaaccacccttgttattcttttttgaattgccgccctttttccctgtggccggcgcagtgcggccgaggttggtttctaggccagccggcgcgttttatttttttcgagcatgatttcacaattatttcttgcatttttaaagttttttattgataaaatagtaaaactaacaacggataatattattttaaaattaaaaaactagtttgttcatttttggatcgatttttagatgttgttcatggattatgcacgcaagaaagtactatcgttcacatttgattgctatattattgaatattgaatttttcacacaaaattgtactatttccagatatttatcatgaccgagccgaagaaggagattatagaggacgaaaatcatggaatatccaagaaaataccaacagatcccaggcaatacgagaaagttacagagggatgccggttattggtcatgatggcttcacaagaagaagaaagttagtttttacatctatttaaacacattttccaattattttcaggatgggccgaagttatttcaagatgccgagctgcaaatggttcaattaaattctatgtccattatatcgattgcaaccgaagacttgacgaatgggttcagtctgataggctcaatttagcgtcgtgtgagctaccaaaaaaaggaggaaagaaaggagcacacttgcgggaagaaaagtgagaaatctataaacttttcaaaagattttaaatagttttatcaattcataattatttcagtcgagattcgaatgaaaatgaaggaaagaaaagcggccgaaaacgaaagattccactacttccgatggatgatctcaaggcggaatccgtagatccattacaagcaatttcaacgatgaccagcggatctactccaagtcttcgaggttccatgtcgatggtcggccatagtgaagatgcaatgacaaggatccgaaatgtcgaatgcattgaactaggaagatcacgaattcagccatggtactttgcaccttatccacaacaattgacaagtttggattgtatttatatttgcgaattttgtctgaaatatctaaagtcgaaaacttgtctgaaacggcacatggtgagtgtttcgagttatagaaaatgaccgaatataaataactgttttcaaaattcaaaaattttcaattttccaaaaatgaaagaatcggtgaattcgaaaaaattcgagttcttgtgtgtttttggctgaatttttcggtttttcttgctttttccgttgatattagttttgaaacaatgtttttaaaattttccggcatcgaaaaaaatcgcaaattctgggaatttgctccaaaaattgcatttttgaaatacttttttgcgaaaacgaaaaaaaaattcacaaacggtgtttcaaaccaaatttatcgtaatcaaaaaagtttcgcaaataggccattattctgcgtgggaattcaaattaaaatcagctactttttctattttgcaaaatggaaaaaaaacgtaaaaaatagacaaatttttaattttttaaacaattacattcggtccatactcttcattttctatcatttaattaaaatgcccaattctaattaattttatttcaggaaaaatgtgcaatgtgtcacccacctggcaatcaaatctacagtcacgataaactttcattttttgaaatcgacggccgcaaaaacaaaagctatgctcagaatctatgcctgcttgccaaactttttctggatcacaagactctttactatgacacggatccatttttgttctatgtgctaaccgaagaagacgagaagggtcatcatatagttggatacttttcaaaagaaaaagaatcagctgaagaatataatgttgcgtgtattcttgtgttacctccatttcaaaagaaaggatacggaagtttgctcatcgaattcagctatgaactctcgaaaattgaacagaagacaggatcacccgaaaaaccactatcagatttgggacttctctcatatcgatcgtactggtcaatggccatcatgaaagagcttttcgcattcaaaagacgacatccaggcgaagatatcacagttcaggacatttcacaaagtacatcgattaaacgagaagatgttgtgtcaacgttacagcaacttgatctatacaaatactataagggatcatacataattgtgattagtgatgaaaagcgtcaagtttatgagaaacggattgaggctgcgaaaaagaagacacgaattaatccagcagctctgcaatggcgacccaaagagtacggaaagaaaagagtgagtttttttcaatcaaaaattcgtgtttacggctaaaaactgaaaattaaaattaaattaaattcgtgataacatttttttttcaaaaaaccaaaaaaaaacaatttcgtttttggcagaaccaaaaaaaaaatttaaaaaaaaacggtttacgccctatttcatacaaacaacagaaattgcacttttttgagcaaatttgaccctacaatttttttccagttttttgctctttttcaaaaaaaaacacctaaacactggaaatactaaatactaaggaaaaaaatggaaatactggtttacagtgtcaaaaaattgaaattttctaataaaatcatttttctttttactaaatttatcaaaaatttataactcaaatctttcagtttttgcgaattttttttcgaaaaaacgaaaaaaaataaacctaattttaaccaaattgtaattttgaaaaatctggaacgtccggaaaactgaaaaattaaaaaaaaaacttttcagaaatttatttttaaaaaaccgtttttttaaatcaaattttgtatatgttgatgagaaaaaaaaatagaaatcaatgtttttaagttttaaaagaaaaatttattttaattattttagttttaataaggtatttaaacagtaacaaggatgtcggtttttcgattttccgaaaaactaaaaaattgtctttttcgattttttaatcgaaaaaaaatagaaatattttcacaaaacatactattcttctaaaaaaaagaatagtggcagattttaaataatttttgaactctcgcaatttttttcgaaatatccaaaaatcgaaaaaccggcacaaaagcaaaaagtctccgggaatatatctttaaattattttatgaacttttttttcaggcgcagatcatgttctagcaacaacgacatgtgttctcgccacgacgatctcaacctgtacattaaaatataacactccgttttatctcgcatctacacaccgaaaagcttacgctatccctttatcattcccacaccgctcagagagcgtacgcctcatttcatttcatttgttctgtgtaataatttgacttattagtcacttatttttttaatgaaattattcttgaatttcataatcttcttgttgcagttcaaataattaaaattcatcatatagacaagtaagtttataactgcaaaagtgaagttttctaatcattaagcgttctgaagatattcggcaaccgcctgagcgatcagatcacggcgggaacgagttgaggcgtagacatgcttgcagccagtgacaacctgaaagatattcaaaaaattaatttcaggactcgaatttttaacaatctgaataaaaaaatccaaaattgtatattatagagttttttgaaatctaagcgaaagcgcgctccaatgtaaaacgaaaagtgctccgcccctaaacgttgggtcccgttaggaatttgttattttttcggttatttctgactatattataatttcgaaacgacaagtattttaaacatcatttcgacataaaaaatatgtaaaacaacaaaaaacaatcgaaaaaatagtgaaaaagtttgaatttacagtctcgccgcctcctaccgagacctaacgttaggaggcggagcgttttcctttggcattgaagcgcgcttgctgcggccccataattaataacttacagcctttgcaaagtccttcttctgttcatcctcaatctcgtcaatgtattgattggacaacttctcaatctcggactgttccgcattttcatccttcaattttttgtattgagccttgaattgagccaccttctcctctccgaaagccttaaccgaatactccttacaagcttctttcaacttgccctcggccttctccttggcatctc

SEQ ID NO: 16 - DNA, 1377 bp, Caenorhabditis elegans
atgaccgagccgaagaaggagattatagaggacgaaaatcatggaatatccaagaaaataccaacagatcccaggcaatacgagaaagttacagagggatgccggttattggtcatgatggcttcacaagaagaagaaagatgggccgaagttatttcaagatgccgagctgcaaatggttcaattaaattctatgtccattatatcgattgcaaccgaagacttgacgaatgggttcagtctgataggctcaatttagcgtcgtgtgagctaccaaaaaaaggaggaaagaaaggagcacacttgcgggaagaaaatcgagattcgaatgaaaatgaaggaaagaaaagcggccgaaaacgaaagattccactacttccgatggatgatctcaaggcggaatccgtagatccattacaagcaatttcaacgatgaccagcggatctactccaagtcttcgaggttccatgtcgatggtcggccatagtgaagatgcaatgacaaggatccgaaatgtcgaatgcattgaactaggaagatcacgaattcagccatggtactttgcaccttatccacaacaattgacaagtttggattgtatttatatttgcgaattttgtctgaaatatctaaagtcgaaaacttgtctgaaacggcacatggaaaaatgtgcaatgtgtcacccacctggcaatcaaatctacagtcacgataaactttcattttttgaaatcgacggccgcaaaaacaaaagctatgctcagaatctatgcctgcttgccaaactttttctggatcacaagactctttactatgacacggatccatttttgttctatgtgctaaccgaagaagacgagaagggtcatcatatagttggatacttttcaaaagaaaaagaatcagctgaagaatataatgttgcgtgtattcttgtgttacctccatttcaaaagaaaggatacggaagtttgctcatcgaattcagctatgaactctcgaaaattgaacagaagacaggatcacccgaaaaaccactatcagatttgggacttctctcatatcgatcgtactggtcaatggccatcatgaaagagcttttcgcattcaaaagacgacatccaggcgaagatatcacagttcaggacatttcacaaagtacatcgattaaacgagaagatgttgtgtcaacgttacagcaacttgatctatacaaatactataagggatcatacataattgtgattagtgatgaaaagcgtcaagtttatgagaaacggattgaggctgcgaaaaagaagacacgaattaatccagcagctctgcaatggcgacccaaagagtacggaaagaaaagagcgcagatcatgttctag

SEQ ID NO: 17 - protein, 458 aa, Caenorhabditis elegans
MTEPKKEIIEDENHGISKKIPTDPRQYEKVTEGCRLLVMMASQEEERWAEVISRCRAANGSIKFYVHYIDCNRRLDEWVQSDRLNLASCELPKKGGKKGAHLREENRDSNENEGKKSGRKRKIPLLPMDDLKAESVDPLQAISTMTSGSTPSLRGSMSMVGHSEDAMTRIRNVECIELGRSRIQPWYFAPYPQQLTSLDCIYICEFCLKYLKSKTCLKRHMEKCAMCHPPGNQIYSHDKLSFFEIDGRKNKSYAQNLCLLAKLFLDHKTLYYDTDPFLFYVLTEEDEKGHHIVGYFSKEKESAEEYNVACILVLPPFQKKGYGSLLIEFSYELSKIEQKTGSPEKPLSDLGLLSYRSYWSMAIMKELFAFKRRHPGEDITVQDISQSTSIKREDVVSTLQQLDLYKYYKGSYIIVISDEKRQVYEKRIEAAKKKTRINPAALQWRPKEYGKKRAQIMF

SEQ ID NO: 18 - DNA, 9890 bp, Caenorhabditis elegans
tttcaaaaaaaaaaaattacctcgtcaatttcactctcctcgatgcgatgattatcctcgtccattttacctgaaaagtgtgattttttcacgaataaaattattttcagatacttctagaaaaaaaaaactgaacggaatgttacgaaattaattttcaaagttgcgaaactgaattttcgacaaaaagtttcactgatattcatttcaagcatattgcaacgtttttaaattaatttctaagagaaaaaactgcaaaacaattcgaaaataatttttacaagttacttttcgaaaaagtaacaaaaatccactaatgaacaagaaatttttgaacaaaaagagcttctcaggctatttttggacgaatattttaataaaactttaaaaaaatcaacgaaaatcccctaaaaatcgctgaaaattccaaaaattaaagttcattctcgaccacacctctcgtaaatcagcacgagactcacgcaacgcgaccgcgccgcactcaacggcattgagtaatgcggagcggcagcgtcgcgtcgtctatttgtgtgtgtgtgcgattgtgtgtggtgcgacgtggccgctctgtgtgcctctctagtgagtgttttccgacgagagacaacacattttcgagagacgaagagagtggcgacgaggaagatagtgtggtaagaggagagtgtgcgcgagggaaagagagcaaagtgtgagtgtctgtgagaagagaaggagacccccccccccccgcgctcaaccagtcgatagttggcctgagtgtagggccttctgttgtattccactgctaaccccccccaaacacacaaaaagactcaaaaagtactgcttaaaacacagtgctcagctcatttcatttttgatttttatgctcgccgtcatcggcggatgaattcatcgcaaagtccgtggcgattcaacacgtgcggcgtcctcgccgctcttcttaaccgtagttacaacgtgggagtacagaaagatggccactacttcgaaggcgtttcgagcccgggcgctcgactcgaaccggtctatgactgtatactggggccacgaacttccggacctatcagaatgcagtgttggaaaccgggcggtgacacaaatgccgtctggcatggaaaaagaagaagaacaggttggtttttggtggattatggattactgctccattttgaaatttttcgagttttaatgtcttttttcgaattcctggtgcttttttctatccgaatcatgttttaattccgttttccgactactttgaagaattttcaaatttttgatccctgatgacgtcactatttttgtctttgcctttctggatcgcttttatagttattttcattttttatttcttttttacacttttaaacttaacaattctcttaattcatcctattctatttaattttaagttttgatttttgatttttgatttttctcttttctcttttagccgccggtgggcctttattacaactcttaaatcataaaaaaaatcagtttaagcagttatacataactcttattatgaaaaaatcgttatttttcgacggaaacttcatactttgaatttatttccaatttagattttattttctcaaagtcagctcaattaactaacttaaaatgttttgtcctacccgcaaaatgtttttttttaatattttaattctattttaatttttggctttaaaaaatcattttgctaagcctgagatgaaggcgaaatctcgagaaaaagcatttaaaaagtaataaattccgttaaaaacgactttttctatcacagaaagtgttctctgagtgctaacaaccttcttctgtccaaattttgacacaatttcccaattatgccgacttattacacctttttccgtcaatcttctagtttttcccaccctcttgacccctggtgacgtcatttgtttgttcttcttccaagacatgccctgtggggtattttttctcaaaatttttgcaaatttattggattctaaataaaattccaggagtctagcaccaggaataataatgcaaatttgaaaaaaaaattaaacagaaataatgattttaaatgattatttaaattttaaattttaaatttccaggaaaaacacctgcaagaagcgattgctgcccagcaagccagtacatcgggtattcagctgaaccatgtcattccaactccaaaagtcgaccgagtcgaagatcaacgctatcactccacttatcacaacaagaataaaatgcaccgttcaaagtatatcaaagttcatggtgagtttttttaaccaaaatttcggcgaaaataatttaatttccggttttttgaaattaatttccgcttgggttttcttgtatttattattttttcaaattcctctctgaattcgaaagaaaataacttgatttttcagacttcctggctaaaaccttcaaaaatgtttgttgattggttccaaattttcgcctgattccgaatttcgatgtgacaaattcaaaaaaaaattccctgattttatattcaagctttgtgtttgtgtgttctttttggagcgcgcttgcatcgtttgattttcttcgtcttttttaaaatttattttcgcttgtttcattcatttttgtcgagtttttttctgccaaaatgaatgaaactggtttaaaaaattgaattcggcgaaaataaattttgaaaaacgaaacaaatcaaacgatgcaagcgcgctccaatgcgatttttttgggcgcggaaattcgtgatttcaagcttaaatataaaatcaggtatattttttcgacttttttcacgttgaaattcggaatcagaggaaaattttgagtcaatcaaaaatatttcccagatttcggtatctttaatgcatcaaaaatgaactttcacccccatactcccagaaaaataagaaaacaaattgcgaaatattgttccctgatcaaattttttctttttttaactacacttctctgttttgaagtgagaaagtacatttttctgcgtttcttatcagttatcatttgaaaaggatcagaatttgatgacgatatatttgtttagttacctcccttttttctgaacagtttttgcgaaaaaaggagaaaaaccggaattttctatgaaaatgtgatttattttcagcctggcaagcactcgaacgagacgaacccgagtatgactacgacacagaagatgaagcatggctatcagatcacactcacattgacccgcgcgttttggaaaagatattcgacacagtggagagccattcatcggagacacagatcgcgagcgaagattcggtgattaatttgcataaatgtaagttgacgaaatttccattgaaaccccccccccccaaaaatatcgtttaattgcagcactggactcatcaatcgtgtacgaaatatacgaatattggctgtcgaagcgaacatcggctgcgacgacgtctggttgtgttggagtcggtggattaattccgagagtcaggacagaatgtcggaaggtaagaatttgactattttgaacgaatttcgtgatgaaacttctctaaaacttttaaagttttttatggcggttcaaaatttcggaaaatttacactgattttagctaaaaacttgaattttggtcatttgtccgtgtcacatctgtccgaaatcgactttttttggaattatcatcctttattgcacatttggctagtttatctcatttaatttcgttgattactaaggtacatttaaagccaataggtaaccaaccaaaaactatcataatttttctacactttttaattttccgacactacttgaataaccccataagtgaccaattttgatagtttttggctggttaccggctttaaatgtaccttattaatcaacaaaattaaatgagataaactagccaaatgtgcaataaaggatgataattccataaaaagtcgattttggacagatgtgacacgggcaaatgaccaaaattcaagtttttagctaaaatcagtgtatttgtttcgaagttttgaaccgctataaaaaaatttttggaatgcttttggcaagtttcattacgaaattcactcattttctatacgcaaaaattagaattttcaattaaaaattcattttacaggatggacaaggtgttatcaatccgtacgttgcattccgtcgacgtgccgagaaaatgcagactcgaaagaatcggaaaaacgatgaagattcgtatgagaagattctcaagttggtacatgacatgtcgaaagctcaacagctcttcgatatgactgcccgacgagaaaagcagaagctcgcgttgattgatatggaatcggagattttagcgaaacgaatggagatgtcagattttggtggttctccgagttcgttcaatgagatcaccgaaaagattcgagcagcagcaacgttggaagtcgtgaaaccaccactggcagaaatcaacggatcagatgaagtgaagaagaggaagaagccgagacgaaagattgctgataaggatttaatatcgaaagcctggcttaaaaagaatgcagaaagttggaatcggccgccgtcgctctttggacaacacagtggaaatgttccgacggttacaacgaagccagttcgagagtcgttggcgaatgggcgatttgcgttcaagcggaggagaggatgtgtttatcgcgcggctctcaccgtttacaatgtgcctacagcgcctgctacagtacctccagtacagactcaagcagcagtggcttcatcatcatcgtcaaaatcaacggatatggtgccgtcgaacatgaagttctttgaaacttttgttcgggattcacaggattcagtttctcgatctcttggctttgtacgccgacgaatgggacgaggtgggcgagttgtattcgatcggatgcctcgcaatcgagacgacaacgacgaacgcacttcgacagatccatgggccgagtattgtgtcgcggatagttcaaggtgagatttttgaataagaatcttaatttcacgagattttggtttttttcgctgctttttctgtaattttgtggtattttttctcgtattttcaattaaaaaacgggttttaaataattttaacctgaaatttcgctaaaaaccaagaaatttcattaaaaaatgcaacaaaaaaaaagactggaggcaccaccgaatggagaacaggagaacccaaaaccacgcccatttttccgtgccgggcggcgaaaatttttgcagaatttgctgcaatttttcgttttacaaacgaaacaacgaagctctgaatgtgttatttcggagcttcgttgtttcgtttgtaaaacgaaaaattgcagcaatttctgcaaaaatttgcgcgcggcacggaaaaatgggcgtagttttaggttctcctgttctcctttcggtggtgcctccagtctttttcgcattcttaatgaaatttctttgttttttagcgaaatttcaggttaaaattatttaaaacccgtttttttttcaattggaaatgcgaggaaaaaccacaaaatcacagagaaagcttttggattttttcgcagctttttctgtgattttgtggtttttcctcgcattttcaattgaaaaaaaaacgggttttaaataattttcacctgaaatttcgctaaaaacgaggaaatttcattacaaatgcaaaaaagactggaggcaccaccgaaaccgaatgcagctcagaacaggatttaccaaaacaggatgcagtaggcggagccaattcgcaaccaccgcatgcttatttcgcatgcctcgcacgttttttttttctcttgaaacaatgcaacaatatcaaggaaaaaacgtgcgagacttgcgaaataagcatgcggtggttgcgaattggctccgcccactgcattctgttttggtaaattctgttctgagctgcattctgttttgttggggcttccagtcttttttgtgcatttttaatggaatttcttcgtttttagcgaaatttcaggttaaaattatttaaaacccgtttttttttcaattggaaatgcgaggaaaaaccacaaaatcacagagatagcgaggccccacgaaaaggggagcagaacaaaaaagggggggggggggctggcactgtgccaaacgcacaaaacgctttttattcttattcaacgcacgactttgttataaccacactccgttattacgcatcgcgcgctgtttagcgtgaaaatacaaaaaaacgtcgtgcgttgaatgagaataaaaaagcgttttgtgcgtttggcacagtgccagctctccttttcgcagatccccttttcgtggggcctcagagaaagctgccataaacttttttcttcgcgctaagaccaataccaataaatccttgcgcctttaatatgcaaactatatttttcttccagaaccttccgtgctcgaaacagttcgcttggtaccgaagaagaaaccgatgatctaagcccgaaatctctgtatttcgctcgcagtaatcggttcgcattcaacgatgatgaaactgaacgggaatggacttcaagatgccaacaatcatcgtggagagatacagaggtggatgatgagctgaaaaagcgggaaacaacgtctgaaagtgagattttgaacgatttacctgggaaaatagattattttgggcctattttaattatttaattgcagaatttaccgaaaccacgacgaatggaagtaccaaaacacacacagaatcggatgatagtgaagttgaacggatggaggttgatgatcaagttgatgaagctcaaataactgtatcatcatcaaaagacgatggaatgaatggaaatgataagaacgaggatgaagaagatgatgatgatgatatggatgtagatgaacatcagactgtcgtgggtgtgcatcagcaccagcagcagcagcatcaccagcaaaaagttcggcatcaaatgaatggtggtggtggtggtggtggagtggtaaaactgaaaccgccgctgcaagaactttcgccgccgctttcgggaaacggaagagcggacagagcggaaccgacgccggttccggcaaaggtagtgaggctttttttttaaatactcgaaaaagaaggaaaaaatcccacttttaaaaatacgattcttaaaaatgcgaattccctccaaaatgagaactctgattggccagggagctctcattttgaatggaaattagtcaaaattgaaaaatcccgttttttttttaagttggatttttcattttctcgcgattttttccgcgtttctgtgtcattcctgaatttaacatttaataaattaaaaatgtctggaatattgacaaattatgcttcaaattttttgcgcgggagttcaaaaataatttggccctttttattttttattttgcaaaaatatataaaaaatcattttaaaaaatttagaaacattttttaatttttttaacagttatattcgctatattgggacggtattctgtcattaaacttggtgttgtcgaattttttttattgctttataagactcaaaattgtctgaaaacaccgaattttataatgaaacttcttggaaacttctcaaaaaaaagttatgacggctcaaaaaatgacctaaaatttgttaaaatttgaaatttgacttgtcgcaacggctggaaacaatttttttttttgaaatcaccgtcaaattttgagtataaaatttaattattttgcgttttcaactcgatttttggtattttcaagtcgatggacggcaagatttggttaaaaaattaaaagccgtccattttctcgccgtccattgactttaaactacctaaatcgagttgaaaacgcaagataattgacatttatacccaaaatttgactgtggttttaaaaaagttagtttccagccgctgcgacaagtcaaatttccaattttaactattttaggccattttttgagccatcataacttttttttgagaagtttttaagaagtttcatcatgaaattcggtgttttcagacaattttgagtctaataaagtaattttaaaaaattcgacagacaccacctttatagcaattttgaatttttttttaaacttgtcttgaaaaatcttgaaaaaagtcgaataaattcccattttcctattttcttttttgcagatgtgcggaacggtgtcggactcagatgattggagagagccgagtggatcaccatcagaatcgaattcatcaaccgaatggggtggctatacgccacaagaacagcatgcagttgttgttgccaacgcggtagctgtcgctttcaaggaaaaattgatgaatggcgtggatgatgatgatgatcaacaaccatcgccggctagaggagcacgagatcattccatcaaagagttcgttagtttttctttgctttttttttttttgatttttgagagcaaatttgaaaagttttacacggtttttgaaaaactgttgaaattaaaatttgttgagaatttgatttcgagcaagttttatttttaaaaaattgaatttttcagaaaattctgagttttctttttaaaaaattgaaattttcagaaaattctgagtagcaagaatctttaagatccttaatttctatgcaagaatacgtaggagttttactttgctcaggaaattttattttttgtcagaggagtatatccgaaaaagaacaaaaaaaatgcacatttctcaaaacgcgtatttttttttcagttcgatgtcaacggtaacactgctggaacggaaaaagttcatgatgccgtcgacaatcggtctataatttgaactctctgctgctgcttctgctactgctgctactgctgctcatcgccaattttcaatcctcctgagattttttgatggtcattcattgttttgtgcatatctctctctctctctctctctcccatgattctcaaatatttcaatgtatttacacccccactctgtccgctgcctaatccccgaccgaataatcagattcgctggaaaaatctgcgattctttaatattgcaaccacccacccaataatatgtgtctcatcatctcggtactctcacttgagccgtgttttctgtagtattttattctctaaaaaaaaatcatttttaatataatatacgtacacatttatatctgtaatatatatttttaaaaatgattcccccctcccctccattcgttgttttttttctgtgggtttcaagcttttgagctgtgaaaaatctcatcccatcatcattttctattgttttttttcacagttgaaatatcctattttatctttttcctttttttttcattttttttttttcatcgtgcgggattcatttttcgtcccgcgaaacgcccgccgccgcccaatcccactctctctctcagtctcttcttaatgatcttcgaaactatttttatttccctcattaacaattacgaggtcgtctttttttttccccaccccccactgtttggtgtaatttttgtgttcggggaggttttttgtgtgtggatttttggattttttggattttttcaacaaaaaattcccccgaaatcaaaattttttcccattttcccctcaatattagtactgttgtataaataaacttgctctctctctctctctcgaaatctcctactattatttttttaaaagatttttccaacaaaaattcaaaaaaccacacaaacgacctctctgcacgcggtaatcctctctctttttgtcccccattttctctgtttctctttttttctatcccctatacctgtgattggaatatc

SEQ ID NO: 19 - DNA, 2388 bp, Caenorhabditis elegans
atggccactacttcgaaggcgtttcgagcccgggcgctcgactcgaaccggtctatgactgtatactggggccacgaacttccggacctatcagaatgcagtgttggaaaccgggcggtgacacaaatgccgtctggcatggaaaaagaagaagaacaggaaaaacacctgcaagaagcgattgctgcccagcaagccagtacatcgggtattcagctgaaccatgtcattccaactccaaaagtcgaccgagtcgaagatcaacgctatcactccacttatcacaacaagaataaaatgcaccgttcaaagtatatcaaagttcatgcctggcaagcactcgaacgagacgaacccgagtatgactacgacacagaagatgaagcatggctatcagatcacactcacattgacccgcgcgttttggaaaagatattcgacacagtggagagccattcatcggagacacagatcgcgagcgaagattcggtgattaatttgcataaatcactggactcatcaatcgtgtacgaaatatacgaatattggctgtcgaagcgaacatcggctgcgacgacgtctggttgtgttggagtcggtggattaattccgagagtcaggacagaatgtcggaaggatggacaaggtgttatcaatccgtacgttgcattccgtcgacgtgccgagaaaatgcagactcgaaagaatcggaaaaacgatgaagattcgtatgagaagattctcaagttggtacatgacatgtcgaaagctcaacagctcttcgatatgactgcccgacgagaaaagcagaagctcgcgttgattgatatggaatcggagattttagcgaaacgaatggagatgtcagattttggtggttctccgagttcgttcaatgagatcaccgaaaagattcgagcagcagcaacgttggaagtcgtgaaaccaccactggcagaaatcaacggatcagatgaagtgaagaagaggaagaagccgagacgaaagattgctgataaggatttaatatcgaaagcctggcttaaaaagaatgcagaaagttggaatcggccgccgtcgctctttggacaacacagtggaaatgttccgacggttacaacgaagccagttcgagagtcgttggcgaatgggcgatttgcgttcaagcggaggagaggatgtgtttatcgcgcggctctcaccgtttacaatgtgcctacagcgcctgctacagtacctccagtacagactcaagcagcagtggcttcatcatcatcgtcaaaatcaacggatatggtgccgtcgaacatgaagttctttgaaacttttgttcgggattcacaggattcagtttctcgatctcttggctttgtacgccgacgaatgggacgaggtgggcgagttgtattcgatcggatgcctcgcaatcgagacgacaacgacgaacgcacttcgacagatccatgggccgagtattgtgtcgcggatagttcaagaaccttccgtgctcgaaacagttcgcttggtaccgaagaagaaaccgatgatctaagcccgaaatctctgtatttcgctcgcagtaatcggttcgcattcaacgatgatgaaactgaacgggaatggacttcaagatgccaacaatcatcgtggagagatacagaggtggatgatgagctgaaaaagcgggaaacaacgtctgaaaaatttaccgaaaccacgacgaatggaagtaccaaaacacacacagaatcggatgatagtgaagttgaacggatggaggttgatgatcaagttgatgaagctcaaataactgtatcatcatcaaaagacgatggaatgaatggaaatgataagaacgaggatgaagaagatgatgatgatgatatggatgtagatgaacatcagactgtcgtgggtgtgcatcagcaccagcagcagcagcatcaccagcaaaaagttcggcatcaaatgaatggtggtggtggtggtggtggagtggtaaaactgaaaccgccgctgcaagaactttcgccgccgctttcgggaaacggaagagcggacagagcggaaccgacgccggttccggcaaagatgtgcggaacggtgtcggactcagatgattggagagagccgagtggatcaccatcagaatcgaattcatcaaccgaatggggtggctatacgccacaagaacagcatgcagttgttgttgccaacgcggtagctgtcgctttcaaggaaaaattgatgaatggcgtggatgatgatgatgatcaacaaccatcgccggctagaggagcacgagatcattccatcaaagattcgatgtcaacggtaacactgctggaacggaaaaagttcatgatgccgtcgacaatcggtctataa

SEQ ID NO: 20 - protein, 795 aa, Caenorhabditis elegans
MATTSKAFRARALDSNRSMTVYWGHELPDLSECSVGNRAVTQMPSGMEKEEEQEKHLQEAIAAQQASTSGIQLNHVIPTPKVDRVEDQRYHSTYHNKNKMHRSKYIKVHAWQALERDEPEYDYDTEDEAWLSDHTHIDPRVLEKIFDTVESHSSETQIASEDSVINLHKSLDSSIVYEIYEYWLSKRTSAATTSGCVGVGGLIPRVRTECRKDGQGVINPYVAFRRRAEKMQTRKNRKNDEDSYEKILKLVHDMSKAQQLFDMTARREKQKLALIDMESEILAKRMEMSDFGGSPSSFNEITEKIRAAATLEVVKPPLAEINGSDEVKKRKKPRRKIADKDLISKAWLKKNAESWNRPPSLFGQHSGNVPTVTTKPVRESLANGRFAFKRRRGCVYRAALTVYNVPTAPATVPPVQTQAAVASSSSSKSTDMVPSNMKFFETFVRDSQDSVSRSLGFVRRRMGRGGRVVFDRMPRNRDDNDERTSTDPWAEYCVADSSRTFRARNSSLGTEEETDDLSPKSLYFARSNRFAFNDDETEREWTSRCQQSSWRDTEVDDELKKRETTSEKFTETTTNGSTKTHTESDDSEVERMEVDDQVDEAQITVSSSKDDGMNGNDKNEDEEDDDDDMDVDEHQTVVGVHQHQQQQHHQQKVRHQMNGGGGGGGVVKLKPPLQELSPPLSGNGRADRAEPTPVPAKMCGTVSDSDDWREPSGSPSESNSSTEWGGYTPQEQHAVVVANAVAVAFKEKLMNGVDDDDDQQPSPARGARDHSIKDSMSTVTLLERKKFMMPSTIGL

SEQ ID NO: 21 - DNA, 37007 bp, Caenorhabditis elegans
cagctgatgttgttgatggaaaaatgacggctgcaaagaagccattggctgcaactgagccaaaagtgcataataaataaatgtgtttctaggatcttctaataattttttttctgttttctagctctaaacttgtatttatttcattcttgttctaccaaattcccacggattctacgctttatgtttctaaattattattcttttttatttatatctgcattttcttctaaaaactctggtcattttcttgtttttttcttggtaattataaaaattagtcatacaaatcttgttaaatatctggctattcagtgaacaaaccattttccgctctaaattcgacccgaatcaatcgaaaaatggctcaaaacgatgccatctggctgcaacccccctgtcgtctctcaattttgtgtactctctcgcagccacgcacgcgacgcaacgcactcgcgtcgcggtcgcagttctttttcaaatttatcgcgccatttttgttttgcctcatatttatcggctcacgattgattttcgtcgaaaaacgcgcttaatcgattcctttttacctgaaaaatgttgttccaattggaaaaccagttgaagatcgatgaattttcaagaaaatcattcaaataggcaaaacccgctgaactttgaaattcgatttttgagttttttgaagaaaatataattatttcatcatttatgttggtcctgttggtcctcagcatagaaaattcggacatgacattagaaattcataataactgctcccaatatcgggattagaacgattttcagctcaaaatatggaaaattggttacataaaccgcatatttgtagcattaatcttgaacagctatatggcattaaaaaaaaatatatatatacattgttttttctctcgaagtttctctttttgtttctaaaatccggaatataatttaaaaaaccacataaatttcaatttgcagtacgagttccccccgaatcacaatgccggcaacaccggtgcgtgcttcaagtactcgaataagcagacgtacatcatcaagatcagtggctgatgatcagccatcaacttcgtctgcggtggctccacctccttcacccattgccatagaaactgatgaagatgcggtagttgaggaggagaaaaagaagaaaaagacatcagatgatttggaaattatcactccaagaactccagtcgatcggcgaattccctacatttgctcgattcttttgactgaaaatcgatcgattcgcgataaattgtacgattttttaaatttaattactttcctcaaatccgaataattattagatcgcgcttcgcgtttctgcatccgcggtattttgccttcccactgaaaatagcagatttatcgaatttttagcttaaaaaaaaaatgttttttctgcatttttcaaacaaaccttttgtaaaacagtgaaaatcgaatttcaaatgactaaaatgaattttttttttgtccactggttgtggaatggtttgaatttgaagaaatcagcgggatttttcgtattttctgaatatttttctattaaaaatcggtttcaaaccattttttgacttttgaatagaaaaatattgagaaaatacgaaaaatccagctaacttccagcttgttcaaattcaaaccattccacaaccagtggacgaaaaaagttcattttagtcatttgaaattcgatttggtttgtttgaaaaatgcaaaaaaaaaatattttttaaagctaaaaatttgataaatctgaaaaaaatctgctattttcagtggaaaggcaaaataccgcgaagcgcagcaagcgcgctctaataattattccgcttcgagaagagcgtgtattatttcattgttacatttcaaaattatgaattaatgtttttcagggttctgagcagcggtccagttcgtcaagaagatcacgaagaacagattgctcgagctcaacggatacagccagttgtcgatcaaattcaacgagtcgagcaaatgtatgtgaagctgaaaaattgcaccacaaatcaattattctaatcttgttttacagcatactcaatggttcagtggaagatattctgaaagatcctcgattcgcagtaatggcagatctcacaaaagaaccaccaccaacacctgcacctcctcctccaatccagaagacaatgcaaccgattgaggtgaaaattgaggattcagagggctcaaatacggctcaaccgagtgttctgcccagttgtggaggaggagagacgaatgtggaaagagccgccaaaagagtgagttttgaagatagattggtgtgtaaaaaatgaatgtttatatattcactgcaactttttcctcacgagggacgaggaaaagtggtttctaggccatggccgaggtgccgacaagtttcagcggccatttatcttgctttgttttccgcctgttttctttcgtttttcatcgatttttttcgttttttcttaataaaactgataaataaatattttttgcagatgctaaaacaatttccaagtaaaaaaattatgtattcagtgggcaagcagcggtgaaagtggtcaatgcaatatgatggattacgggaatacaaaacctaaactttttctgaaacatgatacatacgctgcttaaatgctgagactacctgattttcataacgagaccgctgaaaaagttttgaggttttcaaaattcaaattttttggtgaaaaagtcgagattttcgcacaaaaagttgaattctgaaaacctcaaatttttttcagcggtctcgttatgaaaatcaggtaatttcagcatcatatgtatcatgtttcaaaaaaagtttaggttttgtattcccgtaatccatcatattgcattgaccactttcaccgctgcttgcccactgaatacatgattttttacttggaaattgttttagcatctgcaaaaaatatttatttatcagttttattaagaaaaaacgaaaaaaatcggtgaaaaacgaaagaaaacaggcggaaaacaaagcaagataaatggccgctgaaacttgtcggcccctcggccatggcctagaaaccacttttcctcgtccctcgtgaggaaaaagttgcagtgttattgtaaatctcacaagagtctggcatgatttctcaaaggcgcatggatttattcagccctaaaattaaataaatccatacgactttaaaggtggagttcggaaaatgaggattttactttaaaatgctcaaactagtcccaaatgccgaattaccacaaaagaaaaacggaaaaaaattcatcaagtttgaaaaaaatgcggatgattttgttgaaatttcaacgctcgctaatattcctaatttgaaccgcgcttttgtccgcgccgcactctgtagaattgcatccgcgctgtttccttcctcttccggcgccctacttcttttcgattggaaatgatgaaaaaatgagacaaaactagaattcacgtagcgcgtcggaaatgatgaaaatatcatggatgcagcagatctacggagtgcggcgcggacaaacggcgcggtaattcaaatgaggaatattagcgagagttgaaatttcaacaaaatcagccgcatttttttcaaacttaatgtattttttttcgtttttcttttgtagtaattcggcatttggggctagtgtaagcattttaaagtaaaatcctcattttccgaactccacctttaaaggtggagtaccgaaatttgagactttgcttttttaggcccaaattggtccaaaactaccgaattttgtaatgagacgttctgaaaatttatccaaaaaatgttatggcggttcaaagttcggcaaaatagggcccattttcagctaaaatcaaatttttttttccaactttttcggtgtcgcaacgtctggagcctaatttttatttattaatcactttttaataaatattgtagcctttgattaggcgtttattcgctgatttaagtacatttatggtttttggggcacaaataaaagtttcattttatgccccaaaaaccataaatgtacttaaatcagcgaataaacgcctaatcaaaggctacaatatttattaaagagtgatgaataaataaaaattaggttccagacgttgcgacaccgaaaaagttggaaaaaattttgattttagctgaaaatgtgccttattttgccgcgaactttgaaccgccataactttttttgagaaagaaattttcagaacgtctcattacgaaattcggtagttttaaaccaatttgggtctaaaaagtttcaaattccaataaaacataccaaagtcttgtgaaattacaataaactattcctaaacgtattataatccattctcaattcttgcaggaagcgcatgtattggctcgaatcgccgagctccgtaagaacggcttatggtcgaacagtcgtctgccaaagtgcgtcgaacctgaacgtaataaaacgcattgggattatctactggaagaggtcaaatggatggcagttgatttccgaaccgagacgaatacgaagcgaaaaatcgccaaagttatagctcacgccattgcgaaacagcaccgcgacaagcagatcgagattgagagagccgccgaacgggagatcaaggagaagcgaaaaatgtgtgcaggaatcgcgaagatggtacgggatttctggtcgtctacggataaagttgtggatattcgagcgaaggaagttctggagtcgaggctcaggaaggcgagaaataagcatttgatgtttgtaattggacaagtcgatgaaatgagcaatattgtgcaagaaggacttgtttcatcgtcgaaatccccatcaattgcatcggatcgagatgataaagatgaagaattcaaagcacctggctctgattcagaatctgacgatgagcagacaattgcaaacgcggaaaagtcacagaaaaaggaagatgttcgacaggaagttgatgctcttcaaaacgaggcaactgtggatatggatgactttttgtacactttaccgccggaatatctgaaggcttatggtctgacgcaggaggatttggaggagatgaagcgcgagaaattggaggagcagaaggctcggaaggaagcttgtggtgataatgaggagaaaatggagattgatgaagttcgtaggatgctcctaaaaaaattacctaaaaaaaatcgattttccctggaaaaaatcctctggaaatgacccgaaacgtcatggcggctcgaaattttgaaaaaaaaaaccccccaaatttccagctaaaatctcaaattttattgcatattttggtagttcttttgttgtccgaggtgcgtttttcagctgaaaatgtacctgaatctgcaagtaaacgaccaatatatgcaataaatgatgataattaatttccgatactgaaatgtgggcgaaatttgagatttcgactgaaaacgtcttaaaaatcacccaaaacccggctttaccgcacgaaggtttgaagaaaatggccaatttttagccaaaatctcaaatttcgtccacttttcagtcagaaattagttttttgaaattaattaacaccttttattgcatattttcgtcgtttattcgttgatcgaggtgctttttcggtcgatgggtgcacaaattcggtaattgtgcatccatcggctgaaaatgctccagaatttgcgaatgaacggtgaaaatttaagattttagattgaaataagccgttttttagagaaaattggtcgttttgagacattaaattcaatttaaatcccctctttattttcagagcccatcatcagatgctcaaaagccttccacctcaagctcagatctcaccgccgagcagcttcaagatccaacagctgaagacggcaacggtgatggtcatggtgtacttgaaaacgtggattacgtgaagctcaacagtcaggatagtgatgaacgacaacaagagttggcgaatatcgcagaagaagcgctgaaattccagccaaaaggatatacacttgagacgacacaagtcaagacgcccgtaccattcctgattcgaggacaactgagagaatatcaaatggttggattggattggatggttacactttatgagaagaatttgaatggaattcttgccgacgagatgggcctgggaaagacgattcaaacgatttccctgctggctcatatggcttgtagtgaatcgatttggggaccacacttgattgttgtgccgacgtctgtcattctgaattgggagatggagttcaagaaatggtgtccggctctgaagattttgacgtattttggtacggcgaaggagcgtgccgagaagcggaagggatggatgaagccgaattgtttccatgtgtgcatcacatcatacaagacggttactcaagatattagagcttttaagcagagggtgcgtagaaattttgaagatttgcggcgaatttggcgaatttgcataatttttttaaaaccaattttaccgataattgcgaaatttttcaattttatacagtggtcggaaattgctataattagtataatttttgcaaaaattggtacttttttcgaaattttgaaccaccataaaacatttttgaacaatttttaagaggtttaataacgaaattcgttcatttgaacacattttggcgatatgaatcgcccgaaaatgtcccccaatagacctaatttcttaacaaaaatttaaaaaaaaatggcccaaaattgtctcaaaatttcgaaaaaaaaaccgtaatttcagctgaaatctcaaaatttgccaaattttccgtctcacggagatcagaaaaagttttttgcatttttttgtggtttattttagcgttatttcgttaatttagatacattttagcccaatttttgcaaaaattatactaattatagcaatttctgacccctgacaaactttgaaattatcggtaaacttggtataaatggtttttttccaaatttttaaagcgatattaaaggtggagtaccacaatttgaggctttgtttttttttttggacccaaattggtccaaaactaccgaatttcgtaatgagacgctctgaaaatttctttctcaaaaaaaaagttacggcggttcaaagttcgcggcaaaataaggcccattttcagctaaaatcaaaattttttcccaacttctcggtgtctcaacgcctggaacctaatttttatttattcatcactttttaataaatattgtggtctttgattgggcttttattcgttgatttaagtacatttatggtcagtggggcacaaaatgtaactttttttcccaaagaccataaatgtactttaatcaacgaataaacgcccaatcaaagaccacaatatttatttaaaagtaatgaataaataataattaggttccagacgttgcgacaccgagaagttggaaaatttttttattttagctgaataagggccttattgtctcaaactttgaaccgccataacttttttttgagaacgtctcgttacgaaattcggtagttttggaccaatttgggtctaaaaaaacaaagtctcaaatttcttgttagagattttttaaaaattgatattttttttttcaggcctggcagtacctaattctcgatgaagctcaaaatatcaaaaactggaagtcccaacgttggcaggctcttctgaatgtccgtgctcgacgtcgccttctcctgaccggaactccacttcagaactctctaatggaactgtggtcgttgatgcattttttgatgccaacaatattctcaagtcatgatgatttcaaggattggttctcgaatccgttgacagggatgatggaaggaaatatggaattcaatgctccactaatcggacgacttcacaaagtgctccgtccgtttattctgcggcggctcaagaaggaagttgagaagcagctgccagagaagactgagcatattgtgaattgttcgttgtcaaagcggcagagatacctgtacgatgactttatgagtcgtagatcaacaaaggagaatctaaagtctggaaatatgatgtcggtgctcaacattgtgatgcaactccgaaaatgttgtaatcatccgaatctcttcgagccgcggccagttgttgctccgttcgtcgttgagaagcttcagctcgatgttccggctcgtctctttgaaatttcgcagcaagatccctcctcctcctcagctagtcaaattccggaaattttcaatttatccaaaatcggctatcaatcttccgttcgatctgcaaaaccactcatcgaagagcttgaagcaatgagcacttatccggagccacgagcaccagaagttggcggatttcggttcaatcggacggcttttgttgcaaagaatccgcatacggaagagtcggaggacgaaggtgttatgagaagtcgtgttctggtgaatttttaggaaaattgagaaaatgatctaattgttgaattttttaaagaatttatgggccacaagccgatttgccggaaattttgatttttggcgatttgccgaaaattttgatttttggcgatttgccagaaattttgatttttggcaattatccgatttgccggaaattttgatttttggcgatttgccagaaattttgatttttggcaattatccgatttgccggaaattttgaattttggcaattttccgatttgccggaaattttgatttttggcaatttgccgaattgccggaaattttgatttttggcaatttgccgaattgccggaaattttgatttttggggatttgccggaaattttgatttttggcaatttgcctatttgtcggaaattttgatttttggcaatttgccgatttgtcggaaattttgatttttggcaatttgccgatttgccggaaattttgatttttggcaattttccgatttgccaaaaattttgatttttggcgatttgccgatttgccggaaaaacattttgtgagccaattttctcgaaatttgggcttcaatattttcaaattattccaaattttccactgattccgaatatctaagtaaaaaaaaattccctgattttatatttcagcttaaaatcgctaattttcgcgtcagagacgacgtcatgtgtcgatttactggatttttaatctttgtcggatgctaatttccgtttttcaacgagtttccttcatttccatcggtttttgacgaagttttctttgaaaatatgttcttaaggtcaattaaacgttttattatcaaaaaaaactagcaaaattggctttaaaaacacattttcacagaaaactccgacaaaaaccgacgaaaatgaaggaaaccccccgtttgaaaacagaaattagcatctgataaagattaaaatcccgtaaatcgacacatggcgtctggcgtctctggcacgaaaagtcgcgattttaagctgacatacaaaaaaagagggatatatttttttacgaatttttcacatagatattcgaaatcaggggggaaaatttggagaaatttgagaaaatttctcagatttcggattaaaaatattcaatttttgttttcttatattaaaaaaaaattaacttttataatttttcagccaaaaccaattaatggaacagctcaaccacttcaaaatggaaattcaataccacaaaatgctccaaatcgtccacaaacttcatgcattcgttcaaaaaccgtcgtaaatacagttccactgaccatctccaccgatcgaagtggttttcattttaatatggccaatgttggaagaggtgttgttcgtttggatgattcagcacgtatgagcccaccgctcaaacgtcagaagctcaccggaactgcaacgaattggagtgattatgttccgcgacacgttgttgaaaagatggaagaatcgagaaaaaaccagctggaaattgttcgaaggcgatttgagatgattcgtgctccgattattccactggaaatggttgcgctggttcgagaggaaattattgcagaatttccacgtttggctgtggaagaggacgaggttgtgcaggagaggcttttggagtattgcgagttgttggtgcaaaggtagaattttgaaaattattactttgcttttttttaaaccaaaattggcccaaaactaccgaatttcgtaatgagacattctgaaagcttctcaaaaaaaaagttttggccgctcaaagttcgggaaaataaggcccattttcagctgaaatcaaaattttttccaacttctcggtgtcgcaacgtctggaactaaaattttggaaaacgagaaattttccattttttgcaagctgaaaaatcaaagtttttttttcctcaaaattggacaaacaaaaaaatttttttttgaaaattgatcgaaaaaattcaaaatttctataatttttcgattttttaaataaaactttcatcatttttcttccaaatttagttttctcgattttaacttttttcaaaaaaaaattttttaatacgaaaaaaattcaattttagctctaattcttttttagacccaaattggtccaaaactaccgaatttcgtaatgagacgttctgaacatttctcaaaaaaaagttatgacggttcaaagttcggcaaaataaggcccattttcatataaaatcaaattttttttctaacttctcggtgtcacaacgtctggaacttaatttttatttaattattacttttcaataaatattgtggtcttttattaggcgtttatttgttgatttaagtacatttatggtcaagtggggcccaaataaaagttacattttgtgcccacatgaccataaatgtacttaaatcaacgaataaacgcctaatcaaaggccacaatatttattaaaaagtgttgaataaataaaaattaggttccagacattgtgacaccgagaagttaaaaaaaattttgattttagctgaaaatgggccttattttgctgaactttaaaccgctataacttttttttgagaaattttcagaacgtctcattacgaaattcggtagttttggaccaatttgggtctaaaaaagaattagagctaaaattgaattttcttcgtattaaaaattttttttttgaaaaaagtaaaaatcgagaaaactaaatttggaagaaaaatgatgaaaattttatttaaaaaatcgaaaaattatagaaattttgatcgattttttcgatcaattttcaataaaaaattttttgtttgtccaattttgaggaaaaaaaaaactttgatttttcagcttacaaaaaatggaaagtttctcgttttccaattttttgatgtggatttttatgagaaaaaatatataatgtcacaaaaaatagattattatctaaaaatcgaaaaaattaaattttccagttttcaggaaaaaaatcgttaagaaattgtttttccattaaaggtggagtaccgaattttgagacgctgcttttttagacccaaaatggtccaaaactaccgaatttcgtaatgatacgctctgaaaaattttcaaaaaaaaagttgtgaccgctcaaagttttggaaaaatggcatatttttagctaaaatctcaaattttggcaacttatcggtgtcgcagcggttggaacttaatttttatttaattgtcattcattaatgcatgttttggcatttcattatgtgttatttcgttgattgagatgctttttgtgcctgcatcgaccaaaaaaccatctcaatcaacgaaataacacataataaaatgccaaaatatgcattaaaggatgataatcaaataaaaattaagtttcaaccgctgcgacaccgctaagttgccaaaatttgagattttagctaaaaatggtccatttttctaaaactttgagcggtcacaactttttttttgagaaattttcagagcgtctcattacgaaaattggtaggttcggaccaatttgggtctaaaaaagcagcgtctcaaaattcggtacttcacctttaaagttttcaatttaaagtataaattatccaatcaaaaattgacgaaaaaattttttaaaaattttttcttccgaaaaaaaaattaattttaatttttgttagattcggaatgtacgtcgaaccagtgctgaccgatgcttggcagtgtcgtccatcatcgtctggtcttccatcatatattcgcaacaatttatcaaatatcgagctgaattctcgttctcttctcctcaacacctccactaatttcgatacccgaatgtcgatctcacgtgctcttcaattcccagaactccgtctgatcgagtacgattgtggaaagcttcagacgttggctgttctgcttcgtcagttgtacctgtacaagcacagatgtctgatcttcacgcaaatgtcaaagatgctcgacgttctgcagaccttcctttctcatcacggttatcagtatttccgcctcgacggtaccactggtgtcgaacaaagacaggcgatgatggagcggttcaacgcggatcccaaggtgttttgcttcattctgtcgacgagatccggtggtgttggagtcaatctaaccggtgctgacactgtgatcttctacgattcggattggaatccgacgatggatgctcaggctcaggatagatgtcatcgtatcggacagacgaggaatgtctcgatttatcgattgatttccgagcgaacaattgaggagaatattctgagaaaggcaacacagaagcggcgacttggagagttggcaattgacgaggctggcttcacacccgagttcttcaaacaatctgacagtattcgggatctttttgatggagagaatgtggaagtgactgctgtggcagatgttgcgacgacgatgagcgagaaagaaatggaggttgcgatggcaaagtgtgaagatgaagctgatgtgaatgcggcgaagattgcggtggccgaggcgaacgttgataatgcggagtttgatgagaaatcattgccgccgatgagcaatttgcaaggagatgaggaggctgatgagaagtatatggagttgatacaacaggtaaaattcggcggaaatcggaaattttcccatttagaatatcaaattttgcccgattgtgtcgttttttgatttttcgatttattcgatttgtttttgagggaaaatcggaaaaatgttcagaaaattaaccataacatgtgatctttttaaaatcttagcgcaaatgtcttctaaaaaataaagaatgaccaaaaattttaagctaatttttgaaaaaccaaagaaaaaatttagatttttcgatgttttccgagacaaaaagacaaaaacggaaattgtcgaaaatgaatgaaatttttaatttttcagcaaaaaaaaaatagtacttaattttaaaaaatgtgatcatttcggtaggaaaatctggaaaaatcgattttcaaacaaaaaaaaaccgagcctctacaatctttttttttcccgaaatctccagaacttctcacaataacaactatataaatttcaaaatttcagctcaaaccaatcgaacgatatgccattaactttcttgagacacagtacaagccagaatttgaggaagaatgcaaagaggcagaggtatattattccattcatctgactttttttttttttttttaaatttaaatttcaccaaattaattacaggctcttatcgaccaaaaacgcgaagaatgggacaaaaatctcaacgataccgccgtcattgacctcgacgattcggatagtctgctgctcaacgatccttcgacttctgccgatttttatcagagctcaagtcttttagacgaggtacgcgatcgtcgtcgtcgcagcagcagccttctccaaaaagccgctcaaaaaccggcaaaaaagcctcaaaacttccaaattcgtgctcgctccccgtctaagcgtaaatctcaggctccttccttcgatccatatgtttcgtacgcaccgcacgcgctcgcttctcccccggattccccgcgtaagagaagatcacgtggtgcgcgtagtttaggtagtggtggtggtggtggtggtggtagtagatctgttggaagacctgcccgccgatcagtgaagaaagaagaatcagatgatgatgatgaggattattgccaagaagaggaagtgaagcgaaatccggcagaaaaggtcccgccgaaaagaaaacgagttgtgtttgtggaacctccagaggtgaagccgccggagccgaaaaaacgagttgttgttcctgctccatcatcatcatcatcagctctaactactcttccacaacaaggaccgctgatttcgttgccaaaagctgtgccagttgtacctcggccccaacaacaagcaccaccacagctcatcaaaaagcaccagcagactctgatgcctgtgaaggtgctcaagattagtggtggtggtggtggtactccaggaccatccagtgtatcgccaggtccatcaatcctccgaagaaccgttgttccaggcataggcgctggtggtgttggacgcctaccgcttgtcagaatgcctgttcgccctccatttcctggctcgcaagctcctgctccaccgctgagaagtggtgttgctccaacagctcctgcagcagctccacgccagttcgtcgttccgtcgtcgagagttcgagttatcacgacgagaactccggtcgccaccaccatggtgcaacaacaacaaagcccgagcccgttgatgtttccagtccgggttgtgcaaaggcccgggccatctggaccaccaccacctggacctccagatcgcccaggatttggaatctatgagaagccgagattctcacttggatcacgaagaagccgtggagattcgggcccggaagatccggcgccaccacagccaccaccacccaccacttctaggccaccgccacaagcctaggcgctaggattttccttttttttttgttgatttttgctctttttttgctctctcatgattttataatctcattttgctttaatatttccatttttttggatgtgtggaatttttttttttgaaaatcgggaaaaaacgaaaaatttgaactttttggtgattttcagagaaaaatccgtttttaaatgaaaaaatcggaataattcagatttttcgaaaaaaaaaaccgagaaaatttcaaattttcagttttttttttcaaaaaatcgaaaaaaaaagtaaattttcagaattatcagccaagtttttgcgattttttgaaaaatttcaatttttggcaatttttgggaaaaaatcaatttttaattcagaaaattggaaaaattaagatttttcgaaaaaaaaaacgaagaaagtttcaaatttttagcttttttcaaaaaatcgaaaatcggaatttttttaatttttcgaataaaaaaaatcgaagaaattccaaaactttgcgttttttcttgaaattatctgaaaaccggaattttttttcaaaattcgccattttttgcgaatttttgtaatctttttccgagaaaactcgattttttaaatcttaataattcagatttttcgattttcttttgttccaaaaagtcaaaaaccgaacaattatttatttcaaaaactctaaaaattttcaattttttggaaattttcgggtataaaaaaaacccatttttaaatcaaaaaatcggaaatttttgtgatttttcgatttttttcactccaaaaaaattccacacagcaaaaaataaactccgcgcatttttgagcgcacctttcaatgttttaattcttatcacgacgtcaaaattcggttatttttcacacacacacattttcctcccgagcggttctttttttcatgagttctcccatgttttgtttttatatttgagacatttttttttgttgataagtttcaacttcttcttcttcttctgactataaacgtttttctccatgttttttgcctgttttctgccgattttttgacacccaaaattttttttcattttcgctcgaaaatgcacgtcgttggctctagctttggcaagtttttaacactgattttctggtttttttttttttttgcagaatttttcagagatagggggctcattccagcagggtttcccactatatttcgcattttttccaaaaatttttgtattttcaaaaatttccaaaaagaaaggggttttctttaccaaatttttctcgccacttttggcttaattttggctttagagattcgatcgaaaaaattgcgaaagtggcgagaaatctcactggtttgatgtttgaccccctactatagaaaatttgaaaaaaaaaaaaaaaaaaaaaaactagacgaaatttgtggaaatcttgctggagtttgacgagtcgatggtggatttttcttgaaacgaatgaaacggtgattttggatcggagaaatatggcgaaaaatggtgagaaatgacgaggaggaggaagaagctgaaaatctggaggaacaaaaattgtgtggaagtctcgggaagaaattagaattgaaattttaaagtgttctgagaattttttgtgtgaaatttttttaaatctgtagatcaaatatcaaaaaaaaaaatcagaactattacgtgtttatccacaaagatgagaaaaatcgccatatctggcgcgcaaatgaacccgcgggaagagacaaaactactgtagtttttaaccaatttgtgtagatttacgagctattgcgtcatcgaattgaatttaattttcaggcgtttcacacgtttttatattgaaatttatctatttattgaatcaatcttaaaagaaaacacaaaaaattttttttaaaaattgcggctcaaaattaaattcaattcgatgacgcaatagctcgtaaatctacacaaattggttaaaaactacagtagttttgtctcttcccgcgggttcatttgcgcgccagatatggtgatttttctcatctctggataaacacgtaataacatttctcggcacaataaatttttgctgaaacaagtgcgcgcctttgaagagtactgcaatttcaaacacggttttttggttggaaagcacagtactttttcaaaggtgcacaccttctcgaatttctcttcgtgtcgagaccaagaatgccatttttcgatttttaaaaaatcaaaaaaaaaattacctttttaaaggtggagtaccgaaatttgagactttgtttttttcggcccaaaatggtccaaaactaccgaatttcgtaatgagacgttctgaaaatttctcaaaaaacaacgttatggcggtttaaagttcagcaaaataaggcccattttcagctaaaatcaaaattttttcccagcttctcggtgtcacaacgcctggaacctaatttttatttattcatcactttttgataaatattgtggtcttttattaggcgtttattttattgatttaagcttatttatggtctttgtggcgttacattttgtaccctaaaaaccataaatgtacttaaatcaacgaataaacgcctaatcaaaggctacaatatttagtagaaagtgataaataaataaaaattaggttccagacgttgcgacaccgagaagttggcgaaaactttgattttagctaaaaataagccattttcccaaaactttgagcggtcataacttttttttgagaaagaaattttcagaatgtctcattacgaaattcggtagctttgggccattttgggccgaaaaagcaaagtctcaaatttcagcactccaactttagcctttaccttggtgaaattttttaatctgtagtatactttatttttggccgactttttgaacacaaattcggtgttagtttaaaaaaacaatcaaaactaacatattatccagacgcgaaatttttgtcggttttcttcgcgccaaaaagtacggtaacaggtttcggcacgatacatttttgttaaaaggtgctgctcctttgaagagtgtctaataattttcaactttcgtttctgttggaattttcttcaatttttcatagatgttttcgatgaaacaaaaaattaacacaaaatcgtcgtgtcgagacccgaaaaaattttgcgtctgtgcaacaaacccggaaaattaaagtagcatattgatccaaattgccgatttgccggaaattttgattttcggcaatataccgatttgccggaacatttgattttctggaatataccgatttgccggaatttttggttttcggaaatttgccggaaatttagaattccggcaatatgccgatttgccggaaattttgattttcggcaatatgccgatttgccggaaattttgattttcggcaatataccgatttgccggaacatttgatttccggcaatatgccgatttgccggaatttttgatttccggcaatatgccgatttgccggaaattttgattttcggcaatataccgatttgccggaacatttggtttccggcaatatgccgatttgccggaatttttggttttcggaaatttgccggaaatttagaattccggcaatttgccgatttgccggaaattttgatttccggcaatatgccgatttgccggaaattttggttttcggaaatttgccggaaatttagaattccggcaatatgccgatttgccggaaattttgatttccggcaatatgccgatttgtcagaagaaatcgtttgtcacccacacgtgtattgatttgatttttctagataaaattctacgacgagctggacgatatcatgccaatctggcttccaccatcaccaccagattcggatgcggatttcgacttgagaatggaagatgattgtctcgatctgatgtatgaaattgaacaaatgaacgaggctcgcctaccacaagtttgtcatgaaatgagacgtccgttggctgaaaaacagcagaaacagaacacgttgaatgcgtttaatggtaatattttcaaaaaaaaatttttttgaaaaaattcaattaaattcgattttgagcaatttttatcgtgaagattgcataattttgagattttgcgccaagatttttgttaaattgaaaaaaagagatgtgcgcctttatggagtactgtagttttgaaaattgaaattacagtactctgtttaaaggcgcacacatgtattacgtagcgaaaagaaaagtacagtaattagttaaataagactactgtagcgcttgtgtcgatttacgggctctgaattttatatgaatttttgaaaactagaaacatctcaaattgcataaaattaccatttgaacctcccgccaagtgattttgttcgacggggcgcgcttgcacgttttctattttaatttaattcaattttttttgcttaattctcaccgatttttcatgttttcagtttgattttgatggaaatttggagacaatatcaacataaatgcttttcaatcgaaaatgtgcatttatattgacattttctccgaatttccatcaaaattaaactgaaaacacgaaaaatcggtgagaattaagcgaaaaaattgagttaaatgaaaatagaaaacgtgcaagcgcgctccatcgaacaaaatcaattggcgggaggttcaaatgggaattgtatgcaattttcaaaaggtcgtataaaattttgaagaaagcaaattaaatttaaaaaatcgagctcgtaaatcgacacaggcgctaattttcaaaaaaataaaatgacacccaaaaaatcataagaaaatcataaataaatattacgggaacacaaaactcagagaacccgtattgcacaacatatttgacgcgcaaaatatgaaatatctcgtagcgaaaagaaaactaccgtaatttaaaaacatttaaatgactactgtagcgcttgtgtcgatttacgagatctcgattttctaaataaattttttaaaaaatgatgtcagcgatattccatttgactttgtttcttcgtattattttctcatttttgcttgattttatttaattttataattttatttaaaatcaagcaaaaacgagaaaataatacgaagaaacggagttaaatggaatatcgctgacataatttaaaaaaaaaatttaattagaaaatcgagatcccgtaaatcgacacaagtagtcatagtacagtagtcatttaactaattactgtacttttcttttcgctgcgagatatttcatatttttattcatatttttatttattttcatatttttatatatatatatatatatatatttcttggcgttctaatgcagtttctctcaattaattccagacattctatcggcaaaagaaaaggaatcggtgtacgatgcggtcaacaagtgccttcaaatgccacaatccgaagcgatcacagcagaatctgcagcgtctccagcatacacggaacactcatcattctcgatggatgatacaagccaggatgcgaagattgagccaagtttgactgaaaatcaacaacccaccaccaccgccactactactactacagtaccccaacaacaacaacaacagcagcagcaaaaatcgtcgaaaaagaagagaaatgataatcgaacggtacggaggttactagcgaacaatttcaagaaattttgaatttgtgaaaattcaattccggcaatttttcgatttgccggaacttttaattttcgccgaattgtcaatttgccggaaattttgatttccgccgaattgtcgatttgccggaacttttcattttcggcaaattttcgatttgccggaacttttaatttttgacaaattgtcgatgtgccggaaattttgattttcgacaatttgctgatttgccggaaatttcaatcccaacaattttccgatttgccggaaatttcaatcccaacaattttccgatttgccggaaatttcaatcccaacaattttccgatttgccggaaatttcaatcccaacaattttccgatttgccggaaatttcaatcccagcaattttccgatttgccggaaatttcaattccggcaatttttcgatttgccggaacttttcattttcggcaaagtgtcgatttgccggaacttttcattttcgccgaattgtcgatttgcccgaacttttaatttttgacaaattgtcgttttgctggaaattttgattttcgacaatttgccaatttgccggaacttttaatttttgacaaattgtcgatttgccggaaattttgattttcgacaatttgccaatttgccggaacttttcatttttgccaaattgtcgatttgccggaaattttaattccggcaattttgcgatttgccggaaatttcaattccggcaatttaaaaacactaaaaaccaaaaattttcggttttcccgtttttcgatgtttcagcttttctcaaaaaattgcgattccccgaaaaatcgaaacaattttcggggttaaaaccgggaaattcctaaattcctatttaaaagaattgaaaaaaaactctcaaaattccaggctcaaaatcgaacagctgaaaatggtgtgaaacgagcgacaactccaccaccatcatggcgtgaagagccagattatgatggagccgaatggaatatagttgaagattatgcactacttcaagcagttcaagtcgaatttgcaaatgctcatttagtcgaaaaatcggcgaatgagggaatggtgttgaactgggaattcgtgtcgaatgccgttaataagcagacaagatttttccgctcggcccgtcaatgctcaattcgatatcaaatgtttgttcggccaaaagagctcggacagttggtggcttctgatccgatttccaagaaaacgatgaaagtcgacctatcgcatactgaattatctcatttgagaaaaggacgaatgactacggagagccaatatgctcatgattatggaatattgactgataagaaacatgtgaatagatttaaaagtgttcgagtggcggcaacacggagacctgttcagttttggagaggccctaaaggtagaggaggatggcttcataatagtcactgcaactttttcctcacgagggacgagaaaaagtggtttctaggccatggccgaggtgccgacaagtttcagcggccatttatcttgctttgttttccgcccgttttctttcgtttttcaccgatttttttcgttttttcttaataaaactgataaataaatattttttgcagatgctaaaaaaatttccaagtaaaaaaatcatgtattcagtgggcatgcagcggtgaaagtgggcattgtaatatgatggattacgggtatacaaaacctaaactttttctgaaacatgatacatgtgctgcttaaatgctgagactacctgattttcataacgagaccgctgaaaaagttttgaggtttccaaaattcaacttttttggtgaaaaagtcgagattttcgcacaaaaagttgaattttgaaaacctcaaaactttttcagcggtctcgttatgaaaatcaggtaatttcagcatctaagcatcatatgtatcatgtttcagaaaagtttaggttttgtattcccgtaatccatctatttacattgaccactttcaccgctgcttgcccactgaatacataattttttcacttggaaattgttttagcatctggaaaaagtatttatttatcagttttaataagaaaaaacgggaaaaagctgtgaaaaacaaaagaaaacaggcggaaaacaaagcaagataaatggccgctgaaacttgtcggcccctcggccatggcctagaaaccacttttcttcgtccctcgtgaggaaaaagttgcagtgatagtctaaaattcggaggaattttttaaaattggaaaaaattgttaaattttttttttctggaaattggaaaatcacaaattttcgatttttgtttgttaaaaaaaaaaagaaaattggcataataaaacatttcttttttttttgaaaattgggaacttcttaatatcagattttttaagtaagatttttttgattttccggaaattcggaaaacctgaaaattttcaacatttcaaaataaaaatttccgttttttttttctgaaaatctccaacaaaaaaaggtcaaatcgtcagaattattgttggaagtggcggtttttcacgattagagttcagtattttttcttctgaatttcaaatttgaaaaaaaatcgaataaactgtagaaaaatgatagaaaattaacaaaaattctgattaaaggtaaagggaaaatagaccgtaatgaccgaatataactgttgaaaatatcaacaaaaaaaattctgaattttttgtgactttttcaatttttcaagaataaaaaaaacgaccgaataaaatatttgaattcccgcgcaaatgagtgactggttctggccaatttacagtctttttataaaagaaaaaatctagaaaaaccggcgaatttagccagaaaacgcaaaaaattaaaaatgacgtcactcatttgcgcgcggaatacaaatttaattaggccgtttctttgatttttgaaaaattgaaaaaaccattaaaaaatttagaaatttttttgaattttttacagttttttattcggtcattatggggttattcaagtagtgtcggaaaattaaaaagtgtagaaaaattacgtcacaactctgtattcaagtatataaaaacatgtatttaaatacattttgctacattacttgaataaccccattagggtttattttctttagagcaaaaaaaaacatgtttggctctactccacctttaaatgaaaaaatcgacaatttgtgattttgcaatttccagaaaaaaaagaaaaaagttgctttttggaaaaaaccaaaaaaagccatttgaaaaattttattttccaaaaaaaattattttgcagctctagaatctcgaaatctgcaatctctaaacggcggaatgccaccacgacacgagtcgagactcgccgaattcgacgtaaaaaccaatattcgcctggacgccgaggacattgtcacaatgtccgacgagtcgattgtcgcctatgaagcgagcaagaagaagctactggccagtcgtcaaacaaaaccctcaccacgtcaagatgtccgattccatacgctggttcttcggccgtataccgtacctgtgacaactgagtactcggctgcaccttctcgtcgtgaaatgcgcatcgctgttccaccgcttcagccttcggctttatctacgatttcctcagttgctgctgctgccacgtctgggccactaccatcaattcagcatttgcagtcgtcgtctacgggcttgggatctcagcaaaatttgcaaaattcgcataattctgagcaaagaaataatgtgcaaaatatgcatcaaaatcaatataattcaagtcaaaatccgccaatacctatccgacaaatcggagcagcatcatcacaccaacatgatcaaggatctcaggggcctgggggaaaaccacaagcctatcacctggtgcaacagggatcacagcaacagcagcagcagcagcagcaggcgacgttacagcgaagaaatgcggcggcggcggcagggtcgaatgtgcagtttattcagcagcagcagcagcagcagcaatcgggtaaaaattgtatggatttataggaaattatatgaatttgcgcggggatagccccggcgaaaaacgggaaaaagcgacaatttaaaaaaaaatcgtgtgaaaatctcaattttttacaattttgaaagtaattttttattgaaaaaagtggaatttaggcattcatccagagcagggctgggaccaaaaaaaatttttggaccaaaaaccaaaaaacaaaaaattgaaatttccgaaaaatcaacttaagcatcaaaaattttttgtttttttttttgttttttggttttttttggtattttgacgaaaaaacgattttttggttttttggtttttcgagaccaaaaaaaccaaaaaatccaaaaaaatgtttgccgtgtctagtctcgacctagacacggcaaacattttttttttttggattttttggtttttttggtcccgaaaaaccaaaaaaaccaaaaaatcgatttttcgtcaaaataccaaaaaaaaacaaagaattcccagcccctttcgccaaaattgccggatattttcaaacctcaaaaaaaatttataaaggtggactacatcctgtggggaaattgctttaaaacatgcctatgggctcacaatgaccgaatatcatgattaaaaaattcaacaaaaaaattactagattttatgtgattttttgaaaattaaaaaaatctcagttttcaacctaattcctatttgaatttccgccaatttgatttgttcgatggagcgcgcttgctttatttttttttattcattgattttatttttattagcattatttcactgattttcttcattttttgtgtgtttttggtgggaattgaaatgaaaaaaaacaagataaatgcagaaagtctgttaaaaggtcattgaaaatgcttaaaacggcaacaagcttgaaatttgtatattttacacagttttacgcattttcaatgactttttaacaaactttccgcatttatcttgtttttttcagttcaatttccattaaaaaacacacaaaaaaaaatgaagaaaatcagtgaaataaggttaataaataaaataaatgaataaaaatgatgcaagcgcgctccaacgaacgaattcaattggcggaaattcaaatatggaattaggtgaaaactgagatttttttttcaattttcaaaaaatcatataaaatctagaaccattttttgaattttttaatcatgatattcggtcattgtgaccccataggcgtgttttaaagcaatttccccacagggtgtagtccacctttgacgaggtttgaaaatgtccggcaattttgccgaaattgccggaaacttgagatttttcagtgaaaaattccaaatttcatgtggaaaactgtttttttgttttttggaaaatgcaacaaaaaaaactatttggcgcgaaaacgcggatagttttgccaattttcaaggattttccgctatttttaatgtttttatgccgaattttactttaaaaaatcataattattcggaaaatgctcgaagagcatttccaattgtctgtggagcgcgtttgactaatcagataatattccaggcggtcaaggacaaagcttcgttgtcatgggctcgcagagctcatcaaatgatggacaaggtggagcatcgaccgtcggaggaggaggaggaggatcacaacagcctcaccagcagcagcagcagcagccacaacaaagaatacagtacattccacaagttaccggtagcggaaataacggtggaggtggtggaagaggaggctacggtagtacactggtcatgccaagaggaggacgtgttgtcagggttggtagaaatacaaaatcgcgaaaaaacggcatttccggcttcccgaccaatcagcgatttgctccgcccactttcggaccaatccgctgaccgaggcatttgattggtttgaaattgggcggagcagcgaattgctgatgcgaaatacgggaagttctcattttgatggaaattctgcaaaattctttaaaaaaaacaaaatcttctcaaattcggaaaaaatcacaaaggaaatcgaagaaaatcgcgatttttgattccccgaccaatcagcgatttgctccgcccacttttgaaccaatcagcgttcgaggcatttgattggttcaaaactgggcggagcagcgagttgctgattggatttttcagtttttaaatttttaaagctttttttaacggaaaaattcgagaaaaccatagattttgatgagaaatgatgaaaattttcatgaaaaaatggaaaaatgattggaaattaatcaaaaaatcttgaaaaaaaattttttttcagagaaaatgcttcatttttggctctgaaacgcctcttttttatttgtgcctccccgaccaatcagcaatttgctccgcccacttttgaaccaatcagcgaccgagcgatccgattggtttgaaattgggcggagctaaaatgattttaaaaaaattcccgatttgtttaatctagaaatttagaaaaaagaaatatagaaaaaaaatagaaaaaaattaaaaaaaaaaaaacaaaaaatcggaaaacgtcggaaaatattacgaaaaaaatttttttaattgattttttttcgaaaaaaactaaaattttaaccaaaaattcaaagaaaaaatttgtttttgatttttttttcgaaaaaaaaaaaaattttaaccaaaaattcaaaaaaaaaatgtttttcttgatttttttccaaaaaaactaaaattttgaccaaaaattcagcaaaaaaaaaattttttaattgatttttttttcgaaaaaaaataaaattttaaccaaaaattcaaaaaaaaaattttttattgacttttttcgaaaaaaactcaaattttaaccaaaaattcaaaaaaaaaaattttttttttgattttttccgaaaaaaactaaaattttaaccaaaaattcaaaaaaaaaatgtttttcttgatttttttccaaaaaaactaaaattttgaccaaaaattcagcaaaaaaaaaattttttaattgatttttttttcgaaaaaaaataaaattttaaccaaaaattcaaaaaaaaaattttttattgacttttttcgaaaaaaactcaaattttaaccaaaaattcaaaaaaaaaaattttttttttgattttttccgaaaaaaactaaaattttaaccaaaaattcaaaaaaaaattttttattgatttttttccaaaaaaactaaaattttgaccaaaaattcagcaaaaaaaaaattttttaattgattttttttcgaaaaaaactaaaattttgaccaaaaattcaacaaaaaaaaaattttttattgatttttttcgaaaaaaactaaaattttgaccaaaaattcaacaaaaaaaaatttttccagccagcgggaactctaccaggcggtggacgtctgtatgtcgatcataaccgtcatccatatccaatgtcgtcaaatgttgtgccagtacgtgttctaccagccacgcaacaaggacaacaacgaatgatgacaggacaacgtcgtccggctccagcgcccggtactgtcgccgcaatggtgttgccgaatcgaggagctggtggaattccgcaaatgcgcagtttgcagtgagttttgcacggaaattggacgattttcagcgaaattttcgggaaaaatggctattttgtgtttgaaattgcgaaatttcacgatttcgtcttaaatacggtgccaacctaccccatgacggtttgatctacaaaaaacgcgggaatttttcacacaaaaatatgtgagacgtctgcacgttcttaaccaatcggttgaaaactctgccgcatttttgtagatctacggtagatcactgcagattttaagagagaaaaataaataaataatcccacaaggtttttaaaatttttttttcaatcgtaaaaaatagcgaaaaattgtttttcgcgtcgagaccctacgcacatttttttgcaattttcgcttcaaaattacggtaccgggtctcgacacgacatttttattgtgtaaaatacacaattttttggaattttcatcgattcgaatttaaatatttttaaatgatttaattaattcttaacgaaaaaaaaaaagttcgaaactgcagtactctttaaaggcgcacacatgtatgtatttataaaaaatgtcgtgtcaagaccgtacttttggctcacaaattgcaaaatattgcggaattttttttaattttagataaaaaaaaacatgaaaaatctatggaaactaaacttataatttaaaaaaaaatttttttaaggtggactacgctcagtggggaaattgctttaaaacacgcctatgaggccccaatgactgaatatcatgattaaaacaatcaaaaaaaattttctagattttatatgattttttgaaaattggaaaaatcacagttttcacctaattctttttgaatttccgccaattggattagttcggtggagcgcgcttacattatttttaattatttattttatttattctcgttatttgactgattttcttcattttttgtgtgttttcctcggaaaaaggaagaaataaacaagacaaatgcaaaatgtttgttaaaaagtaattgaaaatgcgtaaaactttgatattctgagttccgacgacaacaagcctgaaattagtatatttcacagtttttctcattttcaattactttttaacaaacattttgcatttgtcttgtgtatttcttccattttccgaggaaaaaacatagaaaatgaagaaaatcggtcaaataacgagaataaataaaattaattttaaaaaagatgcaagtgcgctccaccgaacaaatccaattggcggaaattcaaatatggaattaggggaaaactgtgatttttcccattttcaaaaaatcatataaaatttggaaaatttttttgaatttttttaatcatgatattcggtcattggcgccccataggcgtgttttaaagcaatttccccactgagcgtagtccacatttaattttccaaaacagcacatgctaatcctccaagttattccagacgaggcagttacaccggcggtggtggtcagcaacgaatcaacgtgatggttcaaccacaacaaatgcgcagcaacaatggcggtggagtcggtggccaaggaggcctccagggtggtccaggaggtccgcaaggaattcgtcggccactcgtcggacggccactacaacgaggagtcgataatcaggcgccgacggttgctcaggtcgttgttgctccgccgcaaggaatgcagcaggcatcacaaggaccacccgtacttcatatgcagagagcggtttccatgcaaatgccgacgagtcatcatcatcaaggccaacagcaggctcctccgcagagctcacagcaggcttcgcaacaggctcccacatcggattctgggacgagtgctccgccacgacaagcaccaccaccacaaaactagaattttcccctattatcctattttaccccccaaaactctattaattaaataatttccttcctatttttttcttcgtgtgaagattatttgtcccccaaccaagggtgtcggtttttcgatttttcgacgtttttcaaaaaaatttcgatttttcgaaaaattagcttcatattttggctattactctgctttttagaagaaatttgtatgttttttcttgaaaatataagcaaaattagatttaaaaaaaatcatattttatggttaattttctgaacatatttttcaattttcgattttcacagaaaaacatcgaagaatcgacaaaatcgaaaaatatgttccgaaaattaaccataaaatatgattttttttaaaatctaattgtgattatatttataagaaaaaacatacaaatttcttctaaaaagcagagtaatagccaaaatatgaagctaatttttgaaaaaacgaaaaattttcgattttccaaagaatcgaaaaatcgaaaaatgacacccttgcccccaactatctctgtatattattcatctattattgattgtttctttttgttcctcgaaattttttgaaattaaagttctcttccccaccccgatttccgttgctttattaatcgcgattgattaattgtttttccataaatccccaactatttatctctgtatattattcatttatattatttatcttttatctgtgtcgatttacggtatctccgggccgtatgattttgaattctcttctcaaataaaattgtttttcatctaacatttgatacgtgtttttctgatttttttgtatatatattttccatgtatatatttctttttcttttttctttgctccaactttattttaaataatgcttttttatcaagagattttttaaaaaatcgattttttttaaagccaggaattctgaagaatcgaaaaaaatggaactatttttcaaataatgagaaagtttttttttttcaagaaaaaaataataaaattctgatttttttaataaaaatttaataagtttttgaagattttcattgaaaacatctaaactattcgatttttgattttaaattttgaaaatagaattttttaatatatttttttcaaatcgttaaaaagagaatgccggaattttttaaaaattctttaaatttagaaataatcggaaaattttcgattataaaacgctgtataaaacgaaaaaaagtggattttgatgaaagaaaaaattttcttgtagtttttttcagaaaaaaattactttttattctccattttttgttgttgaatttttgagaaaaaactcattttgaaaaaatcgaattttttatattttttctaatcgtaaaaaaaattttaaaaatgaattccggtaattttttaaaaaataatattaatctatagttttgtagttaaaaaaatgtttcacataaaaatctaaaaatttttgattttaaattaaaaaaaaatcgaattttttaaaatttttttcaaatcgtaaaaaaagaaacaataaacaaaagaattccggaaaaaaattatattatgattataaatttatagttctttacttttttaaaagattttattttaaaaattctaaaatgatcgatttttggttttttaaaataatcaaaaatgtttgattttttttaaacgtgaaaaaaatgcaaagaaaatgaaatccggcaaaaattgtaatataattataaatctatacttttgtggttttttccaatatttctataaattcttgatttttaaaataatcaaaagttttgattttaaattttggaaaaattgaatttttgtatatttttctaatcgtaaaaaaatttttaaaaaaatcgaaagcggatttttttctgctattttgttttttttttgaaaaccggaaaaaataccaaaaattgatagtttcgaccactctggctagactaccaaaattgaattttttttttcgaattgagaatggccgtggtctcatcagtagctagccattctctttttatttcaatttttaagaaaaaagtctctaaaattttgaaaaaatcgattttttttacttactttgatactttttttatatcttttcaaatcttaaaaaacaattttaaaaattgaattccggaaatttttttaaataatataaatctatagttttttagtttttaaaaaatatatttttataaaaatctaaaaagttcggcttttgacttttgaaataatcgaaaatgtttgttttaaattttgaaaaaatataaaaaattcgattttttcaagataaaaaagcgaattttttgaatttttttcaaatcgtaaaaaatgtctgtagtttttttaaagactctcataaaaatctgaaatgttcgattttttatttttaaaataattttaaaaaaattttaatattttttatcgtgcgaattttttaccaactataatttggaataattttcaggatctcaaaatatcccacaatcgcgcaaatatgccaggaagcaatgaagattggataaagaaggaggtcgaggaccaggacaccaacgccaacagctcgagctccagcatagccgtctcgcgtcagctcgaagggaattctgctgttcctgacgccatcgaccttctgtcttctcaaatcaaaagagaagttgaagaggaggatgatcgcaacgatgagactggaccccgttcggagcccgtggatgttaagccgtctccaaaacgcccaacgaagaggtcagccgagacctggacgacggctcggcgccaagcaagaaacggtctacggcgggagacggttcaactcatcgattcgcgtatgtgaatgttggagtccgccatccatacgatccacgccatcttgtcatggaaacttcattgaatgaaattaggtaaggaattattgaaaataattattatatatccattttaattcaattttttttttcagaatcgaagatttcgaaataatccagtatcttccgatgcccttcaggacttcgattcccatgaagctagtgatcttcgcagtgagaagtgaagaatctgccgagaagatccgctcgttaatcgatccttcgatgtggatcgcggcttttggtggcggaaccgaaactcaaaaattcttgtggagcgagctgacggtggaggatttcgtcaaggcacacataatggccagcaggtaagctttcgaacatacttaattttttaaaaactaaaattcagcgcaaccgatgacgtgccatatgaggcagccatggcggatcgagaatcgctcaaacaagctgtaaatgatgccagctctctgaaaggcttgaaggaggtaataatttagaaatgacagaaaatgaaccgtgatgacgaaatacatctgtaaaaaaattataaaaaattctaagctccgtttttaattttttttttcagttatattctgtcatagcggcctatttctctggaaaaaaaaatccaaaatagcctcaaattcggaattatgcttcgattttttttctgcggtagtcctgaatttaagacgattttgaatttttgtagctgcctttcgccacaattacgttaaacatttcagagcatgtcgaaagctggatggaggatcgtgagtaagatgcggaaagatctcaatggagcctgatgatccccttcccagcacacaagacagttttaattttgtgtctgtatagttttatattaagttttgatgataatgaatttttttacggttttatccatcacttggctcgattgaagctcctattgtgcagcacacacggcgtgtaaattagtgcatctaacctaggaaatgcgatttctaggccatggccgaggatccgactagatcttttttgatggtgtttgtacagagttaaatttcattttggagggaaattgaaggaaattgaaagagaaattaatttaataatattaatttgatttaaatgaccagaacaaaacaaataaactgaatgacaagccaatcgatattcgtccagactgggatgatgttatatgaactctttcacctgaaacatttaagtttttttaataaaagagcaagcgcgctcaaacgcgaaaacgctcgatccacttaatctggattttgtgccgattcatttatttcaagctatgctcgtttttttctgttatgtttcattaaaaagaccgaaaacataacaaaaagtgcctgaaaacgaaaaaaaaccggcgacattaattgaaaaattcaaaactacaatttcgccgccaaaacccaacgagacccaaagtttcagcgcggagcgtttccacttggccgtggagcgcgcttgtatataaaaggacttaattttttaaaatacttaccgcagttacttccaatgtatgtcaaattcactcgattctccattgcagggttactaaaatatgctccaaatagttggcaaggcgttgacttgaataaatcgggatggttatcttggatgattgcagttcgatttccttttgtaattatgttctaaaaagtcattgtaatcatttaaaagtggagtagcgccagtggggattttgtctaaatgcacttattatgatccaaaacaaccgaatatcatcataaaacactccaaaaagtttagttttttcataatttcctgtcaaagttttggcaaattggcaaaattttgaaaaatgcgagcttttgaggtaatttaaggaaatgtcgcatgtttcgacccctacaattatttaatacagataatttaaacaaaattaaaacataaaaatgtagaaattttttttgttttggtcgatttccaaaattatgagtggcaaaaactgagtaattgccactttttgacagtaaataaaaaatgttcaaaattttttgaaacgttttatcatgatatttggccattatgggagcaaatgagtggtttatctattttttcactggcgctactccacctttaagcatgtctgcctcaccataatcccatttaatccaacgtttcttagatttggattcgaatatatttgaatgactggaaaatatgttacgttaccattcaatgcaccaatataagtcatttgatcgagaaaattcaaatcggtgagatttgtgtttctgatagtcaatgttccgaataaaaattgtaacactcctaatttggaaacatatttttcatcttcatggtctattaatagatctccaaggatatacatacatgtatctgatagtttgctcattgattcaaatgtgcaataaaatgacgcatccaatggaccaggatctttgcaaagtttcgcttcaatgttttcagtagaaattccaaggttcaatagggcaactatctcagtaatggtgacacaaaaatcaggatgaaggttttcaaaattgaagtattgccttttattgtatgtactgtattgtatcatactggtttgctcaactgtatctataactttctgaaattttatgtcattattttcagaaatcgcactaggcaggcaagcctgccttaccgtcagaattggcagtcccagtcgaatcatttccggattatcttgtacattcaatgctacactagctatatccgagttatattcgatagtttgcaggttttgtaaaaacgacaaactctgtagattagtgttccgaattgcaatagatcctcgaatcattgtgacattcaaaaatgaatcataatcgaaggttgcattaatattcactaaatttagaccagaatctagagttttgcatttggagtactccttaacatttgatacattaactttttcaccatcacatcctgaaatttgactatttttatactgttaaaaaattgtttctcaccacaatcctttaagttccctctgacaatgagctcattatacatgtgtaaaaagccgccatcacaggaaaattccagtttcggattattctcgattctaatatcacacgcctcgataccccgatcacggtacaagtagagatcgtagagcacactggggtcgtttaattgtgaattgtttcggatgtaaacaccgtctgaaatctgaagtttaagaaaaaattaagtaagttttaatctacatgttgatccgtttttgttgaaagtatcaaaaaattaactggagtcagaatgtctcatttcgttttgatcttcaaaaaatgcgggagttcagacctagacatctcgtctgatttcgcatggttaagagcgttctgacgtcacaatttttctgaaaaaatattcccgcattttttgtagatcaaattaaaatgagacagcctgacaccacgtggagttccttatatacaaaaaagttgatttttcgctcgtgatttttcgttgtaacatcatgaaaaatccagtgttctctgcaaaccactaaaatccacttttttgtttcagccgctccgcaagcagcttcgtcgaggtcatggcagcggccgagtttcccactccgctgaaactcggcacttaatatatgaacgactaagctagcagggccgccattctaccttaccagcaaaaatgaattcgttcacttacacacatcacacaccacattaaagtttcctttttctttgtcagctgtaaaaaccgaaaggcttgtcagactagtattctcaatattaaatc

SEQ ID NO: 22 - DNA, 5656 bp, Caenorhabditis elegans
atgccggcaacaccggtgcgtgcttcaagtactcgaataagcagacgtacatcatcaagatcagtggctgatgatcagccatcaacttcgtctgcggtggctccacctccttcacccattgccatagaaactgatgaagatgcggtagttgaggaggagaaaaagaagaaaaagacatcagatgatttggaaattatcactccaagaactccagtcgatcggcgaattccctacatttgctcgattcttttgactgaaaatcgatcgattcgcgataaattggttctgagcagcggtccagttcgtcaagaagatcacgaagaacagattgctcgagctcaacggatacagccagttgtcgatcaaattcaacgagtcgagcaaatcatactcaatggttcagtggaagatattctgaaagatcctcgattcgcagtaatggcagatctcacaaaagaaccaccaccaacacctgcacctcctcctccaatccagaagacaatgcaaccgattgaggtgaaaattgaggattcagagggctcaaatacggctcaaccgagtgttctgcccagttgtggaggaggagagacgaatgtggaaagagccgccaaaagagaagcgcatgtattggctcgaatcgccgagctccgtaagaacggcttatggtcgaacagtcgtctgccaaagtgcgtcgaacctgaacgtaataaaacgcattgggattatctactggaagaggtcaaatggatggcagttgatttccgaaccgagacgaatacgaagcgaaaaatcgccaaagttatagctcacgccattgcgaaacagcaccgcgacaagcagatcgagattgagagagccgccgaacgggagatcaaggagaagcgaaaaatgtgtgcaggaatcgcgaagatggtacgggatttctggtcgtctacggataaagttgtggatattcgagcgaaggaagttctggagtcgaggctcaggaaggcgagaaataagcatttgatgtttgtaattggacaagtcgatgaaatgagcaatattgtgcaagaaggacttgtttcatcgtcgaaatccccatcaattgcatcggatcgagatgataaagatgaagaattcaaagcacctggctctgattcagaatctgacgatgagcagacaattgcaaacgcggaaaagtcacagaaaaaggaagatgttcgacaggaagttgatgctcttcaaaacgaggcaactgtggatatggatgactttttgtacactttaccgccggaatatctgaaggcttatggtctgacgcaggaggatttggaggagatgaagcgcgagaaattggaggagcagaaggctcggaaggaagcttgtggtgataatgaggagaaaatggagattgatgaaagcccatcatcagatgctcaaaagccttccacctcaagctcagatctcaccgccgagcagcttcaagatccaacagctgaagacggcaacggtgatggtcatggtgtacttgaaaacgtggattacgtgaagctcaacagtcaggatagtgatgaacgacaacaagagttggcgaatatcgcagaagaagcgctgaaattccagccaaaaggatatacacttgagacgacacaagtcaagacgcccgtaccattcctgattcgaggacaactgagagaatatcaaatggttggattggattggatggttacactttatgagaagaatttgaatggaattcttgccgacgagatgggcctgggaaagacgattcaaacgatttccctgctggctcatatggcttgtagtgaatcgatttggggaccacacttgattgttgtgccgacgtctgtcattctgaattgggagatggagttcaagaaatggtgtccggctctgaagattttgacgtattttggtacggcgaaggagcgtgccgagaagcggaagggatggatgaagccgaattgtttccatgtgtgcatcacatcatacaagacggttactcaagatattagagcttttaagcagagggcctggcagtacctaattctcgatgaagctcaaaatatcaaaaactggaagtcccaacgttggcaggctcttctgaatgtccgtgctcgacgtcgccttctcctgaccggaactccacttcagaactctctaatggaactgtggtcgttgatgcattttttgatgccaacaatattctcaagtcatgatgatttcaaggattggttctcgaatccgttgacagggatgatggaaggaaatatggaattcaatgctccactaatcggacgacttcacaaagtgctccgtccgtttattctgcggcggctcaagaaggaagttgagaagcagctgccagagaagactgagcatattgtgaattgttcgttgtcaaagcggcagagatacctgtacgatgactttatgagtcgtagatcaacaaaggagaatctaaagtctggaaatatgatgtcggtgctcaacattgtgatgcaactccgaaaatgttgtaatcatccgaatctcttcgagccgcggccagttgttgctccgttcgtcgttgagaagcttcagctcgatgttccggctcgtctctttgaaatttcgcagcaagatccctcctcctcctcagctagtcaaattccggaaattttcaatttatccaaaatcggctatcaatcttccgttcgatctgcaaaaccactcatcgaagagcttgaagcaatgagcacttatccggagccacgagcaccagaagttggcggatttcggttcaatcggacggcttttgttgcaaagaatccgcatacggaagagtcggaggacgaaggtgttatgagaagtcgtgttctgccaaaaccaattaatggaacagctcaaccacttcaaaatggaaattcaataccacaaaatgctccaaatcgtccacaaacttcatgcattcgttcaaaaaccgtcgtaaatacagttccactgaccatctccaccgatcgaagtggttttcattttaatatggccaatgttggaagaggtgttgttcgtttggatgattcagcacgtatgagcccaccgctcaaacgtcagaagctcaccggaactgcaacgaattggagtgattatgttccgcgacacgttgttgaaaagatggaagaatcgagaaaaaaccagctggaaattgttcgaaggcgatttgagatgattcgtgctccgattattccactggaaatggttgcgctggttcgagaggaaattattgcagaatttccacgtttggctgtggaagaggacgaggttgtgcaggagaggcttttggagtattgcgagttgttggtgcaaagattcggaatgtacgtcgaaccagtgctgaccgatgcttggcagtgtcgtccatcatcgtctggtcttccatcatatattcgcaacaatttatcaaatatcgagctgaattctcgttctcttctcctcaacacctccactaatttcgatacccgaatgtcgatctcacgtgctcttcaattcccagaactccgtctgatcgagtacgattgtggaaagcttcagacgttggctgttctgcttcgtcagttgtacctgtacaagcacagatgtctgatcttcacgcaaatgtcaaagatgctcgacgttctgcagaccttcctttctcatcacggttatcagtatttccgcctcgacggtaccactggtgtcgaacaaagacaggcgatgatggagcggttcaacgcggatcccaaggtgttttgcttcattctgtcgacgagatccggtggtgttggagtcaatctaaccggtgctgacactgtgatcttctacgattcggattggaatccgacgatggatgctcaggctcaggatagatgtcatcgtatcggacagacgaggaatgtctcgatttatcgattgatttccgagcgaacaattgaggagaatattctgagaaaggcaacacagaagcggcgacttggagagttggcaattgacgaggctggcttcacacccgagttcttcaaacaatctgacagtattcgggatctttttgatggagagaatgtggaagtgactgctgtggcagatgttgcgacgacgatgagcgagaaagaaatggaggttgcgatggcaaagtgtgaagatgaagctgatgtgaatgcggcgaagattgcggtggccgaggcgaacgttgataatgcggagtttgatgagaaatcattgccgccgatgagcaatttgcaaggagatgaggaggctgatgagaagtatatggagttgatacaacagctcaaaccaatcgaacgatatgccattaactttcttgagacacagtacaagccagaatttgaggaagaatgcaaagaggcagaggctcttatcgaccaaaaacgcgaagaatgggacaaaaatctcaacgataccgccgtcattgacctcgacgattcggatagtctgctgctcaacgatccttcgacttctgccgatttttatcagagctcaagtcttttagacgagataaaattctacgacgagctggacgatatcatgccaatctggcttccaccatcaccaccagattcggatgcggatttcgacttgagaatggaagatgattgtctcgatctgatgtatgaaattgaacaaatgaacgaggctcgcctaccacaagtttgtcatgaaatgagacgtccgttggctgaaaaacagcagaaacagaacacgttgaatgcgtttaatgacattctatcggcaaaagaaaaggaatcggtgtacgatgcggtcaacaagtgccttcaaatgccacaatccgaagcgatcacagcagaatctgcagcgtctccagcatacacggaacactcatcattctcgatggatgatacaagccaggatgcgaagattgagccaagtttgactgaaaatcaacaacccaccaccaccgccactactactactacagtaccccaacaacaacaacaacagcagcagcaaaaatcgtcgaaaaagaagagaaatgataatcgaacggctcaaaatcgaacagctgaaaatggtgtgaaacgagcgacaactccaccaccatcatggcgtgaagagccagattatgatggagccgaatggaatatagttgaagattatgcactacttcaagcagttcaagtcgaatttgcaaatgctcatttagtcgaaaaatcggcgaatgagggaatggtgttgaactgggaattcgtgtcgaatgccgttaataagcagacaagatttttccgctcggcccgtcaatgctcaattcgatatcaaatgtttgttcggccaaaagagctcggacagttggtggcttctgatccgatttccaagaaaacgatgaaagtcgacctatcgcatactgaattatctcatttgagaaaaggacgaatgactacggagagccaatatgctcatgattatggaatattgactgataagaaacatgtgaatagatttaaaagtgttcgagtggcggcaacacggagacctgttcagttttggagaggccctaaaggtagaggaggatggcttcataatagtcactgcaactttttcctcacgagggacgagaaaaagtggtttctaggccatggccgaggtgccgacaagtttcagc

SEQ ID NO: 23 - protein, 1885 aa, Caenorhabditis elegans
MPATPVRASSTRISRRTSSRSVADDQPSTSSAVAPPPSPIAIETDEDAVVEEEKKKKKTSDDLEIITPRTPVDRRIPYICSILLTENRSIRDKLVLSSGPVRQEDHEEQIARAQRIQPVVDQIQRVEQIILNGSVEDILKDPRFAVMADLTKEPPPTPAPPPPIQKTMQPIEVKIEDSEGSNTAQPSVLPSCGGGETNVERAAKREAHVLARIAELRKNGLWSNSRLPKCVEPERNKTHWDYLLEEVKWMAVDFRTETNTKRKIAKVIAHAIAKQHRDKQIEIERAAEREIKEKRKMCAGIAKMVRDFWSSTDKVVDIRAKEVLESRLRKARNKHLMFVIGQVDEMSNIVQEGLVSSSKSPSIASDRDDKDEEFKAPGSDSESDDEQTIANAEKSQKKEDVRQEVDALQNEATVDMDDFLYTLPPEYLKAYGLTQEDLEEMKREKLEEQKARKEACGDNEEKMEIDESPSSDAQKPSTSSSDLTAEQLQDPTAEDGNGDGHGVLENVDYVKLNSQDSDERQQELANIAEEALKFQPKGYTLETTQVKTPVPFLIRGQLREYQMVGLDWMVTLYEKNLNGILADEMGLGKTIQTISLLAHMACSESIWGPHLIVVPTSVILNWEMEFKKWCPALKILTYFGTAKERAEKRKGWMKPNCFHVCITSYKTVTQDIRAFKQRAWQYLILDEAQNIKNWKSQRWQALLNVRARRRLLLTGTPLQNSLMELWSLMHFLMPTIFSSHDDFKDWFSNPLTGMMEGNMEFNAPLIGRLHKVLRPFILRRLKKEVEKQLPEKTEHIVNCSLSKRQRYLYDDFMSRRSTKENLKSGNMMSVLNIVMQLRKCCNHPNLFEPRPVVAPFVVEKLQLDVPARLFEISQQDPSSSSASQIPEIFNLSKIGYQSSVRSAKPLIEELEAMSTYPEPRAPEVGGFRFNRTAFVAKNPHTEESEDEGVMRSRVLPKPINGTAQPLQNGNSIPQNAPNRPQTSCIRSKTVVNTVPLTISTDRSGFHFNMANVGRGVVRLDDSARMSPPLKRQKLTGTATNWSDYVPRHVVEKMEESRKNQLEIVRRRFEMIRAPIIPLEMVALVREEIIAEFPRLAVEEDEVVQERLLEYCELLVQRFGMYVEPVLTDAWQCRPSSSGLPSYIRNNLSNIELNSRSLLLNTSTNFDTRMSISRALQFPELRLIEYDCGKLQTLAVLLRQLYLYKHRCLIFTQMSKMLDVLQTFLSHHGYQYFRLDGTTGVEQRQAMMERFNADPKVFCFILSTRSGGVGVNLTGADTVIFYDSDWNPTMDAQAQDRCHRIGQTRNVSIYRLISERTIEENILRKATQKRRLGELAIDEAGFTPEFFKQSDSIRDLFDGENVEVTAVADVATTMSEKEMEVAMAKCEDEADVNAAKIAVAEANVDNAEFDEKSLPPMSNLQGDEEADEKYMELIQQLKPIERYAINFLETQYKPEFEEECKEAEALIDQKREEWDKNLNDTAVIDLDDSDSLLLNDPSTSADFYQSSSLLDEIKFYDELDDIMPIWLPPSPPDSDADFDLRMEDDCLDLMYEIEQMNEARLPQVCHEMRRPLAEKQQKQNTLNAFNDILSAKEKESVYDAVNKCLQMPQSEAITAESAASPAYTEHSSFSMDDTSQDAKIEPSLTENQQPTTTATTTTTVPQQQQQQQQQKSSKKKRNDNRTAQNRTAENGVKRATTPPPSWREEPDYDGAEWNIVEDYALLQAVQVEFANAHLVEKSANEGMVLNWEFVSNAVNKQTRFFRSARQCSIRYQMFVRPKELGQLVASDPISKKTMKVDLSHTELSHLRKGRMTTESQYAHDYGILTDKKHVNRFKSVRVAATRRPVQFWRGPKGRGGWLHNSHCNFFLTRDEKKWFLGHGRGADKFQ

SEQ ID NO: 24 - DNA, 11851 bp, Caenorhabditis elegans
  CDS (1001)...(1035)
  CDS (1920)...(2062)
  CDS (2114)...(2190)
  CDS (2241)...(2501)
  CDS (2551)...(2903)
  CDS (2955)...(3405)
  CDS (3497)...(3631)
  CDS (4227)...(4690)
  CDS (5293)...(6058)
  CDS (6696)...(7058)
  CDS (7609)...(8338)
  CDS (8771)...(8933)
  CDS (9511)...(10306)
  CDS (10774)...(10851)
gtcaatggaattctcgacgcggatcttgttagagatgccgtcgagagagatttgatcaaattgcggtacgctgaaacggatgcaccagttttacaggtaaaatggaaatatacaaactcaaaagtaaaattttatgaatttcagatcaacaactcactatacacggcatcctgggagcaagatctcggaacaaatatggttctgcagtcaaaaggaaaagagatggaagtgatttcgtgtacatcgaccatgatgactgcagaaaaagccctgttgacctcgttaagcaccgaaggatctacactagccgccaatgcagagactgctccgaaatctgatctcagtcgaactcaaccacgtcaacaatgattttcaaaatataaattaacatgaagctctgaaataaactcatataactgctaaaataaaactgttgcttttgaaaccaacatttgttagacaacctgcgtctcacagtcatttttcaatatattggcgccgcgcacacacaaagaagaagaattcgtcctcatggcatggcatgtgcagtcagcggccaccctgtgtaaccactgcgtatcgcatctttccacgtgtttttgcaatcttgctgtcacgttcatttcctcgtacaaccatctcttctacccccgttgcctcctccaccatctcatctcaattgtgtcgttgccctccctctccccaagtctttctgcgtctcttagtgctcttcgagaaaagaacgaggagagctgtgagacgctagtaggaaacgcattctcaattcgatataggcacattgagagagagcgagcgccgtttcgacgtcttctagccttcacatcatccagacgacgttcacacgcacacacagccaaccccacccttctgacaacgaatagacgacgaagaagagaagaagaaaaagaagaaggtacccatttttcattccctttttgcctccacacttcactattatcgattttgtgagcgagctctaatgtttcaacgcaaagtggtattgcctaaaaagcggtgagaatttgcttcagacagaaattcgttttttttaacaagaaaaatccggtttcaattgtcgtagaaggtcaatttttactttcaacgctcttcattgacggaaaactcgtttttctttcaaattttaaattacagaggcattttactcaaggtttgttttaatttaaattaaaaataaattttaaaatagaaatatggataatataaaatgttttcttcaaaaaatgcactcaggttcaccaaaaaatcgataattaaaaatacggtcgcaaaggagcgtcgttagctgctaatcaatggtcttaaaacgaaatctatcgatttttgtgtactacacacggacaagtgctccaccgttattttttgaacgagtgcgttgcaattccatcccattttgacgtttttcttttttttttcatcaaattttttagcatttaaagtaaagtcaatgataacctgcaaataataatgtaaaattcattaaaaaccgagagaaaaagtctaaagtcataaatttttgataaaaaagtgattttcgaaactaaaaatcattcaaattaaagttgaacctgattcttcaatttttattatatattaaaagcttgatccactcaaataaaaggagtttttaattgagaaaaaaagcaaatgaaaaaatcgataattaaattgggcgccaacctagattttaatatgtttttgttagaaatttgtatattttcatcactctctgactttaagcattcgtattttaaggaagtgtgagctttctaatatgttttttattaaaaaaaacatgtttttaacaatctccctgtcatccccatcacctaatgcactcaaataatcaataatcacaatacttttattttttcttgcagaacagaaatggtccaaacgagacgaaagacagctgcagctgtacaggacggtggtgccgttaaggagaacaaagccaagccacctgcccctcaaacgcctacaaaacgagcaaaacgaggtcgtcccccgaaaattaagactggtgagcgaatgactatacggaagattgaaaattcacgtggaatacttgcagatgccaatactttgaatacgccaagcacttcttccaacttggtcgatgacaaacttctcattgagtctgaatcacaggtaaattgattcttttctattcaaaaattaatctaaactatacattccaggactcgattctcacaaacgaagccgactcttttctggaaaaagaagtggaagaaatcgaagatagttcagatatacttcccgataaaattaattctccagaaaaaccaagtgttttggtgaagcggagatcgagtacgcggttaaaagtgaagactgatgaagatgaaaaagatgttcctgtgaacatagaagtagccgttttagaagaaaaatcaattcaaatcgagccaacatctcccgctcacccggaagatcctcaggtgagctttttttaaaaatatgtattaatcaaaattccttcatttccagccttcgacttcttctcttccactggtagaaccaattgaagacattgtggagccaaatgagccaacaagctctgccgatcctccagtatcaaatattaaggatgaggatattaaagaagaagagccactgattaaaaagccagcttccgatgagtcagaatctatggatatagctaactctgaaagtggaaatgattccgattcaagtgaagctgatcctaggacgataccatctttctctatacctcttcccgacacaccacctccaaattttgcgaaaagaggagaaatacatgtagatgtagatcagaaaaattccaagcaatcaggagaatcacaatcgccttgggagcggtaagaatatttatcctagccaggtgttataacaaaattgaatagtttcagagcaagagaaaagtctgcatcgaacccattgtcctctccaacaatgagccgacccaggatacacttccttcatccagcatatcaaagtttcacaaatgattcagtttcacctctaccaccaccgccaccagagccggctccagctcgtgaaaaagtggaaaatggtggtccaactactttcaaaatgactttcaaaaaagctgcaaatattcctatcttgaagacatcggcatttgaacaaccatcatcacctccaccttcctcatcagtttcttcatcaatttcattatctgaagtgaattcttctacatcgatagcctccgagtcttctccagcgaaaagaagctcaaatttcgatttaactgcctcaaatgagcttccaccacctcagatggttgaacttcccaagctctcatttttcaatatgcctccagccgttcgctccgcagaggttagttaactttttcccggtttcatgaaatttcagcggtatctgtcctccttttggtgtgtgccctcacaacctaacctcttttatccaggacgattctgcgatgacgtcggaagaaccgatccttctcctccgttctccgaattccgccactcctgatgatgatgcacttttcctcacgaccccaccaccacccaagatgaccgaatcagaaattcaagcactggtgagccagatcacacatttcgatgtcgtgtgtggaacccaggaatttcagaccgtttttctttacacctcatccccttttgtgttatgttaacattcattttgtgtctcaaacactgcatgcttttgcacttggaaattaaaaaataatgcgttctgggattttgtgtgttaaggtggagtagagtttgtgaggctagaaagtatgcctttttcgtttctccactgcaaaatttcgtttgaaaaaaacaaaaaatttactaaaatttgaaatttcaccaacttgccgttgtcacagctgctgaaatacagtttttattgcattttcaccctttattgcatattattattagacaccttttaggtcaataggcaaccgaaaatatccgaatttgacttaaaatgtacctaaattaaggaactaacttgagatatacgactaaaaatgcaataaattgtgagaattattgttatgaaattcagccgttttaggctagttttagccaaaaaccgacaaactctattccaattaattttccactcctgcacctcgattagtgattttttgaagaaaaaaaattatcttcttatttcagaaagtagcgacggaaaaagtgaatcaagtaattgctcgacgtgaagattctgaaaaagatgtacgtcacagagaagatcgagatgattatgatagacgacgtgacgaccgtgacagaagatccagaaagactgattcggaacgaaatgatcaaagaggacgacaacgtgaagatgatgaacgaagagctcgagaacgagaaagagaagttacgaaacgacatgatcgggaaagggaagagatgcgattacagaaacaaaaagatgaggaaagaagaaagaaagatgaagaggaaaggatacaaaaagagaatgatgagaaaaaacaaaaagaggatgaagccaaaatggaggaggagaaaaagaagattaaagaggaggaaatgaagattcctgaatttgagttgattagcgaatcaaaatatttgacgaggaatgcgaataaaaagaagactgaatccttaacgtaagttattatttataaatttgacttaaaaattgataactttcaaaattaagtgattcaatagactcaaaagaatgaaaaactagagtgcgcctttaaagagtactgtaatttcaaacttttgttgctgctcatttttcatcgatttttcttagtttttcgttaaaaataattcaaccattggattaaaaaaaattaaaaacacataaattttattttgaaaagtaatgagaaaaactatagaaattcgccgaaaattctacagcaacaaaagctcaaaattacagtactttttaaaggagcacatctttctgaatttaacaaaaattcggagatttttctttttttcgtgtttttctggcgaaaaaacgatttttcgcttttaccggaaacggtatccggaggaaaaaaaaaacgaaaaaagcgaaaaattttaagaagtttcaagattagttacaaactcttttcaaaagcagattctacagttttttggggttttgccaaaaaatttatgaaatataatgttttttagactagaaaaataaactaattttaattttcaatcaaaagctcattattatatttatatttatataattcagttgcgaatgccatcgaactggtggaaactgttcggacaatacttgtgtgaatcgtgcaatgctcaccgagtgcccatcatcatgtcaggtcaaatgcaagaatcaacgatttgcaaagaaaaagtacgcggctgttgaagcattccacactggaaccgccaaaggatgtggacttcgagcagtgaaagacataaaaaaaggaagattcatcattgaatatataggagaagttgtggaaagagatgattatgagaagagaaaaacgaaatatgcagctgataaaaagcacaaacatcattatctctgtgatactggagtctacacgatcgacgcaacagtctacggaaatccatctcgatttgtgaatcatagttgtgatcctaatgctatatgtgagaaatggtctgtaccaagaactcctggagacgttaatcgagttggtttcttctcgaaacgattcattaaagccggcgaagaaatcacatttgattatcaatttgtcaactacggacgtgacgctcaacaatgtttctgtggaagtgcttcatgtagtggatggattgggcagaaaccggaagaattttcatctgatgaggatgatgatattgtgactacaaggcatattaatatggatgaagaagaagaagaaaagttggaaggtcttgatcatcttggaaatcatgaacggaatgaagtgatcaaggatatgttggatgatttggtcattcggaataagaagcatgctaggaaggttatcacaattgcggtaagcatttatttgtagagaaaatttaaaaattaaagatggagtaccgaaatccgagaaatatatttaattgactccaatttttcctctgattccgaatttttaaatgaaaaaattcaaaaaaatttccttgattttatgttttaacttgaaattgcgaatttcatttgtacagatttttgaaacgccgaattttcgcgccagagaagccatgtgtcgatttttgagatttgtgtatatttacaagattttgaatcttcatcggatgctgatttgcgtttttcatcattatattatcaaaaaactaacaatttgttcggttttacggaaattaacaatatagactagacatttcgtaaatatacacaaatctcgtaaatcgacacatggcgtctctggcgcgaaaattcggcatttgaaaaatcttatgcgggcactaatgaaattcgtgatttcaagctgaaatataaaatcagggaattttccttgcattttttcactcagaacttcggaatcagttgcaaatttggagtcatttgaaaatatttctcagatttcggtactccacctttattataatttttaaaattttttaaatgattttttttccatgttcaacaaaaaaataaattttcagtctgcaatgaccgattactctcaacgtgtggatgtcattcaagaaatcttctcctcagacacctccgtaaccgttcaaaaattctatgcaaaagagggaatggctacattgatggctgaatggttgtctgaagatgattattcgctggataatctgaaacttgttcaagctattctcaaagctcttcacactgaactattcgattcgtgcgccaaaaatgatcgactcttacgagattctacatcacgatgggtcaatgcgaaaatggatgaatatgttgatatacaagtgatagctgattcacttattgcttgtgttgaagatcccgtacaggagtacaaggatgtttgcaaagttatagaggtatatacatattaatttttaaaaaagaatattttttgcatgtcacaaaatatttggaaattttcccgaaaaacccatgaaatcaaaaaacaaattaaatagtaaaattatttcctcctacgaacatttttcgatttttcgttttccgatattccttttaaaaatctgatttaaaaaaaaaaaacttaaattttaggtctttttgctcctttttagaagcaatttatatgttttttaaaacaaaacttaaaattagcatttttatgggtaattttctgaacacatttttttttcgaaaaaaatggccagaatttcaaccacttctccgtaaaatcgaaattaactaattttttctctatacatttttcaaaaaaagactcctcatttattgtattagatacaaatatatgttttcctcatcaaaatttacgaaatttgttataattttgaattttttttgtttttttttcgaaaaattgaaaattttctaattttgaaacgatattatacaatttcagcgccatcaatttaactaattaaataatttcagaaaggtctcgtcgaaaacttcacaagagccaaagagatggcctatcggttaaatcaatactggttcaatcgatcagtgagcttcaaaattccaaaaaagatacgtgatcctgtgccaaaagatgttccagtcagacaagaagatgctacaacatcatcacaatctcatgataatagtagtagaactgtatcaccgaatcatcgacatcattcatcttcatattcaaattcatgttatcaagaacgagaaccatctcatatacgattctttaataatggaaatgatgttcatcaatatcgttttggaggttatcatggaaataactacaatgataactatttcagtagaaggcccaataaggattcatatcgagatcgccgtcgatttaatggacgtcgttcgagaagtcgatcaagaagtgtctcaccacagaactataaaagaagaaaactcgatgaacatgacaataatcatcgtcagcgttctccaattcgtgatcgtcacacatctcccggcggcgaaaagactcctagctcgaataattctggagaacgaaactataaaagactggatattcgaggagctcgtataaaaactataaaagaagatttggaagctgctgctgctgctgctgctgctgctgctgtaccatcagaagtgcaagcttatcctcatgaacatacagctgtacatcagagtgtttatcagatgccaggttatgagtcttatggttggtttagtttttttaaaaatatcatttaccagggtgccatttttaaaaataaaaataactcggaaaatatgtttttaaaaaatttcagaatttctctcatcaacataaaacttgataaaaatcgaatttttattattttctaaacattttttcggtttttccgaaaatcaaaaaaaaagtttagaaaatagcaaaaaatcagtttattagaaatcaaattttgttcgttttgataagaaaaaacataagaaaacatgttattttcttctgaaaaaagaaaaaaatcgaaaaatctatggccttttggcaaaatgttttggaccaaaaaacaaaacaaatagcattaaaattattagttcttttgttttcttctaaagttaattttctgaaagtcttgcttgtcgtatatcaaataaaaacatttttcaggagtatatgatcctgtaaatggtgtctacatgtatcctcatcctggcgctggttactatccacctgcctatccacaacaaccgattatgttaacaatggacactcttccaccgaatgatcgtcttggtgaactttacgagaaagccagtatcgagcagctagcgtgagcattttttagtttaaacctttcggatttacctagaaaaatgttacctttgacgcaaaattacggtagcaggtctcgtcgcgaccgaaatttttcagcggagtacggtagcttcccatgaatttttttgctgaacttatctttctgataacaaatagtaactaaaacatgaaaaactgaataaaaattgatatctttaccttataggctctttaagggcgcagacacaaaaactgaccggctaccgtaatttttcgtcaaaagtcacacatttctcaactggtgaaatccgaaaaaattgaaatttttactactcgtccgactgtttagaaaagattaaaaaaaaagaaaaaaagaatgtcggtttttcgaattttcgattttcaaagaaaaaaatcaatatttaaaaatcattttcggtaatttccctaaatttgtaaaatataatttccaataaatgttttttgttttccggaattttaataaaaaatcaattttcgcgtaacaaaaatgcgaaaaaatgactagccactcgaatataataacacatgaaataaaattaaaattattacagtcaacgagatgcaattgtgagacaagaacttgagctgatacgtattcaaatcgaaagaaaaactgctcaaaaagaagcgatcaaggccgcttgccgtcgtgctaacgaagaagaagctaaacgacaagaggcacttgcaaagacgaaatatgtttgggcgattgcaaagtcagaagctggagagacgtattactacaacaaaataacaaaagagacgcagtggacagcaccaacaccagttcaaggtcttctcgaaccggcttgtggtgcatctcctgatactacagttgtcattgctgacgagattactgaagaagagcaacaagctgaagttctggagaagccgcgtgttgttaaggaagaagttatcgagccaggttcacaatctgaaactcaaaaagaatctccggagaaagttcgagttgttgtaccgaaagttgaagttgaaagatcaccgtcgccaaaatcttctcgtgatcgtgagaaggatcgagagaaatctcgtgagaaagatcgtgaaagagatcgtgacagaagagaaggttcaaaacatcgtgatagttatcatggacatcgaaacggcagcagttctgtcagtgaacgacgtatgcgagagttcaaacatgagctggaacgatccactcgatctgccgttcgttctcgtctacaacatcaacgtgacgcttctagtgataagactacttggcttattaagttaatatatcgagagattttcaaacgagaaagtgcgcagagtggatttgattatcgattcagtgagaatactgataagaaggtaatattatggaccaaaaaataaacaattgaaaaaaaaaccaaaaaaatctgatgcttgaatttaaaaaaaaacaatgaaagagtgcaattttttaggttttttggtctttttttttggaaaaaccaaaaaataaatttttttccaaagtaccaaacttcattttaaaaaattttatttgacataaaaattgataatttaaaactaatttgaacatttttccgcaaaaattatagatttttctgccaattttagatttttaacgttttttttcggacaattaatgtttcgaatcatcaatcagaatgaatatgatatctgatgaaattcaaaaataatgcaatttaaatagaaaacggtacaaaagttttgaaaaatttagaagaattctaaaaaaaatcctgtccttcaggacaaaattcaacctttttctcaaaacacaaaaattactttatattatttttcaggtgaaaaactacgtcaagtcatatatcgaccgaaaactcgaatcaaacgatctctggaaagaatactctcggccatgagctttattttttaatttaaattttataaaaaaatgtttatgcttgtttttttctctatagttccctcctatcccccccctcccctatcgcctaaaaattgatctctgtctgatttcaccgatttccgttttatttgatcccattgaacgagtatatcatcatgttcctgaacttcaacgttcgcacattttattcccctagttttatgtccccagaattgttttatactatcctgtaatccacctcaaaatgacagccatgaaaagctgtttttcatgttttctattttcttgttgatcgtatttgcgccgctctttgtcgccaaatttttttttgtaattaaaaaatgaattacggatgttgaatttttaaatttatttttttaaagaaaaattgtggaagtttttcagattctatactgcttatttttacgctaaattttttttcgaagtccccttttttcaaatcgaagtgtaactgcgctccacgatcaatagagactctccgccctcgaaccatgggtctcgttaggtatttggcagacttaccgtaaattcaaatgttttattacttcgcgactaatttttttattcatgactcaattttttatcaattccaacgaaaaactaattaaaaacaacggaaaacataacgaaaaatgcttgaaaattgcagacatttccgaaattaattaaattcctaacgagacccatggctcgggggcggagtgttttcgattagccatggagcgcgttgagatattcctaaatttttctattcagatgtcgaatcaatcaaaacgggtcacagtgagaattgagcattcgaagaacacttttttcgaaaagtaattttcaaattttgatccaaagaaattattcgtcaattttcagagttttaaaattccaacatcaagagcaagaagatcggaagctcaaatatgttctgcacaaagctcacgagaatctgagaaagtgcccattcgagattctgacaattg

SEQ ID NO: 25 - protein, 1604 aa, Caenorhabditis elegans
MFQRKVVLPKKRTEMVQTRRKTAAAVQDGGAVKENKAKPPAPQTPTKRAKRGRPPKIKTDANTLNTPSTSSNLVDDKLLIESESQDSILTNEADSFLEKEVEEIEDSSDILPDKINSPEKPSVLVKRRSSTRLKVKTDEDEKDVPVNIEVAVLEEKSIQIEPTSPAHPEDPQPSTSSLPLVEPIEDIVEPNEPTSSADPPVSNIKDEDIKEEEPLIKKPASDESESMDIANSESGNDSDSSEADPRTIPSFSIPLPDTPPPNFAKRGEIHVDVDQKNSKQSGESQSPWERAREKSASNPLSSPTMSRPRIHFLHPAYQSFTNDSVSPLPPPPPEPAPAREKVENGGPTTFKMTFKKAANIPILKTSAFEQPSSPPPSSSVSSSISLSEVNSSTSIASESSPAKRSSNFDLTASNELPPPQMVELPKLSFFNMPPAVRSAEDDSAMTSEEPILLLRSPNSATPDDDALFLTTPPPPKMTESEIQALKVATEKVNQVIARREDSEKDVRHREDRDDYDRRRDDRDRRSRKTDSERNDQRGRQREDDERRAREREREVTKRHDREREEMRLQKQKDEERRKKDEEERIQKENDEKKQKEDEAKMEEEKKKIKEEEMKIPEFELISESKYLTRNANKKKTESLTCECHRTGGNCSDNTCVNRAMLTECPSSCQVKCKNQRFAKKKYAAVEAFHTGTAKGCGLRAVKDIKKGRFIIEYIGEVVERDDYEKRKTKYAADKKHKHHYLCDTGVYTIDATVYGNPSRFVNHSCDPNAICEKWSVPRTPGDVNRVGFFSKRFIKAGEEITFDYQFVNYGRDAQQCFCGSASCSGWIGQKPEEFSSDEDDDIVTTRHINMDEEEEEKLEGLDHLGNHERNEVIKDMLDDLVIRNKKHARKVITIASAMTDYSQRVDVIQEIFSSDTSVTVQKFYAKEGMATLMAEWLSEDDYSLDNLKLVQAILKALHTELFDSCAKNDRLLRDSTSRWVNAKMDEYVDIQVIADSLIACVEDPVQEYKDVCKVIEKGLVENFTRAKEMAYRLNQYWFNRSVSFKIPKKIRDPVPKDVPVRQEDATTSSQSHDNSSRTVSPNHRHHSSSYSNSCYQEREPSHIRFFNNGNDVHQYRFGGYHGNNYNDNYFSRRPNKDSYRDRRRFNGRRSRSRSRSVSPQNYKRRKLDEHDNNHRQRSPIRDRHTSPGGEKTPSSNNSGERNYKRLDIRGARIKTIKEDLEAAAAAAAAAAVPSEVQAYPHEHTAVHQSVYQMPGYESYGVYDPVNGVYMYPHPGAGYYPPAYPQQPIMLTMDTLPPNDRLGELYEKASIEQLAQRDAIVRQELELIRIQIERKTAQKEAIKAACRRANEEEAKRQEALAKTKYVWAIAKSEAGETYYYNKITKETQWTAPTPVQGLLEPACGASPDTTVVIADEITEEEQQAEVLEKPRVVKEEVIEPGSQSETQKESPEKVRVVVPKVEVERSPSPKSSRDREKDREKSREKDRERDRDRREGSKHRDSYHGHRNGSSSVSERRMREFKHELERSTRSAVRSRLQHQRDASSDKTTWLIKLIYREIFKRESAQSGFDYRFSENTDKKVKNYVKSYIDRKLESNDLWKEYSRP

SEQ ID NO: 26 - DNA, 7333 bp, Caenorhabditis elegans
  CDS (1001)...(1096)
  CDS (1166)...(1453)
  CDS (1501)...(2199)
  CDS (2298)...(2730)
  CDS (3234)...(3847)
  CDS (4148)...(5778)
  CDS (6111)...(6333)
gcttgcatcgaaactcttctcattatttacgtgatgatcacatctttcgttgggctgtactcccttccggttcttcgttctcttcgacctgttcgaaaagatactccaatgccaacgataattattaattcttcaatagttcttgttgttgcatccgctctcccagtagctgttaacacagttggaatgacaacttttgatcttctcggctcccactcatcgctccaatggcttggatcatttcgagtcgttgttgcctataatactctattcgtcgtgttgtctgtcgcatttctcttcaatcaattgactgcttcaatgagaaggcaaatctggaagtggtaagctgtgcaatttaaagtttaaattcttattaatttttttgcaggatatgtcaactacgatgtggaatcagacgggagagtgatgcggatgaaaccattgagatccttagaggcgataagaaaagcaattgaatttctttcctttttcaacacttcttacccatgttcatcattttaatcttttcattacaaaaacaaggtcctattttttttctcgggtactactcgccttttctaataattcagaatcatcaatttttgccaacctctagctttacatgtctgtttttcatcattttctctcaagcattctcctaatatattatgttccctagtatttcccctcagtcagcaattttctcgtcgtcgaaaccgtttagctttactttcaatcaaaacgtggaacatttttcaaactatttgaagccaaaaaaaaccagggcttttgtatatgtaccatattttccctctgattttctttatcgccttctcttttcatgtagaataactgaaatacaaaccattttaattttttcttttaattatcaatactgtccgtataggtaaaaattatttcttcaggtttgaaaaaatccgaaatatgtatctgcaactcttcagggcattgcctcaattaatttttatctaatattcagatggaccaacaagaaccatcgaataacgtagatacgagcagtattctttcggatgatgggatggaaacacaggaacaaagttcattcgtcactgctgtgagtgaaattatttaaaatttcgcttcggagattcattgtcatataattcaatttatcgattttcagacaattgacctaacagtggacgactacgatgaaacagaaatacaggagattctggataatggaaaagcagaagaaggaacagatgaagattctgatttagttgaagggattcttaacgctaattcagatgtccaagcgctccttgatgcgccatctgagcaagtagctcaagctcttaattcgttcttcggaaatgagagtgaacaagaagctgttgcagcacaaagacgggttgatgcggagaagactgccaaagatgaagctgaactcaagcaacaggaagaggcggttagattgcaataaaggaaacaataataaaattattttattttcaggaagatcttattatagaagattcgatagtcaaaactgatgaagaaaaacaagcagttcgaagactgaaaatcaacgaatttttatcgtggttcacaaggctccttccagaacaatttaaaaatttcgaattcacaaatccgaactatctgacagaatctatcagcgattcaccggttgtaaatgtcgataaatgcaaggaaattgtcaaatcgttcaaggaaagtgaatcacttgagggactttcacagaaatacgaattaattgatgaagacgtgctagtcgctgctatttgtattggcgttctcgataccaacaacgaagaagatgtcgactttaatgttctatgtgatgatcgtatcgacgattggagtatagaaaaatgtgtcacttttcttgattatccaaatactggattgaattcgaaaaatggaccgttgagattcatgcagtttactgtcacatcacctgcatcagcaattctcatgctcactctgattcgattacgcgaagaagggcatccgtgtcgattagattttgattcaaatccgactgatgatttactcttgaatttcgatcaagtggaattttctaataatatcattgatacggcagtcaaatactgggatgatcagaaggaaaacggtgcgcaggataaaattggcaggcgagtattaatcaaactcacaactgttttgaaagtattttcataattatcacttaaataccttttagagagctcaacgacttcttccacgaaatcgagtcaacatcagcagaattcaaacaacattttgagaacgccgttggcagccgtaatgaaataattcaacttgtcaacgagaaaattcccgattttgatggcactgaggctgctgtgaatgagagttttacatccgatcaacgaaccgaaattatcaactctcgtgcaataatggagacattaaaagccgagatgaagctcgccatcgccgaagctcagaaagtttacgacaccaagactgacttcgaaaaattcttcgttttgacagttggagatttctgtctggctcgcgccaatccttctgacgatgcagaattaacatacgccatagttcaggatcgtgtggatgcaatgacctataaggttaaatttatcgacacaagtcagatcagagagtgtaacatcagagatttagccatgactacgcagggaatgtatgacccgagtttgaatacatttggtgatgttggtgagttttaagttaaaattgatatttaatattacatctgttatgtagaataagggtttcggtttttcgattttattagaaaatcgaaaattttagtttttgtgttaaatttaaaaaaatcaaaatttgattcactatcaagtccgtttttctcttctcaaaattgacaaaattttgataatctagaattttcgtcccgtatatttttcaacgaaaaaccatttaaaattttccatgattggattttcggttgatctagaaaaaaatggtgctaaacactaaatttgaaaaagtttgaaacaaattcaaatccaaatatttcatgaaaaacttgtaaaatatattatgtacacaaaaaaacgtttcaagtgtagcagttgttttttgtggtcccaaaaaagcagatgtttgtcagaatccattaaacaacaaaaaaatccaaaaactcaacctggcctagatatcagtttcatgatcgaagtatctaaaatcattgttttcaggtcttcgagttgcctgtcgccaagttatttcctcgagccaatttggaaaaaaaacaatttggcttaccggtacagctgccggacgtcgcagagctcatagatccgattttctaattttcttcgacaacggaaccgatgcatacgtgtcagctccgacaatgcctggtgaaccaggttatgaagttgcttctgaaaagaaaagtgtattttctctcaaagaaatgattgcgaagatgaatgctgctcagattgctattatggttggacagccagtaggaaaggaaggaaatctggattattttttgacatttcattggattcgacaatctcacagatcagcgtatattcgggattttatgaaagaatttccggaatggccacttctcaagatgccagttggaatgcgaatctgtttgtacaattctcttgttgatcgacgtaagaaaatggtgacagtgattggaactgatcgagcttttgctattgtgagacacgaagcaccgaatccattggctcctgggaatagatgtacagactttccgtgcaatgatagaaatcatcagcatattgacgagaaaatctatagaggatctcatagattggaaggcgcagcggtaagattttatttgaaaaattgatacaaaacgaggattttctaaaattattttatttttatttgatttgatttcttataattgataatcaaggttttttggatgttttgttagagaaatcgaaaagggaaacttccaaaaaaaagctgtgaaatcaatttttgcttttaataatatccaagtttcatcttcaaagttttttctataaaatggacacaaacttttcaacgttttcaaaaaaaaggttccgaaaatatgaaaaaaggagaaagaaatcatgaaaattttgtattatttcagcacaagaagcacatgatctcgacaaataacaatctgtcgcaacgcagaaaagaccagcttcaatcacagttcgagccaaccgacatgattcgttcgatgccagagaggaatcaccaacaagtcgttaaaaagaaaacgacgggcaccaatcagaatgtcgcttcgacaaatgatgcaaaatcgaagagagaaattgaaataagaaagaaaaatcaattcttatttaacaagattattgttccaatacccgtcctaacaccattggaaaatctcaaggctcatgctcaatgtggtccagattgtctacagaaaatggatgcggatccgtatgaagcaagattccatcgaaattcaccaatacatactcctcttttgtgtggttggagacgaattatgtacacaatgagtactggaaagaagcggggagcagtgaagaaaaacattatttacttttctccatgcggagccgctcttcaccagatcagcgacgtctctgaatatattcatgtcaccagaagtttattgacgattgattgtttttcatttgatgcacgaatcgatactgccacttatattactgttgacgataaatatttgaaggttgctgatttttcgcttggaaccgaaggaatcccaattccactagtgaacagcgtggataacgatgagcctccatcattggaatattcgaaacgacgattccaatacaatgatcaagtggatatatcgagtgttagccgagatttctgttctggatgctcttgtgatggtgattgcagtgacgcatcgaagtgtgaatgccaacaattgtccattgaagcaatgaaacgactcccccataatttacaattcgacggacacgacgaattgtatgagagttcagaaaaacaaaataaatttttaaaactattttttttcagagttcctcactatcaaaatcgtcttctcagcagtaaggttatcagtggactctatgaatgcaacgatcagtgttcatgccatcgaaagtcttgttacaacagagttgttcagaacaatatcaagtatcctatgcatgtgagtttatttaacgatgatacataccaattattgttttttcttcagatcttcaaaactgctcaatccggatggggagtccgagctttgacggatattcctcaaagtacgttcatttgcacgtatgtaggtgctatactgacggatgatttggctgatgaactaagaaatgcggatcaatacttcgctgatttggacttgaaggataccgtggagctggaaaagggtcgcgaagatcatgaaactgattttggttacggaggagacgagtcagattatgatgacgaagaaggaagtgatggtgactccggtgatgatgtaatgaacaaaatggtgaaacgtcaagactcttcggagagtggtgaagaaacaaaacggctgacaagacagaaaagaaagcaatctaaaaaatccggtaaaggaggaagtgtggagaaagatgacaccactccaagagattcaatggaaaaggataatattgaaagtaaagacgaacccgttttcaattgggataagtattttgagccgtttccattgtatgttatagatgcaaaacagagaggaaatcttggaaggtaagatcacaattttattcattaaaaaaattttttagagattttgctttaaatgataaaaaatggacaaaccaaccgtttgcctcttcttttggtttatcaacctttctctatggaaaaaattctgaaaaattaacaaacagtatttcacgttgaaaagtgaagaaaaaagcaaaaaaaggaaacaaatttcaaaacggttctactccatcttaaaaaaactaaaattcgtaaaaagtcatttggtatgttttggagactataatacaattgagaaaatttgaaaaaccggcactccaaagatacaatcataaattttcgataactttcagattcttgaatcactcttgcgatccgaatgtgcacgttcaacacgtcatgtacgatacgcatgatcttcgtcttccatgggtcgcgtttttcacacgaaaatacgtgaaagccggcgatgagctaacctgggactatcaatatactcaagatcagacggctaccacacaactcacatgccactgcggagctgaaaactgcaccggccgtttgctgaaaagttaaagaattgttgttatttccttcccagttatgttttcctttttttttaagtatttatttatttatttaatttttattttgtttattgttcaatcgtttaaaatctccctttgaaaacagcatctcatatgtatgatctaaacacgtatttacctcgtaagggtttgccaaatagtttctttggttttcattttgattttctctgcgaataaaatgttttaaaaaagacattatattttttaatagtcagtacagttttgatgtctccaatctatttcagtttacaattttaaaatatagaatatatatatttaggtttcataagttatgcatcgattacgggttctacgtcacttgaagttctgcatttccacgtcacataggactactgtagttttaaaaaatactcgttcattttgtaataatattccttctactagttttgcttctggtaataatcgaatttcaaaactttagctaaaatatttctttttgaagaggctgcagcaaaatatgaaaagaaaagtccaactgaacatgtattacttcgacccgatacatatattggaggtgtcgccatgcgagaagatcaaattatttggctcagagactcagaaaatagaaaaatgattgcaaaagaagtcacttatccacctggattattgaagattttcgatgagattctagtgaatgcggctgataataaagcaagagattccagtatgaatcggttggaagtatggttagataggtaaatatattgcaggaatttatgttctgcgacaaagctacgatacgctgtctcgccacgacaattgttttggtaaatgcatgaaaatcgacgtgcacctttaaataatactgtagttttaaattctcgtttcttcaatttttcataaatggttttccgatgaatatatgattttaaaaaaatctaaaattcacattaatttataagaaacaaaattcctcaaaaacgaaagtttggcgatacagtactatc

SEQ ID NO: 27 - protein, 1327 aa, Caenorhabditis elegans
MDQQEPSNNVDTSSILSDDGMETQEQSSFVTATIDLTVDDYDETEIQEILDNGKAEEGTDEDSDLVEGILNANSDVQALLDAPSEQVAQALNSFFGNESEQEAVAAQRRVDAEKTAKDEAELKQQEEAEDLIIEDSIVKTDEEKQAVRRLKINEFLSWFTRLLPEQFKNFEFTNPNYLTESISDSPVVNVDKCKEIVKSFKESESLEGLSQKYELIDEDVLVAAICIGVLDTNNEEDVDFNVLCDDRIDDWSIEKCVTFLDYPNTGLNSKNGPLRFMQFTVTSPASAILMLTLIRLREEGHPCRLDFDSNPTDDLLLNFDQVEFSNNIIDTAVKYWDDQKENGAQDKIGRRVLIKLTTVLKNAVGSRNEIIQLVNEKIPDFDGTEAAVNESFTSDQRTEIINSRAIMETLKAEMKLAIAEAQKVYDTKTDFEKFFVLTVGDFCLARANPSDDAELTYAIVQDRVDAMTYKVKFIDTSQIRECNIRDLAMTTQGMYDPSLNTFGDVGLRVACRQVISSSQFGKKTIWLTGTAAGRRRAHRSDFLIFFDNGTDAYVSAPTMPGEPGYEVASEKKSVFSLKEMIAKMNAAQIAIMVGQPVGKEGNLDYFLTFHWIRQSHRSAYIRDFMKEFPEWPLLKMPVGMRICLYNSLVDRRKKMVTVIGTDRAFAIVRHEAPNPLAPGNRCTDFPCNDRNHQHIDEKIYRGSHRLEGAAHKKHMISTNNNLSQRRKDQLQSQFEPTDMIRSMPERNHQQVVKKKTTGTNQNVASTNDAKSKREIEIRKKNQFLFNKIIVPIPVLTPLENLKAHAQCGPDCLQKMDADPYEARFHRNSPIHTPLLCGWRRIMYTMSTGKKRGAVKKNIIYFSPCGAALHQISDVSEYIHVTRSLLTIDCFSFDARIDTATYITVDDKYLKVADFSLGTEGIPIPLVNSVDNDEPPSLEYSKRRFQYNDQVDISSVSRDFCSGCSCDGDCSDASKCECQQLSIEAMKRLPHNLQFDGHDELYESSEKQNKFLKLFFFRVPHYQNRLLSSKVISGLYECNDQCSCHRKSCYNRVVQNNIKYPMHVSLFNDDTYQLLFFLQIFKTAQSGWGVRALTDIPQSTFICTYVGAILTDDLADELRNADQYFADLDLKDTVELEKGREDHETDFGYGGDESDYDDEEGSDGDSGDDVMNKMVKRQDSSESGEETKRLTRQKRKQSKKSGKGGSVEKDDTTPRDSMEKDNIESKDEPVFNWDKYFEPFPLYVIDAKQRGNLGRFLNHSCDPNVHVQHVMYDTHDLRLPWVAFFTRKYVKAGDELTWDYQYTQDQTATTQLTCHCGAENCTGRLLKS

SEQ ID NO: 28 - DNA, 12700 bp, Caenorhabditis elegans
  CDS (1001)...(1133)
  CDS (4522)...(5208)
  CDS (6128)...(6361)
  CDS (7962)...(8350)
  CDS (8706)...(8928)
  CDS (9260)...(9516)
  CDS (10328)...(10567)
  CDS (11677)...(11700)
aaaaatttaaaaaaatttttaaaaattcgtgtaaaaattaccccggttgtttaggaaataataaagagattagagacttttttcagatttttattttcttgagttttgccggttttcagccgatttctatcttttttttctcattttttgtgattttttttcgctagttttcccctcaatttctcgattttttcacgattttttgaaaattttcggaaaattgaattgtttgcaaaaaaaaaaattcaaaaaccgcatttttctcagaatttttctgggattttgtacaaatttttgaattatttctcaaaaaaaagcaggtttttaccgatttttttggttttttccccaaaattttccgattttttccgagttttgccggttttcagccgaattctactctcgatttttttacgattttttggaaattttcggaaaattatttgaaaaaaaatcaaaaaaccgcatttttttttctgaattttctgggattttgtacgaaattttgaaatttttctcgaaaaaagcaagttattccccaaaattttctgattttcccccaaaaatttagatttttcccgagttttccccagttctcagctgatttctatatttttttctcaatttttgtgattttttgttgctagttttcccttcaattcctcgagtttttcacgattttttggagattttcgaaaaattgtttgaaaaaaatcaagaaaccacatttttctctggattttctcgaaatttgcacaaaatttttgaattttttcgtaaaaaaaaactgttttccccaaaaatttcagatttgtttttgatttttttcgagattttcccctgatttcaaagttttttcctgaatttttcgaatatttcctgaaaaatcggctatttctaactttttaaataattttttttgaatttctgactttttaaatccttttttttttgccattttttcccatctaaaattctaaattattcaaaattttacagaatgtcagaagtaatcgacgaaagtatcttaaatacagaagcttcagatgatccaatacctccattaaatgatgatcagattgctgagcttttgggtgaagatggagaaattatggagataactgagcagaaaggtgagattttttgagtaaaaccttgaattttgcactaaaaatttgcaattttcgctaaaaattaccttaaaactcgaaaattggaatttctagctgagaaaatggccaaaaatgtcgaaaaatgcctccgaaacctgtgaaaaaaaaaaccaccaaaaaggtttctaggccaccaaaaagatttctaggccaccaaaaatgtttctaggccaccaaaaatgtttctaggccaccaaaaatgtttctaggccaccaaaaatgtttctaggccaccaaaaatgtttctaggccaccaaacaggtttcaatgccaccaaaaatgtttctaggccaccaaaaatgtttctaggcccccaaaaaatttttctaggccaccaaaaaggtttctaggccaccaaaaatgtttctaggccaccaaaaaggtttctaggccaccaaacaggtttcaatgccaccaaaaaggtttctaggccaccaaccaggtttcaatgccaccaaaaatgtttctaggccaccaaaaaggtttctaggccaccaaaaatgtttctaggccaccaaaaatgtttctaggccaccaaaaaggtttctaggccaccaaacaggtttcaatgccaccaaaaatgtttctaggccaccaaacaggtttcaatgccaccaaaaaggtttctaggccaccaaaaaggtttctaggccaccaaaaatgtttctaggccaccaaaaaggtttctaggccaccaaacaggtttcaatgccaccaaaaatgtttctaggccaccaaacaggtttcaatgccaccaaaaatgtttctaggccaccaaacaggtttcaatgccaccaaaaatgtttctaggccaccaaaaaggtttctaggccaccaaaaatgtttctaggccaccaaaaatgtttctaggccaccaaaaaggtttctaggccaccaaacaggtttcaatgccaccaaaaatgtttctaggccaccaaacaggtttcaatgccaccaaaaatgtttctaggccaccaaaaatgtttctaggcccccaaaaaatttttctaggccaccaaaaaggtttctaggccaccaaaaatgtttctaggccaccaaaaaggtttctaggccaccaaacaggtttcaatgccaccaaaaaggtttctaggccaccaaccaggtttcaatgccaccaaaaatgtttctaggccaccaaaaaggtttctaggccaccaaaaatgtttctaggccaccaaaaatgtttctaggccaccaaaaaggtttctaggccaccaaaaaggtttcaaggccaccaaaaaggtttcaatgccaccaaaaatgtttctaggccaccaaacaggtttcaatgccaccaaaaaggtttctaggccaccaaaaatgtttctagaccaccaaaaaggtttctaggccaccaaacaggtttcaatgccaccaaaaaggtttctaggccaccaaacaggtttcaatgccaccaaaaatgtttctaggccaccaaaaaggtttctaggccaccaaaaatgtttctaggccaccaaaaatgtttctaggccaccaaaaaggtttctaggccaccaaacaggtttcaatgccaccaaaaatgtttctaggccaccaaacaggtttcaatgcccccaaaaaatttttctaggccaccaaaaaggtttctaggccatcaaaaatgtttctagaccaccaaaaaggtttctaggccaccaaaaatgtttctagaccaccaaaaaggtttctaggccaccaaaaatgtttctaggccaccaaaaaggtttctaggccaccaaaaatgtttctaggccaccaaaaaggtttctaggccaccaaacaggtttcaatgccaccaaaaaggtttctaggccaccaaccaggtttcaatgccaccaaaaatgtttctaggccaccaaaaaggtttctaggccaccaaaaatgtttctaggccaccaaaaatgtttctaggccaccaaaaaggtttctaggccaccaaaaaggtttcaaggccaccaaaaaggtttcaatgccaccaaaaatgtttctaggccaccaaacaggtttcaatgccaccaaaaaggtttctaggccaccaaacaggtttcaatgccaccaaaaaggtttctagaccaccaaaaaggtttctaggccaccaaacaggtttcaatgccaccaaaaaggtttctaggccaccaaacaggtttcaatgccaccaaaaatgtttctaggccaccaaaaaggtttctaggccaccaaaaatgtttctaggccaccaaaaatgtttctaggccaccaaaaaggtttctaggccaccaaacaggtttcaatgccaccaaaaatgtttctaggccaccaaacaggtttcaatgcccccaaaaaatttttctaggccaccaaaaaggtttctaggccaccaaaaatgtttctagaccaccaaaaaggtttctaggccaccaaaaatgtttctagaccaccaaaaaggtttctaggccaccaaaaatgtttctaggccaccaaaaaggtttctaggccaccaaacaggtttcaatgccaccaaaaatgtttctaggccaccaaaaatgtttctaggcccccaaaaaatttttctaggccaccaaaaaggtttcaatgccaccaaaaatgtttctaggccaccaaaaaggtttctaggccaccaaaaatgtttctaggccaccaaaaatgtttctaggccaccaaaaaggtttctaggccaccaaacaggtttcaatgccaccaaaaatgtttctaggccaccaaacaggtttcaatgccaccaaaaaggtttctaggccaccaaaaatgtttctagaccaccaaaaaggtttctaggccaccaaacaggtttcaatgccaccaaaaaggtttctaggccaccaaacaggtttcaatgccaccaaaaatgtttctaggccaccaaaaaggtttctaggccaccaaaaatgtttctaggccaccaaaaatgtttctaggccaccaaaaaggtttctaggccaccaaacaggtttcaatgccaccaaaaatgtttctaggccaccaaacaggtttcaatgccaccaaaaatgtttctaggccaccaaaaatgtttctaggcccccaaaaaatttttctaggccaccaaaaaggtttctaggccaccaaaaatgtttctagaccaccaaaaaggtttctaggccaccaaaaatgtttctagaccaccaaaaaggtttctaggccaccaaaaatgtttctaggccaccaaaaaggtttctaggccaccaaaaatgcttctaggccaccaaaaatgtttctacgccaccaaaagccgcctcaagcccgaaaaatttgaatttcccgctcaaaaaatctaaaattttccgattttcagacgaatcagatgatgtggtgatgctggacgacgatgatgacgacactccggaaccgattctcgtgattgatatggatgaggatgaggatgttactacagatggtcctgaatctcaggaagagctggctgcagatgctccggctccaggagctccagaagcttcagctccagctcaagaagcctcagaagcttcagctccggatcaagaagctccagaagttcaggatgttccggattcttcgggagctccagatgcttcagctcaggcttcagaggcttctgatgcttcagctccagaagttccaggatctacagaagctcaggatgctcaggatgttccggattctttgggagcttcagatgcttcagctcaagaaattccagaagctccagaagccccagaagctccagaaatcgccgctgaaatcgacgaagaagtgctgctcgccgagcaaaatggagttttggacgaaggatttgatgagactgacgatattatcatagaagaagaagctgtagaagaagctgaagccgtggagccaccaattaacactgaaaatcaggaaaacgcgctggaaatgctcgaagagcgcctcaagaagaatgaagaaaaggaaattgtggagaaaagtgatgtgaagccagaggatgaagatattatacatatggagacggattcagttgaaagtatgggcttttttagctggaaaacaggaaaaaagagcaaaaaattgatacatttccagcttaaccaatctttttttgagttgtaaagcctgaaaattgagatttttgtaccaacttttatgataaagctgaaaaaaaaattaattttttgacgaatttttagcggaaaccctgaaaacatgttttgtctgaaaaatacagaaaatcgtcactttttacaataaattcgagatttttagctcaaaaatacaacattatagtgcaaaaatctcagaaaaagccaaaaatttcattcaaacatctcaaaaaaagcagaaattttactcaaaatatctcagaaaaagctaaaattttcccaaaaaatcccagaaaaagcagaattttcattcaaaattcccagaaaaagctgataatttactaaacaatctcagaaaatgctgaaattttactcaaaagtcttcataaaaagctgaaattttactttaaaagtttaggaaatgctgcaatttcacttaaaaatcccaaaaaagctaaaattttcccaaaaaatcccagaaaaagcagaaattttactcgaatatctcaaaaaaaaaaaagctgaaatttcactcaaaaatcccagaaaaagctaaaaatttactaaaaaatctcaaaaaaaaaaacgctaaaatttcactcaaaaatctcagaaaaagctaaaattttactcgaatatctcaaaaaaaaaaactgaaattttcctaaaaaatttatgaaaaaccgaaatttcacttaaaagtctcataaaaagccgaattttcccaaaaaaatcccagaaaaagctaaaaatttactttaaaatctcatctgtaattttagtttaaaatctcagaaaaacccgaaatttctctcaaaaatttgctgattttcaaattttcagcgtcaagccgcaaacgtactggcggagccacaagtccgcggagcccggctcaaaaacgaccaaaacgacgtgttcaaacgttattaaagatgcgtcagaatgcaattgaactattgacacgactttatggctcatgggatgcacaattgagcctctcaaatcttgagacaattcgattgttgggtgtcaataataataggaagcttatcgaaatttttgaggagaatgagcaaggttaaagcgtttttaaatgctatgaaaactgacaaattttcgataaaaaaacggatttttggaagaaaatcgcctgaaaattcatgtttttctgcaaattttgaccaaattcccaagaaaaatacgattttttagtccgaaaatcctccaaaaagatttctaggccaccaaaaaggtttctaggccaccaagaaagtttctaggccaccaaagtatttataggccacctaagatgtttctaggccacctgagatgtttctaggtcaccaaaaatgtttctcggtcaccaaaaatgtttcaaggccaccgaaaaggtttctaggccacctaagtatttctaggccacctaagatgtttctaggccacctgagatgtttctaggtcaccaaaaatgtttctaggttaccaaaaatgtttcaaggccatcgaaaaggtttctaggccaccaaagtatttctaggccacctaagatgtttctaggccacctgagatgtttctaggtcaccaaaaatgtttcaaggccaccgaaaaggtttctaggccaccaaaaaggtttctaggccaccaaaaatatttctaggccacctaagatgtttctaggccacctgagatgtttctaggccacctgagatgtttctaggccacctgagatgtttctaggtcaccaaaaatgtttctcggtcaccaaaaatgtttcaaggccaccgaaaaggtttctaggccacctaagtatttctaggccacctaagatgtttctaggccacctgagatgtttctaggtcaccaaaaatgtttctaggttaccaaaaatgtttcaaggccatcgaaaaggtttctaggccaccaaagtatttctaggccacctaagatgtttctaggccacctgagatgtttctaggtcaccaaaaatgtttcaaggccaccgaaaaggtttctaggccaccaaaaaggtttctaggccaccaaaaatatttctaggccaccaaaaatgtttctaggtcaccaaaaatgtttctaggtcaccaaaaatgtatcaaggccaccaaaaaggtttctaggtcaccaaaaatgtttctaggccaccaaaaatgtttctaggtcaccaaaaatgtttctaggccaccaaaaaggtttctaggccaccaaaaaggtttctaggccaccaaaaaggtttctaggccaccaaaaaggtttcaaggccaccaaaaaggtttctaggccaccaaaaatgtttctaggtcaccaaaaatgtttctaggccaccaaagtatttctaggccacctaaaaggtttctaggccatcaaaaaggtttctaggccatcaaaaaggattctaggccaccaaaaatatttctaggccacctaagatgtttctaggccaccagagtatttctaggccacctaagaggtttctgggccatcaaaaaggtttcaagtccatcaaaaaggtttctaggccaccaaaaaggtttctaggccaccgaaaaggtttctaggccaccaaaaaggtttctagaccacctaagacatttctaggccaacaaaaaggtttctaggccaccaagaagccgaaaaactgtctcaaattcgaattttgcagtgctcaaacaaaaagtgtccgcactgacagaagagctgaaaaaggagaagctggctcacgcgggaacccgttcagcattgaaagaattgactaatgaaataactggaatgcgtgtacaaatgaataaactacgttcaatggtcactcagcctacgacttcgaaaattattgatagttttgttcaacgtcatcaggctttcgagcagcaacaacaattccaacaccaacaccaccaacaccgaccaataatgttggctccacgtcatcatccgccgccgcccccgcattttacaccgaatcaacgggcggcggctccgtatcatccgaatatggttcaaccgaatcgtcttgctgctatgccacatagaagaccgattattggaatgcaggtgaaaatggaatgccatgaaaatttcgggccggaaaattttggaaaatcctctaaattttcaatatttgtcgaaaaaatctgacaaaaatcgtgtcaaaattcagatttccgggagaaaaatcgcatttttgagtaaaaattcgaagaaaagcgtcttaaattctagatttattagttaaaatttttttcaaattttagtcaagaaaattaagaaaaatgcgaaaatttcgagcaaaaaatatagttttttggagccgaaattgtgaaaaatgcgatttttttcgaaaaatctggacaaaaaatttcaaacaagaaaaaccacttttttaaaaaaattttcacacaatttccagcaacaaaattcggctccaccacaattcaacggtcaccaagctctcgtcccatcacctcaatcatcatctgcattttctcgtccaccaccaactcaacttgcaacacagagaagagctccaccattggcaagtaccggccttccggcaacagtcagatgggaagcaattccaccgccaaaaaatccgaatgtcgggcacaatgagccaccgcttaacaatggaggttcgtcgtgtgcaacaaaaagagcaccgcttttccacgacgagtttttgcgatgatgattttggtgtgaaaattgaaaaactcatttttttaaagtctgaaatttgaaaatttgagaaaagttttttaaaaaaagttttatgagggattttctgacaattttttataaacggaaaattacgaaaactccaaaatttgtgttctttcggaaaacgaatttgaaatttgaaccaaaattttgacaattttctggggatttttgactggaaattcgtttttcatcgatttttcctcctttaattttcggtaaaacccctgtctccaattccaggccgtgcacagccactaatcgataatacacgtgtacacgacaatacaattatgctgtgtgtaccacttgtctccactgcaaatacaatatcatcgggcgattcgacacgtctaccaaaagtaccacgaatctacgagaatctcacggcaaatcccgatttgagtgtgacgattcattcgagtgcacaggatttccgagagaattatcaaattggtggaaagattaactatgaatatctcggaggatttgatcaatatgtaggtgatgatgtttttttattgagagataaatacgaaattccattacaatcgatattttttgactgaaaaatgtctgaaaaatcaaaaattttagctaaaaattgagaatatttttgtttaaaaaaaatcattgaaattgatttttttttattccataaaaatctcggaaaagtcaattttcagtcataaatcttctgaaaattatccaaacaatgggattttctgaaattttagcttaaaaattgaggatttcccggttttttcagagaaattccattacaatcgatttttttactgaaaaatcctctggaaattaacaaaaaccaaataaaatgccctaatttttttttaaatccaaaaattgttggattttttcagaaaaaaatattttttcaattgactggtgtccaaaaaatatagaaaattcaaattttccaagaaaaatagccaaaaaaatgtaatttttgtctaacaaaaaaattgaatagcgcaaaattaaattgtcgttttttttaatttccctccggttttgaaaggaaaaaattccataaaaatcgaaattttttgactgaaaaatccatgaaaactcgaattttgagtcaaaaatcctctgaaaatgctccaaaatatgagattttctgaaatttcatcaaaaattaagaatttcacggtttaaaaaaaattccattaaaatcgatatttttcaagtgaaaaatctctggaaaactcgatgtttgagtcaaaattcgtctgaaaatgctcctttaaattgaaaaattgaaaaaaaaaccgcccacaatatttgcagaatatccaagtgttcgtccaagtgtcatctcttaaattcactggaatgaacggttacccggatccagaagatcgtatatcaattgactggggatgctcgaaattgtggccttgtaagccgaaatctcatcacaaattccgtgtacgcttccatcaagcacaactgctgccgaagaacgatcgaattacgattgtggctgtggcgaaggataaaactagcggaattattcacatttcgcaggtgaaaaattggaaaatttgcacaaatccagacaaaaaaaactgaaaaatcgaaaaaatttttgtaattttttgccgaaaacgaaaattaaaaactgataaaaattgatttttaaccggaaaatccctgaaaaatcaaacattttttgctaaaaattgagaattatacggttttgggtaaaaaaaaactatttaaaaaaaatattttttctttaaaaatctcaacaaaaaaaaaaccaattttcattcagaaatccccccggagaattgtcaaaattttgggaatactctgaaatttcgataaacacctcatttttgattaaaattgattttttaactgaaaaatcccttaaaaaacgaatattttagttttttcacaaaaaaatgtgcaatttatctgaaatttcagcaaaaaaaatgaaaaaaaaaaattccgaaattaaaaactgataaaaatcgattttttacttgaaaaattcgtgaaaaatcaaacacatttttgctaaccattgagaatattacgattttgtgaaaaaaaaaaccattaaaattgattttttattcctaaaaaatgccagaaaaatcaattttcagtcaaaaatcaccggaaaattatcaaaattttgaggttttctgtgaaatttcaagctgaaatttccatttttgaataaaaaaaatgtggctggatttaaaaaaaaaccattaaaattgattttttaactgaaaaatccgtatttctctgaaatttcaggcaaaaaatgtcatttccgaaattaaaaattgcgacaaaatcaaataaaattgatcaaatttgcaaaaaaaaaaaaactttcgcaaaaaatccttaaaatttacatttttcaacaaaaactcgaattttcagtcaaaaattcgtctgaaaatgctccaaaatatgggattttttgaaattttagctaaaaattgagaattgcacggtatttagagagggaaaaattccataaaaatcgatattttcctctttaaaatctcgaaaaaaatcatcaattttcattcaaaaatcccccccggaaaattgtcaaaattttgagatttttctgaaatttcacgcaaaaattttcattttttcagcccaccttcatcactctcgaatgatcgatctcttcacgtcaaatgcacttttttctggatttttttgttaaaaaatttgaaattctcgtgttttttcttctgaaaaattgctttttttgattttttctgtaattttttttttgttgattttcttaatttttttaattttcaaaaaatctttttcatctctttctctctctctctgaatctcaattttttcctgaatttccccgtttttttctgataattttcaatatttctctgaatttttctattccccccgttgtaatgccaaaatatgtggtaatttctccccattttttcgctttattactatttattctattcaattggtgcctctctcaatgtgttgtatgaaaaacactgttttatggaggttttggagaattttgaattttttcgtcgtgatttttattggttttctttaccaattcaattttttttttaattcgaaaatttgtagaaattcacttttgtagcttaaaaaattaaaaattgagaaaatttgttcaaaaatggcaaagttttcgaaattttagtctaaaaaaagatttttttaatatagaattttaaaaaattagcacagaaaaatgccgaaaaattcgtaatttttcatttaaaaatgaaaaaaaaaaaaaacaaaaaaaaaaaaaaaaaagagggaaaaatcccattaaaagtagttttttgactgcaaaatcgtctggaaattaacaaaatttaaaaaaatcttttttacagcccatcgtttccaaaaaccaaataaaatgccaaaaaaaaatttttatgcaaaaattctggatttttttccgattttttcaaaaaattcccccttctaaaaaaaatggtgaatttgttcccaaaaacccaaaatttgagattttctaaaattttggcaaaaattaagaatttcacggttttgagagggaaaaactccattaaaattgatgattttatgactaaaaattcctaaaaaatcaattttcagtcaaaaattaaattt

SEQ ID NO: 29 - protein, 728 aa, Caenorhabditis elegans
MSEVIDESILNTEASDDPIPPLNDDQIAELLGEDGEIMEITEQKDESDDVVMLDDDDDDTPEPILVIDMDEDEDVTTDGPESQEELAADAPAPGAPEASAPAQEASEASAPDQEAPEVQDVPDSSGAPDASAQASEASDASAPEVPGSTEAQDAQDVPDSLGASDASAQEIPEAPEAPEAPEIAAEIDEEVLLAEQNGVLDEGFDETDDIIIEEEAVEEAEAVEPPINTENQENALEMLEERLKKNEEKEIVEKSDVKPEDEDIIHMETDSVETSSRKRTGGATSPRSPAQKRPKRRVQTLLKMRQNAIELLTRLYGSWDAQLSLSNLETIRLLGVNNNRKLIEIFEENEQVLKQKVSALTEELKKEKLAHAGTRSALKELTNEITGMRVQMNKLRSMVTQPTTSKIIDSFVQRHQAFEQQQQFQHQHHQHRPIMLAPRHHPPPPPHFTPNQRAAAPYHPNMVQPNRLAAMPHRRPIIGMQQQNSAPPQFNGHQALVPSPQSSSAFSRPPPTQLATQRRAPPLASTGLPATVRWEAIPPPKNPNVGHNEPPLNNGGRAQPLIDNTRVHDNTIMLCVPLVSTANTISSGDSTRLPKVPRIYENLTANPDLSVTIHSSAQDFRENYQIGGKINYEYLGGFDQYNIQVFVQVSSLKFTGMNGYPDPEDRISIDWGCSKLWPCKPKSHHKFRVRFHQAQLLPKNDRITIVAVAKDKTSGIIHISQPTFITLE

SEQ ID NO: 30 - DNA, 11 bp, Caenorhabditis elegans
aagatatgtgt

SEQ ID NO: 31 - DNA, 11 bp, Caenorhabditis elegans
aacttcaaaat

SEQ ID NO: 32 - DNA, 11 bp, Caenorhabditis elegans
cttataagttt

SEQ ID NO: 33 - DNA, 11 bp, Caenorhabditis elegans
ttttccaaaaa

SEQ ID NO: 34 - DNA, 11 bp, Caenorhabditis elegans
ttttttaagat

SEQ ID NO: 35 - DNA, 6403 bp, Caenorhabditis elegans
aaggaattagactctttatctaaagtgaagaatgatcaattaagaagtttttgtcccatagaattaaatataaatggatctcctggggcagaatctgatttggcaacattttgcacttctaaaactgatgctgttttaatgacttctgatgatagtgtgactggatcggaattatcccctttggtcaaagcatgcatgctttcatcaaatggatttcagaatattagtaggtgcaaagaaaaagacttggatgatacctgcatgctgcataagaagtcagaaagcccatttagagaaacagaacctctggtgtcaccacaccaagataaactcatgtctatgccagttatgactgtggattattccaaaacagtagttaaagaaccagttgatacgagggtttcttgctgcaaaaccaaagattcagacatatactgtactttgaacgatagcaacccttctttgtgtaactctgaagctgaaaatattgagccttcagttatgaagatttcttcaaatagctttatgaatgtgcatttggaatcaaaaccagttatatgtgatagtagaaatttgacagatcactcaaaatttgcatgtgaagaatataagcagagcatcggtagcactagttcagcttctgttaatcattttgatgatttatatcaacctattgggagttcaggtattgcttcatctcttcagagtcttccaccaggaataaaggtggacagtctaactctcttgaaatgcggagagaacacatctccagttctggatgcagtgctaaagagtaaaaaaagttcagagtttttaaagcatgcagggaaagaaacaatagtagaagtaggtagtgaccttcctgattcaggaaagggatttgcttccagggagaacaggcgtaataatgggttatctgggaaatgtttgcaagaggctcaagaagaagggaattccatattgcctgaaagaagaggaagaccagaaatctctttagatgaaagaggagaaggaggacatgtgcatacttctgatgactcagaagttgtattttcttcttgtgatttgaatttaaccatggaagacagtgatggtgtaacttatgcattaaagtgtgacagtagtggtcatgccccagaaattgtgtctacagttcatgaagattattctggctcttctgaaagttcaaatgatgaaagtgattcagaagatacagattcggatgatagcagtattccaagaaaccgtctccagtctgttgtggttgtgccaaagaattctactttgcccatggaagaaacaagtccttgttcttctcggagcagtcaaagttatagacactattctgaccattgggaagatgagagattggagtcaaggagacatttgtatgaggaaaaatttgaaagtatagcaagtaaagcctgtcctcaaactgataagtttttccttcataaaggaacagagaagaatccggaaatttcttttacacagtccagtagaaaacaaatagataaccgcctgcctgaactttctcatcctcagagtgatggggttgatagtacaagtcatacagatgtgaaatctgaccctctgggtcacccaaattcagaggaaaccgtgaaagccaaaataccttctaggcagcaagaagagctgccaatttattcttctgattttgaagatgtcccaaataagtcttggcaacagaccactttccaaaacaggccagatagtagactgggaaaaacagaattgagtttttcttcctcttgtgagataccacatgtggatggcttgcactcatcagaagagctcagaaacttaggttgggacttctctcaagaaaagccttctaccacgtatcagcaacctgacagtagctatggagcttgtggtggacacaagtatcagcaaaatgcagaacagtatggtgggacacgtgattactggcaaggcaatggttactgggatccaagatcaggtagacctcctggaactggggttgtgtatgatcgaactcaaggacaagtaccagattccctaacagatgatcgtgaagaagaggagaattgggatcaacaggatggatcccatttttcagaccagtccgataaatttcttctatcccttcagaaagacaaggggtcagtgcaagcacctgaaataagcagcaattccattaaggacactttagctgtgaatgaaaagaaagatttttcaaaaaacttagaaaaaaatgatatcaaagatagagggcctcttaaaaaaaggaggcaggaaatagagagtgattctgaaagtgatggtgagcttcaggacagaaagaaagttagagtggaggtagagcagggagagacatcagtgcccccaggttcagcactggttgggccctcctgtgtcatggatgacttcagggacccacagcgatggaaggaatgtgccaagcaagggaaaatgccatgttactttgatcttattgaagaaaatgtttatttaacagaaagaaagaagaataaatctcatcgagatattaagcgaatgcagtgtgagtgtacacctctttctaaagatgaaagagctcaaggtgaaatagcatgtggggaagattgtcttaatcgtcttctcatgattgaatgttcttctcggtgtccaaatggggattattgttccaatagacggtttcagagaaaacagcatgcagatgtggaagtcatactcacagaaaagaaaggctggggcttgagagctgccaaagaccttccttcgaacacctttgtcctagaatattgtggagaggtactcgatcataaagagtttaaagctcgagtgaaggagtatgcacgaaacaaaaacatccattactatttcatggccctgaagaatgatgagataatagatgccactcaaaaaggaaattgctctcgtttcatgaatcacagctgtgaaccaaattgtgaaacccaaaaatggactgtgaacggacaactgagggttgggttttttaccaccaaactggttccttcaggctcagagttaacgtttgactatcagttccagagatatggaaaagaagcccagaaatgtttctgcggatcagccaattgccggggttacctgggaggagaaaacagagtcagcatcagagcagcaggagggaaaatgaagaaggaacgatctcgtaagaaggattcagtggatggagagctagaagctctgatggaaaatggtgagggtctctctgataaaaaccaggtgctcagcttatcccggctaatggttagaattgaaactttggagcagaaacttacctgtctggaactcatacagaacacacactcacagtcctgcctgaagtcctttctggaacgtcatgggctgtctttgttgtggatctggatggcagagctaggtgacggccgggaaagtaaccagaagcttcaggaagagattataaagactttggaacacttgcccattcctactaaaaatatgttggaggaaagcaaagtacttccaattattcaacgctggtctcagactaagactgctgtccctccgttgagtgaaggagatgggtattctagtgagaatacatcgcgtgctcatacaccactcaacacacctgatccttccaccaagctgagcacagaagctgacacagacactcccaagaaactaatgtttcgcagactgaaaattataagtgaaaatagcatggacagtgcaatctctgatgcaaccagtgagctagaaggcaaggatggcaaagaggatcttgatcaattagaaaatgtccctgtagaggaagaggaagaattgcagtcacaacagctactcccacaacagctgcctgaatgcaaagttgatagtgaaaccaacatagaagctagtaagctacctacatctgaaccagaagctgacgctgaaatagagctcaaagagagcaacggcacaaaactagaagaacctattaatgaagaaacaccatcccaagatgaagaggagggtgtgtctgatgtggagagtgaaaggagccaagaacagccagataaaacagtggatataagtgatttggccaccaaactcctggacagttggaaagacctaaaggaggtatatcgaattccaaagaaaagtcaaactgaaaaggaaaacacaacaactgaacgaggaagggatgctgttggcttcagagatcaaacacctgccccgaagactcctaataggtcaagagagagagacccagacaagcaaactcaaaataaagagaaaaggaaacgaagaagctccctctcaccaccctcttctgcctatgagcggggaacaaaaaggccagatgacagatatgatacaccaacttctaaaaagaaagtacgaattaaagaccgcaataaactttctacagaggaacgccggaagttgtttgagcaagaggtggctcaacgggaggctcagaaacaacagcaacagatgcagaacctgggaatgacatcaccactgccctatgactctcttggttataatgccccgcatcatccctttgctggttacccaccaggttatcccatgcaggcctatgtggatcccagcaaccctaatgctggaaaggtgctcctgcccacacccagcatggacccagtgtgttctcctgctccttatgatcatgctcagcccttggtgggacattctacagaacccctttctgcccctccaccagtaccagtggtgccacatgtggcagctcctgtggaagtttccagttcccagtatgtggcccagagtgatggtgtagtacaccaagactccagcgttgctgtcttgccagtgccggcccccggcccagttcagggacagaattatagtgtttgggattcaaaccaacagtctgtcagtgtacagcagcagtactctcctgcacagtctcaagcaaccatatattatcaaggacagacatgtccaacagtctatggtgtgacatcaccttattcacagacaactccaccaattgtacagagttatgcccagccaagtcttcagtatatccaggggcaacagattttcacagctcatccacaaggagtggtggtacagccagccgcagcagtgactacaatagttgcaccagggcagcctcagcccttgcagccatctgaaatggttgtgacaaataatctcttggatctgccgcccccctctcctcccaaaccaaaaaccattgtcttacctcccaactggaagacagctcgagatccagaagggaagatttattactaccatgtgatcacaaggcagactcagtgggatcctcctacttgggaaagcccaggagatgatgccagccttgagcatgaagctgagatggacctgggaactccaacatatgatgaaaaccccatgaaggcctcgaaaaagcccaagacagcagaagcagacacctccagtgaactagcaaagaaaagcaaagaagtattcagaaaagagatgtcccagttcatcgtccagtgcctgaacccttaccggaaacctgactgcaaagtgggaagaattaccacaactgaagactttaaacatctggctcgcaagctgactcacggtgttatgaataaggagctgaagtactgtaagaatcctgaggacctggagtgcaatgagaatgtgaaacacaaaaccaaggagtacattaagaagtacatgcagaagtttggggctgtttacaaacccaaagaggacactgaattagagtgactgttgggccagggtgggaggatgggtggtcaggtaagacagactctagggagaggaaatcctgtgggcctttctgtcccacccctgtcagcactgtgctactgatgatacatcaccctggggaattcaaccctgcagatgtcaactgaaggccacaaaaatgaactccatctacaagtgattacctagttgtgagctgttggcatgtggttagaagccatcagaggtgcaagggcttagaaaagaccctggccagacctgactccactcttaaacctgggtcttctccttggcggtgctgtcagcgcacagacccatgcgcatccccacccacaaccctttaccctgatgatctgtattatattttaatgtatatgtgaatatattgaaaataatttgttttttcctggtttttgtttggttttcgttttgcttttagcctctacatgctaggatcacaggaagactttgtaaggacagtttaagttctcctgcaaggtttaatttgttatcatgtaaatattccaaagcaggctgccttgtggttttggccagccttgtgctatgttgataagattgatttactgcttaaaatcactttactttatccaatttttactgaactttttatgtaaaaaaataaaatcaattaaag

SEQ ID NO: 36 - protein, 1915 aa, Caenorhabditis elegans
KELDSLSKVKNDQLRSFCPIELNINGSPGAESDLATFCTSKTDAVLMTSDDSVTGSELSPLVKACMLSSNGFQNISRCKEKDLDDTCMLHKKSESPFRETEPLVSPHQDKLMSMPVMTVDYSKTVVKEPVDTRVSCCKTKDSDIYCTLNDSNPSLCNSEAENIEPSVMKISSNSFMNVHLESKPVICDSRNLTDHSKFACEEYKQSIGSTSSASVNHFDDLYQPIGSSGIASSLQSLPPGIKVDSLTLLKCGENTSPVLDAVLKSKKSSEFLKHAGKETIVEVGSDLPDSGKGFASRENRRNNGLSGKCLQEAQEEGNSILPERRGRPEISLDERGEGGHVHTSDDSEVVFSSCDLNLTMEDSDGVTYALKCDSSGHAPEIVSTVHEDYSGSSESSNDESDSEDTDSDDSSIPRNRLQSVVVVPKNSTLPMEETSPCSSRSSQSYRHYSDHWEDERLESRRHLYEEKFESIASKACPQTDKFFLHKGTEKNPEISFTQSSRKQIDNRLPELSHPQSDGVDSTSHTDVKSDPLGHPNSEETVKAKIPSRQQEELPIYSSDFEDVPNKSWQQTTFQNRPDSRLGKTELSFSSSCEIPHVDGLHSSEELRNLGWDFSQEKPSTTYQQPDSSYGACGGHKYQQNAEQYGGTRDYWQGNGYWDPRSGRPPGTGVVYDRTQGQVPDSLTDDREEEENWDQQDGSHFSDQSDKFLLSLQKDKGSVQAPEISSNSIKDTLAVNEKKDFSKNLEKNDIKDRGPLKKRRQEIESDSESDGELQDRKKVRVEVEQGETSVPPGSALVGPSCVMDDFRDPQRWKECAKQGKMPCYFDLIEENVYLTERKKNKSHRDIKRMQCECTPLSKDERAQGEIACGEDCLNRLLMIECSSRCPNGDYCSNRRFQRKQHADVEVILTEKKGWGLRAAKDLPSNTFVLEYCGEVLDHKEFKARVKEYARNKNIHYYFMALKNDEIIDATQKGNCSRFMNHSCEPNCETQKWTVNGQLRVGFFTTKLVPSGSELTFDYQFQRYGKEAQKCFCGSANCRGYLGGENRVSIRAAGGKMKKERSRKKDSVDGELEALMENGEGLSDKNQVLSLSRLMVRIETLEQKLTCLELIQNTHSQSCLKSFLERHGLSLLWIWMAELGDGRESNQKLQEEIIKTLEHLPIPTKNMLEESKVLPIIQRWSQTKTAVPPLSEGDGYSSENTSRAHTPLNTPDPSTKLSTEADTDTPKKLMFRRLKIISENSMDSAISDATSELEGKDGKEDLDQLENVPVEEEEELQSQQLLPQQLPECKVDSETNIEASKLPTSEPEADAEIELKESNGTKLEEPINEETPSQDEEEGVSDVESERSQEQPDKTVDISDLATKLLDSWKDLKEVYRIPKKSQTEKENTTTERGRDAVGFRDQTPAPKTPNRSRERDPDKQTQNKEKRKRRSSLSPPSSAYERGTKRPDDRYDTPTSKKKVRIKDRNKLSTEERRKLFEQEVAQREAQKQQQQMQNLGMTSPLPYDSLGYNAPHHPFAGYPPGYPMQAYVDPSNPNAGKVLLPTPSMDPVCSPAPYDHAQPLVGHSTEPLSAPPPVPVVPHVAAPVEVSSSQYVAQSDGVVHQDSSVAVLPVPAPGPVQGQNYSVWDSNQQSVSVQQQYSPAQSQATIYYQGQTCPTVYGVTSPYSQTTPPIVQSYAQPSLQYIQGQQIFTAHPQGVVVQPAAAVTTIVAPGQPQPLQPSEMVVTNNLLDLPPPSPPKPKTIVLPPNWKTARDPEGKIYYYHVITRQTQWDPPTWESPGDDASLEHEAEMDLGTPTYDENPMKASKKPKTAEADTSSELAKKSKEVFRKEMSQFIVQCLNPYRKPDCKVGRITTTEDFKHLARKLTHGVMNKELKYCKNPEDLECNENVKHKTKEYIKKYMQKFGAVYKPKEDTELE

(19) United States
(12) Patent Application Publication
(10) Pub. No.: US 2005/0069896 A1
(43) Pub. Date: Mar. 31, 2005

(54) Rb pathway and chromatin remodeling genes that antagonize let-60 Ras signaling

(76) Inventors: H. Robert Horvitz, Auburndale, MA (US); Craig Ceol, Roslindale, MA (US); Erik Andersen, Somerville, MA (US)

Correspondence Address: CLARK & ELBING LLP, 101 FEDERAL STREET, BOSTON, MA, 02110, US

(21) Appl. No.: 10/661,398

(22) Filed: Sep. 12, 2003

Related U.S. Application Data
(60) Provisional application No. 60/437,821, filed on Jan. 2, 2003.
(60) Provisional application No. 60/410,160, filed on Sep. 12, 2002.

(51) Int. Cl. C12Q 1/68; G01N 33/574

(52) U.S. Cl. 435/6; 435/7.23

(57) ABSTRACT

In general, the invention provides methods and compositions useful in the treatment of a neoplasia. These compositions include new components of the Rb pathway that function in chromatin remodeling and antagonize Ras signaling.

### CROSS-REFERENCE TO RELATED APPLICATIONS

This application claims the benefit of U.S. Provisional Application Nos: 60/437,821, filed Jan. 2, 2003, and 60/410,160, filed Sep. 12, 2002.

### STATEMENT AS TO FEDERALLY SPONSORED RESEARCH

This work was supported in part by the National Institutes of Health (Grant No. GM24663). The government may have certain rights to this invention.

### BACKGROUND OF THE INVENTION

In general, the invention features methods and compositions useful in the treatment of a neoplasia.

Retinoblastoma (Rb) family proteins are mammalian tumor suppressors that regulate cell proliferation. This pathway is conserved among a variety of species, including the nematode, Caenorhabditis elegans. LIN-35 Rb, which is the nematode C. elegans counterpart of mammalian Rb, is required for normal vulval development in C. elegans. C. elegans vulval development also requires the activity of a conserved Ras signaling pathway. Mutations that disable let-60 Ras and other genes in this pathway result in a vulvaless (Vul) phenotype. Mutations that overactivate this pathway, for instance mutations that create the same G1 3E substitution found in oncogenic forms of human Ras, cause a multivulva (Muv) phenotype that is characterized by excessive induction of vulval cell fates, leading to worms having multiple vulvae.

Lin-35 Rb is a synthetic multivulva synMuv gene. The synthetic multivulva (synMuv) genes antagonize the Ras signaling pathway that induces vulval development in the nematode C. elegans. The synMuv genes are grouped into two classes, A and B, such that a mutation in a gene of each class is required to produce a multivulva phenotype. The class B synMuv genes include homologs of other genes that function with Rb in transcriptional regulation. Many synMuv genes have been cloned and molecularly characterized. Loss-of-function mutations in two functionally redundant pathways that are encoded by the class A and class B synthetic multivulva (synMuv) genes also cause a Muv phenotype.

In addition to LIN-35 Rb, other proteins with class B synMuv activity are homologous to mammalian Rb-associated proteins. These other proteins include DPL-1 and EFL-1, homologs of DP and E2F transcription factors, LIN-53, a homolog of the Rb-binding proteins RbAp46 and RbAp48, HDA-1, a histone deacetylase homolog and HPL-2, a heterochromatin protein 1 homolog. The class B synMuv proteins act together to negatively regulate the transcription of genes that promote vulval development. Initially, DPL-1 and EFL-1 heterodimers bind DNA at specific regulatory sequences of vulval cell-fate determination genes. DNA-bound DPL-1 and EFL-1 heterodimers recruit LIN-35 Rb, which in turn recruits proteins that act to remodel chromatin. One of these proteins, HDA-1, is predicted to deacetylate lysines of nucleosomal histones. Deacetylation of lysine residues is required for their subsequent methylation. HPL-2, another protein that may be recruited by LIN-35 Rb, is expected to act like other HP1 family proteins and bind, via its chromodomain, to methylated lysine residues of nucleosomal histones.

Given the similarities that exist between C. elegans and mammalian Rb and Ras pathways, C. elegans provides an efficient, inexpensive, and facile screening tool to identify novel clinical targets and chemotherapeutics useful in the treatment of neoplasia.

### SUMMARY OF THE INVENTION

The invention provides compositions useful in treating a neoplasia and methods for identifying chemotherapeutic agents.

In one aspect, the invention features a method for identifying a compound that treats a neoplasia, the method involves (a) contacting a cell containing a mutation in a Class B synMuv gene selected from the group consisting of: mep-1, lin(n3628), lin(n4256), and lin-65 and a second mutation in a synthetic muiltivulval gene, or an ortholog thereof, with a candidate compound; and (b) detecting a phenotypic alteration in the contacted cell relative to a control cell; where a candidate compound that alters the phenotype of the contacted cell relative to the control cell is a compound that treats a neoplasia. In one embodiment, the cell is in a nematode. In another embodiment, the phenotypic alteration is an alteration in a multivulval phenotype. In another embodiment, the phenotypic alteration is an alteration in sterility. In another embodiment, the second mutation is in a synMuv class A gene. In another embodiment, the cell is an isolated mammalian cell. In another embodiment, the phenotypic alteration is a decrease in cell proliferation.

In another aspect, the invention provides a method for identifying a candidate compound that treats a neoplasia, the method involves (a) providing a cell having a mutation in a Class B synMuv gene selected from the group consisting of mep-1, lin(n3628), lin(n4256), and lin-65 and having a second mutation in a synMuv nucleic acid or ortholog thereof; (b) contacting the cell with a candidate compound; and (c) detecting a decrease in proliferation of the cell contacted with the candidate compound relative to a control cell not contacted with the candidate compound, where a decrease in proliferation identifies the candidate compound as a candidate compound that treats a neoplasia. In one embodiment, the cell is in a nematode. In another embodiment, the decrease in proliferation is detected by detecting inhibition of a Muv phenotype. In another embodiment, the cell has a mutation in Dp, E2F, or histone deaceytlase. In another embodiment, the cell is an isolated mammalian cell.

In another aspect, the invention provides a method of identifying a compound that treats a neoplasia, the method involves (a) providing a cell expressing a nucleic acid having at least 95% identity to a Class B synMuv gene selected from the group consisting of: mep-1, lin(n3628), lin(n4256), and lin-65; (b) contacting the cell with a candidate compound; and (c) monitoring the expression of the nucleic acid, an alteration in the level of expression of the nucleic acid indicates that the candidate compound is a compound that treats a neoplasia. In one embodiment, the gene contains a reporter gene (e.g., lacZ, gfpf CAT, or luciferase). In another embodiment, expression is monitored by assaying protein level. In another embodiment, the expression is monitored by assaying nucleic acid level. In yet another embodiment, the cell is in a nematode.

In another aspect, the invention features a method for identifying a candidate compound that treats a neoplasia, the method involves (a) providing a cell expressing a Class B synMuv gene selected from the group consisting of: mep-1, lin(n3628), lin(n4256), and lin-65; (b) contacting the cell with a candidate compound; and (c) comparing the expression of the polypeptide in the cell contacted with the candidate compound to a control cell not contacted with the candidate compound, where an increase in the expression of the polypeptide identifies the candidate compound as a candidate compound that treats a neoplasia. In one embodiment, the cell is in a nematode. In another embodiment, the expression is monitored with an immunological assay.

In another aspect, the invention features a method for identifying a candidate compound that treats a neoplasia, the method involves (a) providing a cell expressing a Class B synMuv polypeptide selected from the group consisting of: MEP-1, LIN(n3628), LIN(n4256), and LIN-65, the method involves; (b) contacting the cell with a candidate compound; and (c) comparing the biological activity of the polypeptide in the cell contacted with the candidate compound to a control cell not contacted with the candidate compound, where an increase in the biological activity of the polypeptide identifies the candidate compound as a candidate compound-that treats a-neoplasia. In another embodiment, the biological activity is monitored with an enzymatic assay. In another embodiment, the biological activity is monitored with an immunological assay. In yet another embodiment, the biological activity is monitored with a nematode bioassay.

In another aspect, the invention features a method of identifying a nucleic acid target of class B synMuv biological activity, the method involves (a) mutagenizing a C. elegans containing mutations in a Class B synMuv gene selected from the group consisting of: mep-1, lin(n3628), lin(n4256), and lin-65 and in a Class A synMuv gene; (b) allowing the C. elegans to reproduce; and (c) selecting a C. elegans containing a mutation that suppresses a synMuv phenotype; where the mutation identifies a nucleic acid target of class B synMuv biological activity.

In another aspect, the invention features a method of identifying a nucleic acid target of class B synMuv biological activity, the method involves (a) providing a microarray containing fragments of nematode nucleic acids; (b) contacting the microarray with detectably labeled nucleic acids derived from a nematode containing a mutation in a Class B synMuv gene selected from the group consisting of: mep-1, lin(n3628), lin(n4256), and lin-65 gene; (c) detecting an alteration in the expression of at least one nucleic acid of a C. elegans containing a mutation in the Class B synMuv gene relative to the expression of the nucleic acid in a control nematode, where an alteration in the expression identifies the nucleic acid as a nucleic acid target of class B synMuv biological activity. In one embodiment, the C. elegans further contains a mutation in a second synMuv gene. In another embodiment, the C. elegans further contains a mutation in a gene that results in a Vulvaless (Vul) phenotype.

In another aspect, the invention features a method for identifying a nucleic acid that binds a synMuv class B polypeptide, the method involves (a) providing nucleic acids derived from a nematode cell; (b) crosslinking the nucleic acids and their associated proteins to form a nucleic acid-protein complex; (c) contacting the nucleic acid-protein complex with an antibody against a polypeptide selected from the group consisting of MEP-1, LIN(n3628), LIN(n4256), and LIN-65; (d) purifying the nucleic acid-protein complex using an immunological -method; and (e) isolating the nucleic acid, where the isolated nucleic acid is a nucleic acid that binds a synMuv class B polypeptide. In one embodiment, the method further involves the following steps: (f) detectably labeling the nucleic acid of step (e); (g) contacting a microarray containing C. elegans nucleic acid fragments with the detectably labeled nucleic acid; and (h) detecting binding of the detectably labeled nucleic acid, where the binding identifies the nucleic acid as a nucleic acid that binds a synMuv class B polypeptide.

In another aspect, the invention provides a vector containing a nucleic acid having at least 95% identity to a Class B synMuv gene selected from the group consisting of: mep-1, lin(n3628), lin(n4256), and lin-65. In one embodiment, the synMuv gene is mep-1 (SEQ ID NO:2). In one embodiment, the synMuv gene contains a mutation selected from the group consisting of n3680, n3702, and n3703. In other embodiments, the synMuv gene is lin(n3628(SEQ ID NO:24), lin(n4256) (SEQ ID NO:26), or lin-65 (SEQ ID NO:28).

In another aspect, the invention provides an isolated cell containing the vector of the previous aspect.

In a related aspect, the invention provides a nematode containing the nucleic acid of the previous aspect.

In another aspect, the invention provides a nematode containing a mutation in a Class B synMuv gene selected from the group consisting of: mep-1, lin(n3628), lin(n4256), and lin-65. In one embodiment, the mutation is a mep-1 mutation selected from the group consisting of n3680, n3702, and n3703.

In another aspect, the invention features a purified nucleic acid containing a sequence that hybridizes under high stringency conditions to a Class B synMuv nucleic acid selected from the group consisting of: mep-1, lin(n3628), lin(n4256), and lin-65.

In another aspect, the invention features an antibody against a Class B synMuv polypeptide selected from the group consisting of: MEP-1, LIN(n3628), LIN(n4256), and LIN-65.

In another-aspect, the invention provides a method -for identifying a-compound that treats a condition characterized by inappropriate cell death, the method involves (a) contacting a nematode containing a mutation in a Class B synMuv gene selected from the group consisting of: mep-1, lin(n3628), lin(n4256), and lin-65 with a candidate compound; and (b) detecting a muv phenotype in the contacted nematode relative to a control nematode; where a candidate compound that alters the phenotype of the contacted nematode relative to the control nematode is a compound that treats a condition characterized by inappropriate cell death. In one embodiment, the cell is in a nematode. In another embodiment, the alteration is an alteration in a synMuv phenotype. In another aspect, the invention provides a method for identifying a compound that treats a neoplasia, the method involves (a) contacting a cell containing a mutation in a gene encoding KIAA1732 and a second mutation in a synMuv nucleic acid, or an ortholog thereof, with a candidate compound; (b) detecting a phenotypic alteration in the contacted cell relative to a control cell; where a candidate compound that alters the phenotype of the contacted cell relative to the control cell is a compound that treats a neoplasia. In one embodiment, the synthetic multivulval gene is a synMuv class A gene. In another embodiment, the cell is an isolated mammalian cell. In another embodiment, the phenotypic alteration is a decrease in cell proliferation.

In another aspect, the invention features a method for identifying a candidate compound that treats a neoplasia, the method involves (a) providing a cell having a mutation in a nucleic acid encoding KIAA1732 and having a second mutation in a synMuv nucleic acid, or ortholog thereof; (b) contacting the cell with a candidate compound; and (c) detecting a decrease in proliferation of the cell contacted with the candidate compound relative to a control cell not contacted with the candidate compound, where a decrease in proliferation identifies the candidate compound as a candidate compound that treats a neoplasia. In one embodiment, the cell has a mutation in Dp, E2F, or histone deaceytlase. In another embodiment, the cell is an isolated mammalian cell.

In another aspect, the invention provides a method of identifying a compound that treats aneoplasia,t-he-method involves (a) providing a cell expressing a nucleic acid having at least 95% identity to a nucleic acid that encodes KIAA1732; (b) contacting the cell with a candidate compound; and (c) monitoring the expression of the nucleic acid, an alteration in the level of expression of the nucleic acid indicates that the candidate compound is a compound that treats a neoplasia. In one embodiment, the gene contains a reporter gene (e.g., lacZ, gfp, CAT, or luciferase). In another embodiment, expression is monitored by assaying protein level. In another embodiment, the expression is monitored by assaying nucleic acid level. In another embodiment, the cell is an isolated mammalian cell.

In another aspect, the invention provides a method for identifying n candidate compound that treats a neoplasia, the method involves (a) providing a cell expressing a KIAA1732 polypeptide; (b) contacting the cell with a candidate compound; and (c) comparing the expression of the polypeptide in the cell contacted with the candidate compound to a control cell not contacted with the candidate compound, where an increase in the expression of the polypeptide identifies the candidate compound as a candidate compound that treats a neoplasia. In one embodiment, the cell is an isolated mammalian cell. In another embodiment, the expression is monitored with an immunological assay.

In another aspect, the invention features a method for identifying a candidate compound that treats a neoplasia, the method involves (a) providing a cell expressing a KIAA1732 polypeptide; (b) contacting the cell with a candidate compound; and (c) comparing the biological activity of the polypeptide in the cell contacted with the candidate compound to a control cell not contacted with the candidate compound, where an increase in the biological activity of the polypeptide identifies the candidate compound as a candidate compound that treats a neoplasia. In one embodiment, the biological activity is monitored with an enzymatic assay. In another embodiment, the biological activity is monitored with an immunological assay. In another embodiment, the biological activity is methyl transferase activity.

In another aspect, the invention features a method for identifying a nucleic acid that binds KIAA1732, the method involves(a) providing nucleic acids derived from a mammalian cell; (b) crosslinking the nucleic acids and their associated proteins to form a nucleic acid-protein complex; (c) contacting the nucleic acid-protein complex with an anti-KIAA1732 antibody; (d) purifying the nucleic acid-protein complex using an immunological method; and (e) isolating the nucleic acid, where the isolated nucleic acid is a nucleic acid that binds KIAA1732. In one embodiment, the method further involves the following steps: (f) detectably labeling the nucleic acid of step (e); (g) contacting a microarray containing human nucleic acid fragments with the detectably labeled nucleic acid; and (h) detecting binding of the detectably labeled nucleic acid, where the binding identifies the nucleic acid as a nucleic acid that binds KIAA1732.

In another aspect, the invention provides a vector containing a nucleic acid having at least 95% identity to SEQ ID NO:36.

In another aspect, the invention provides an isolated cell containing the vector of the previous aspect.

In another aspect, the invention provides a method for identifying a compound that treats a neoplasia, the method involves (a) contacting a nematode containing a mutation in a Class C synMuv gene selected from the group consisting of trr-1, hat-1, epc-1, and ssl-1 with a candidate compound; and (b) detecting an alterated phenotype in the contacted nematode relative to a control nematode; where a candidate compound that alters the phenotype of the contacted nematode relative to the control nematode is a compound that treats a neoplasia. In one embodiment, the alteration is an alteration in vulval phenotype. In another embodiment, the alteration is an alteration in sterility. In another embodiment, the synMuv class C gene is trr-1. In another embodiment, the mutations are selected from the group consisting of n3630, n3637, n3704, n3708, n3709, and n3712.

In another aspect, the invention provides a method for identifying a candidate compound that treats a neoplasia, the method involves (a) providing a cell having a mutation in a Class C synMuv gene selected from the group consisting of trr-1, hat-1, epc-1, and ssl-1 and having a second mutation in a synMuv nucleic acid or ortholog thereof; (b) contacting the cell with-a-candidate compound; and (c) detecting a decreased proliferation of the cell contacted with the candidate compound relative to a control cell not contacted with the candidate compound, where a decrease in proliferation identifies the candidate compound as a candidate compound that treats a neoplasia. In one embodiment,the cell is in a nematode. In another embodiment, the nematode displays an alteration in a synMuv phenotype. In another embodiment, the cell contains a mutation in a class A or class B synMuv gene.

In another aspect, the invention provides a method for identifying a compound that treats a neoplasia, the method involves (a) contacting a nematode containing a mutation in a Class C synMuv gene selected from the group consisting of trr-1, hat-1, epc-1, and ssl-1 and a second mutation in a Class A synthetic multivulval gene with a candidate compound; and (b) detecting an altered phenotype in the contacted nematode relative to a control nematode; where a candidate compound that alters the phenotype of the contacted nematode relative to the control nematode is a compound that treats a neoplasia. In one embodiment,the alteration is an alteration in synMuv phenotype. In another embodiment, the alteration is an alteration in sterility.

In another aspect, the invention provides a method for identifying a compound that treats a neoplasia, the method involves (a) contacting a nematode containing a mutation in a Class C synMuv gene selected from the group consisting of trr-1, hat-1, epc-1, and ssl-1 and a second mutation in a Class B synthetic multivulval gene with a candidate compound; (b) detecting an altered phenotype in the contacted nematode relative to a control nematode; where a candidate compound that alters the phenotype of the contacted nematode relative to the control nematode is a compound that treats a neoplasia. In another embodiment, the alteration is an alteration in synMuv phenotype. In another embodiment, the alteration is an alteration in sterility.

In another aspect, the invention features a method for identifying a candidate compound that treats a neoplasia, the method involves (a) providing a cell having a mutation in a Class C synMuv gene selected from the group consisting of trr-1, hat-1, epc-1, and ssl-1 and having a second mutation in a synMuv gene or ortholog thereof;(b) contacting the cell with a candidate compound; and (c) detecting a decreased proliferation of the cell contacted with the candidate compound relative to a control cell not contacted with the candidate compound, where a decrease in proliferation identifies the candidate compound as a candidate compound that treats a neoplasia. In one embodiment, the cell is in a nematode. In another embodiment, the nematode displays an alteration in a synMuv phenotype.

In another aspect, the invention provides a method of identifying a compound that treats a neoplasia, the method involves (a) providing a cell expressing a nucleic acid having at least 95% identity to a Class C synMuv nucleic acid selected from the group consisting of trr-1, hat-1, epc-1, and ssl-1; (b) contacting the cell with a candidate compound; and (c) monitoring the expression of the nucleic acid, an alteration in the level of expression of the nucleic acid indicates that the candidate compound is a compound that treats a neoplasia. In one embodiment, the gene contains a reporter gene. In another embodiment, the reporter gene contains lacZ, gfp, CAT, or luciferase. In another embodiment, the expression is monitored by assaying protein level. In yet another embodiment, the expression is monitored by assaying nucleic acid level. In yet another embodiment, the nucleic acid is in a nematode.

In another aspect, the invention provides a method for identifying a candidate compound that treats a neoplasia, the method involves (a) providing a cell expressing a a Class C synMuv polypeptide selected from the group consisting of TRR-1, HAT-1, EPC-1, and SSL-1 polypeptide; (b) contacting the cell with a candidate compound; and (c) comparing the expression of the polypeptide in the cell contacted with the candidate compound to a control cell not contacted with the candidate compound, where an increase in the expression of the polypeptide identifies the candidate compound as a candidate compound that treats a neoplasia. In one embodiment, the cell is in a nematode. In another embodiment,the expression is monitored with an immunological assay.

In another-aspect, the invention provides a method for identifying a candidate compound that treats a neoplasia, the method involves (a) providing a cell expressing a Class C synMuv polypeptide selected from the group consisting of TRR-1, HAT-1, EPC-1, and SSL-1;(b) contacting the cell with a candidate compound; and (c) comparing the biological activity of the polypeptide in the cell contacted with the candidate compound to a control cell not contacted with the candidate compound, where an increase in the biological activity of the polypeptide identifies the candidate compound as a candidate compound that treats a neoplasia. In one embodiment, the cell is in a nematode. In another embodiment, the biological activity is monitored with an enzymatic assay. In another embodiment, the biological activity is monitored with an immunological assay.

In another aspect, the invention provides a method of identifying a nucleic acid target of a synMuv Class C polypeptide, the method involves (a) mutagenizing a C. elegans containing a first mutation in a Class C synMuv gene selected from the group consisting of (yr-1, halt-1, epc-1, and ssl-1 and a second mutation in a Class A or Class B synMuv gene; (b) allowing the C. elegans to reproduce; (c) selecting a C. elegans containing a mutation that suppresses a synMuv phenotype; where the mutation identifies a nucleic acid target of a synMuv class C polypeptide. In one embodiment, the second mutation is in a class A synMuv gene. In another embodiment, the second mutation is in a Class B synMuv gene.

In another aspect, the invention provides a method for identifying a a nucleic acid target of a synMuv Class C polypeptide, the method involves (a) providing a C. elegans containing a mutations in a Class C synMuv gene selected from the group consisting of trr-1, hat-1, epc-1, and ssl-1; (b) growing the C. elegans on bacteria expressing a dsRNA; and (c) identifying a dsRNA that suppresses a synMuv phenotype; where the dsRNA identifies a nucleic acid target of a synMuv class C polypeptide.

In another aspect, the invention provides a method for identifying a a nucleic acid target of a synMuv class C polypeptide, the method involves (a) providing a C. elegans containing mutations in a Class C synMuv gene selected from the group consisting of trr-1, hat-1, epc-1, and ssl-1 and in a Class A or Class B synMuv gene; (b) growing the C. elegans-on bacteria expressing a dsRNA; and (c) identifying-a dsRNA that suppresses a synMuv phenotype; where the dsRNA identifies a nucleic acid target of a synMuv class C polypeptide.

In another aspect, the invention features a method of identifying a nucleic acid whose expression is modulated by a synMuv class C polypeptide, the method involves (a) providing a microarray containing fragments of nematode nucleic acids; (b) contacting the microarray with detectably labeled nucleic acids derived from a nematode containing a mutation in a Class C synMuv gene selected from the group consisting of trr-1, hat-1, epc-1, and ssl-1 gene; (c) detecting an alteration in the expression of at least one nucleic acid of a C. elegans containing a mutation in the synMuv class C gene relative to the expression of the nucleic acid in a control nematode, where an alteration in the expression identifies the nucleic acid as a nucleic acid modulated by a synMuv class C polypeptide. In one embodiment,the C. elegans further contains a mutation in a synMuv A or synMuv B gene In another embodiment, the C. elegans further contains a mutation in a gene that results in a Vulvaless (Vul) phenotype. In another embodiment, the gene encodes LET-60.

In another aspect, the invention provides a method for identifying a nucleic acid target of a synMuv class C polypeptide, the method involves (a) providing nucleic acids derived from a nematode cell; (b) crosslinking the nucleic acids and their associated proteins to form a nucleic acid-protein complex; (c) contacting the nucleic acid-protein complex with an antibody that binds a polypeptide selected from the group consisting of TRR-1, HAT-1, EPC-1, AND SSL1; (d) purifying the nucleic acid-protein complex using an immunological method; and (e) isolating the nucleic acid, where the isolated nucleic acid is a nucleic acid that binds a synMuv class C polypeptide. In another embodiment, further containing the following steps: (f) detectably labeling the nucleic acid of step (e); (g) contacting the detectably labeled nucleic acid with a microarray containing C. elegans nucleic acid fragments; and (h) detecting binding of the detectably labeled nucleic acid, where the binding identifies the nucleic acid as a nucleic acid target of a synMuv class C polypeptide.

By “binds” is-meant a compound or antibody which recognizes and binds a polypeptide of the invention, but which does not substantially recognize and bind other different molecules in a sample, for example, a biological sample, which naturally includes a polypeptide of the invention.

By “cell” is meant a single-cellular organism, cell from a multi-cellular organism, or it may be a cell contained in a multi-cellular organism.

By “derived from” is meant isolated from or having the sequence of a naturally-occurring sequence (e.g., a cDNA, genomic DNA, synthetic, or combination thereof).

“Differentially expressed” means a difference in the expression level of a nucleic acid. This difference may be either an increase or a decrease in expression; when compared to control conditions.

By “epc-1 nucleic acid” is meant a synMuv Class C nucleic acid substantially identical to Y111B2A.11, which is identified by C. elegans cosmid name and open reading frame number.

By “EPC-1 polypeptide” is meant an amino acid sequence substantially identical to a polypeptide expressed by an epc-1nucleic acid that that functions in vulval development and associates with a MYST family histone acetyltransferase.

By “fragment” is meant a portion of a protein or nucleic acid that is substantially identical to a reference protein or nucleic acid (e.g., one of those listed in Tables 2 or 3), and retains at least 50% or 75%, more preferably 80%, 90%, or 95%, or even 99% of the biological activity of the reference protein or nucleic acid using a nematode bioassay as described herein or a standard biochemical or enzymatic assay.

By “hybridize” is meant pair to form a double-stranded molecule between complementary polynucleotide sequences (e.g., genes listed in Tables 1-4 and 7), or portions thereof, under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 m NaCl, 25 mM trisodium citrate, 1% SDS, 50% formamidc, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloninig: A Laboratory Manual, Cold Spring Harbor Laboratory Press, New York.

By “hat-1 nucleic acid” is meant a a synMuv Class C nucleic acid substantially identical to VC5.4, which is identified by C. elegans cosmid name and open reading frame number.

By “HAT-1 polypeptide” is meant an amino acid sequence substantially identical to a polypeptide expressed by a hat-1 nucleic acid that functions in vulval development and contains a chromodomain and an acetyltransferase catalytic domain.

By “lin(n3628) nucleic acid” is meant a nucleic acid substantially identical to SEQ ID NO:24 that encodes a histone methyltransferase.

By “LIN(n3628) polypeptide” is meant an amino acid sequence having substantial identity to a polypeptide expressed by a lin(n3628) nucleic acid that has histone methyltransferase activity and includes a SET domain.

By “lin(n4256) nucleic acid” is meant a synMuv class B nucleic acid substantially identical to SEQ ID NO:27.

By “LIN(n4256) polypeptide” is meant an amino acid sequence having substantial identity to a polypeptide expressed by a lin(n4256) nucleic acid and having histone methyltransferase activity.

By “lin-65 nucleic acid” is meant a synMuv class B nucleic acid substantially identical to SEQ ID NO:28.

By “LIN-65 polypeptide” is meant an amino acid sequence having substantial identity to a polypeptide expressed by a lin-65 nucleic acid that is rich in acidic amino acids.

By “immunological assay” is-meant an assay that relies on an immunological reaction, for example, antibody binding to an antigen. Examples of immunological assays include ELISAs, Western blots, immunoprecipitations, and other assays known to the skilled artisan.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a eDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “KIAAA1732 nucleic acid” is meant a human nucleic acid sequence having substantial identity to SEQ ID NO:30 and encoding a histone methyltransferase.

By “KIAAA1732 polypeptide” is meant an amino acid sequence encoded by a nucleic acid substantially identical to SEQ ID NO:30, having histone methyltransferase activity, and including a SET domain.

By “mep-1 nucleic acid” is meant a a synMuv Class B nucleic-acid substantially identical to M04B2.1, which is identified by C. elegans cosmid name and open reading frame number.

By “MEP-1 polypeptide” is meant an amino acid sequence substantially identical to a polypeptide expressed by a mep-1 nucleic acid that functions in vulval development and contains multiple Zn finger motifs.

By “multivulva” is meant having one vulva and one additional vulva-like structure.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No: 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P.E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃, OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines.

By “ortholog” is meant a polypeptide or nucleic acid molecule of an organism that is highly related to a reference protein, or nucleic- acid sequence, from another organism. An ortholog is functionally related to the reference protein or nucleic acid sequence. In other words, the ortholog and its reference molecule would be expected to fulfill similar, if not equivalent, functional roles in their respective organisms. It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs may be identified using methods provided herein. The functional role of an ortholog may be assayed using methods well known to the skilled artisan, and described herein. For example, function might be assayed in vivo or in vitro using a biochemical, immunological, or enzymatic assay; transformation rescue, or in a nematode bioassay for the effect of gene inactivation on nematode phenotype (e.g., fertility), as described herein. Alternatively, bioassays may be carried out in tissue culture; function may also be assayed by gene inactivation-(e.g., by RNAi, siRNA,; or gene knockout), or gene over-expression, as well as by other methods.

By “polypeptide” is meant any chain of amino acids, or analogs thereof, regardless of length or post-translational modification (for example, glycosylation or phosphorylation).

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “purified antibody” is meant an antibody that is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “ssl-1 nucleic acid” is meant a nucleic acid substantially identical to SEQ ID NO:21, which is identified by C. elegans cosmid name and open reading frame number.

By “SSL-1 polypeptide” is meant an amino acid sequence substantially identical to a polypeptide expressed by a ssl-1 nucleic acid that functions in embryonic development and has homology to p400 a SW12/SNF2 family member having ATPase activity.

By “synthetic multivulva (synMuv) gene” is meant a gene that when mutated, interacts synergistically with a second synMuv gene to cause a synthetic multivulval phenotype. For example, trr-I and mep-1 are synMuv genes because worms containing a mutation in trr-1 or mep-1, and also-having-a mutation in lin-15A (e.g., lin-15A(n767)) display a synthetic multivulval phenotype.

By “trr-1 nucleic acid” is meant a nucleic acid substantially identical to SEQ ID NO:12, which is identified by C. elegans cosmid name and open reading frame number. Nucleic acid and polypeptide sequence information is available at wormbase (www.wormbase.org), a central repository of data on C. elegans.

By “TRR-1 polypeptide” is meant an amino acid sequence substantially identical to a polypeptide expressed by a trr-1 nucleic acid that functions in transcriptional regulation and vulval development.

“Therapeutic compound” means a substance that has the potential of affecting the function of an organism. Such a compound may be, for example, a naturally occurring, semi-synthetic, or synthetic agent. For example, the test compound may be a drug that targets a specific function of an organism. A test compound may also be an antibiotic or a nutrient. A therapeutic compound may decrease, suppress, attenuate, diminish, arrest, or stabilize the development or progression of disease, disorder, or infection in a eukaryotic host organism.

The invention provides a number of targets that are useful for the development of highly specific drugs to treat neoplasia or a disorder characterized by the misregulation of the cell cycle (e.g., a hyperproliferative disorder). In addition, the methods of the invention provide a facile means to identify therapies that are safe for use in eukaryotic host organisms (i.e., compounds that do not adversely affect the normal development, physiology, or fertility of the organism). In addition, the methods of the invention provide a route for analyzing virtually any number of compounds for effects on cell proliferation and cell cycle regulation with inexpensively and with high-volume throughput in a living animal.

Other features and advantages of the invention will be apparent from the detailed description, and from the claims.

The invention provides methods and compositions useful in treating a neoplasia and in identifying chemotherapeutic agents. Other features and advantages of the invention will be apparent from the detailed description, and from the claims.

### BRIEF DESCRIPTION OF THE DRAWINGS

FIG. 1A is a schematic diagram the location of mep-1 on the LGIV physical map in between sem-3 and dpy-20. The mep-1 rescuing cosmid M04B2 is shown in bold.

FIG. 1B shows the predicted MEP-1 protein (SEQ ID NO: 1). Zinc finger motifs are shaded, and the positions of mep-1 mutations are indicated by arrowheads.

FIG. 2 shows the genomic sequence of mep-1 (SEQ ID NO:2). The start and stop codons are indicated by highlighting.

FIG. 3 shows the nucleic acid sequence of the mep-1 open reading frame (SEQ ID NO:3).

FIG. 4 shows the deduced amino acid sequence of MEP-1.

FIGS. 5A and 5B are bar graphs showing that trr-1 singe mutants are defective in P(8).p fate specification. Induction of individual P(3-8).p cells was scored in wild-type animals (FIG. 5A) and trr-1(n3712) mutants (FIG. 5B). Certain cells in trr-1 mutants adopted hybrid fates in which one of two Pn.p daughters divided like daughters of induced Pn.p cells and the other daughter remained undivided as in uninduced Pn.p cells. Ectopic induction in single mutant animals containing each of the other five trr-1 mutations was similarly restricted to P8.p.

FIG. 6 is a bar graph showing that. trr-1 and class B synMuv mutations are synthetically defective in P8.p cell-fate specification. P8.p induction was scored. We recognized trr-1 homozygous mutants as non-Gfp progeny of trr-1/mIn1[dpy-10(e128) mIs14] heterozygous parents. lin-15B(n744), lin-35(n745), lin-36(n766) and lin-37(n758) are the strongest mutations of their corresponding genes. Strains homozygous for these mutations are viable. trr-1; synmuvB double mutant strains with these mutations were derived from parents that were homozygous for the synmuvB mutation and hence lacked maternal and zygotic function of the class B synMuv gene in question. The dpl-1(n3316) null mutation causes sterility. We combined dpl-1(RNAi) with the dpl-1-(n3316) mutation to generate mutants that lacked both maternal and zygotic dpl-1 activity and recognized these mutants as non-Gfp progeny of dpl-1(n3316) trr-1/mIn1[dpy-10(e128) mIs14] heterozygous parents that were injected with dpl-1 dsRNA.

FIG. 7A shows the trr-1 gene structure as derived from CDNA and genomic sequences. Shaded boxes indicate coding sequence and open boxes indicate 5′ and 3′ untranslated regions. Predicted translation initiation and termination codons and the poly(A) tail are indicated. Positions of alternative splicing are indicated by asterisks. In all cases, the use of alternative splice acceptors creates small differences in the trr-1 coding sequence: alternative splicings of the fourth (ag/TTTCAGAC (SEQ ID NO:4) versus agtttcag/AC (LEO ID NO:5)), fifth (ag/AATCTTCAGTC (SEQ ID NO:6) versus (agaatcttcag/CC (SEQ ID NO:7)), eleventh (ag/AACTTTAAGAT (SEQ ID NO:8) versus agaactttaag/AT (SEQ ID NO:9) and twelfth introns (ag/TTGCAGAA (SEQ ID NO:10) versus agttgcag/AA (SEQ ID NO:11)) differ by either six or nine nucleotides.

FIG. 7B is a schematic diagram of the TRR-1 protein. The positions of substitutions caused by TRR-1 mutations are indicated above. TRR-1 is similar to mammalian TRRAP and yeast Tralp thoughout the lengths of the proteins. Domains of similarity (e.g., FAT and ATM/PI-3 kinase-like domains) that these three proteins share are indicated.

FIG. 8 shows the genomic nucleic acid sequence of trr-1 (SEQ ID NO:12). The start and stop codons are indicated by highlighting.

FIG. 9 shows the nucleic acid sequence of the trr-1 open reading frame (SEQ ID NO: 13).

FIG. 10 shows the deduced amino acid sequence of TRR-1 (SEQ ID NO:14).

FIG. 11A is a schematic diagram showing the hat-1 gene structure as derived from cDNA and genomic sequences. Shaded boxes indicate coding sequence and open boxes indicate 5′ and 3′ untranslated regions. Predicted translation initiation and termination codons and the poly(A) tail are shown.

FIG. 11B is a schematic diagram of the HAT-1 protein. HAT-1 is similar to MYST family acetyltransferases, all of which contain a MOZ/SAS acetyltransferase domain and some of which contain a chromodomain. Nematodes expressing the hat-1(n4075) deletion are expected to produce only the first 35 amino acids of the wild-type HAT-1 protein and additional frameshifted amino acids prior to truncation.

FIG. 11C is a bar graph showing that hat-1 single mutants were defective in P(8).p fate specification. Induction of individual P(3-8).p cells was scored in wild-type animals (left) and hat-1(n4075) mutants (right). hat-1 homozygous mutants were recognized as non-Unc progeny of +/nT1n754; hat-1(n4075)/nT1n754 heterozygous parents.

FIG. 11D is a bar graph showing that hat-1 is synthetically defective in P8.p cell-fate specification with the class B synMuv mutation lin-15B(n744). P8.p induction was scored as described below. hat-1 homozygous mutants were recognized as in (C).

FIG. 12 shows the genomic nucleic acid sequence of hat-1 (SEQ ID NO: 15). The start and stop codons are indicated by highlighting.

FIG. 13 shows the nucleic acid sequence of the hat-1 open reading frame (SEQ ID NO: 16).

FIG. 14 shows the deduced amino acid sequence of HAT-1 (SEQ ID NO: 17).

FIG. 15A is a schematic diagram showing epc-1 and ssl-1 gene structures and deletion mutations. The gene structure of epc-1 was derived by comparing CDNA and genomic sequences.

FIG. 15B is a schematic showing the ssl-1 gene structure and deletion mutation. The gene structure of ssl-1 is partially derived from comparison of CDNA and genomic sequences (SLI splice leader, 5′ untranslated region, exons 1-12 and the beginning of exon 13) and partially predicted solely from genomic sequence (the end of exon 13). As we do not have CDNA clones representing the 3′ end of ssl-1, we are unable to reliably assign a 3′ untranslated region and poly(A) tail. Filled boxes indicate coding sequence and open boxes indicate 5′ and 3′ untranslated regions. SLI splice leaders, predicted translation start and stop codons and poly(A) tail are shown. The regions of genomic sequence removed by the epc-1(n4076) and ssl-1(n4077) deletions are indicated.

FIG. 16 shows the genomic nucleic acid sequence of epc-1 (SEQ ID NO: 18).

FIG. 17 shows the nucleic acid sequence of the epc-1 open reading frame (SEQ ID NO:19).

FIG. 18 shows the deduced amino acid sequence of EPC-1 (SEQ ID NO:20).

FIG. 19 shows the genomic nucleic acid sequence of ssl-1 (SEQ ID NO:21) and the deduced amino acid sequence.

FIG. 20A shows the exon boundaries of the ssl-1 genomic nucleic acid sequence.

FIG. 20B shows the cDNA nucleic acid sequence of ssl:1 (SEQ ID NO:22).

FIG. 21 shows the amino acid sequence of SSL-1 (SEQ ID NO:23).

FIGS. 22A and 22B are schematic diagrams showing two models of TRR-1/HAT-1/EPC-1 function with respect to class B synMuv proteins

FIG. 22A is a schematic diagram showing that a TRR-1/HAT-1/EPC-1 complex and the class B synMuv proteins act on different targets and differentially regulate transcription. In this model a putative TRR-1/HAT-1/EPC-1 complex acts on targets that are different from those of a putative class B synMuv protein complex. A TRR-1/HAT-1/EPC-1 complex may promote transcription of genes that negatively regulate vulval development, whereas class B synMuv proteins may repress transcription of genes that promote vulval development.

FIG. 22B is a schematic diagram showing a second model. In this second model, a TRR-1/HAT-1/EPC-1 complex acts on the same targets as do the class B synMuv proteins. Together these two putative protein complexes may specify an acetylation pattern on histones that is required for efficient silencing of genes that promote vulval development. A TRR-1/HAT-1/EPC-1 complex may act through DPL-1 and EFL-1, although genetic interactions suggest that not all TRR-1/HAT-1/EPC-1 complex activity goes through DPL-1 and EFL-1.

FIG. 23 shows the genomic sequence of lin(n3628) including 1 kb of upstream and downstream genomic sequences (SEQ ID NO:24). The exon boundaries are also defined.

FIG. 24 shows the amino acid sequence of LIN(n3628) (SEQ ID NO:25).

FIG. 25 shows the genomic sequence of lin(n4256) (SEQ ID NO:26). The exon boundaries are also defined.

FIG. 26 shows the amino acid sequence of LIN(n4256) (SEQ ID NO:27) FIG. 27 shows the genomic sequence of lin-65 (SEQ ID NO:28). The exon boundaries are also defined.

FIG. 28 shows the amino acid sequence of LIN-65 (SEQ ID NO:29). The exon boundaries are also defined.

FIG. 29 shows the mRNA sequence that encodes the LIN(n3628) human ortholog, KIAA1732.

FIG. 30 shows the amino acid sequence of KIAA1732 (SEQ ID NO:35).

FIG. 31 shows the genomic sequence of KIAA1732 (SEQ ID NO:36).

FIG. 32 defines the domains of LIN(n3628), including the SET catalytic domain.

FIG. 33 defines the domains of KIAA1732, including the SET catalytic domain.

### DESCRIPTION OF THE INVENTION

As reported in more detail below, we have identified new components of the Rb pathway that function in chromatin remodeling and antagonize Ras signaling, and methods for using such components for the identification of chemotherapeutics and the identification of new clinical targets for the treatment of neoplasia.

### EXAMPLE 1

Isolation of New synMuv Mutants

A variety of genetic studies revealed that sterility is often associated with a severe reduction of class B synMuv gene function. For example, in a genetic screen for alleles that did not complement the synMuv phenotype of lin-9(n112), (Ferguson et al., Genetics 123: 109-21, 1989) recovered the alleles lin-9(n942) and lin-9(n943), which caused sterility when homozygous. In another example, we performed gene dosage studies and heterozygoteshad markedly reduced brood sizes. In addition, deletion mutations of synMuv genes that showed recessive sterility were recovered by reverse genetic approaches (e.g. alleles of lin-53 (Lu 1999), lin-54, and dpl-1 (Ceol et al., Mol Cell 7: 461-73, 2001).

Previous genetic screens for synMuv mutants (Ferguson et al., Genetics 123: 109-21, 1989) were performed before a link between loss of synMuv gene function and sterility was well established. These screens required that isolates be fertile and viable in order to recover mutant alleles. In addition to failing to recover recessive sterile mutations of the genes described above, these screens failed to recover mutations of the class B synMuv genes efl-1 and let-418, both of which can mutate to a sterile phenotype (Von Zelewsky et al., Development 127: 5277-84, 2000; Ceol et al., Mol Cell 7: 461-73, 2001). Given this failure, we undertook a genetic screen to identify additional synMuv genes that would allow the recovery of homozygous sterile mutations through phenotypically wild-type heterozygous siblings.

To screen for new synMuv mutants, we examined the F₂ progeny of individually plated F₁ animals after EMS mutagenesis-of lin-15A(n 767) mutants. This screen represented 6760 haploid genomes examined for mutations that either alone or in combination with lin-15A(n767) showed a recessive Muv phenotype. Using this strategy we identified 95 Muv mutations, 24 of which were maintained as heterozygotes due to recessive sterility that cosegregated with the Muv phenotype. Three mutations caused a Muv phenotype in the absence of lin-15A(n767) and were found to affect lin-1 and lin-31, both of which function downstream of let-60 Ras in vulval induction (Ferguson et al., Nature 326:259-67, 1987). These mutations, lin-1(n3443), lin-1(n3522), and lin-31(n3440) were not characterized further. Additionally, we recovered 29 mutations that, together with lin-15A(n767), caused a weakly penetrant (<30%) Muv phenotype. The remaining 63 mutations were assigned to 21 complementation groups, which include the previously known genes ark-1, dpl-1, efl-1, gap-1, let-418, lin-9, lin-13, lin-15B, lin-35, lin-36, lin-52, lin-53, lin-61, and sli-1, and the new genes lin(n3441), lin(n3542), lin(n3628), lin(n3681), lin(n3707), mep-1, and trr-1.

Phenotypes of New Mutants

We characterized the penetrance of the Muv phenotype for each strain at 15° C. and 20° C. The results of this study are described in Table 1.

TABLE 1
Penetrance of Muv phenotype (n)

| | | | Additional |
|---|---|---|---|
| Genotype | 15° C. | 20° C. | phenotypes |
| ark-1(n3524) lin-15A(n767) | 0 (251) | 80 (171) | |
| ark-1(n3701); lin-15A(n767) | 12 (190) | 95 (160) | |
| dpl-1(n3643); lin-15A(n767) | 99 (154) | 100 (252) | |
| efl-1(n3639); lin-15A(n767) | 93 (74) | 100 (78) | Ste |
| gap-1(n3535) lin-15A(n767) | 1.4 (143) | 50 (236) | |
| let-418(n3536); lin-15A(n767) | 0 (201) | 55 (183) | hs Ste |
| let-418(n3626); lin-15A(n767) | 1.6 (62) | 97 (76) | Ste |
| let-418(n3629); lin-15A(n767) | 0 (52) | 86 (58) | Ste |
| let-418(n3634); lin-15A(n767) | 0 (87) | 92 (48) | Ste |
| let-418(n3635); lin-15A(n767) | 0 (76) | 71 (70) | Ste |
| let-418(n3636); lin-15A(n767) | 0 (77) | 92 (78) | Ste |
| let-418(n3719); lin-15A(n767) | 0 (101) | 100 (60) | Ste |
| lin-9(n3631); lin-15A(n767) | 100 (42) | 100 (72) | Ste |
| lin-9(n3675); lin-15A(n767) | 43 (166) | 100 (105) | |
| lin-9(n3767); lin-15A(n767) | 100 (67) | 100 (56) | Ste |
| lin-13(n3642); lin-15A(n767) | 3.3 (60) | 100 (63) | Ste |
| lin-13(n3673); lin-15A(n767) | 61 (145) | 97 (129) | |
| lin-13(n3674); lin-15A(n767) | 78 (131) | 100 (191) | hs Ste |
| lin-13(n3726); lin-15A(n767) | 31 (225) | 99 (149) | hs Ste |
| lin-15B(n3436) lin-15A(n767) | 100 (193) | 100 (212) | |
| lin-15B(n3676) lin-15A(n767) | 18 (167) | 72 (130) | |
| lin-15B(n3677) lin-15A(n767) | 99 (111) | 100 (122) | |
| lin-15B(n3711) lin-15A(n767) | 100 (186) | 100 (156) | |
| lin-15B(n3760) lin-15A(n767) | 32 (171) | 100 (150) | |
| lin-15B(n3762) lin-15A(n767) | 63 (113) | 97 (116) | |
| lin-15B(n3764) lin-15A(n767) | 96 (232) | 100 (199) | |
| lin-15B(n3766) lin-15A(n767) | 55 (132) | 100 (173) | |
| lin-15B(n3768) lin-15A(n767) | 80 (159) | 100 (302) | |
| lin-15B(n3772) lin-15A(n767) | 100 (220) | 100 (191) | |
| lin-35(n3438); lin-15A(n767) | 100 (153) | 100 (126) | partial Ste at |
| | | | 20° C., Rup |
| lin-35(n3763); lin-15A(n767) | 100 (108) | 100 (160) | partial Ste at |
| | | | 20° C., Rup |
| lin-36(n3671); lin-15A(n767) | 65 (191) | 100 (151) | |
| lin-36(n3672); lin-15A(n767) | 98 (198) | 100 (178) | |
| lin-36(n3765); lin-15A(n767) | 0 (184) | 37 (202) | |
| lin-52(n3718); lin-15A(n767) | 100 (41) | 100 (82) | Ste |
| lin-53(n3448); lin-15A(n767) | 67 (130) | 100 (211) | partial Ste at |
| | | | 20° C. |
| lin-53(n3521); lin-15A(n767) | 100 (34) | 100 (125) | partial Ste at |
| | | | 20° C. |
| lin-53(n3622); lin-15A(n767) | 85 (61) | 100 (66) | Ste |
| lin-53(n3623); lin-15A(n767) | 24 (55) | 100 (51) | Ste |
| lin-61(n3442); lin-15A(n767) | 22 (130) | 100 (152) | |
| lin-61(n3446); lin-15A(n767) | 36 (124) | 99 (191) | |
| lin-61(n3447); lin-15A(n767) | 11 (121) | 87 (207) | |
| lin-61(n3624); lin-15A(n767) | 0 (152) | 89 (231) | |
| lin-61(n3736); lin-15A(n767) | 0 (193) | 100 (201) | |
| n3441; lin-15A(n767) | 80 (165) | 99 (195) | |
| n3541; lin-15A(n767) | 79 (242) | 98 (137) | |
| n3543; lin-15A(n767) | 85 (177) | 100 (121) | |
| n3628; lin-15A(n767) | 2.9 (103) | 84 (188) | |
| n3681; lin-15A(n767) | 0 (214) | 72 (192) | |
| n3542 lin-15A(n767) | 0 (127) | 35 (218) | |
| n3707 lin-15A(n767) | 3.8 (80) | 77 (26) | |
| mep-1(n3680); lin-15A(n767) | 4.9 (122) | 97 (105) | hs Ste |
| mep-1(n3702); lin-15A(n767) | 30 (61) | 100 (141) | Ste |
| mep-1(n3703); lin-15A(n767) | 25 (72) | 100 (107) | Ste |
| sli-1(n3538) lin-15A(n767) | 4.3 (138) | 90 (173) | |
| sli-1(n3544) lin-15A(n767) | 4.6 (153) | 80 (265) | cs embryonic |
| | | | lethality |
| sli-1(n3683) lin-15A(n767) | 5.0 (80) | 88 (148) | cs embryonic |
| | | | lethality |
| trr-1(n3630); lin-15A(n767) | 3.1 (131) | 85 (212) | Ste, Gro |
| trr-1(n3637); lin-15A(n767) | 1.1 (92) | 80 (200) | Ste, Gro |
| trr-1(n3704); lin-15A(n767) | 3.1 (96) | 79 (244) | Ste, Gro |
| trr-1(n3708); lin-15A(n767) | 2.0 (151) | 84 (228) | Ste, Gro |
| trr-1(n3709); lin-15A(n767) | 1.0 (97) | 77 (154) | Ste, Gro |
| trr-1(n3712); lin-15A(n767) | 5.8 (121) | 77 (192) | Ste, Gro |

Ste: sterile; Gro: growth rate abnormal; Rup: rupture at the vulva; cs: cold sensitive; hs: heat sensitive.

The penetrance of the Muv phenotype was determined after growing synMuv mutant strains at the indicated temperature for two or more generations. For most strains in which a fully penetrant sterile phenotype was associated with the Muv phenotype, we scored the penetrance of the Muv phenotype by examining sterile progeny of heterozygous mutant parents. For trr-I mutant strains, we scored the penetrance of the Muv phenotype by examining non-Gfp progeny of trr-1/mIn1[dpy-10(e128)mIs14]; lin-15A(n767) heterozygous parents. All strains were backcrossed to lin-15A(n767) twice prior to phenotypic characterization. In addition to the phenotypes described above, many of the strains exhibited heat sensitive inviability due to frequent rupture, sterility, and/or general sickness.

The penetrance at 25° C. is not shown because all strains had a highly penetrant (>90%) Muv phenotype at this temperature. Since a heat-sensitive Muv phenotype is characteristic of most synMuv strains, including those with null mutations in synMuv genes (Ferguson et al., Genetics 123: 109-21, 1989), it is likely that many synMuv mutations are not particularly temperature sensitive, but rather that the synMuv genes regulate a temperature sensitive process.

A subset of our synMuv strains also exhibited a sterile phenotype. In these strains, the sterile phenotype cosegregated with the Muv phenotype during backcrosses and two- and three-factor mapping experiments. For those mutations tested, we found that our new mutations did not complement the sterile phenotypes caused by previously isolated, allelic synMuv mutations. These observations suggest that the sterile and Muv phenotypes of these strains were caused by the same mutation.

We observed an unusual aspect to the sterility of one of our strains. We examined the mep-1(n3680); lin-15A(n767) strain and found that its sterile phenotype showed maternal-effect rescue. When derived from heterozygous parents, the sterility of the mep-1 (n3680), lin-15A(n767) animals was 3.2% penetrant (n=62), but was 55% penetrant (n=69) when these animals were derived from homozygous parents. Mutations that affect the Mes (Mes, maternal-effect sterility) genes also show maternal-effect rescue of sterility (Capowski et al., Genetics 129: 1061-72, 1991). Some Mes genes encode homologs of Drosophila polycomb group proteins and are proposed to function in X chromosome transcriptional silencing in the germline (Holdeman et al., Development 125: 2457-67, 1998; Korf et al., Development 125: 2469-78, 1998; Fong et al., Science 296: 2235-8, 2002). A functional relationship between the synMuv and Mes genes has not been previously reported.

New synMuv Genes

Using two-factor crosses and sex chromosome transmission tests, we mapped the new mutations to linkage groups (Table 2).

TABLE 2
Chromosomal linkages of new synMuv mutations
A. Autosomal mutations

| | | Genotype of selected |
|---|---|---|
| | Mutation used for | F₂ hermaphrodites |
| | selection of homozygous | withrespect to the |
| New mutation | F₂ hermaphrodites | linked, unselected mutation |
| ark-1(n3524) | dpy-20(e1282) IV | 2/19 ark-1(n3524)/+ |
| ark-1(n3701) | ark-1(n3701) | 1/14 dpy-20(e1282)/+ IV |
| dpl-1(n3643) | dpl-1(n3643) | 0/20 rol-6(e187)/+ II |
| efl-1(n3639) | rol-4(sc8) V | 4/20 efl-1(n3639)/+ |
| let-418(n3536) | let-418(n3536) | 4/21 rol-4(sc8)/+ V |
| let-418(n3626) | rol-4(sc8) V | 0/19 let-418(n3626)/+ |
| let-418(n3629) | rol-4(sc8) V | 1/20 let-418(n3629)/+ |
| let-418(n3634) | rol-4(sc8) V | 2/19 let-418(n3634)/+ |
| let-418(n3635) | rol-4(sc8) V | 5/20 let-418(n3635)/+ |
| let-418(n3636) | rol-4(sc8) V | 3/20 let-418(n3636)/+ |
| let-418(n3719) | rol-4(sc8) V | 2/30 let-418(n3719)/+ |
| lin-9(n3631) | unc-32(e189) III | 0/20 lin-9(n3631)/+ |
| lin-9(n3675) | lin-9(n3675) | 0/22 unc-32(e189)/+ III |
| lin-9(n3767) | lin-9(n3767) | 0/16 mgP21/+ III |
| lin-13(n3642) | unc-32(e189) III | 1/20 lin-13(n3642)/+ |
| lin-13(n3673) | lin-13(n3673) | 0/25 unc-32(e189)/+ III |
| lin-13(n3674) | lin-13(n3674) | 0/25 unc-32(e189)/+ III |
| lin-13(n3726) | lin-13(n3726) | 1/26 unc-32(e189)/+ III |
| lin-35(n3438) | lin-35(n3438) | 0/30 dpy-5(e61)/+ I |
| lin-35(n3763) | lin-35(n3763) | 0/22 dpy-5(e61)/+ I |
| lin-36(n3671) | lin-36(n3671) | 1/23 unc-32(e189)/+ III |
| lin-36(n3672) | lin-36(n3672) | 0/16 unc-32(e189)/+ III |
| lin-36(n3765) | lin-36(n3765) | 0/9 unc-32(e189)/+ III |
| lin-52(n3718) | lin-52(n3718) | 1/16 mgP21/+ III |
| lin-53(n3448) | lin-53(n3448) | 1/22 dpy-5(e61)/+ I |
| lin-53(n3521) | dpy-5(e61) I | 0/20 lin-53(n3521)/+ |
| lin-53(n3622) | dpy-5(e61) I | 5/30 lin-53(n3622)/+ |
| lin-53(n3623) | lin-53(n3623) | 4/16 hP4/+ I |
| lin-61(n3442) | lin-61(n3442) | 0/20 dpy-5(e61)/+ I |
| lin-61(n3446) | lin-61(n3446) | 1/23 dpy-5/+ I |
| lin-61(n3447) | lin-61(n3447) | 0/13 dpy-5(e61)/+ I |
| lin-61(n3624) | lin-61(n3624) | 0/15 dpy-5(e61)/+ I |
| lin-61(n3736) | dpy-5(e61) I | 1/19 lin-61(n3736)/+ |
| lin(n3441) | lin(n3441) | 5/20 dpy-5(e61)/+ I |
| lin(n3541) | lin(n3541) | 9/31 dpy-5(e61)/+ I |
| lin(n3543) | lin(n3543) | 9/27 dpy-5(e61)/+ I |
| lin(n3628) | lin(n3628) | 1/29 dpy-5(e61)/+ I |
| lin(n3681) | lin(n3681) | 3/22 rol-4(sc8)/+ V |
| mep-1(n3680) | mep-1(n3680) | 0/30 dpy-20(e1282)/+ IV |
| mep-1(n3702) | mep-1(n3702) | 0/16 sP4/+ IV |
| mep-1(n3703) | mep-1(n3703) | 0/16 sP4/+ IV |
| trr-1(n3630) | rol-6(e187) II | 0/20 trr-1(n3630)/+ |
| trr-1(n3637) | rol-6(e187) II | 1/20 trr-1(n3637)/+ |
| trr-1(n3704) | rol-6(e187) II | 1/30 trr-1(n3704)/+ |
| trr-1(n3708) | rol-6(e187) II | 0/20 trr-1(n3708)/+ |
| trr-1(n3709) | rol-6(e187) II | 2/30 trr-1(n3709)/+ |
| trr-1(n3712) | rol-6(e187) II | 1/19 trr-1(n3712)/+ |

B. X-linked mutations

| New mutation | Criteria for X linkage |
|---|---|
| lin(n3542) | transmission test |
| lin(n3707) | transmission test |
| gap-1(n3535) | transmission test |
| lin-15B(n3436) | males with pseudovulva |
| lin-15B(n3676) | transmission test, males with pseudovulva |
| lin-15B(n3677) | males with pseudovulva |
| lin-15B(n3711) | males with pseudovulva |
| lin-15B(n3760) | transmission test, males with pseudovulva |
| lin-15B(n3762) | males with pseudovulva |
| lin-15B(n3764) | transmission test, males with pseudovulva |
| lin-15B(n3766) | transmission test, males with pseudovulva |
| lin-15B(n3768) | transmission test, males with pseudovulva |
| lin-15B(n3772) | transmission test, males with pseudovulva |
| sli-1(n3538) | transmission test |
| sli-1(n3544) | transmission test |
| sli-1(n3683) | transmission test |

Autosomal and sex chromosome linkages were determined as described below. lin(n3541) was also mapped relative to bli-3(e767) and unc-54(e1092), mutations present on the extreme left and right arms, respectively, of linkage group I. Of 16 Muv progeny selected from a lin(n3541)/bli-3(e767) unc-54(e1092), lin-15A(n767) parent, none were bli-3(e767)/+ whereas six were unc-54(e1092)/+, indicating lin(n3541) lies nearer to bli-3(e767).

We then determined if a given mutation failed to complement mutations of known synMuv genes on the same linkage group. Mutations that were not assigned to known synMuv complementation groups were tested against unassigned mutations within the same linkage group for complementation. These tests defined seven new synMuv loci: trr-1, mep-1, lin(n3441), lin(n3628), lin(n3681), lin(n3707), and lin(n3542). We used three-factor crosses to map most of these new synMuv genes within their respective linkage groups (Table 3).

TABLE 3
Map data for newly-identified synMuv loci
A. Three- and four-factor mapping

| | | | Genotype of selected |
|---|---|---|---|
| | | Phenotype of | recombinants (with |
| | | selected | respect to unselected |
| Gene | Genotype of heterozygote | recombinants | markers) |
| ark-1 | + + ark-1 / unc-5 dpy-20 +; lin-15A(n767) | Unc | 10/10 ark-1 / + |
| | | Dpy | 0/1 ark-1 / + |
| | + ark-1 + / dpy-20 + unc-30; lin-15A(n767) | Dpy | 15/35 ark-1 / + |
| | | Unc | 17/33 ark-1 / + |
| | dpy-20 + + ark-1 / + lin-3 unc-22 +; lin-15A(n767) | Dpy | 3/9 unc-22 / + |
| | | Muv | 3/3 unc-22 / + |
| | dpy-20 + ark-1 + / + unc-22 + unc-30; lin- | Dpy | 1/3 unc-22 / + |
| | 15A(n767) | | |
| | | Muv | 1/2 unc-22 / + |
| | | Unc-22 | 2/3 ark-1 / + |
| | | Unc-30 | 5/6 ark-1 / + |
| | dpy-20 + ark-1 + / + dpy-26 + unc-30; lin- | Dpy-20 | 4/7 dpy-26 / + |
| | 15A(n767) | | |
| | | Muv | 3/8 dpy-26 / + |
| gap-1 | + + gap-1 lin-15A(n767) / unc-1 dpy-3 + lin- | Unc | 17/l7 gap-1 / + |
| | 15A(n767) | | |
| | | Dpy | 0/8 gap-1 / + |
| | gap-1 + + lin-15A(n767) / + unc-2 lon-2 lin- | Unc | 0/2 gap-1 / + |
| | 15A(n767) | | |
| | | Lon | 6/6 gap-1 / + |
| | + gap-1 + lin-15A(n767) / dpy-3 + unc-2 lin- | Unc | 14/18 gap-1 / + |
| | 15A(n767) | | |
| lin-52 | + lin-52 + / unc-16 + unc-47; lin-15A(n767) | Unc-47 | 7/9 lin-52 / + |
| | lin-52 + unc-69 / + stP127 +; lin-15A(n767) | Muv | 3/12 stP127 / + |
| | sma-3 + lin-52 + / + sqv-3 + unc-69; lin- | Sma | 9/9 sqv-3 / + |
| | 15A(n767) | | |
| | | Muv | 1/27 sqv-3 / + |
| | | Unc | 14/16 lin-52 / + |
| lin(n3441) | + lin(n3441) + / bli-3 + lin-17; lin-15A(n767) | Lin-17 | 9/19 lin(n3441) / + |
| | bli-3 + lin(n3441) / + spe-15 +; lin-15A(n767) | Muv | 10/18 spe-15 / + |
| | + lin(n3441) lin-17 / spe-15 + +; lin-15A(n767) | Lin-17 | 11/11 spe-15 / + |
| lin(n3628) | lin(n3628) + + / + dpy-5 unc-13; lin-15A(n767) | Dpy | 0/6 lin(n3628) / + |
| | | Unc | 6/6 lin(n3628) / + |
| | + lin(n3628) + / unc-11 + dpy-5; lin-15A(n767) | Unc | 1/11 lin(n3628) / + |
| | | Dpy | 5/11 lin(n3628) / + |
| | unc-11 + + lin(n3628) / + unc-73 lin-44 +; lin- | Muv | 3/9 unc-73 lin-44 / + + |
| | 15A(n767) | | |
| | + + lin(n3628) dpy-5 / unc-73 lin-44 + +; lin- | Muv | 0/21 unc-73 lin-44 / + + |
| | 15A(n767) | | |
| | lin(n3628) + dpy-5 / + unc-38 +; lin-15A(n767) | Muv | 3/7 unc-38 / + |
| | unc-11 lin(n3628) + / + + unc-38; lin-15A(n767) | Muv | 0/9 unc-38 / + |
| lin(n3542) | + + + lin(n3542) lin-15A(n767) / unc-10 dpy-6 lin- | Unc | 8/8 lin(n3542) / + |
| | 15A(n767) | | |
| | + lin(n3542) + lin-15A(n767) / dpy-6 + unc-9 lin- | Unc | 4/40 lin(n3542) / + |
| | 15A(n767) | | |
| mep-1 | + mep-1 + / unc-5 + dpy-20; lin-15A(n767) | Unc | 56/57 mep-1 / + |
| | | Dpy | 2/61 mep-1 / + |
| | mep-1 + + / + dpy-20 unc-30; lin-15A(n767) | Dpy | 0/51 mep-1 / + |
| | | Unc | 58/58 mep-1 / + |
| | + + mep-1 + / unc-24 mec-3 + dpy-20; lin- | UncMec | 10/12 mep-1 / + |
| | 15A(n767) | | |
| | | Unc | 17/17 mep-1 / + |
| | | MecDpy | 0/8 mep-1 / + |
| | | Dpy | 2/8 mep-1 / + |
| | + mep-1 dpy-20 + / lin-3 + + unc-22; lin- | Dpy | 5/5 lin-3 / + |
| | 15A(n767) | | |
| | | Vul | 3/10 mep-1 / + |
| | + + mep-1 + / mec-3 sem-3 + dpy-20; lin- | Mec | 17/17 mep-1 / + |
| | 15A(n767) | | |
| | | Dpy | 6/13 mep-1 / + |
| sli-1 | sli-1 + + lin-15A(n767) / + lon-2 unc-6 lin- | Lon | 0/6 sli-1 / + |
| | 15A(n767) | | |
| | sli-1 + + lin-15A(n767) / + unc-2 lon-2 lin- | Lon | 5/5 sli-1 / + |
| | 15A(n767) | | |
| | sli-1 + + lin-15A(n767) / + dpy-3 unc-2 lin- | Dpy | 0/10 sli-1 / + |
| | 15A(n767) | | |
| | | Unc | 6/6 sli-1 / + |
| | sli-1 + + lin-15A(n767) / + unc-1 dpy-3 lin- | Unc | 0/14 sli-1 / + |
| | 15A(n767) | | |
| | | Dpy | 10/10 sli-1 / + |
| trr-1 | + rol-6 + trr-1 / dpy-10 + unc-4 +; lin-15A(n767) | Rol | 3/14 unc-4 / + |
| | | Dpy | 3/3 trr-1 / + |
| | | Unc | 0/8 trr-1 / + |
| | + trr-1 + / dpy-10 + rol-1; lin-15A(n767) | Rol | 9/20 trr-1 / + |
| | + + trr-1 / dpy-10 unc-53 +; lin-15A(n767) | Unc | 0/17 trr-1 / + |
| | + trr-1 + / unc-53 + rol-1; lin-15A(n767) | Unc | 7/10 trr-1 / + |
| | | Rol | 7/10 trr-1 / + |
| | + trr-1 + rol-1 / unc-4 + mex-1 +; lin-15A(n767) | Rol | 12/14 mex-1 / + |

B. Deficiency mapping

| Gene | Genotype of heterozygote | Phenotype of heterozygote |
|---|---|---|
| lin-52 | unc-36 lin-52 / nDf40 dpy-18; lin-15A(n767) | Muv |
| mep-1 | mep-1 / sDf63 unc-31; lin-15A(n767) / + | PvlSte |
| | mep-1 / sDf62 unc-31; lin-15A(n767) / + | PvlSte |
| | mep-1 / sDf10; lin-15A(n767) / + | WT |
| trr-1 | rol-6 trr-1 / mnDf57; lin-15A(n767) | WT |
| | rol-6 trr-1 / unc-4 mnDf90; lin-15A(n767) | WT |
| | rol-6 trr-1 / mnDf29; lin-15A(n767) | WT |
| | trr-1 / unc-4 mnDf87; lin-15A(n767) | Muv |

WT: wild-type; Pvl: protruding vulva; Ste: sterile.

Three- and four-factor crosses were performed using standard methods (Brenner, Genetics 77: 71-94, 1974). Deficiency heterozygotes were constructed as described below. In addition, we have isolated trr-1, mep-1, lin(n3628), and lin(n3681) mutations away from the parental lin-15A(n767) mutation. mep-1, lin(n3628), and lin(n3681) mutations alone do not cause a Muv phenotype, and trr-1 mutations alone cause only weak ectopic vulval induction. Thus, these mutations synergize with lin-15A(n767) and are indeed synMuv mutations.

We identified mutations in gap-1 and sli-1, two genes that were originally identified in screens for mutations that suppressed the Vul phenotype caused by a reduction in let-60 Ras pathway signaling (Jongeward et al., Genetics 139: 1553-66, 1995; Hajnal et al., Genes Dev 11: 2715-28, 1997). We also identified mutations in ark-1, a gene that was first identified in a screen for mutations that caused ectopic vulval induction in a sli-1 mutant background (Hopper et al., Mol Cell 6: 65-75, 2000). gap-1, sli-1, and ark-1 single mutants were previously isolated and found to have no (sli-1, gap-1) or subtle (ark-1) defects in vulval development. Our results indicate that sli-1, gap-1, and ark-1 act redundantly with lin-15A to negatively regulate let-60 Ras signaling.

Molecular identification of mep-1

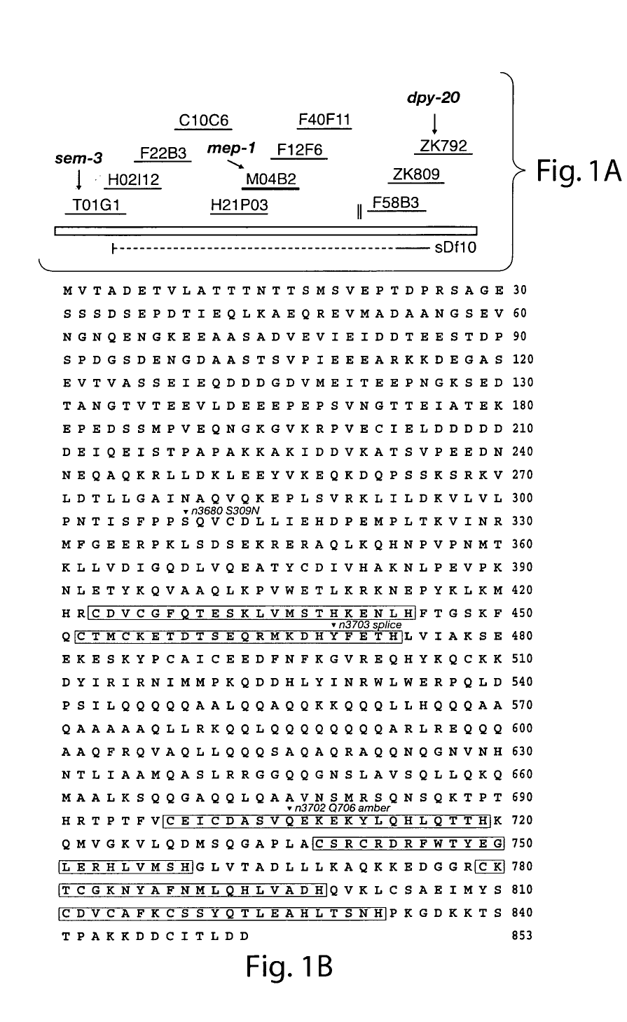

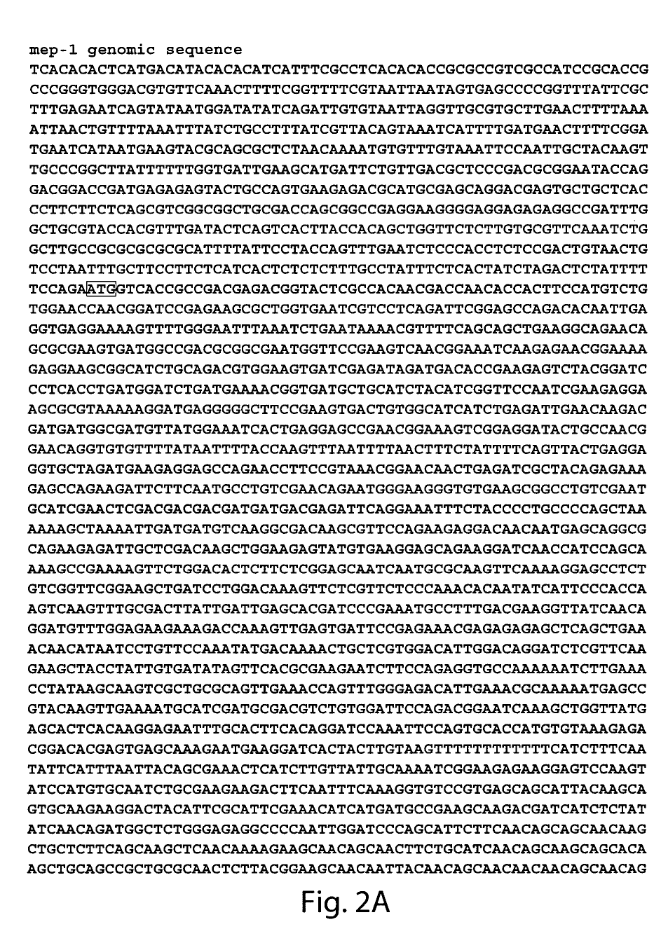

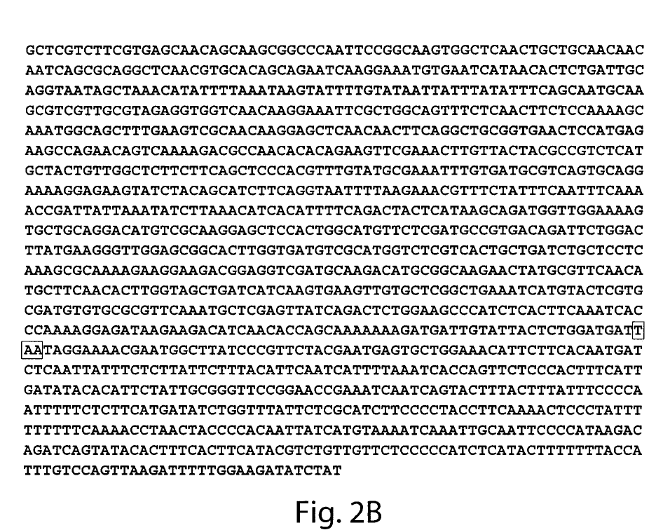

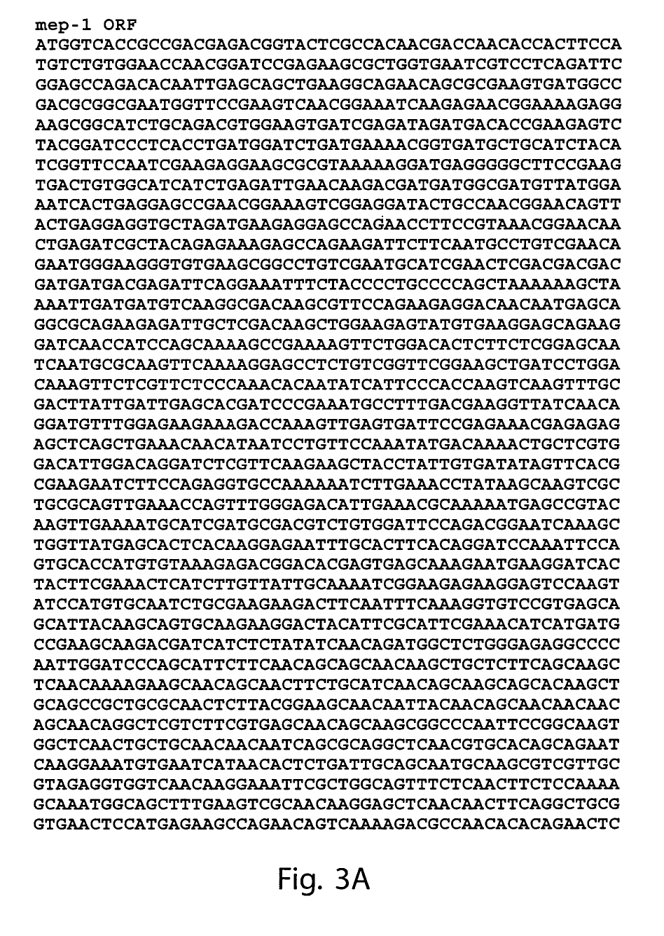

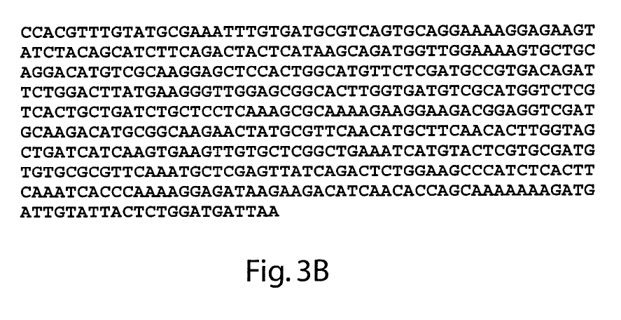

We isolated three mutations, n3680, n3702 and n3703, in a gene that we mapped to a small interval on linkage group IV in between sem-3 and dpy-20 as shown in FIG. 1. We attempted to rescue the Muv phenotype of n3680; lin-15A(n767) mutants using cosmid clones from this interval. Transgenic animals containing the cosmid M04B2 were rescued for the Muv phenotype and also showed improved fertility relative to non-transgenic animals. The genomic sequence of mep-1 is shown in FIG. 2. The mep-1 open reading frame sequence is shown in FIG. 3. This gene was originally identified based on its interaction with the germline specification genes mog-1, mog-4, mog-5 and pie-1 in yeast two-hybrid screens (Belfiore et al. RNA. 8:725-39, 2002). Because somatic tissues adopt germ cell-specific characteristics in mep-1 mutants, mep-1 is thought to repress germ cell fates in the soma. We sequenced mep-1 in our mutant strains to determine if the mutations we isolated affected this gene. These mutations identify functionally important amino acid residues or domains. n3680 mutants have a missense mutation that, in the predicted MEP-1 protein, changes a polar serine residue to an asparagine. n3702 mutants have a nonsense mutation and n3703 mutants a splice acceptor mutation in the mep-1 gene. Our genetic mapping data, cosmid rescue, and DNA sequence results indicate that n3680, n3702, and n3703 are mep-1 mutations.

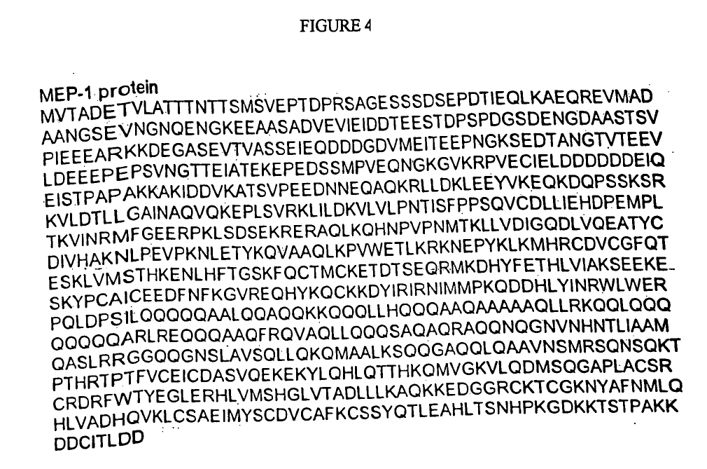

The deduced amino acid sequence of MEP-1 is shown in FIG. 4. mep-1 encodes a protein containing six zinc-finger motifs. Zinc fingers are known to mediate interactions of proteins with DNA and with other proteins. The zinc fingers of MEP-1 likely mediate interactions with LET-418 or other synMuv proteins.

Sequences of synMuv Mutations

We determined sequences of mutations that affected additional synMuv genes (Table 4).

TABLE 4
Selected synMuv proteins and allele sequences
A. Features of selected synMuv proteins

| Protein | No. amino acids | Protein similarities and domains |
|---|---|---|
| DPL-1 | 598 | Similar to DP family transcription factors; Contains DNA- |
| | | and E2F-binding domains |
| EFL-1 | 342 | Similar to E2F family transcription factors; Contains |
| | | DNA-binding, DP-binding and transactivation domains |
| LET-418 | 1829 | Similar to Mi-2 family ATP-dependent chromatin |
| | | remodeling enzymes; Contains chromodomains, PHD |
| | | finger motifs and a helicase domain* |
| LIN-9 | LIN-9L: 644 | Similar to Drosophila Aly cell cycle regulator and |
| | LIN-9S: 642 | mammalian proteins of unknown function |
| LIN-13 | 2248 | Protein has 24 Zn-finger motifs |
| LIN-35 | 961 | Similar to Retinoblastoma (pRb) family transcriptional |
| | | regulators; Contains “pocket” interaction domain |
| LIN-36 | 962 | Novel protein with C/H-rich and Q-rich regions |
| LIN-52 | 161 | Similar to Drosophila and mammalian proteins of |
| | | unknown function |
| LIN-53 | 417 | Similar to Drosophila p55, mammalian RbAp48 subunits |
| | | of chromatin remodeling and histone deacetylase |
| | | complexes; Contains WD repeats |
| LIN-61 | 491 | Similar to Drosophila 1(3)mbt and other MBT repeat- |
| | | containing proteins |
| MEP-1 | 853 | Protein has six Zn finger motifs |
| SLI-1 | 582 | Similar to Cb1 family ubiquitination-promoting proteins; |
| | | Contains SH2 domain and RING finger motif |
| TRR-1 | 4064^{‡} | Similar to mammalian TRRAP transcriptional regulator |

B. Allele sequences

| | | | | Domain affected |
|---|---|---|---|---|
| | Wild-type | | Substitution, splice site | by missense |
| Mutation | sequence | Mutant sequence | change or aberration | mutation |
| dpl-1(n3643) | TAT | TAA | Y341ochre | — |
| efl-1(n3639) | CAA | TAA | Q175ochre | — |
| let-418(n3536) | CCT | CTT | P675L | helicase/ATPase |
| let-418(n3626) | GGT | AGT | G1006S | helicase/ATPase |
| let-418(n3629) | TCC | TTC | S925F | helicase/ATPase |
| let-418(n3634) | TGG | TAG | W1128amber | — |
| let-418(n3635) | CAG | TAG | Q1594amber | — |
| let-418(n3636) | ACT | TCT | T807S | helicase/ATPase |
| | TGG | TGA | W1329opal | — |
| let-418(n3719) | TGG | TAG | W295amber | — |
| lin-9(n3631) | CAA | TAA | LIN-9L: Q594ochre | — |
| | | | LIN-9S: Q592ochre | — |
| lin-9(n3675) | GAT | AAT | LIN-9L: D305N | none predicted |
| | | | LIN-9S: D303N | none predicted |
| lin-9(n3767) | CAG | TAG | LIN-9L: Q509amber | — |
| | | | LIN-9S: Q507amber | — |
| lin-13(n3642) | CAT | TAT | H832Y | Zn finger |
| lin-13(n3673) | CAG | TAG | Q1988amber | — |
| lin-13(n3674) | CGA | TGA | R1250opal | — |
| lin-13(n3726) | GGA | GAA | G229E | none predicted |
| lin-35(n3763)^{⋄} | GCA | GTA | A555V | Pocket |
| | TTG AAA | TTG AAA AAA | K594frameshift and | — |
| | AAG | G | truncation after 611a.a. | |
| lin-36(n3671) | CAT | CCT | H284P | C/H-rich region |
| | GAA | AAA | E424K | none predicted |
| lin-36(n3672) | CAG | TAG | Q467amber | — |
| lin-36(n3765)^{†} | GCT | GTT | A242V | C/H-rich region |
| lin-52(n3718) | CAG | TAG | Q31amber | — |
| lin-53(n3448) | AGT | ATT | S3841 | WD repeat |
| lin-53(n3521) | GAA | AAA | E174K | WD repeat |
| | | AAG/atatgtgt | | |
| lin-53(n3622) | AAG/gtatgtgt | (SEQ ID NO:30) | Exon 1 donor | — |
| lin-53(n3623) | TGG | TAG | W337amber | — |
| | | aacttcaa/AAT | | |
| lin-61(n3442) | aacttcag/AAT | (SEQ ID NO: 31) | Exon 4 acceptor | — |
| lin-61(n3446) | CAA | TAA | Q412ochre | — |
| lin-61(n3447) | AGT | AAT | S354N | MBT repeat |
| lin-61(n3624) | CCG | TCG | P132S | none predicted |
| lin-61(n3736) | TTT | TCT | F247S | MBT repeat |
| mep-1(n3680) | AGT | AAT | S309N | none predicted |
| mep-1(n3702) | CAG | TAG | Q706amber | — |
| | | CTT/ataagttt | | |
| mep-1(n3703) | CTT/gtaagttt | (SEQ ID NO: 32) | Exon 3 donor | — |
| sli-1(n3538) | TCA | TTA | S305L | SH2 |
| | | ttttccaa/AAA | | |
| sli-1(n3544) | ttttccag/AAA | (SEQ ID NO: 33) | Exon 6 acceptor | — |
| | | ttttttaa/GAT | | |
| sli-1(n3683) | ttttttag/GAT | (SEQ ID NO: 34) | Exon 4 acceptor | — |
| trr-1(n3630) | TGG | TAG | W2064amber | — |
| trr-1(n3637) | CAG | TAG | Q3444amber | — |
| trr-1(n3704) | CAA | TAA | Q694ochre | — |
| trr-1(n3708) | CGA | TGA | R1248opal | — |
| trr-1(n3709) | CGA | TGA | R2550opal | — |
| trr-1(n3712) | TGG | TAG | W2505amber | — |
| In the “Wild-type sequence” and “Mutant sequence” columns, exon and intron sequences are denoted by uppercase and lowercase script, respectively. Nucleotides altered by mutation are underlined. | | | | |
| *The predicted LET-418 protein contains a sequence described as a helicase domain. This domain was originally identified in helicases, but has since been found in non-helicase proteins. | | | | |
| # Many of these proteins share a common ATPase activity, and this domain contains residues that are important for ATP binding and hydrolysis. | | | | |
| ^{⋄}The adenosine inserted by the lin-35(n3763) frameshift mutation is not underlined because it is unclear which nucleotide in the adenosine repeat was inserted. | | | | |
| ^{†}In addition to the missense mutation described, we found an additional mutation associated with lin-36(n3765). This mutation, AG/gtaagaagaaaagc to AG/gtaagaagaaaagt, | | | | |
| # is present in the third intron of lin-36 and creates a possible splice donor sequence. If this splice donor were used, an inframe ochre (TAA) stop codon would be encountered, truncating the LIN-36 protein after 261 amino acids. | | | | |
| ^{‡}Due to alternative splicing, trr-1 encodes proteins that range in length between 4051 and 4061 amino acids | | | | |
| DPL-1 and EFL-1 are described by (Ceol et al., Mol Cell 7: 461-73, 2001 and (Page et al., Mol Cell 7: 451-60, 2001). LIN-9 is described by Beitel et al., Gene 254: 253-63, 2000); LIN-13 is described by Melendez et al., | | | | |
| # Genetics 155: 1127-37, 2000);; LIN-35 and LIN-53 are described by (Lu et al., Cell 95: 981-91, 1998); LIN-36 is described by (Thomas et al., Development 126: 3449-59, 1999); and SLI-1 is described by (Yoon et al., Science 269: 1102-5, 1995). | | | | |

Most mutations are GC-to-AT transitions that are characteristic of EMS mutagenesis (Anderson, Methods Cell Bio pp.31-58, 1995). MnN any of these mutations are predicted to truncate the corresponding synMuv proteins. The truncations predicted by efl-1(n3639), let-418(n3719), and lin-52(n3718) are particularly severe, and the synMuv and sterile phenotypes caused by these mutations may represent the null phenotypes of these genes. In addition, we found missense mutations that disrupt predicted functional domains of synMuv proteins. For example, n3536, n3626, n3629 and one of the two mutations of n3636 affect the ATPase/helicase domain of LET-418. LET-418 is a member of the Mi-2 family of ATP-dependent chromatin remodeling enzymes (Solari et al., CurrBiol 10: 223-6, 2000; Von Zelewsky et al., Development 127: 5277-84, 2000), and the LET-418 missense mutations suggest that LET-418 function is similarly dependent on ATP hydrolysis. At least one mutation affecting the LIN-13 protein, n3642, is predicted to disrupt a canonical zinc-finger motif. This missense mutation indicates that at least some of the twenty-four LIN-13 zinc fingers are important for its synMuv activity. Missense mutations affecting other synMuv proteins are not as easily linked to the disruption of predicted functional domains. These mutations may provide a useful starting point in identifying functional motifs within synMuv proteins that are not predicted by sequence comparisons.

Frequency of Mutant Isolation

The rate at which we isolated mutations was much higher than that observed in previous synMuv screens: including those 63 mutations described in this study, we recovered one synMuv mutation per 107 haploid genomes screened versus 1/750 (Ferguson et al., Genetics 123: 109-21, 1989), 1/400 and 1/667 in previous screens. We believe the reasons for this difference are threefold. First, our screen design allowed the isolation of synMuv mutations that also caused sterility. Sterile synMuv mutants were observed previously, but because the heterozygous siblings of these mutants were present in a sea of genotypically unrelated animals, the underlying mutations could not be recovered. Second, our parental strain carried the strong class A mutation, lin-15A(n767). The penetrance of a strain's Muv phenotype is dependent on the aggregate strengths of the component synMuv mutations. Therefore, even weak mutations may be identified in a strong synMuv background such as lin-15A(n767). Although we have not formally tested this possibility, we believe that some of the mutations we recovered only weakly affect synMuv activity. Such mutations may not have been recovcrcd in previous screens that were performed in partial loss-of-function synMuv backgrounds. Third, in screening a plate of many F₂ progeny derived from a single F₁ animal, we observed many genotypically identical animals per haploid genome screened. This type of screening likely accounts for our isolation of a number of partially penetrant synMuv mutations. Such mutations may not have been identified in earlier synMuv screens that typically observed fewer genotypically identical animals per haploid genome screened.

Our high rate of recovery indicates many genes can mutate to a synMuv phenotype. Including the ten genes we identified in this study, a total of 25 genes can act redundantly with class A synMuv genes. Many of these genes are represented by one or a few mutant alleles, indicating that screens for synMuv genes are not saturated.

The synMuv Genes we Identified likely Act in Different Pathways

Class B synMuv mutations synergize with class A synMuv mutations, but not with other class B synMuv mutations. Such genetic behavior led to the hypothesis that class B synMuv genes are part of a single genetic pathway (Ferguson et al., Genetics 123:109-21, 1989). In support of this hypothesis, mutations affecting different class B synMuv genes are similarly suppressed by loss-of-function mutations in the let-23 receptor tyrosine kinase and other let-60 Ras pathway loss-of-function mutations (Ferguson et al., Nature 326:259-67, 1987), a subset of class B synMuv gene products have been shown to interact in vitro, and their homologs are known function together in other systems (Lu et al., Cell 95: 981-91, 1998; Ceol et al., Mol Cell 7: 461-73, 2001). Because we conducted our screen in a class A synMuv background, we anticipated recovering mutations that affected genes of the class B synMuv pathway. In addition to Class B synMuv mutations, our results suggest that we recovered mutations that disable distinct genetic pathways. We recovered six mutations that affect the trr-1 gene. Unlike typical class B synMuv mutations, trr-1((n3712) synergize not only with class A synMuv mutations, but also with class B synMuv mutations. trr-1(n3712) single mutants also atypically show ectopic vulval induction. Because of its unusual genetic interactions, we propose that trr-1 functions;a pathway distinct front the class B synMuv pathway. We also recovered mutations affecting the sli-1, gap-1, and ark-1 genes. These genes were previously characterized as negative regulators of let-60 Ras pathway activity, acting genetically downstream of the let-23 receptor tyrosine kinase (Jongeward et al., Genetics 139:1553-66, 1995; Hajnal, et al., Genes Dev 11:2715-28 1997; Hopperet al., Mol Cell 6: 65-75, 2000). The molecular identities of sli-1, gap-1, and ark-1 support their action downstream of let-23. sli-1 encodes a homolog of the c-cbl proto-oncoprotein, which is thought to downregulate receptor tyrosine kinase levels through ubiquitin-mediated degradation (Yoon et al., Science 269: 1102-5, 1995; Levkowitz et al., Mol Cell 4: 1029-40, 1999). gap-1 is a member of the GTPase-activating protein family (Hajnal, et al., Genes Dev 11: 2715-28 1997). GAPs enhance the catalytic function of Ras family GTPases, thereby facilitating the switch from active GTP-bound to inactive GDP-bound Ras. ark-1 encodes a predicted cytoplasmic tyrosine kinase that interacts with the SEM-5 SH2/SH3 adaptor protein (Hopper et al., Mol Cell 6: 65-75, 2000). Since sem-5 acts downstream of the let-23 receptor tyrosine kinase, ark-1 is proposed to inhibit let-60 Ras signaling downstream of let-23. These genetic and molecular data suggest that sli-1, gap-1, and ark-1 directly regulatelet-60Ras pathway members and are likely not part of the canonical class B synMuv pathway, which is thought to regulate the let-60 Ras pathway either upstream of, or in parallel to, the let-23 receptor tyrosine kinase. We are currently placing our synMuv mutations into different genetic classes by examining interactions with class B synMuv and let-23 mutations.

lin-52 Encodes a New Putative Rb Pathway Protein

lin-35, a member of the class B synMuv pathway, encodes a protein similar to the mammalian tumor suppressor pRb (Lu et al., Cell 95: 981-91, 1998). Other genes with class B synMuv activity encode DP, E2F, RbAp48, histone deacctylase and HP1 family proteins (Lu et al., Cell 95: 981-91, 1998; Ceol et al., Mol Cell, 7: 461-73, 2001; Couteau et al., EMBO Rep 3: 235-41, 2002). Mammalian homologs of these proteins are known to functionally, and in some cases physically, interact with pRb. These and other parallels indicate that the classB synMuv pathway is an analog of Rb pathways in other systems. Consequently, additional class B synMuv genes may have homologs with analogous functions in other systems. One such gene is lin-52. By the genetic criteria outlined above, lin-52 is a class B synMuv gene. lin-52 mutations synthetically interact with class A mutations, but not with class B mutations. Furthermore, preliminary experiments indicate that the Vul phenotype of a let-23 loss-of-function mutation is epistatic to the Muv phenotype caused by lin-52 and lin-15A loss of function. lin-52 encodes a small protein, portions of which are conserved in similarly small proteins predicted by the human, mouse and Drosophila genome sequences. The characterization of these and other class B synMuv protein homologs should help to determine whether they too function in Rb-mediated signaling.

The experiments described above were carried out as follows

Strains and General Techniques

Strains were cultured as described by (Brenner, Genetics 77: 71-94, 1974), and grown at 20° C. unless otherwise indicated. The wild-type parent of all the strains described in this study was the Caenorhabditis elegans Bristol strain N2. For some two and three-factor mapping experiments we used the polymorphic strain RW7000 (Williams et al., Genetics 131: 609-24, 1992). We also used strains containing the following mutations:
LGI: bli-3(e767), lin-17(n677), unc-11(e47), unc-73(e936),lin-44(n1792), unc-38(x20), dpy-5(e61), lin-35(n745), lin-61(sy223), unc-13(e1091), lin-53(n833) (Ferguson et al., Genetics 123: 109-21 (1989), unc-54(e1092) (Dibb et al., J Mol Biol 183: 543-51, 1985).
LGII: lin-31(n301), dpy-10(e128), tra-2(q276), rol-6(e187), dpl-1(n2994), unc-4(e120), unc-53(n569), mex-1(it9), rol-1(e91)
LGIII: dpy-17(e164), lin-1(e185), sma-3(e491), lin-13(n770) (Ferguson et al., Genetics 123: 109-21 (1989), lin-37(n758), lin-36(n766), unc-36(e251), lin-9(n112), unc-32(e189), unc-16(e109), sqv-3(n2842), lin-52(n771) (Ferguson et al., Genetics 123: 109-21 (1989), unc-47(e307), unc-69(e587), dpy-18(e364)
LGIV: lin-1(e1275), unc-5(e53), unc-24(e138), mec-3(e1338), lin-3(n378), sem-3(n1900), dpy-20(e1282),unc-22(e66), dpy-26(n198), unc-31(e169), unc-30(e191), lin-54(n2231), dpy-4(e1166)LGV: tam-1 (cc567) (Hsieh et al., Genes Dev 13: 2958-70, 1999), unc-46(el77), let-418(s1617), dpy-11(e224), rol-4(sc8), unc-76(e911), efl-1 (n3318) Ceol et al., Mol Cell 7: 461-73 (2001). dpy-21(e428) LGX: sli-1(syl43), aex-3(ad418), unc-1(e1598n1201), dpy-3(e27), gap-1(ga133) (Hajnal et al., Genes Dev 11: 2715-28, 1997), unc-2(e55), lon-2(e678), unc-10(e102), dpy-6(e14), unc-9(e101), unc-3(e151), lin-15A(n767),lin-15AB(n765). Unless otherwise noted, the mutations used are described by (Riddle et al., C. elegans II (Cold Spring Harbor, N.Y., Cold Spring Harbor Laboratory Press 1997). In addition, we used strains containing the following chromosomal aberrations: mnDf57 II (Sigurdson, et al., Genetics108: 331-45, 1984), mnDf90II(Sigurdson, et al., Genetics108: 331-45, 1984), mnDJ29II(Sigurdson, et al., Genetics108: 331-45, 1984), mnDf87 II(Sigurdson, et al., Genetics 108: 331-45, 1984), mIn1[dpy-10(e128)mIs14] II (Edgley et al., Mol Genet Genomics 266: 385-95, 2001), mmC1[dpy-10(e128) unc-52(e444)] II (Herman, Genetics 88: 49-65, 1978), nDf40 III (Hengartner et al., Nature 356: 494-9, 1992), qC1[dpy-19(e1259)glp-1(q339)] III (Austin, et al., Cell 58: 565-571, 1989), sDf63 IV, sDf62 IV (Clark et al., Mol Gen Genet 232: 97-105, 1992), sDf10 IV (Rogalski et al., Genetics 102: 725-36, 1982), eT1(III;V) (Rosenbluth et al., Genetics 99: 415-28, 1981), nT1(IV;V) (Ferguson et al., Genetics 110: 17-72, 1985). mIs14, an integrated transgene linked to the chromosomal inversion mIn1, consists of a combination of GFP-expressing transgenes that allow mIn14-containing animals to be scored beginning at the 4-cell stage of embryogenesis (Edgley et al., Mol Genet Genonmics 266: 385-95, 2001).
   Isolation of New Alleles

We mutagenized lin-15A (n767) hermaphrodites with ethyl methanesulfonate (EMS) as described by (Brenner, Genetics 77: 71-94, 1974). We allowed these animals to recover on food for between 15 minutes to one hour, and then transferred individual P₀ larvae in L4 lethargus to 50 mm plates. After three to five days, 20 F₁ L4 larvae per P₀ were individually transferred to 50 mm plates, and, subsequently, F₂ animals on these plates were screened for a Muv phenotype. We screened the progeny of 3380 F₁ animals using this procedure.

Linkage Group Assignment

We used the following markers to determine linkage of newly isolated synMuv mutations to autosomes: dpy-5 I, rol-6 II, unc-32 III, dpy-20 IV, rol-4 V. We generated animals heterozygous for the new synMuv mutation and for at least two of these markers. For fertile synMuv mutants we picked Muv progeny and determined if these progeny segregated the markers, whereas for sterile synMuv mutants we picked single marker homozygotes and determined if these animals segregated the synMuv mutation. We also mapped some mutations using polymorphisms present in the RW7000 strain. We generated animals heterozygous for the new synMuv mutation and for RW7000 markers. We picked individual Muv progeny of these animals, performed lysis and used the resulting template DNA to monitor linkage to each of the autosomes by PCR (Williams et al., Genetics 131: 609-24, 1992). We tested for sex linkage to assign some new synMuv mutations to the X chromosome. Briefly, we generated heterozygous or hemizygous mutant males and mated them with marked lin-15A(n767) hermaphrodites. We then determined whether all, indicating sex linkage, or roughly half, indicating autosomal linkage, of the cross progeny hermaphrodites of this mating segregated the synMuv mutation. Some lin-15B mutations were not tested for sex linkage. Instead, we tentatively assigned X-chromosome linkage based on the presence, when lin-15A(n767) males were mated with these mutants, of cross-progeny males with pseudovulval ventral protrusions. Such protrusions are often observed in hemizygous lin-15AB mutant males (Ferguson et al., Genetics 110: 17-72, 1985) but are found at a much lower penetrance in lin-15A(n767) males that are hemizygous for an X-linked synMuv mutation affecting genes other than lin-15B. The mutations we assigned in this manner were later determined by complementation tests to affect lin-15B.

Complementation Tests

We typically performed complementation tests by mating males heterozygous for the new mutation and hemizygous for lin-15A(n767), or, if X-linked, males hemizygous for both the new mutation and lin-15A(n767), into marked synMuv mutant hermaphrodites, all of which contained a lin-15A mutation. Hemizygous lin-15B(n3711)lin-15A(n767) males could not mate. To perform complementation tests with this mutation, we mated tra-2(q276), lin-15B(n3711)lin-15A(n767)/++ XX males into marked lin-15A-B hermaphrodites. For new mutations that caused recessive sterility, we generated heterozygous males by starting matings with wild-type L4 males and individual gravid, putative heterozygous mutant hermaphrodites. For complementation tests we used cross-progeny males derived from plates that had self-progeny Muv animals present. In all complementation tests, unmarked cross-progeny hermaphrodites were scored.

Construction of Deficiency Heterozygotes

To construct trr-1(n3712) heterozygotes with mnD57, mnDf90 and m7nDf29, Df/mIn; lin-15A(n767) males were generated. These males were mated into rol-6 trr-1(n3712)/mIn1; lin-15A(n767) hermaphrodites and non-Rol, non-Gfp cross-progeny were scored. mnDf87 heterozygous males do not mate so in this case we generated lin(n3712)mnDf87; lin-15A(n767) animals by mating lin(n3712)mIn1; lin-15A(n767) males into unc-4 mnDf87/mIn1; lin-15A(n767) hermaphrodites. To construct the lin-52 heterozygote with nDf40, we mated nDf40 dpy-18/unc-36,lin-15A(n767) males into unc-36 lin-52(n771); lin-15A(n767) hermaphrodites and scored non-Unc cross-progeny. mep-1/Df animals were constructed by mating Df/nT1;+/nT1 males into dpy-20 mep-1; lin-15A(n767) hermaphrodites and scoring non-Dpy cross-progeny.

Transgenic Animals

Germline transformation was performed, as described by (Mello et al., Embo J 10: 3959-70, 1991), by injecting cosmid (5-10 ng/μL) or plasmid (50-80 ng/μL) DNA into lin-52 or mep-1 mutants. Either pRF4, which causes a dominant Rol phenotype, or pPD93.97, which expresses gfp under the control of the myo-3 promoter, was used as a coinjection marker.

lin-52 cDNA Isolation

We obtained a partial lin-52 cDNA clone, yk253b12, that included 249 nucleotides of the lin-52 open reading frame and also included the 3′ untranslated region and a polyA tail. We used the 5′ RACE system v2.0 for rapid amplification of chromosome ends (GIBCO-BRL, LIFE TECHNOLOGIES, Inc. Gaithersburg, Md.) to determine the 5′ end of the lin-52 transcript. We ligated the two portions of the lin-52 cDNA together to generate a full-length cDNA clone. The lin-52 5′ RACE products were trans-spliced to the SL2 leader sequence consistent with observations made by (Zorio et al., Nature 372: 270-2, 1994).

Allele Sequence

We used PCR-amplified regions of genomic DNA as templates in determining gene sequences. For each gene invsetigateu, we determined the sequences of all exons and splice junctions. Whenever observed, the sequence of a mutation was confirmed using an independently-derived PCR product. All sequences were determined using an automated ABI 373 DNA sequencer.

### EXAMPLE II

As detailed below, we have identified a distinct class of genes, termed the class C synMuv genes, that negatively regulate vulval induction.

Proper vulval development in the nematode C. elegans requires that specific ectodermal cells, termed Pn.p cells, adopt different cell fates. The specification of Pn.p cells that eventually make vulval tissue occurs in two steps, each of which involves the selection of a subset of Pn.p cells from a larger Pn.p field (Sulston, Dev Biol 56: 110-56, 1977). In the first step, which occurs in the L1 larval stage shortly after the Pn.p cells are generated, anterior and posterior Pn.p cells fuse with the syncytial hypodermis. After this first step, the unfused midbody P(3-8).p cells each have the capacity to adopt a vulval cell fate (Sternberg et al., Cell 44: 761-72, 1986). In a second step, however, only three of these cells, P(5-7).p, adopt such fates in which they undergo three rounds of division to generate seven or eight descendants. P3.p, P4.p and P8.p adopt non-vulval fates, typically dividing only once to generate two descendants that eventually fuse with the syncytial hypodermis. The decision to adopt vulval cell fates occurs during the L2 and early L3 larval stages and is followed by cell divisions and differentiation in the L3 and L4 larval stages, respectively (Sternberg et al., Cell 44: 761-72, 1986; Ferguson et al., Nature 326: 259-67, 1987). While mutations in class C synMuv genes alone cause mild defects, when a class C gene mutation is combined with either a class A or class B mutation, the two mutations synergize to produce more severe vulval induction and other developmental defects. Class C synMuv genes, trr-1, hat-1, and epc-1, encode homologs of the transcriptional coactivator TRRAP, the MYST family acetyltransferases TIP60 and Esalp and the Drosophila Enhancer of Polycomb (E(Pc)) protein, respectively. Because of the predicted acetyltransferase activity of the HAT-1 protein and because orthologs TRRAP and E(Pc) family proteins have been copurified in histone acetyitransferase complexes, we propose that a combination of histone acetyltransferase and histone deacetylase activities is required to properly specify vulval cell fates in C. elegans.

trr-1 Interacts with Class A and Class B synMuv Mutations

We performed a genetic screen for synMuv mutants in a lin-15A(n767) background and identified six mutations in our pool of isolates that failed to complement each other and that defined the gene trr-1. To quantitate the synMuv phenotype in these mutants, we scored the number of cells that were induced to become vulva.

To more precisely quantitate the Muv phenotype of trr-1; lin-15A strains, we scored the numbers of P(3-8).p cells induced per animal and found that all strains had a similarly penetrant, temperature-sensitive hyperinduced phenotype (Table 5A).

TABLE 5
trr-1 mutations cause a hyperinduced phenotype

| | | | | |
|---|---|---|---|---|
| | | Ave. # | | |
| | Temp | P(3-8).p | % animals | |
| Genotype | (° C.) | induced (±SE) | hyperinduced | n |

A. trr-1 interactions with synMuv mutations

| | | | | |
|---|---|---|---|---|
| wild-type | 20 | 3.00 (±0) | 0 | 31 |
| lin-15A(n767) | 20 | 3.00 (±0) | 0 | 24 |
| lin-38(n751) | 20 | 3.00 (±0) | 0 | 27 |
| trr-1(n3630); lin-15A(n767) | 20 | 4.52 (±0.15) | 82 | 45 |
| trr-1(n3637); lin-15A(n767) | 20 | 4.52 (±0.14) | 83 | 54 |
| trr-1(n3704); lin-15A(n767) | 20 | 4.20 (±0.13) | 79 | 43 |
| trr-1(n3708); lin-15A(n767) | 20 | 4.71 (±0.14) | 92 | 36 |
| trr-1(n3709); lin-15A(n767) | 20 | 4.81 (±0.13) | 95 | 39 |
| trr-1(n3712); lin-15A(n767) | 20 | 4.07 (±0.12) | 74 | 54 |
| lin-15A(n767); trr-1(RNAi) | 20 | 5.60 (±0.08) | 100 | 44 |
| trr-1(n3712) lin-38(n751) | 20 | 4.14 (±0.23) | 79 | 14 |
| lin-38(n751); trr-1(RNAi) | 20 | 5.66 (±0.08) | 100 | 32 |
| wild-type | 15 | 3.00 (±0) | 0 | 29 |
| lin-15A(n767) | 15 | 3.00 (±0) | 0 | 32 |
| trr-1(n3704); lin-15A(n767) | 15 | 3.13 (±0.05) | 21 | 24 |
| trr-1(n3712); lin-15A(n767) | 15 | 3.06 (±0.03) | 13 | 32 |
| wild-type | 25 | 3.00 (±0) | 0 | 36 |
| lin-15A(n767) | 25 | 3.02 (±0.02) | 3.6 | 28 |
| trr-1(n3704); lin-15A(n767) | 25 | 5.87 (±0.06) | 100 | 38 |
| trr-1(n3712); lin-15A(n767) | 25 | 5.47 (±0.14) | 100 | 17 |

B. trr-1 single mutants

| | | | | |
|---|---|---|---|---|
| wild-type | 20 | 3.00 (±0) | 0 | 31 |
| trr-1(n3630) | 20 | 3.03 (±0.02) | 6.1 | 33 |
| trr-1(n3637) | 20 | 3.08 (±0.04) | 13 | 30 |
| trr-1(n3704) | 20 | 3.01 (±0.01) | 2.6 | 39 |
| trr-1(n3708) | 20 | 3.05 (±0.03) | 8.1 | 37 |
| trr-1(n3709) | 20 | 3.03 (±0.02) | 6.3 | 32 |
| trr-1(n3712) | 20 | 3.10 (±0.03) | 13 | 89 |
| trr-1(RNAi) | 20 | 3.09 (±0.05) | 13 | 32 |
| wild-type | 15 | 3.00 (±0) | 0 | 29 |
| trr-1(n3704) | 15 | 3.08 (±0.05) | 12 | 26 |
| trr-1(n3712) | 15 | 3.06 (±0.03) | 12 | 25 |
| wild-type | 25 | 3.00 (±0) | 0 | 36 |
| trr-1(n3704) | 25 | 3.04 (±0.03) | 3.9 | 51 |
| trr-1(n3712) | 25 | 3.07 (±0.03) | 13 | 48 |

The number of P(3-8).p cells induced was scored as described below. Induction was scored after raising strains at the indicated temperature for two generations. trr-1 mutant homozygotes were scored by examining the non-Gfp progeny of trr-1/mIn1[dpy-10(e128)mIs14] heterozygous parents.

The hyperinduction we observed occurred in P3.p, P4.p and P8.p to similar extents. To determine if trr-1 interacted with other class A synMuv genes, we constructed a trr-1(n3712) lin-38 double mutant. These double mutant animals were also hyperinduced (Table 5A), suggesting that trr-1 functions in parallel not only to lin-15A, but to the class A synMuv pathway in general.

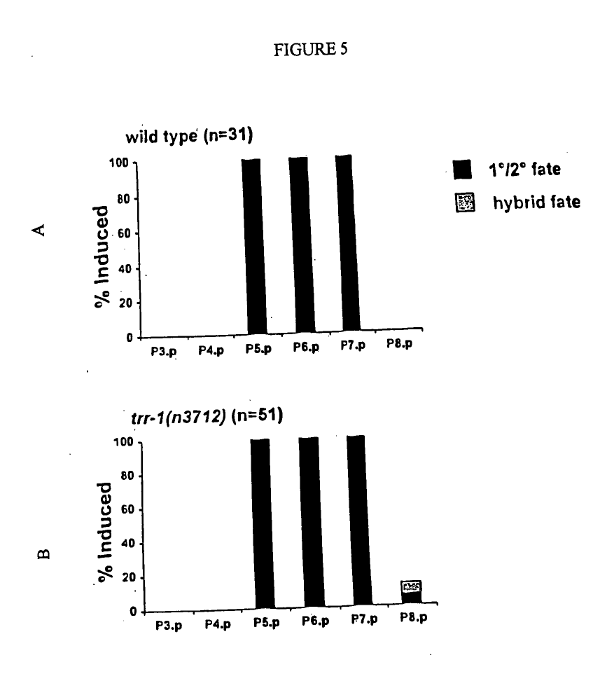

We also isolated trr-1(n3712) and the other trr-1 mutations away from any other synMuv mutations. Nearly all class A and class B synMuv single mutants adopt a wild-type pattern of P(3-8).p fates (Table 5B), however trr-1 adults had a weakly penetrant hyperinduced phenotype (Table 5B). By examining the cell fates adopted by individual P(3-8).p cells in L4 animals, we determined that the vulval cell-fate transformations of trr-1 single mutants always occurred in P8.p (FIG. 5). In addition to ectopic vulval cell-fate transformations, all trr-1 mutations caused slow growth and sterility, although some mutant animals occasionally produced a small number of eggs (<10, as compared to ˜300 for the wild-type), all of which died during embryogenesis.

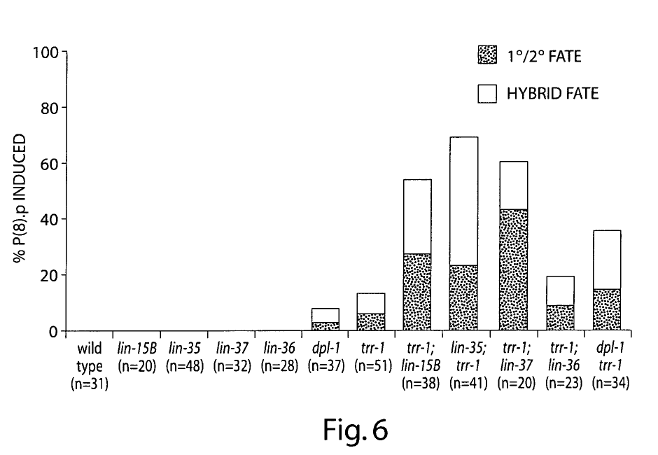

To determine if trr-1 interacts with class B synMuv genes, we constructed double mutant strains containing trr-1(n3712) and mutations of class B synMuv genes. Interestingly, double mutant strains combining trr-1(z3712) with mutations of lin-15B, lin-35 Rb, and lin-37 showed a significant increase in the penetrance of P8.p transformation (FIG. 6). In addition to the increase in P8.p transformation, we occasionally observed ectopic transformations of P3.p and P4.p. Since lin-15B(n744), lin-35(n745) and lin-37(n758) are strong loss-of-function and possibly null mutations of their corresponding genes, these results indicate that trr-1 functions redundantly with at least a subset of class B synMuv genes.

No significant increase was observed in trr-1(n3712), lin-36(n766) double mutants (FIG. 6). By various genetic criteria, this loss-of-function lin-36 mutation behaves unlike mutations in other class B synMuv genes (Hsieh et al., Genes Dev 13: 2958-70, 1999; Fay et al., Genes Dev 16: 503-17, 2002). There are at least two possibilities to explain the unusual behavior of lin-36(n766). First, the lack of enhancement could be allele specific, with the lin-36(n766) mutation disrupting a function that is redundant with a class A synMuv function but not disrupting a separable lin-36 function that is redundant with trr-1 activity. Alternatively, our observations with lin-36 could reflect a gene-specific lack of enhancement. For example, the strength of the lin-36 defect may not be equivalent to that of other class B synMuv gene defects such that lack of lin-36 activity may be readily observable in a class A synMuv background but, unlike other class B synMuv defects, not observable in a trr-1 background. Enhancement tests using additional lin-36 alleles will help to resolve this issue.

trr-1 Encodes a Protein Similar to Mammalian TRRAP

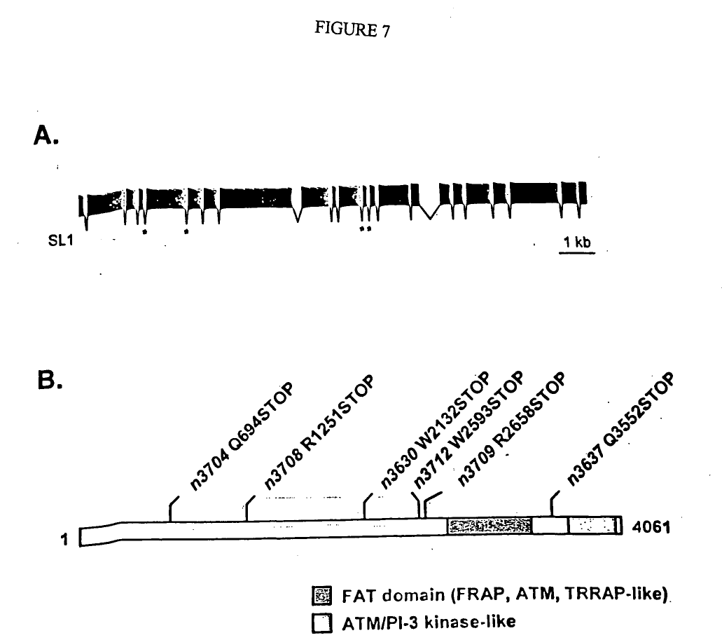

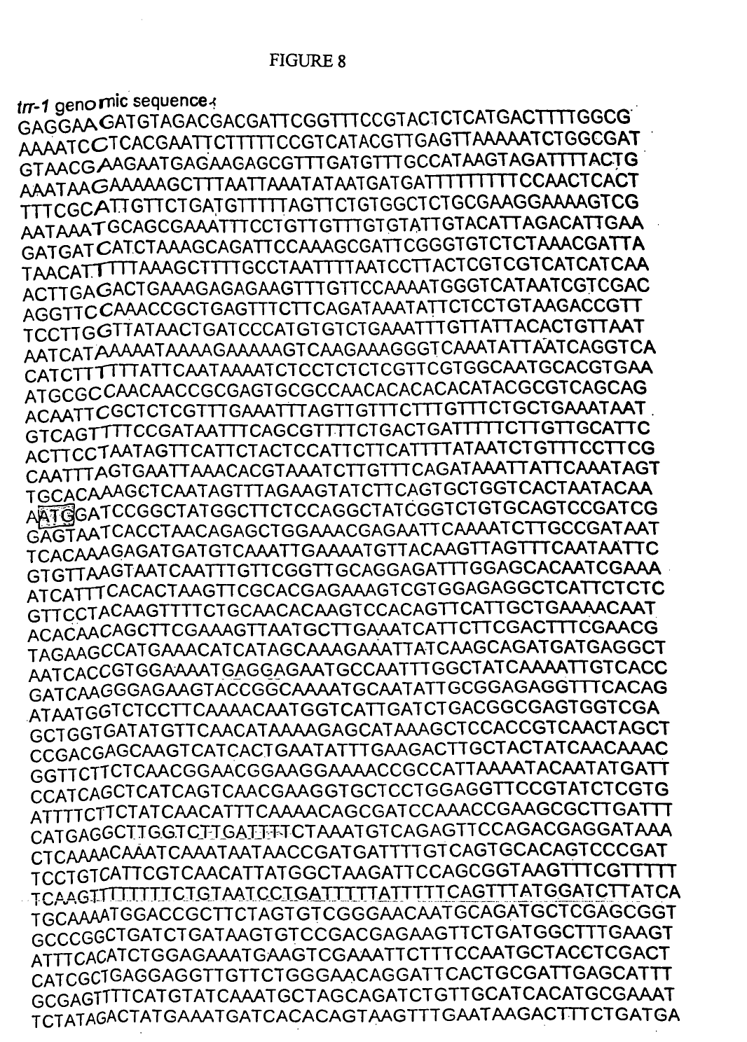

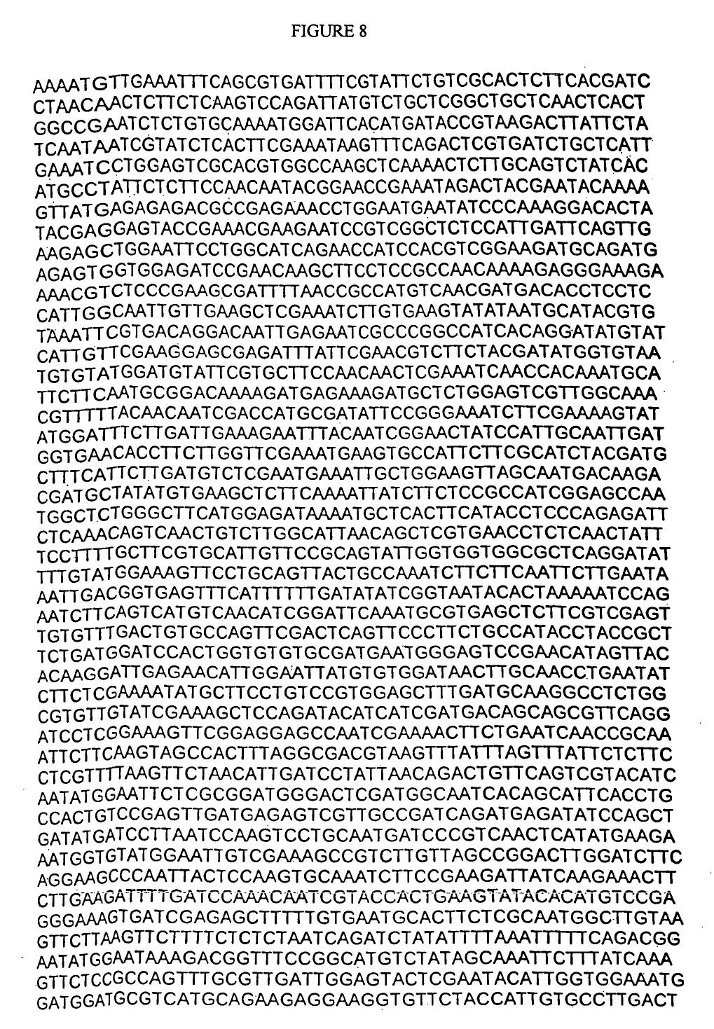

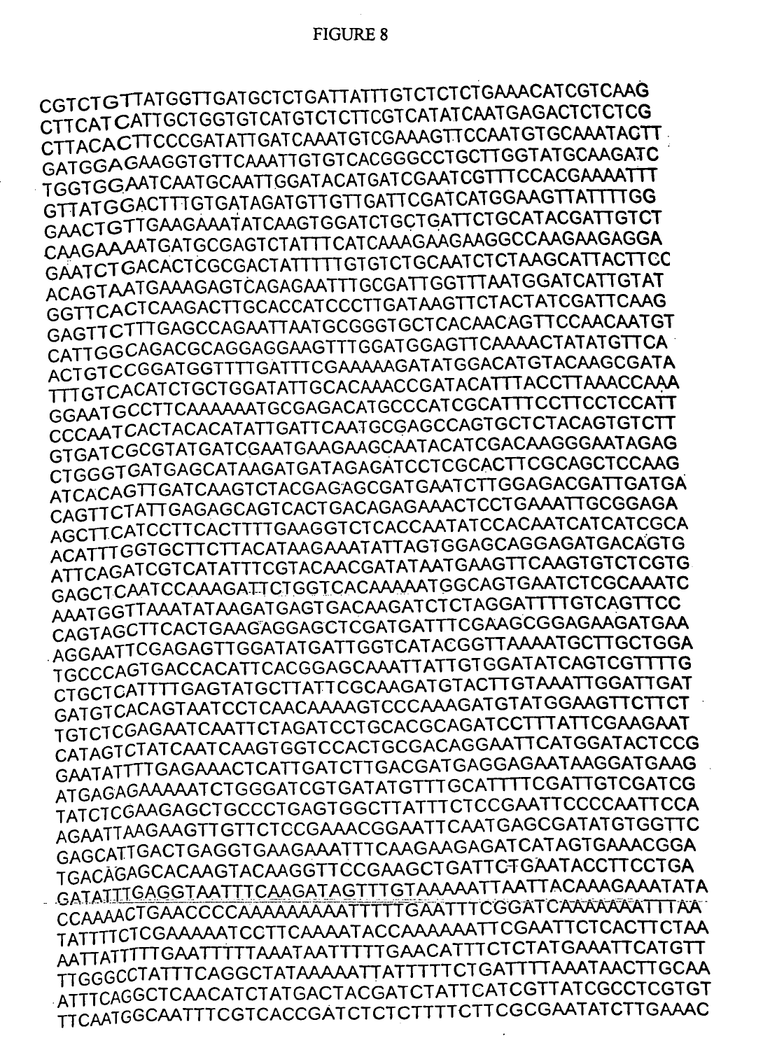

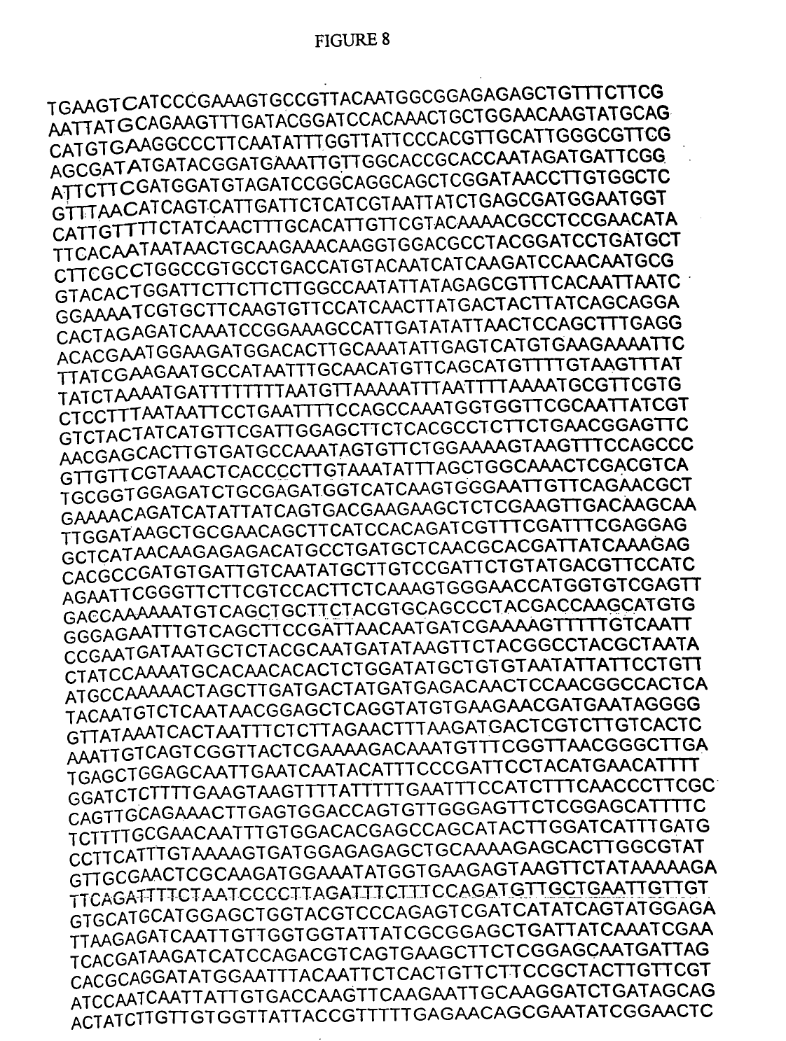

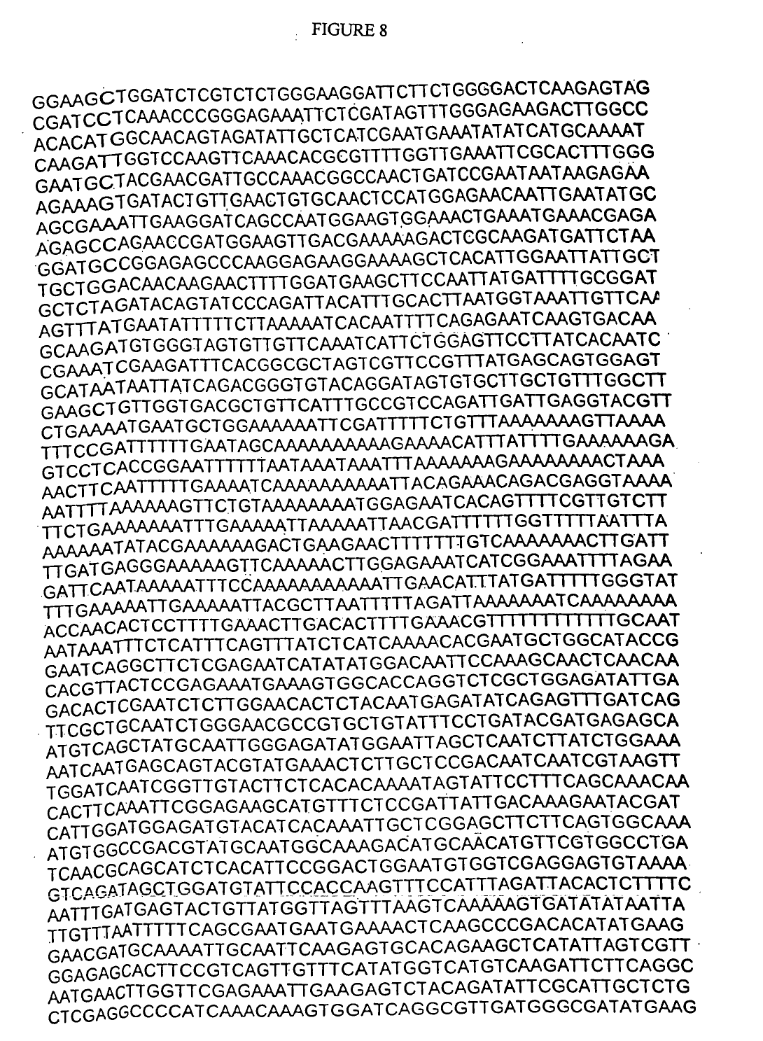

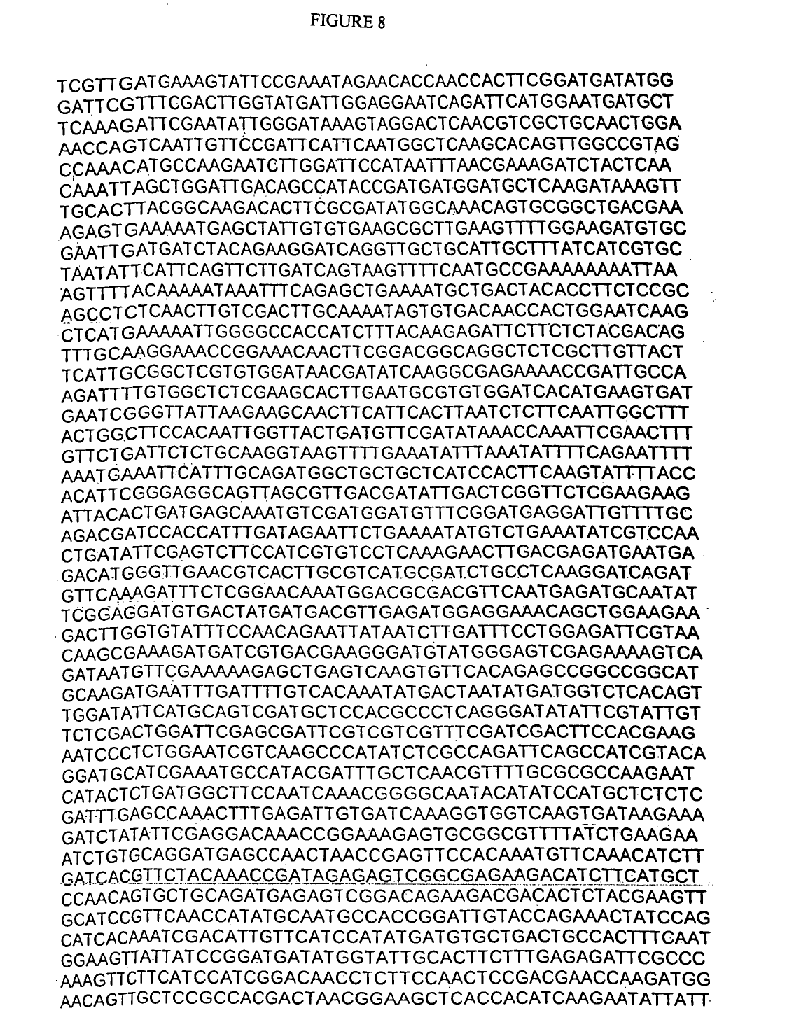

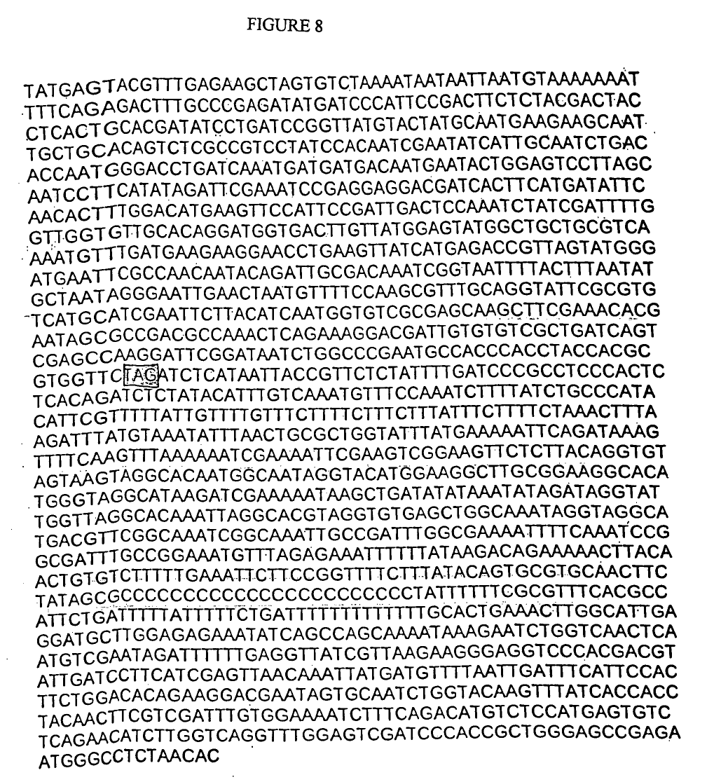

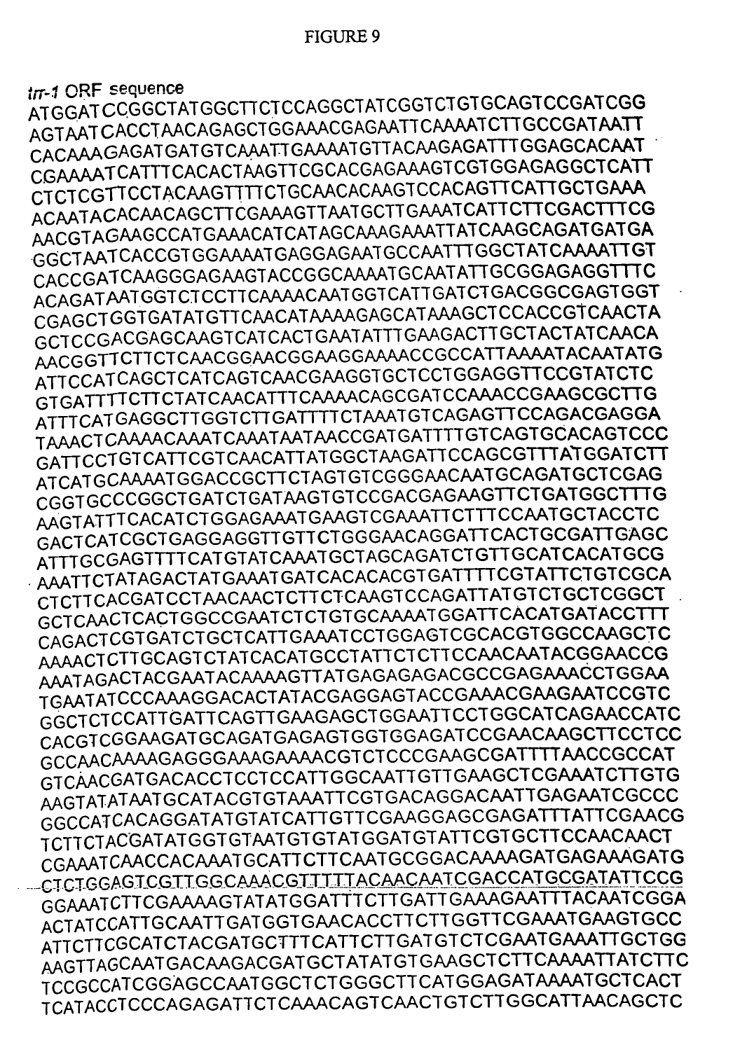

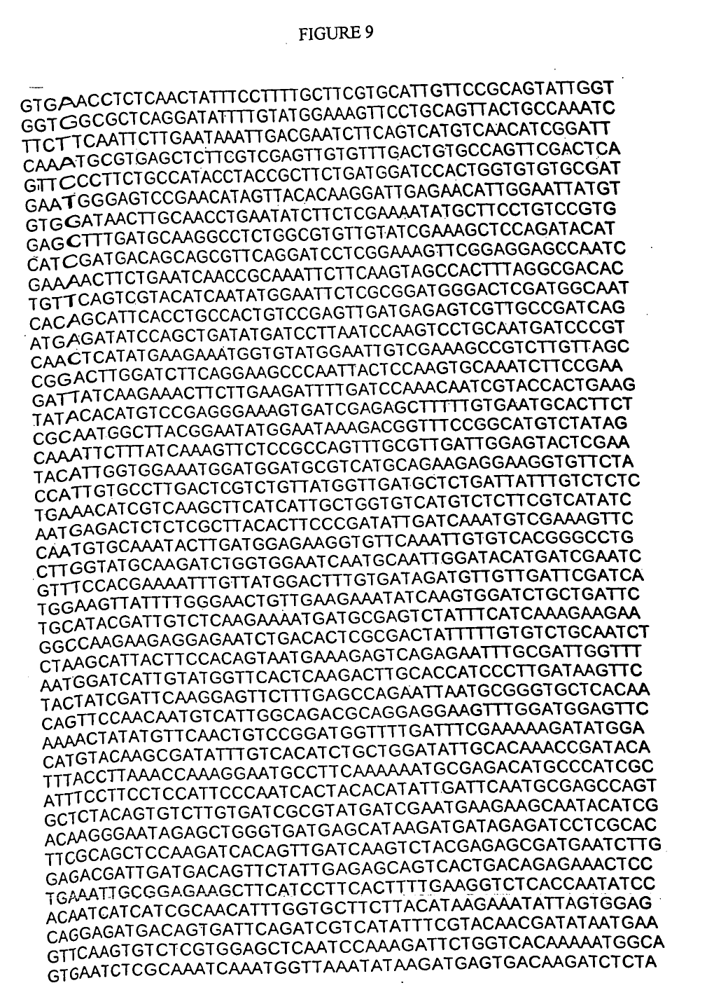

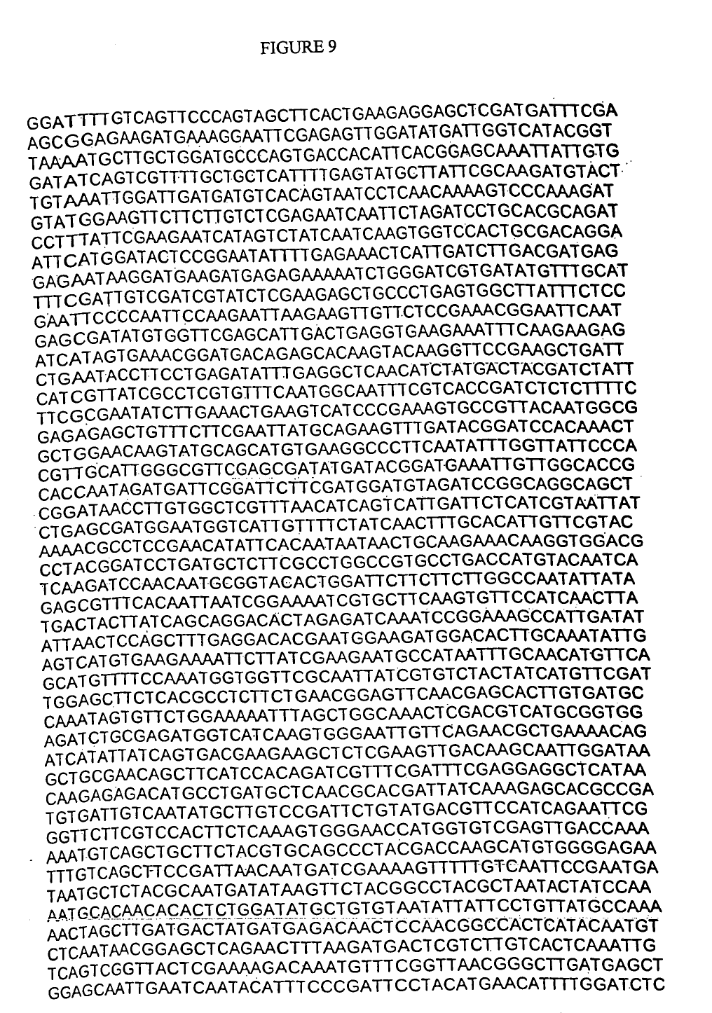

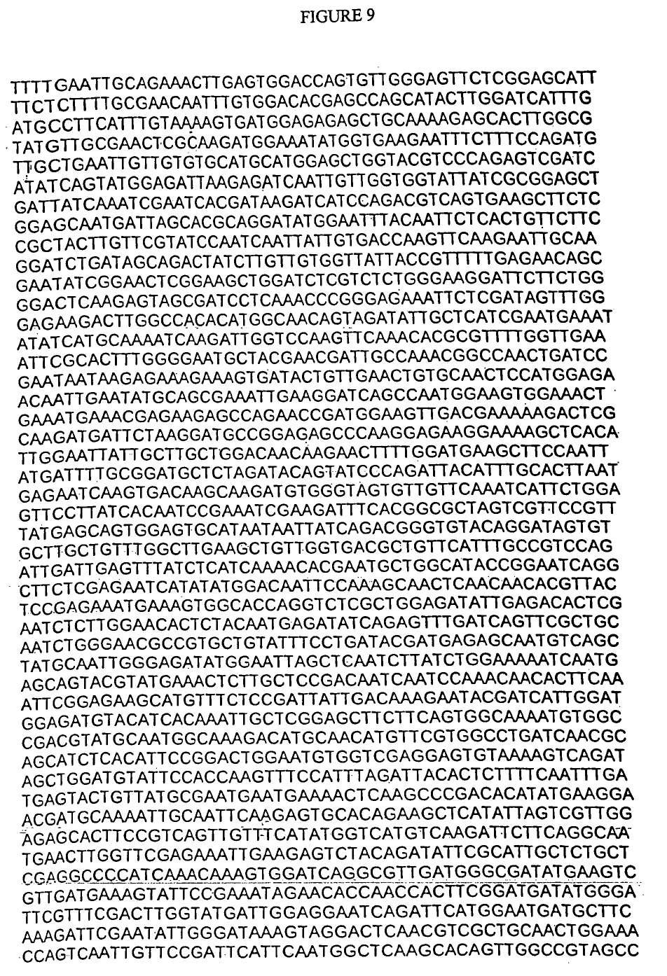

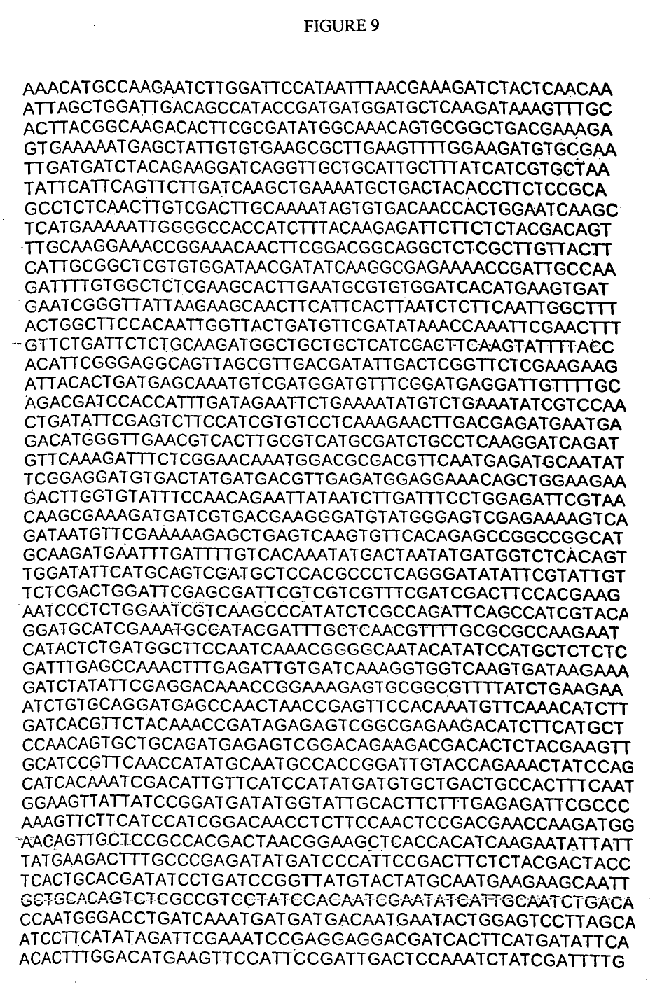

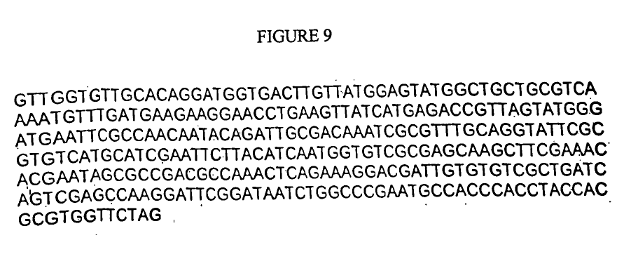

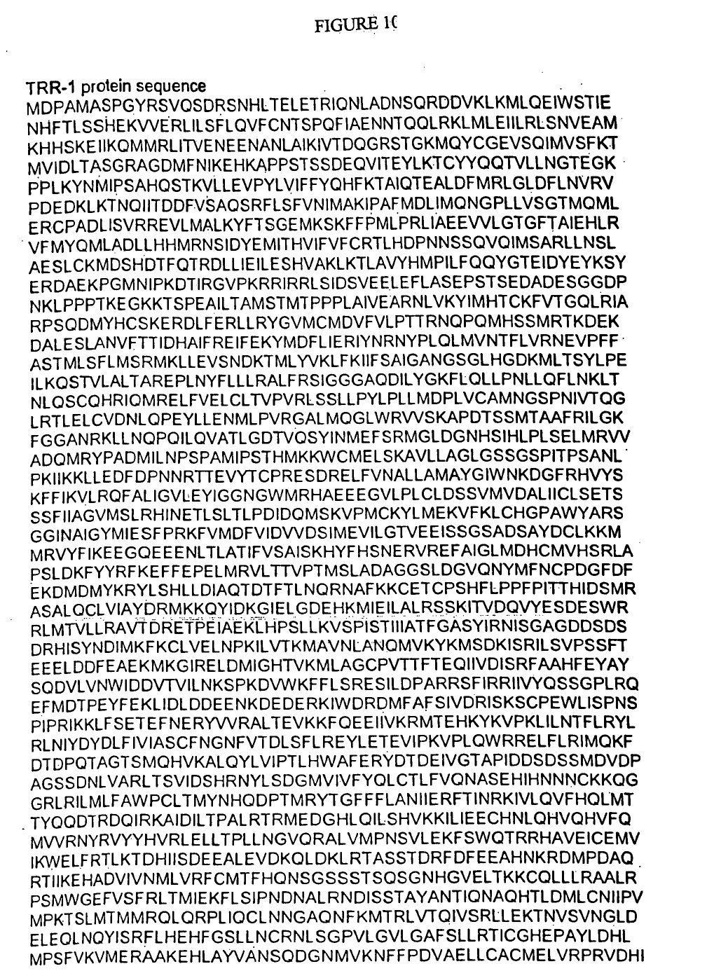

We mapped trr-1 to a small region of LGII and cloned the gene using transformation rescue as detailed below. To confirm the identity of trr-1, we obtained a partial CDNA and, using RNA derived from this cDNA, found that RTNA-mediated interference (RNAi) of this gene caused a highly penetrant hyperinduced phenotype in lin-15A and lin-38 mutant backgrounds (Table 5). As determined by RT-PCR and 5′ RACE, the trr-1 gene consists of 22 exons, four of which are alternatively spliced (FIG. 7A). Since the sites of alternative splicing are separated by only six or nine nucleotides, the most exclusive (4054 amino acids) and inclusive (4064 amino acids) isoforms differ slightly in size. The genomic sequence of trr-1 is shown in FIG. 8. The sequence of the trr-1 open reading frame is shown in FIG. 9.

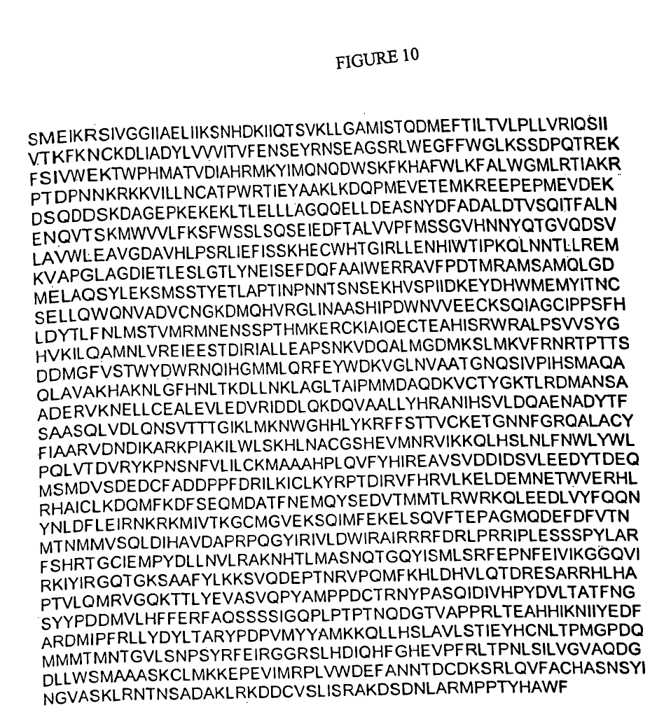

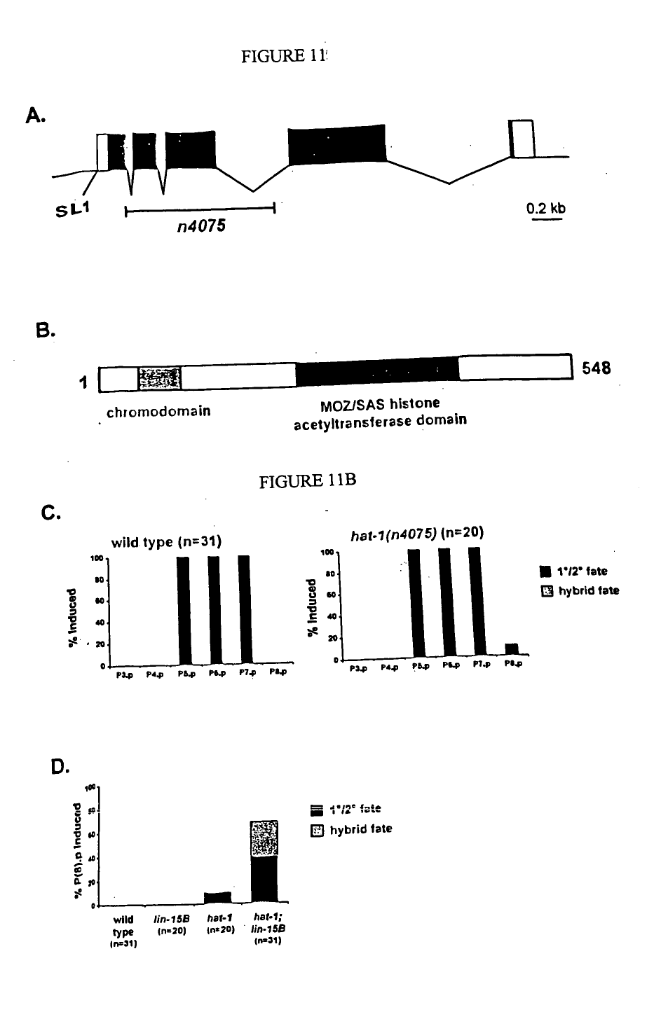

The deduced amino acid sequence of TRR-1 is shown in FIG. 10. The predicted TRR-1 proteins are similar to mammalian myc-associated protein TRRAP (transformation/transcription domain-associated protein) and its yeast homolog Tralp throughout most of their lengths (McMahon et al., Cell 94: 363-74, 1998; McMahon et al., Cell 94: 363-74, 1998; Saleh et al., J Biol Chem 273: 26559-65, 1998). TRRAP and Tra1p are similarly large proteins, extending 3828 and 3744 amino acids, respectively. The largest predicted TRR-1 isoform is 25 percent identical to TRRAP and 19 percent identical to Tra1p. TRR-1, TRRAP, and Tra1p share limited regions of homology with other proteins (FIG. 7B). One of these regions is located at the carboxy terminus and is similar to the catalytic domains of ATM and PI-3-like kinases. Interestingly, the DXXXXN (SEQ ID NO:29) and DFG motifs critical for kinase activity are not present in TRR-1, TRRAP, or Tralp (Hunter et al., Cell 83: 1-4, 1995). Instead of having an enzymatic function, this domain of T-RRAP has been proposed to mediate protein-protein interactions (McMahon et al., Cell 94: 363-74, 1998). All six trr-1 mutations introduce nonsense codons (FIG. 7B). trr-1(n3637) is predicted to truncate the protein just prior to the ATM/PI-3 kinase-like domain. The phenotypic strength of trr-1(n3637) is similar to that of other alleles, suggesting that deletion of the ATM/PI-3 kinase-like domain alone results in a severe loss of protein function. Finally, trr-1(n3630), trr-1(n3637), and trr-1(n3712) introduce amber stop codons, and we observed that the sterility associated with these alleles was reduced by the sup-5(e1464) informational suppressor tRNA mutation. This suppression, along with the partially penetrant sterility caused by trr-1(RNAi), confirms that the sterility observed in trr-1 mutants is truly due to loss of trr-1 function.

trr-1(RNAi) is Synthetically Lethal with Mutations in lin-35 Rb and Other Class B synMuv Genes

trr-1(RNAi) caused more severe phenotypic consequences than did trr-1 mutations. For example, the ectopic induction phenotype of lin-15A, trr-1(RNAi) mutants was much stronger than that of trr-1; lin-15A mutant strains (Table 5). We do not believe this difference is reflective of a partial loss of gene function caused by all of the trr-1 mutations. Instead we propose that at least some of the mutations cause a severe loss of gene function and that the difference is due to an effect of trr-1(RNAi) on maternally-provided gene activity. In support of this proposal, trr-1(n3704)/mnDf87; lin-15A and trr-1(n3712)/mnDf87; lin-15A mutants that were severely deficient in zygotically-provided trr-1 activity but retained maternally-provided trr-1 activity had phenotypic penetrances that were similar to those of trr-1; lin-15A homozygotes and were weaker than those of lin-15A; trr-1(RNAi) mutants. Also arguing that trr-1; lin-15A homozygotes have significantly reduced zygotically-provided trr-1 gene activity, the protein truncations predicted by trr-1(n3704) and other trr-1 mutations are likely to remove functional domains and compromise TRR-1 activity.

We further characterized the effects of trr-1(RNAi). In wild-type and class A synMuv genetic backgrounds, trr-1(RNAi) caused retarded growth, adult sterility and weakly penetrant embryonic and larval lethalities (Table 6).

TABLE 6
trr-1(RNAi) is synthetically lethal with class B but not with
class A synMuv mutations

| | % dead | % dead | Total % |
|---|---|---|---|
| Genotype | embryos | L1 larvae | lethality (n) |
| wild-type | 0 | 0 | 0 (1062) |
| trr-1(RNAi) | 6.6 | 1.2 | 7.8 (726) |
| lin-15A(n767) | 0 | 0 | 0 (823) |
| lin-38(n751) | 0.1 | 0 | 0.1 (1003) |
| lin-15B(n744) | 0.2 | 0 | 0.2 (1002) |
| lin-35(n745) | 0.6 | 0.2 | 0.8 (482) |
| lin-36(n766) | 0.3 | 0 | 0.3 (890) |
| dpl-1(n2994) | 14 | 1.1 | 15.1 (265) |
| lin-15A(n767); trr- | 3.2 | 0.9 | 4.1 (470) |
| 1(RNAi) | | | |
| lin-38(n751); trr- | 3.8 | 1.3 | 5.1 (628) |
| 1(RNAi) | | | |
| lin-15B(n744); trr- | 62.5 | 36.0 | 98.5 (469) |
| 1(RNAi) | | | |
| lin-35(n745); trr- | 66.2 | 33.8 | 100 (263) |
| 1(RNAi) | | | |
| lin-36(n766); trr- | 19.4 | 21.6 | 41.0 (444) |
| 1(RNAi) | | | |
| dpl-1(n2994); trr- | 45.1 | 53.6 | 98.7 (304) |
| 1(RNAi) | | | |

Animals injected with trr-1 dsRNA were individually plated 10-15 hours following injection. Injected animals were subsequently transferred to new plates every 24,hours until egg laying had ceased. Dead embryos and larvae on a plate were counted at least two days after eggs were laid. All of the mutant strains in which trr-1(RNAi) was performed are homozygous viable.

Interestingly, trr-1(RNAi) caused highly penetrant embryonic and larval lethalities in combination with many class B synMuv mutations. Most of the dead embryos arrested at the late embryonic pretzel stage and those that hatched died shortly thereafter. We have not yet determined a basis for this lethality. It is important to note that many of the class B synMuv mutations tested are predicted to have severe effects on their cognate class B synMuv proteins. Since trr-1(RNAi) can synthetically interact with strong reduction-of-function or null class B synMuv mutations, these data indicate that trr-1 functions redundantly with class B synMuv genes not only in vulval cell-fate determination but also in an essential process earlier in development.

trr-1(RNAi) causes synthetic lethality in a lin-36(n766) background although the penetrance of this lethality is not as high as in other class B synMuv backgrounds. This assay therefore unmasks a redundancy between trr-1 and lin-36 that we did not observe in the P8.p induction assay. As discussed above, the strength of the lin-36 defect may not be equivalent to the strengths of defects of other class B synMuv genes. This difference in strengths may explain why, relative to other class B synMuv genes, lin-36 shows weaker interactions with trr-1 in terms of synthetic lethality and synthetic P8.p induction.

trr-1 Synthetically Interacts with dpl-1 DP

Mammalian TRRAP and yeast Tralp are thought to function as coactivator proteins that bridge transcription factors to histone acetyltransferases (McMahon et al., Cell 94: 363-74, 1998; Brown et al., Science 292, 2333-7, 2001). Based on coimmunoprecipitation and functional assays, E2F transcription factors were linked to TRRAP (McMahon et al., Cell 94: 363-74, 1998; Lang et al., J Biol Chem 276: 32627-34, 2001). In vivo E2F and DP family proteins form heterodimers that are bound by Rb family proteins via a direct interaction with the E2F subunit reviewed by (Dyson, Genes Dev 12: 2245-62, 1998; (Trimarchi et al., Nat Rev Mol Cell Biol 3: 11-20, 2002). We previously determined that one of two C. elegans E2E family members, efl-1, and the sole DP family member, dpl-1, are class B synMuv genes Ceol et al., Mol Cell 7: 461-73 (2001). As noted above, lin-35 Rb was also characterized as a class B synMuv gene, and the LIN-35 Rb protein was found to form a complex with DPL-1 and EFL-1 in vitro (Lu et al., Cell 95: 981-91, 1998; Ceol et al., Mol Cell 7: 461-73, 2001).

LIN-35 Rb and Rb proteins in other species are thought to recruit histone deacetylase complexes to regulate E2F-dependent transcription

(Brehm et al., Nature 391: 597-601, 1998; (Luo et al., Cell 92, 463-73, 1998; Magnaghi-Jaulin et al., Nature 391: 601-5, 1998). Coupling these results with our genetic finding that trr-1 acts redundantly with lin-35 Rb to negatively regulate vulval induction, one might speculate that EFL-1 and DPL-1 recruit distinct LIN-35-containing and TRR-1-containing complexes to appropriately regulate vulval cell fate determination. To examine this possibility, we wished to determine if trr-1 acted through efl-1 and dpl-1 to negatively regulate vulval development.

Without being tied to a particular theory, three lines of evidence suggest that trr-1 does not act solely through transcription factors, efl-1 and dpl-1; first, the ectopic induction of P8.p in dpl-1 trr-1 double mutants is greater than that observed in either single mutant (FIG. 6). Because of the sterility conferred by the dpl-1(n3316) null and trr-1(n3712) mutations, these mutants were derived from dpl-1(n3316) trr-1(n3712)/++ mothers. It is notable that in this test we substantially reduced maternally-provided dpl-1 activity by injecting mothers with dpl-1 dsRNA and scoring dpl-1 (n3316 RNAi) trr-1(n3712) progeny; second, in a weak lin-15A mutant background at 15° C., trr-1(RNAi) greatly enhanced the ectopic induction observed in dpl-1 mutant animals that were derived from dpl-1 heterozygous mutant mothers (Table 7);

TABLE 7
trr-1 acts redundantly with dpl-1

| | Ave. # P(3-8).p | % animals |
|---|---|---|
| Genotype | induced (±SE) | mutant (n) |
| lin-15A(n433); trr-1(RNAi) | 3.17 (±) | 20 (15) |
| dpl-1(n3316); lin-15A(n433) | 3.00 (±0) | 0 (35) |
| dpl-1(n3316); lin-15A(n433); | 4.98 (±) | 89 (45) |
| trr-1(RNAi) | | |

Animals were raised at 15° C., a temperature at which dpl-1(n3316); lin-15A(n433) mutants do not show hyperinduction. dpl-1(n3316) homozygous mutants were recognized as the Unc non-Gfp progeny of dpl-1 (n3316) unc-4(e120)/mIn1[dpy-10(e128) mIs14] heterozygous parents.

third, when performed in a homozygous dpl-1 mutant background, trr-1(RNAi) caused synthetic lethality with dpl-1 (Table 6). Since viable trr-1(RNAi) dpl-1 progeny could be derived from heterozygous, but not homozygous dpl-1 mutant mothers, this synthetic lethality apparently required a lack of maternally-provided dpl-1 activity. These results indicate that trr-1 does not act only through dpl-1 to regulate vulval development and embryonic and larval viability. Although all of these assays were conducted in dpl-1 mutant backgrounds, we expect that, since reduction of dpl-1 function is predicted to affect all C. elegans DP/E2F activity, these results similarly apply to efl -1.

In addition to these data, one other observation argues against the model that trr-1 acts solely through dpl-1. Whereas double mutants containing lin-35(n745), a putative null allele of lin-35, and trr-1(n3712) display highly penetrant ectopic induction of P8.p, the ectopic induction in dpl-1(n3316RNAi) mutants is relatively weak (FIG. 6). If both lin-35 and trr-1 were acting solely through dpl-1, defects of equivalent strengths would be expected.

The Muv Phenotype of trr-1 Mutants Requires let-60 Ras Pathway Activity

Previous studies determined that a conserved Ras pathway induces vulval development in C. elegans reviewed by (Sternberg et al., Trends Genet 14: 466-72, 1998). Loss-of-function mutations affecting genes in this pathway cause a vulvaless (Vul) phenotype characterized by P(3-8).p adopting hypodermal instead of vulval cell fates. To determine if Ras pathway activity is required for the trr-1 mutant phenotype, we constructed strains in which the functions of trr-1, lin-15A and a Ras pathway gene were reduced. The uninduced phenotype caused by let-23 receptor tyrosine kinase and let-60 Ras mutations was epistatic to the hyperinduced phenotype caused by trr-1 and lin-15A loss of function (Table 8).

TABLE 8
trr-1 epistasis with let-23 RTK, let-60 Ras and lin-3 EGF

| | Ave. # | % | |
|---|---|---|---|
| | P(3-8).p induced | animals | |
| Genotype | (±SE) | hyperinduced | n |
| wild-type | 3.00 (±0) | 0 | 31 |
| lin-15A(n767) | 3.00 (±0) | 0 | 24 |
| lin-15A(n767); trr-1(RNAi) | 5.60 (±0.08) | 100 | 44 |
| let-23(sy97); lin-15A(n767) | 0.02 (±0.02) | 0 | 28 |
| let-23(sy97); lin-15A(n767); | 0.05 (±0.03) | 0 | 42 |
| trr-1(RNAi) | | | |
| let-60(n1876); lin-15A(n767) | 0 (±0) | 0 | 17 |
| let-60(n1876); lin-15A(n767); | 0 (±0) | 0 | 23 |
| trr-1(RNAi) | | | |
| lin-3(n378); lin-15A(n767) | 0.30 (±0.07) | 0 | 40 |
| lin-3(n378); lin-15A(n767); | 4.35 (±0.20) | 85 | 20 |
| trr-1(RNAi) | | | |

let-23(sy97) homozygous mutants were recognized as Rol Unc non-Gfp progeny of rol-6(e187) let-23(sy97) unc-4(e120)/mIn1[dpy-10(e128) mIs14]; lin-15A(n767) heterozygous parents, and let-60(n1876) homozygous mutants were recognized as Unc progeny of let-23(n1876) unc-22(e66)/nT1; +/nT1; lin-15A(n767) heterozygous parents.

These results indicate that Ras pathway activity is required to produce the trr-1; lin-15A Muv phenotype. By contrast, trr-1; lin-3; lin-15A triple mutants showed a wild-type level of induction in P(5-7).p and ectopic induction in P3.p, P4.p and P8.p. lin-3 encodes an EGF-like protein that is produced by the gonadal anchor cell and is thought to act non-cell autonomously to stimulate Ras pathway activity in P(5-7).p (Hill et al., Nature 358: 470-6, 1992). These findings suggest that a basal level of lin-3-independent Ras pathway activity, when combined with mutations in trr-1 and lin-15A, is sufficient to induce vulval cell fates in P(3-8).p.
hat-1 and epc-1, but not ssl-1, Loss of Function Phenocopies trr-1

TRRAP and Tralp are components of protein complexes that acetylate histones (Allard et al., Embo J 18: 5108-19, 1999; reviewed by Brown et al., Trends Biochem Sci 25:15-9, 2000). These complexes are distinguished by their histone acetyltransferase subunits: the mammalian TFTC and p/CAF and the yeast SAGA complexes contain Gcn5 family acetyltransferases, whereas the mammalian TIP60 and the yeast NuA4 complexes contain MYST family acetyltransferases.

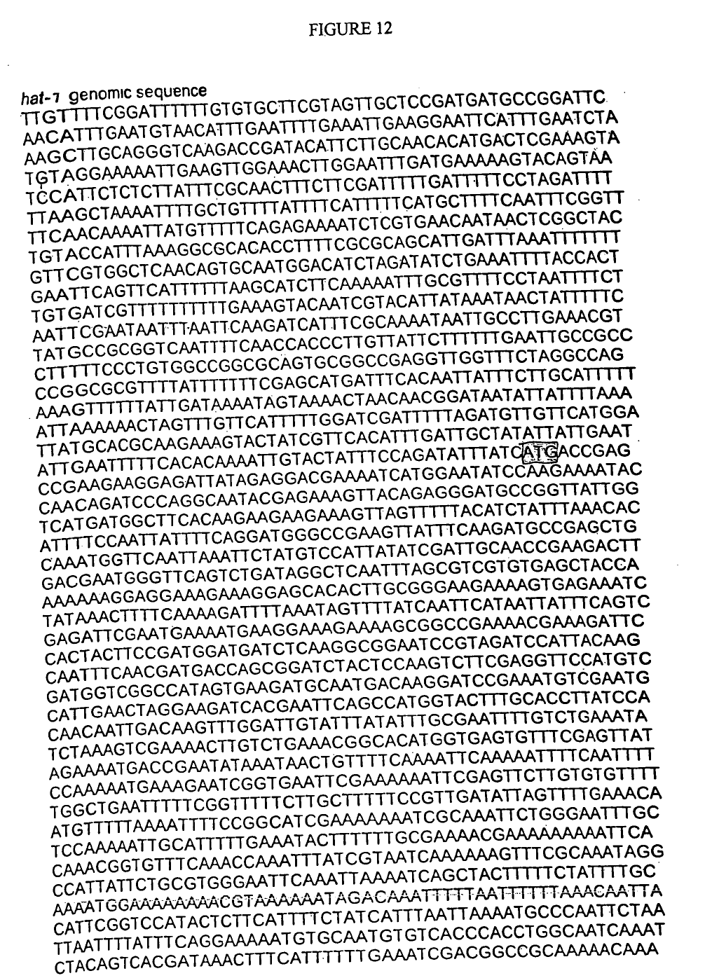

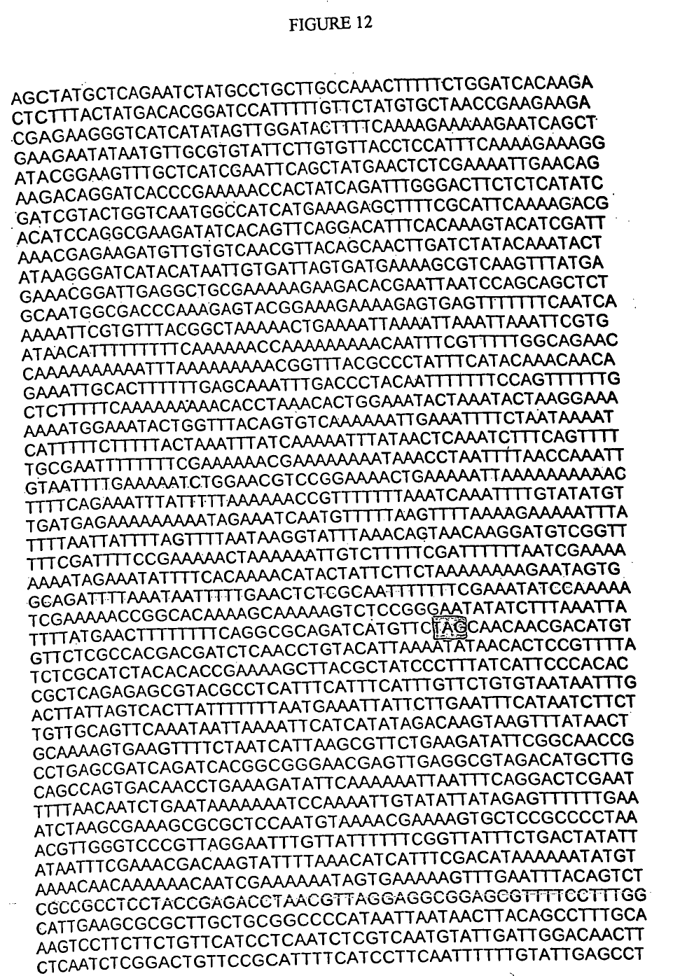

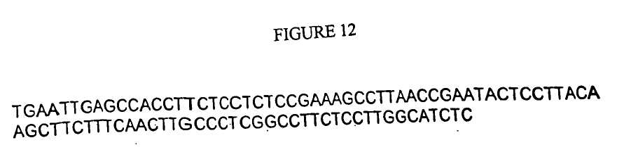

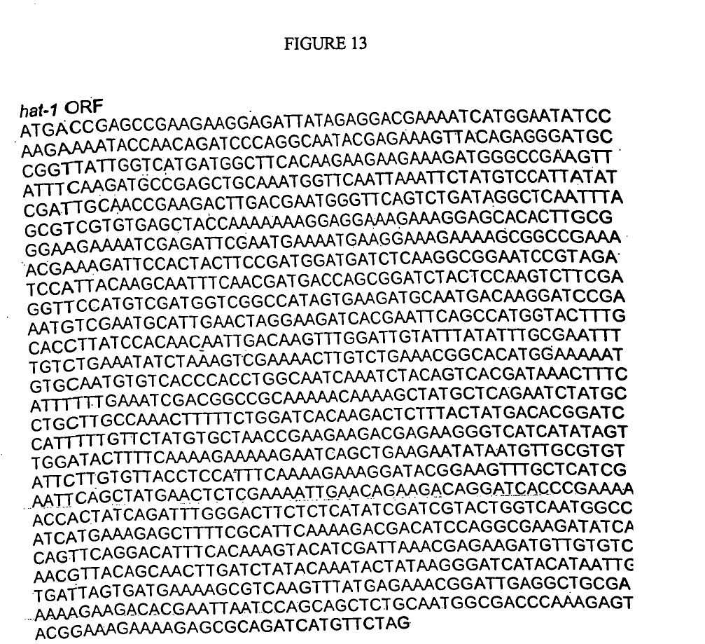

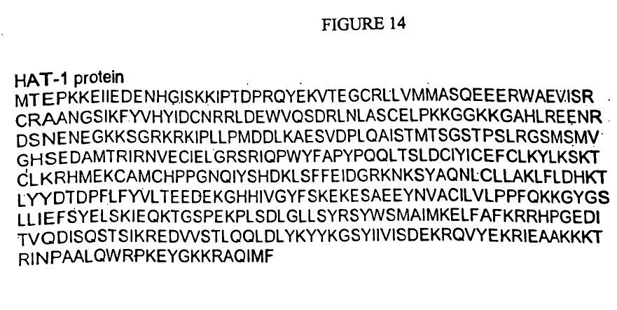

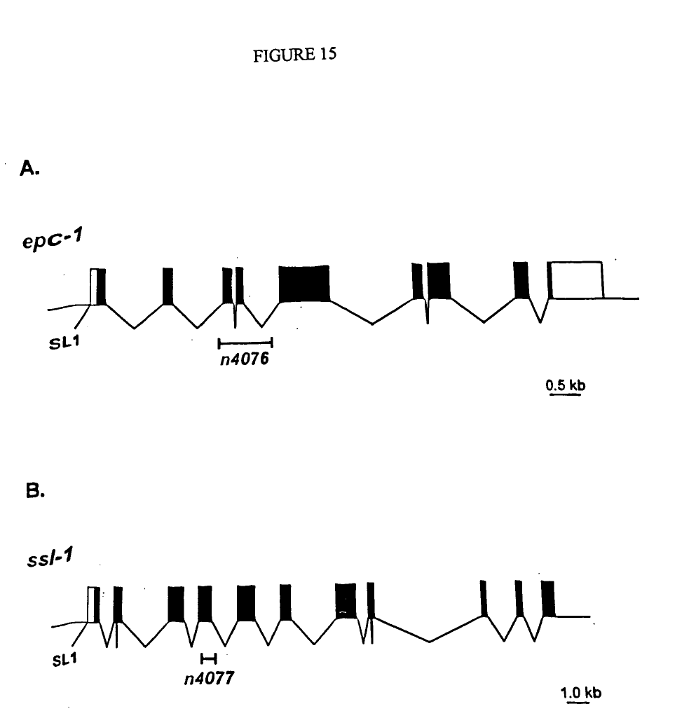

To determine if TRR-1 might function with a histone acetyltransferase in C. elegans, we used RNA-mediated interference to inactivate such genes. Whereas inactivation of a Gcn5 homolog Y47G6A.6 had no effect, inactivation of a MYST family gene we have named hat-1 produced a highly penetrant NMuv phenotype in a lin-15A background. To further characterize hat-1, we isolated a deletion allele, n4075, that removes 1010 base pairs from the hat-1 locus and is predicted to produce a protein that contains the first 35 amino acids of HAT-1 followed by 52 unrelated amino acids prior to termination (FIG. 11A). The genomic nucleic acid sequence of hat-1 is shown in FIG. 12. The nucleic acid sequence of the hat-1 open reading frame is shown in FIG. 13. The predicted full-length HAT-1 protein is 458 amino acids long, and this deletion is expected to remove the conserved chromodomain and acetyltransferase catalytic domain (FIG. 11B). The amino acid sequence of the wild-type HAT-1 protein is shown in FIG. 14. hat-1(n4075) mutants exhibited the same spectrum of phenotypes and genetic interactions as trr-1 mutants. hat-1(n4075) single mutants were slow growing and sterile. In combination with class A synMuv mutations, hat-1(n4075) caused a severe Muv phenotype characterized by P3.p, P4.p and P8.p ectopic induction (Table 8). Alone, hat-1(n4075) caused ectopic induction of P8.p (FIG. 11C). In combination with a lin-15B mutation, the penetrance of this ectopic induction was greatly increased (FIG. 11D).

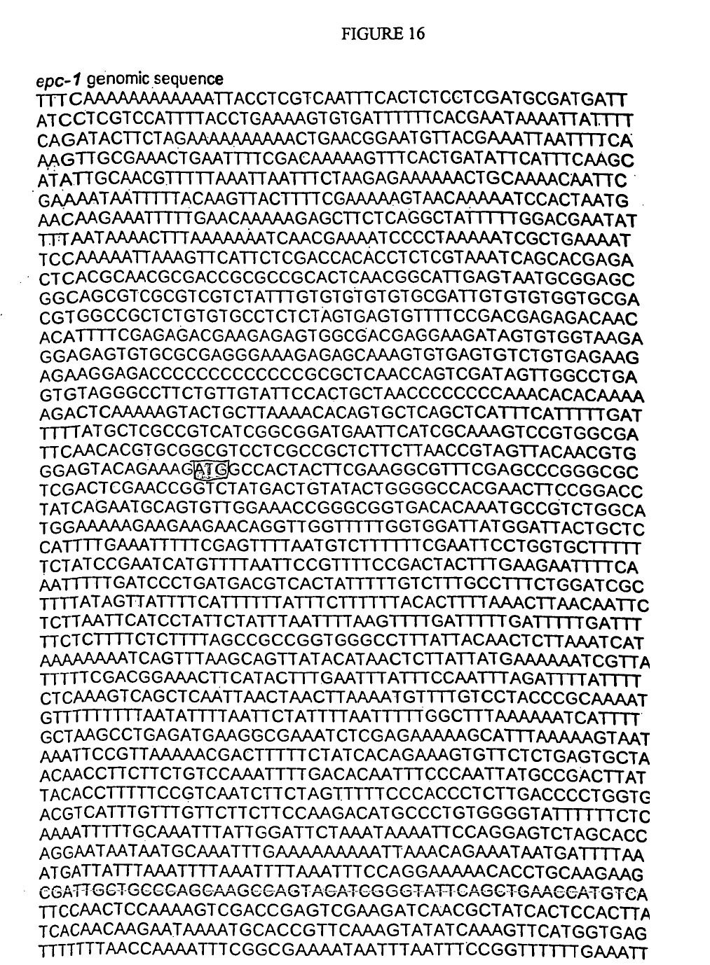

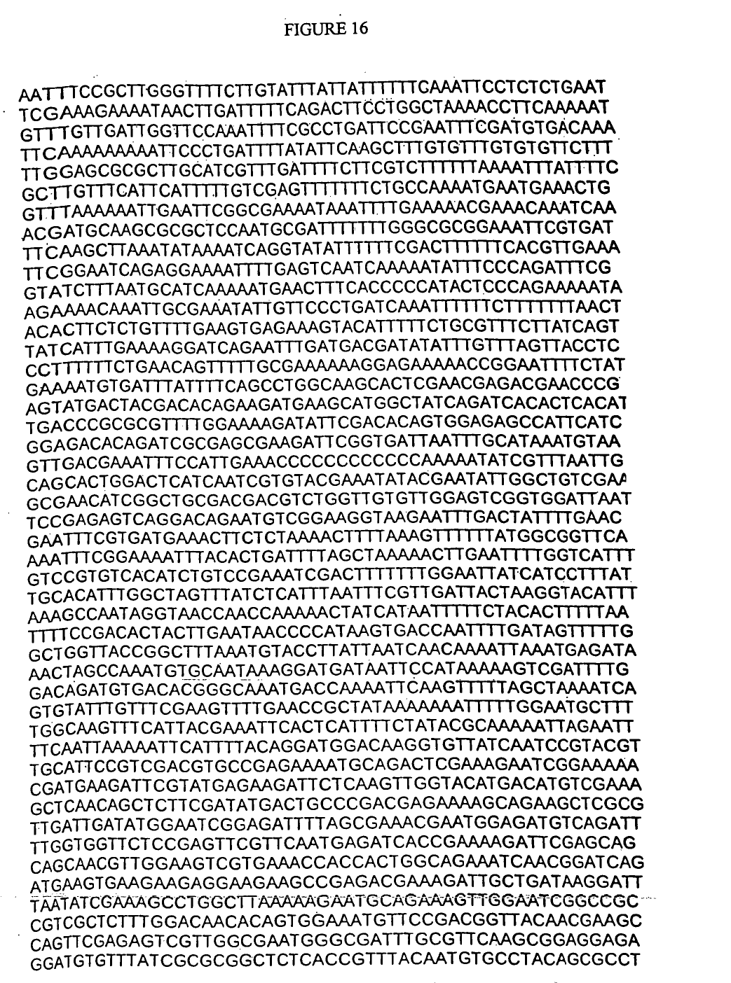

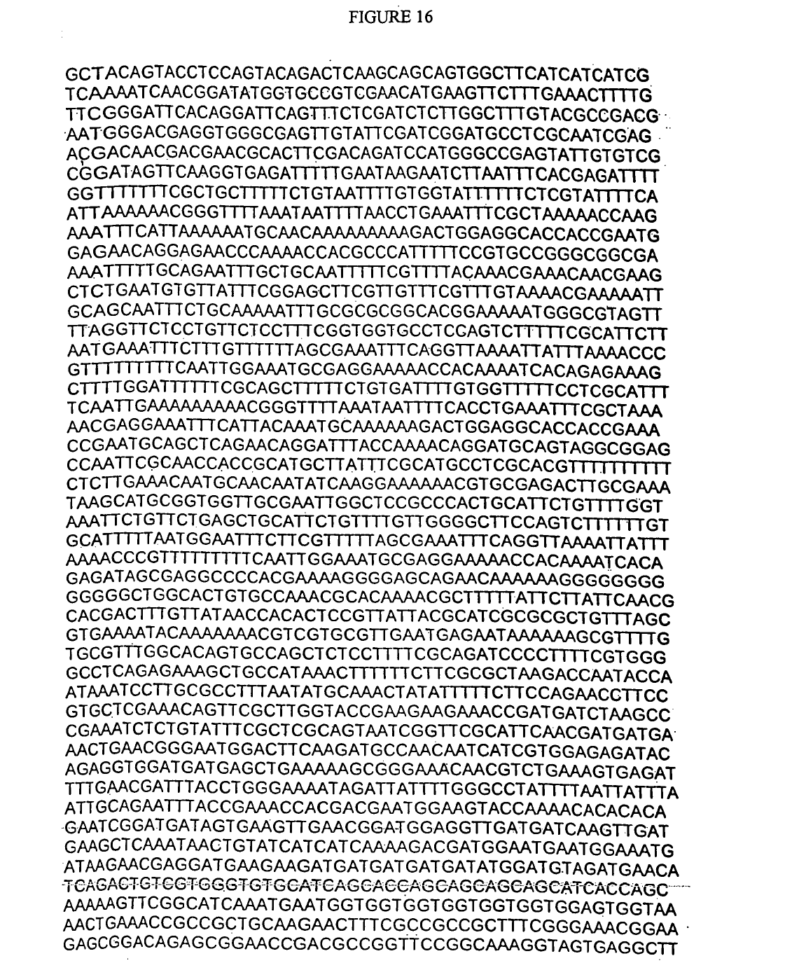

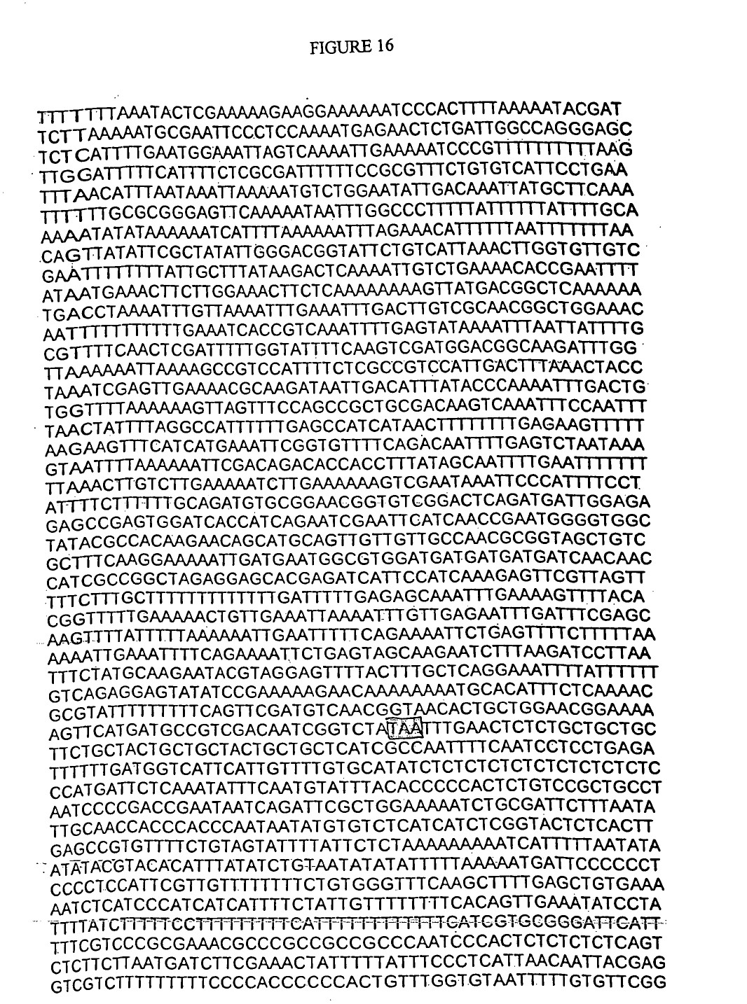

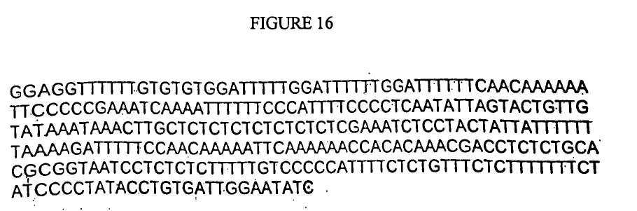

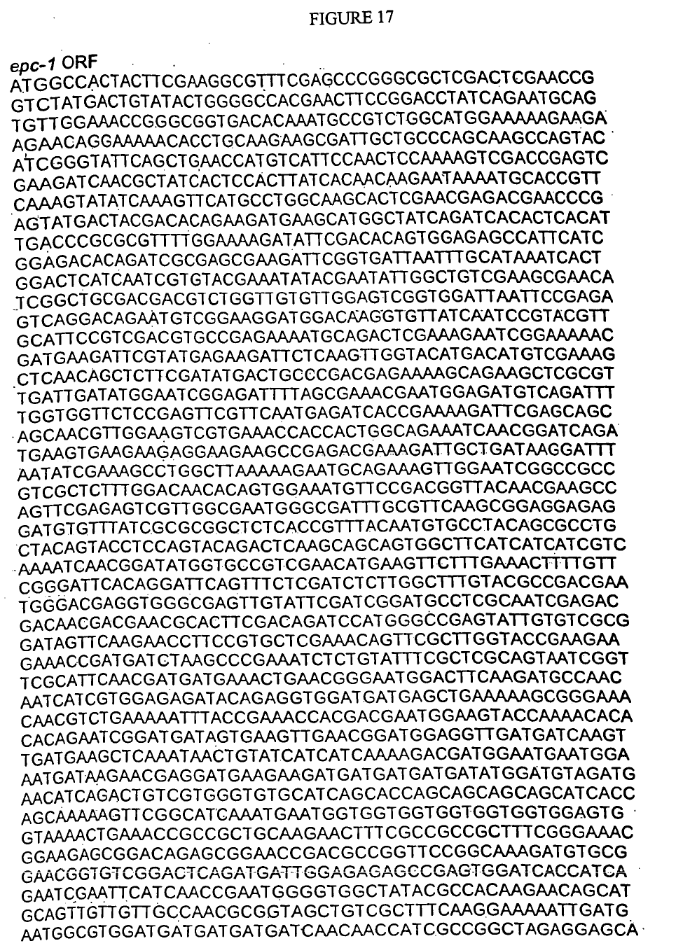

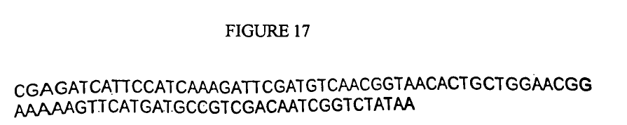

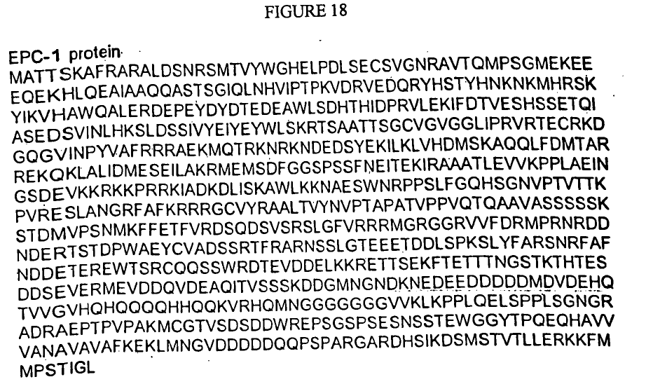

The TIP60 and NuA4 complexes contain other proteins in addition to MYST family acetyltransferases. We inactivated C. elegans genes encoding homologs of these proteins and identified epc-1 as a negative regulator of vulval induction. The genomic sequence of epc-1 is shown in FIG. 16. The nucleic acid sequence of the epc-1 open reading frame is shown in FIG. 17. epc-1 encodes a homolog of the Drosophila Enhancer of Polycomb (E(Pc)) protein and similarly named mammalian and yeast proteins. The deduced amino acid sequence of EPC-1 is shown in FIG. 18. Aside from their association with MYST family histone acetyltransferases, little is known about the molecular interactions of E(Pc)-like proteins. Inactivation of epc-1 caused fully penetrant embryonic lethality in the broods of animals injected with RNA. To study the effects of epc-1inactivation during postembryonic development, we injected epc-1 RNA into RNAi-deficient hermaphrodites and subsequently mated these animals with RNAi-competent males, a procedure referred to as “zygotic RNAi” (Herman, Development 128: 581-90, 2001). For many genes that act during multiple stages of development, this schemc has beer shown to provide sufficient gene activity for embryonic functions, but inadequate gene activity for postembryonic functions. epc-1 (RNAi) performed in this manner did not affect vulval induction in wild-type animals, but produced a Muv phenotype in lin-15A and lin-38 mutant backgrounds (Table 9).

TABLE 9
hat-1 and epc-1 but not ssl-1 loss of function phenocopies trr-1
loss of function

| | Ave. # P(3-8).p | % animals | |
|---|---|---|---|
| Genotype | induced (±SE) | mutant | n |
| wild-type | 3.00 (±0) | 0 | 31 |
| lin-15A(n767) | 3.00 (±0) | 0 | 24 |
| lin-38(n751) | 3.00 (±0) | 0 | 27 |
| lin-15B(n744) | 3.00 (±0) | 0 | 20 |
| hat-1(n4075) | 3.15 (±0.08) | 15 | 20 |
| hat-1(n4075); lin-15A(n767) | 3.76 (±0.14) | 76 | 25 |
| hat-1(n4075); lin-15B(n744) | 3.71 (±0.10) | 77 | 31 |
| rde-1/+; epc-1(RNAi) | 3.00 (±0) | 0 | 65 |
| rde-1/+; lin-15A(n767); epc-1(RNAi) | 3.32 (±0.10) | 36 | 33 |
| lin-38(n751); rde-1/+; epc-1(RNAi) | 3.29 (±0.02) | 31 | 65 |
| rde-1/+; lin-15B(n744); epc-1(RNAi) | 3.03 (±0.02) | 4.2 | 48 |
| rde-1/+; ssl-1 (RNAi) | 3.00 (±0) | 0 | 37 |
| rde-1/+; lin-15A(n767); ssl-1(RNAi) | 3.00 (±0) | 0 | 42 |
| rde-1/+; lin-15B(n744); ssl-1(RNAi) | 3.01 (±0.01) | 2.9 | 70 |

hat-1(n4075) homozygous mutants were recognized as the non-Unc progeny of +/nT1n754; hat-1(n4075)/nT1n754 heterozygous parents. Since RNAi of epc-1 and ssl-1 using standard methods causes highly penetrant embryonic lethality, we performed “zygotic RNAi” as described below.

A low percentage of P8.p induction was observed in a lin-15B background. We recently obtained a deletion allele that removes 886 bases from the epc-1 locus, including the third and fourth epc-1exons (FIG. 5A). If the second exon were spliced to the fifth exon, a 137 amino acid protein would be produced that contains the first 109 amino acids of the 795 amino acid predicted EPC-1 protein. Preliminary studies indicate that epc-1(n4076) homozygotes are sterile and, with respect to vulva induction, show genetic interactions similar to those of epc-1(RNAi), trr-1 and hat-1 mutants.

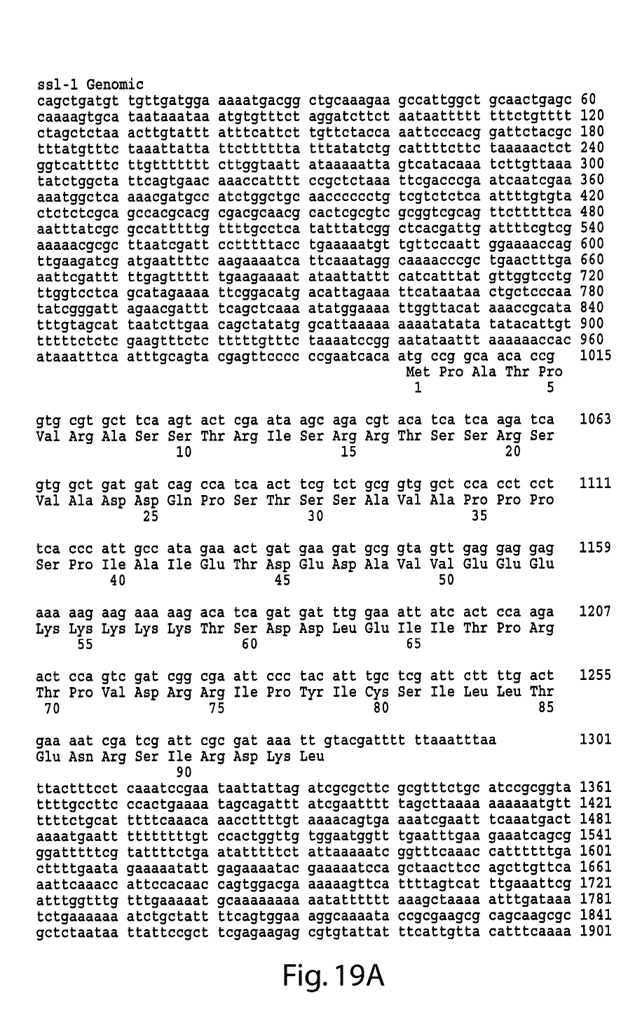

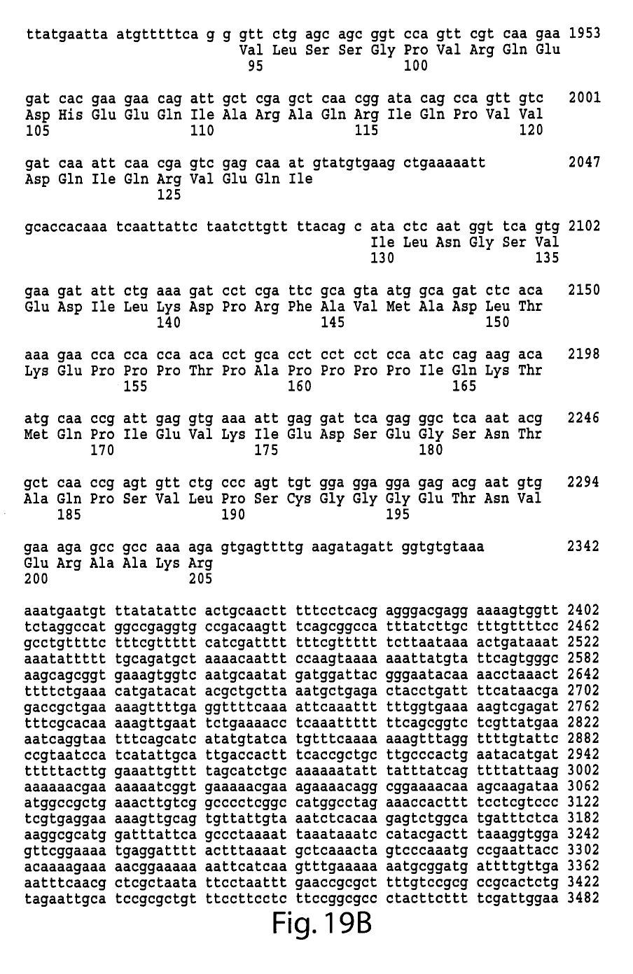

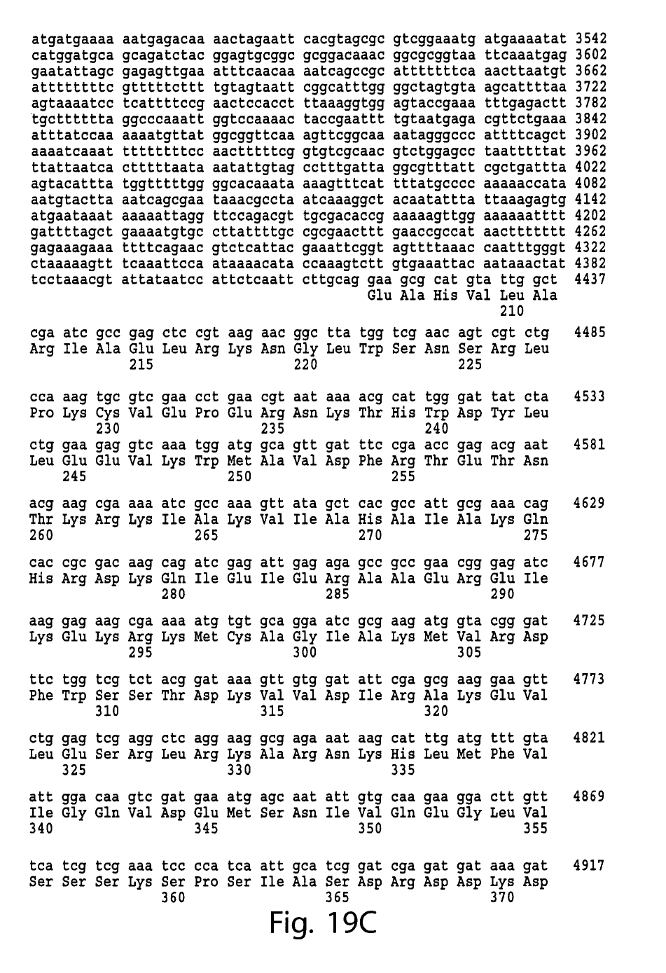

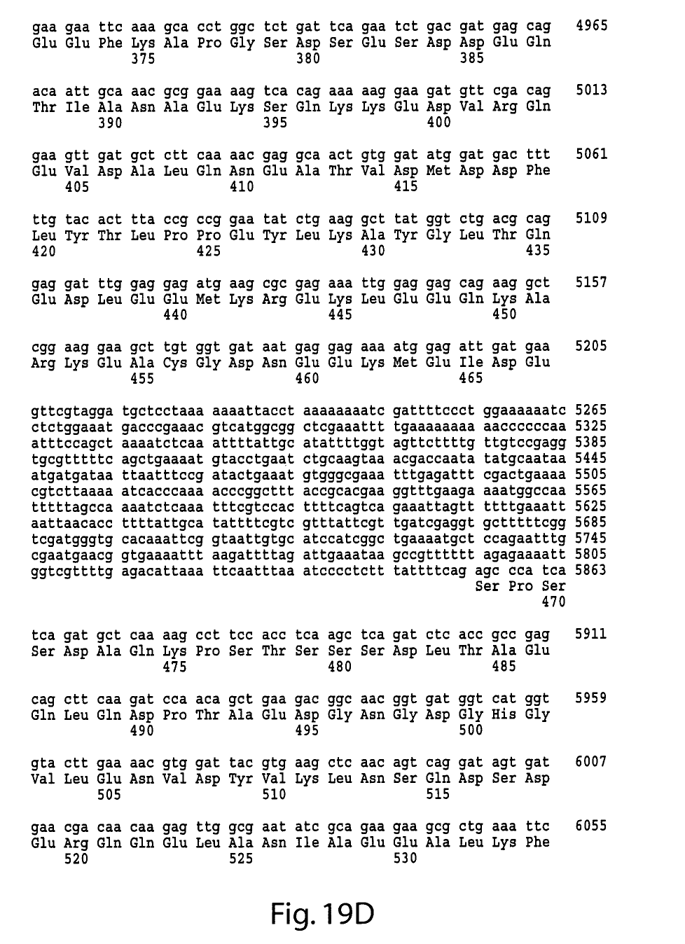

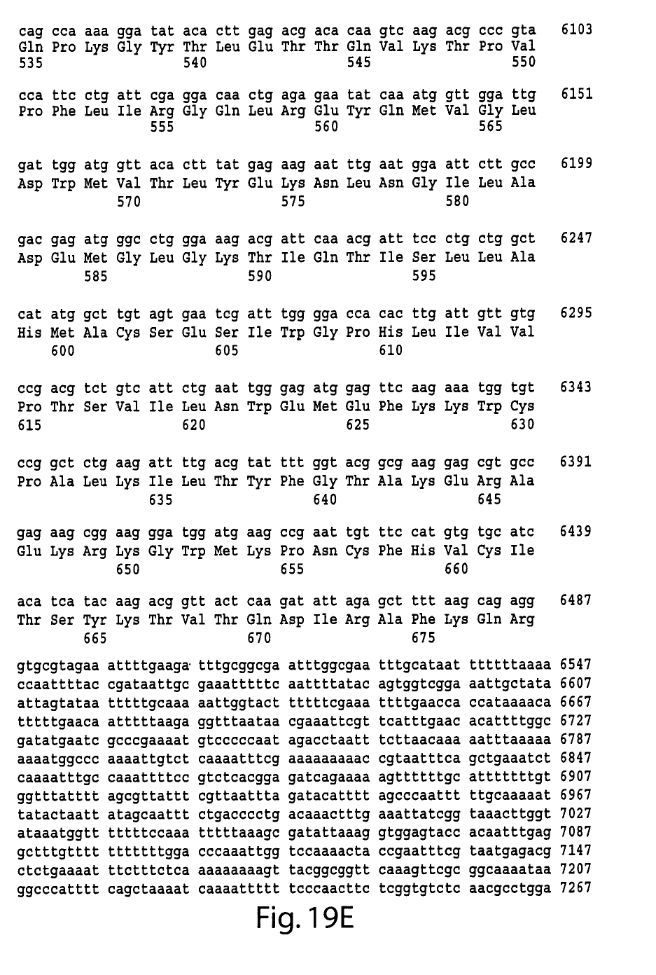

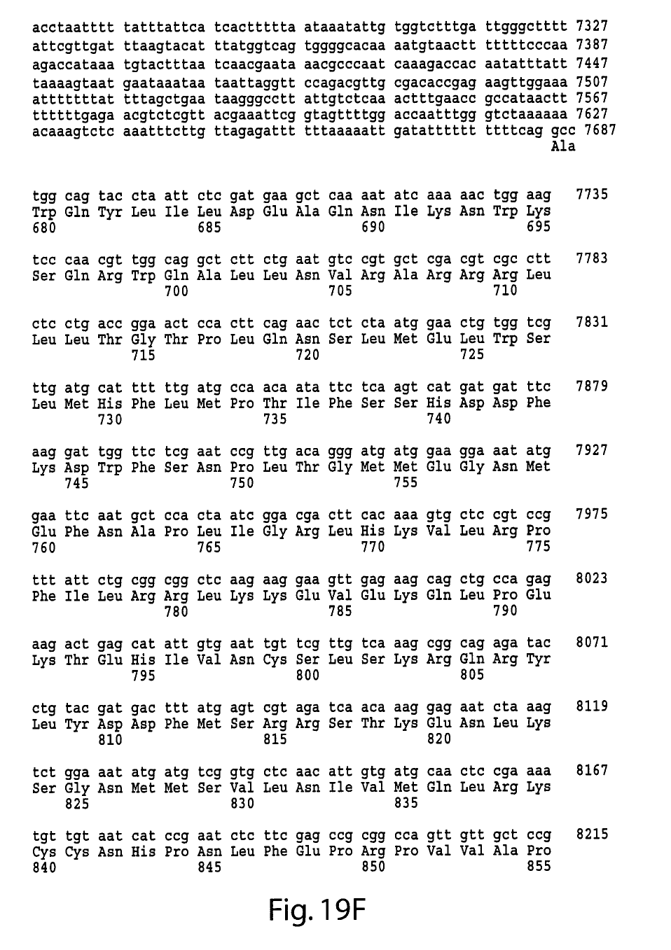

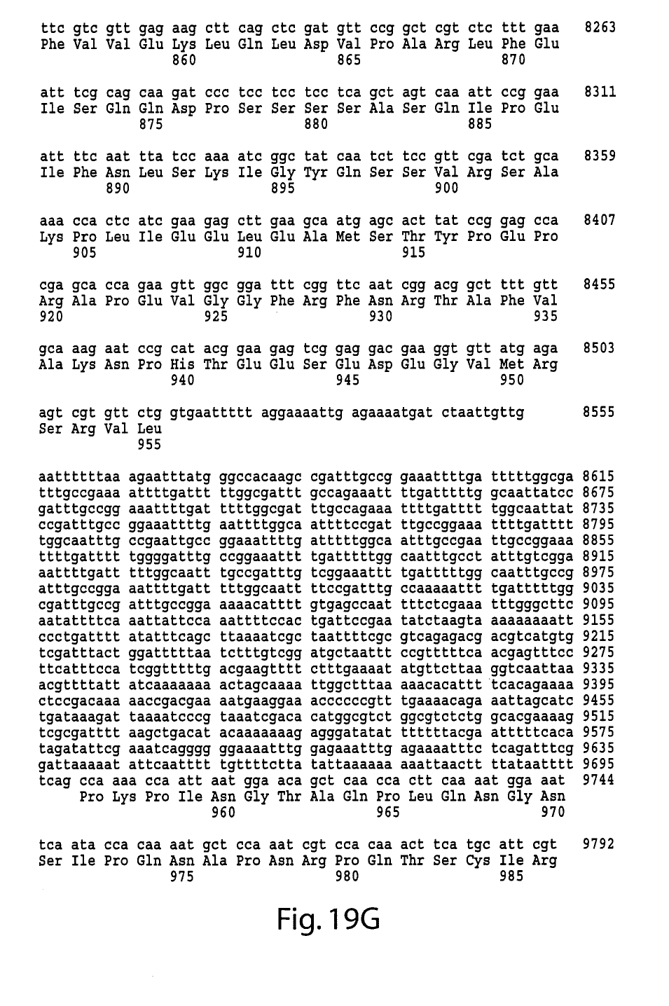

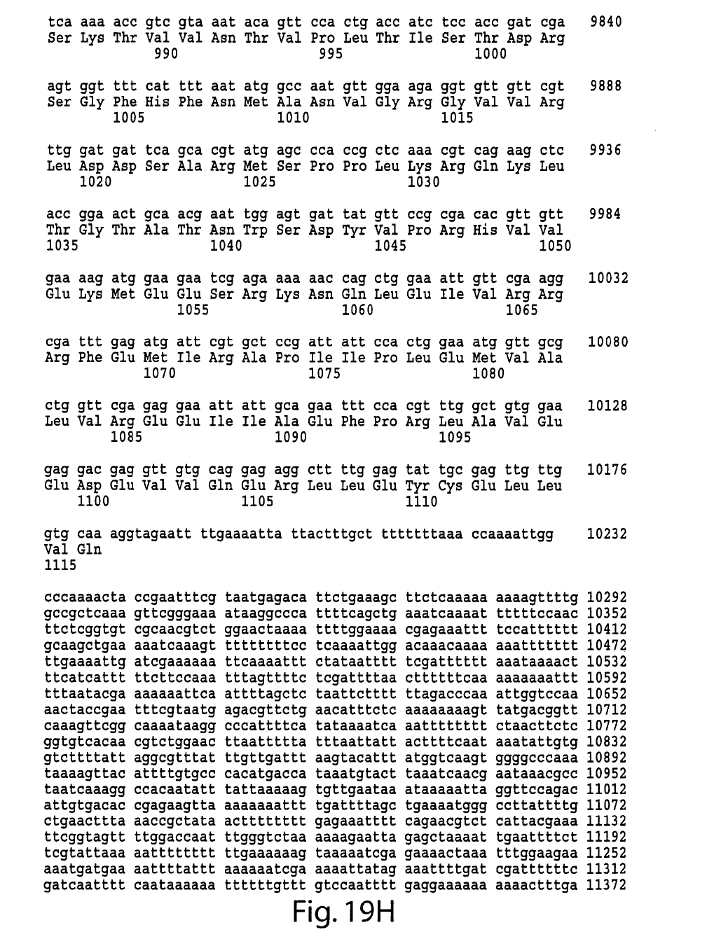

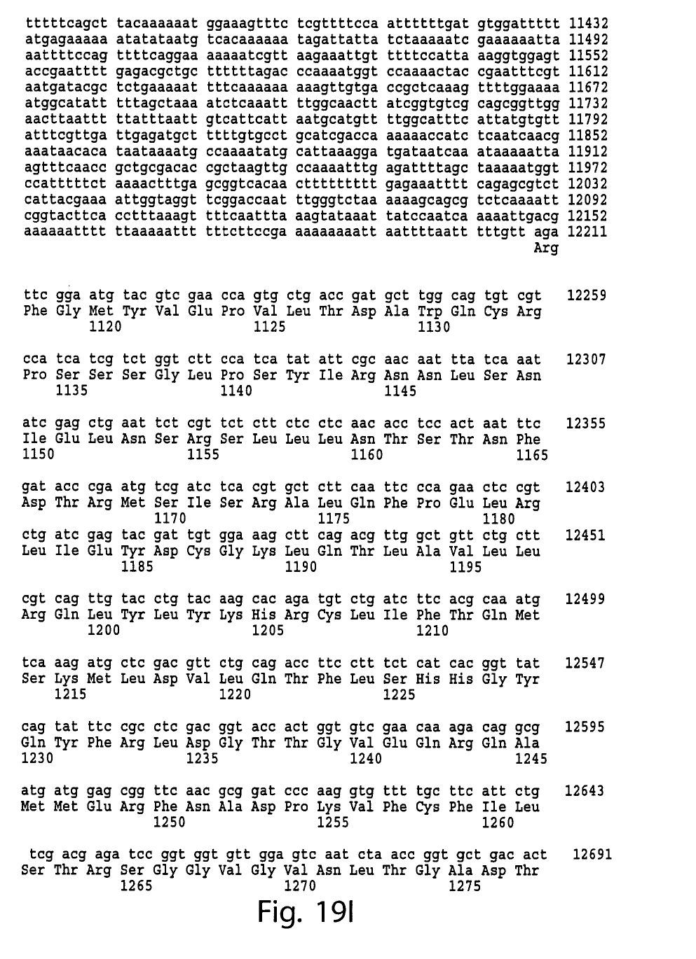

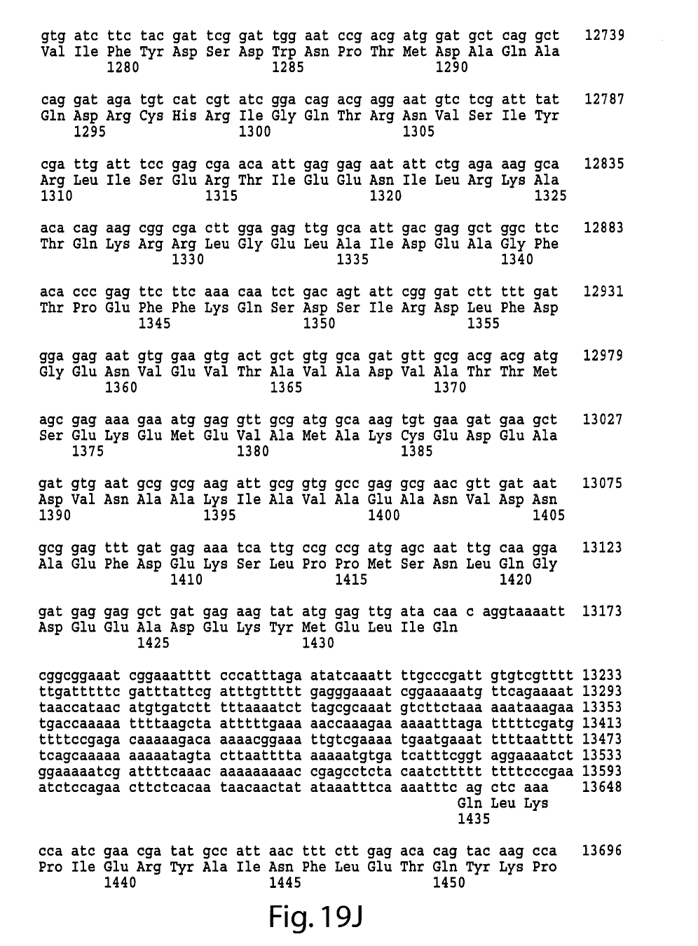

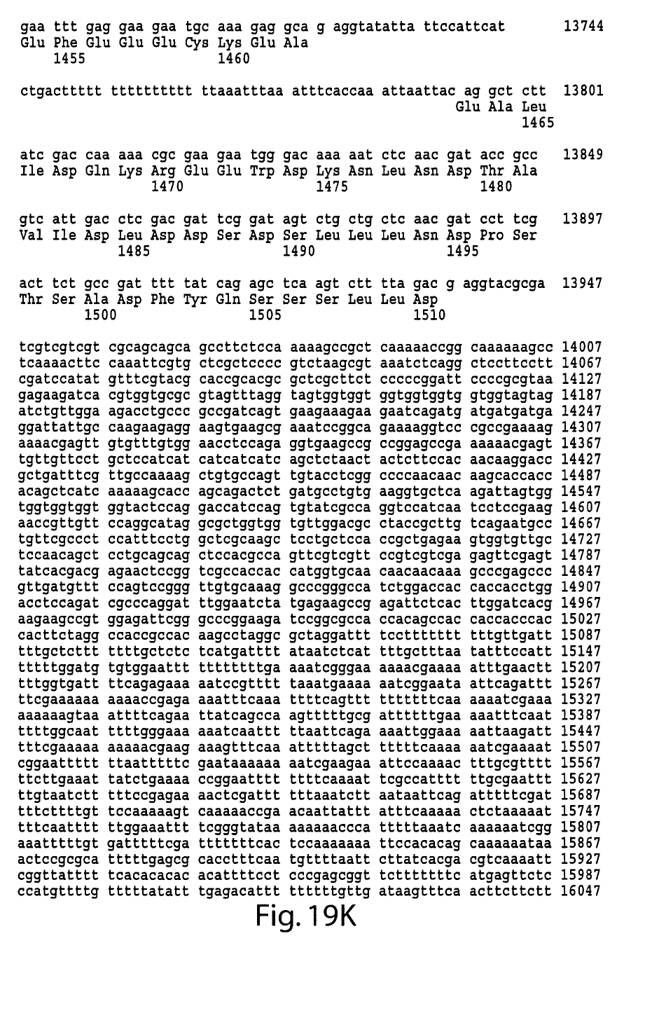

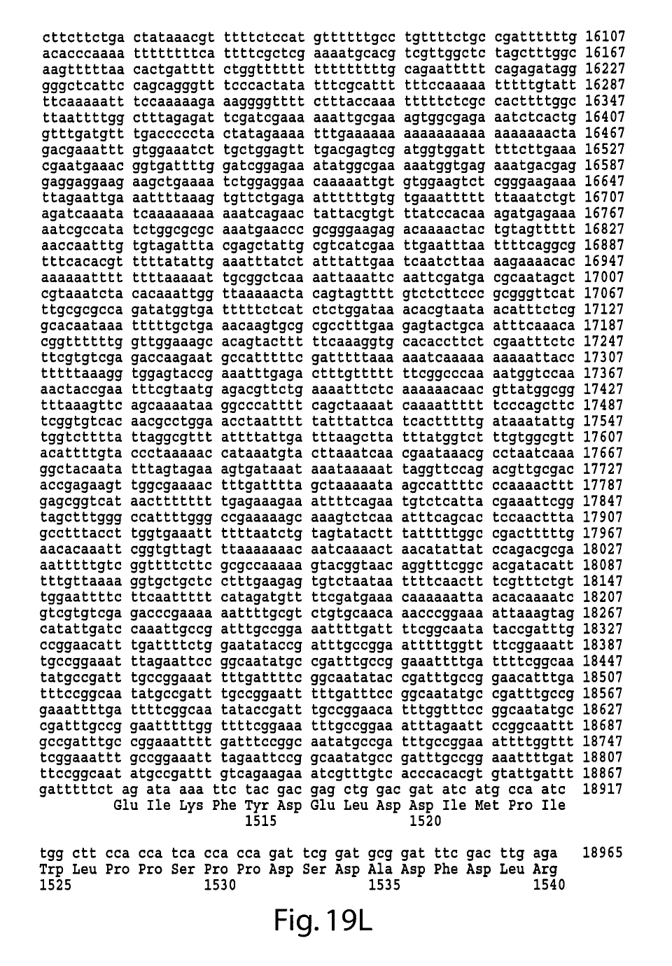

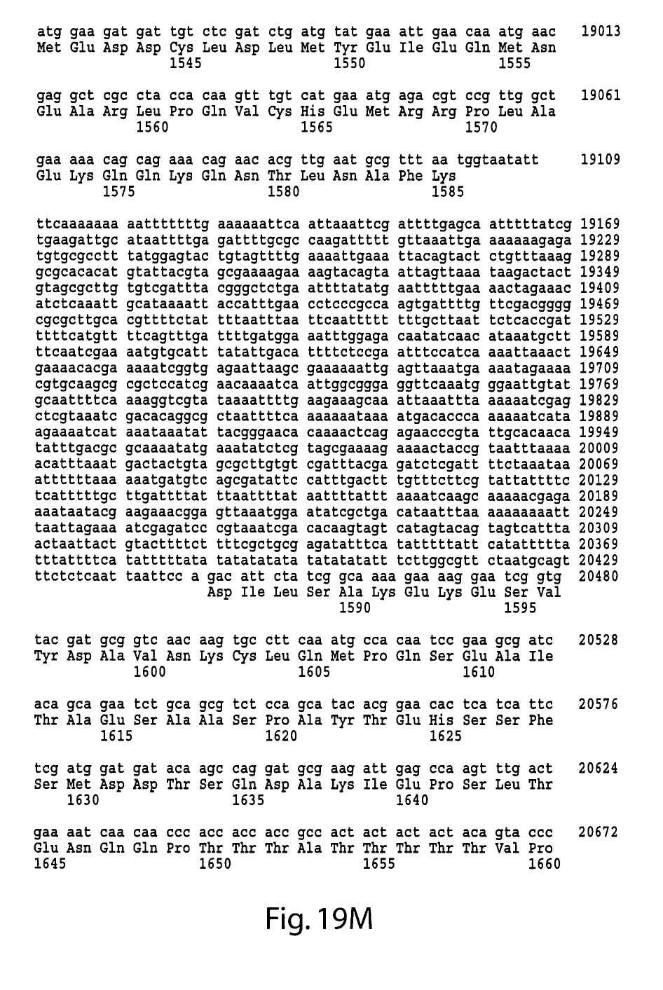

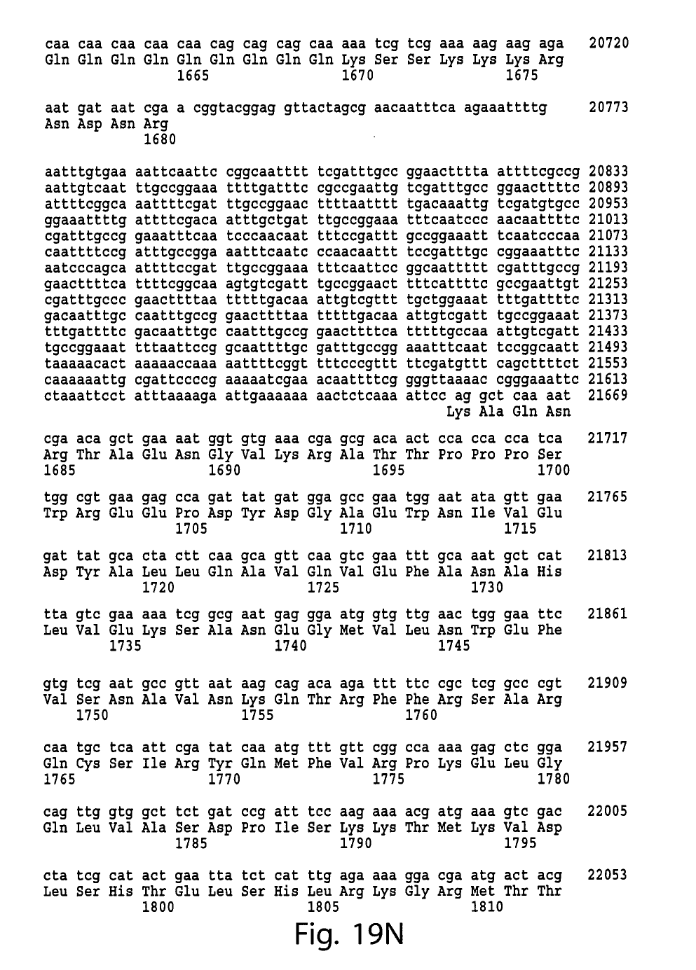

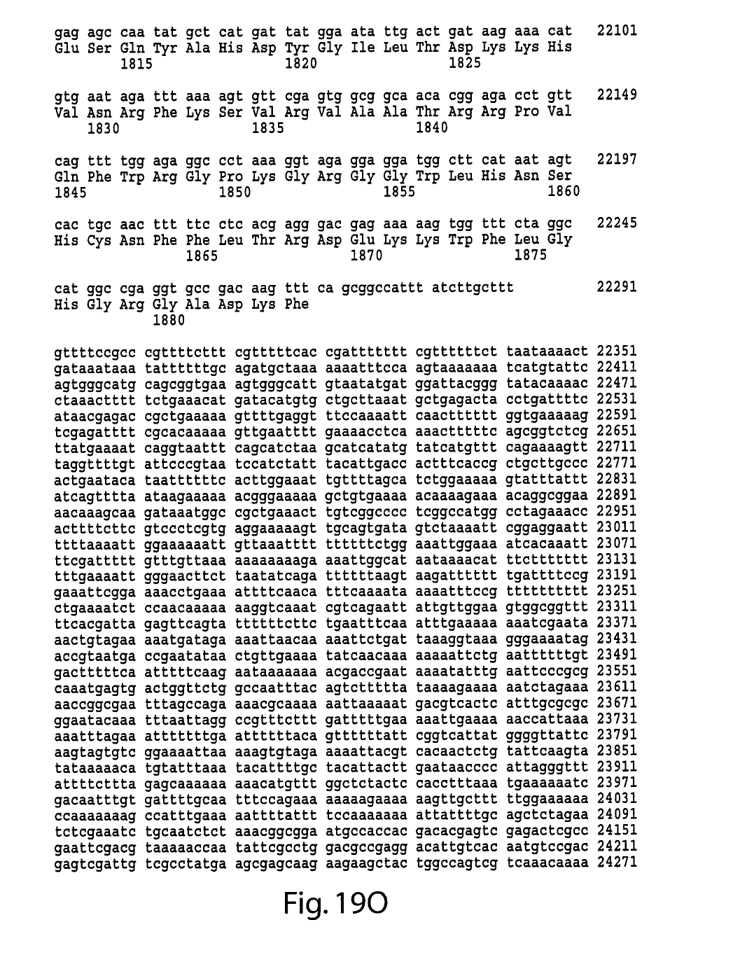

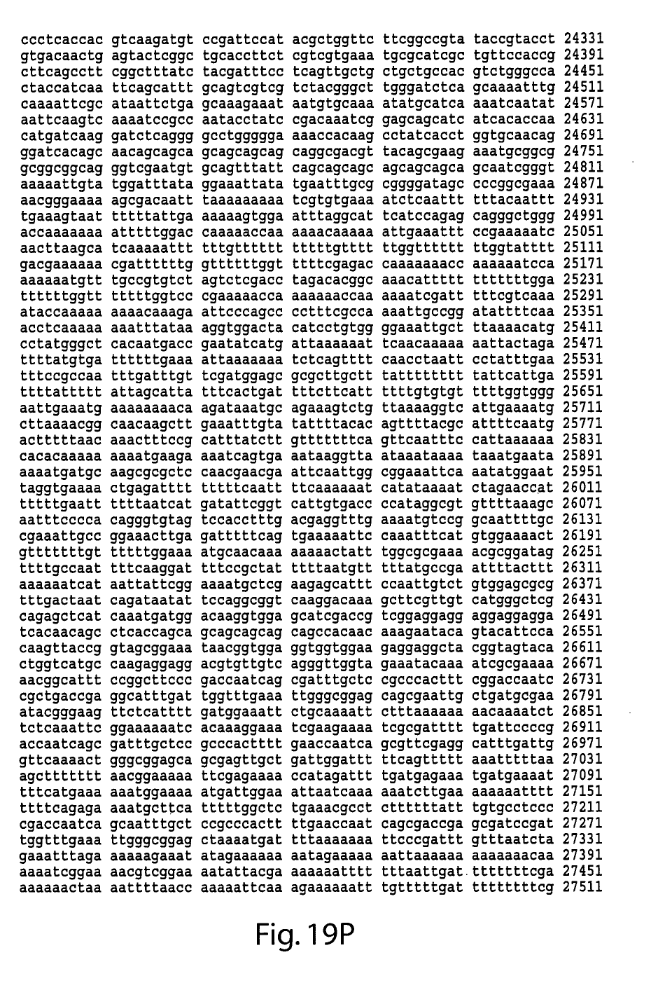

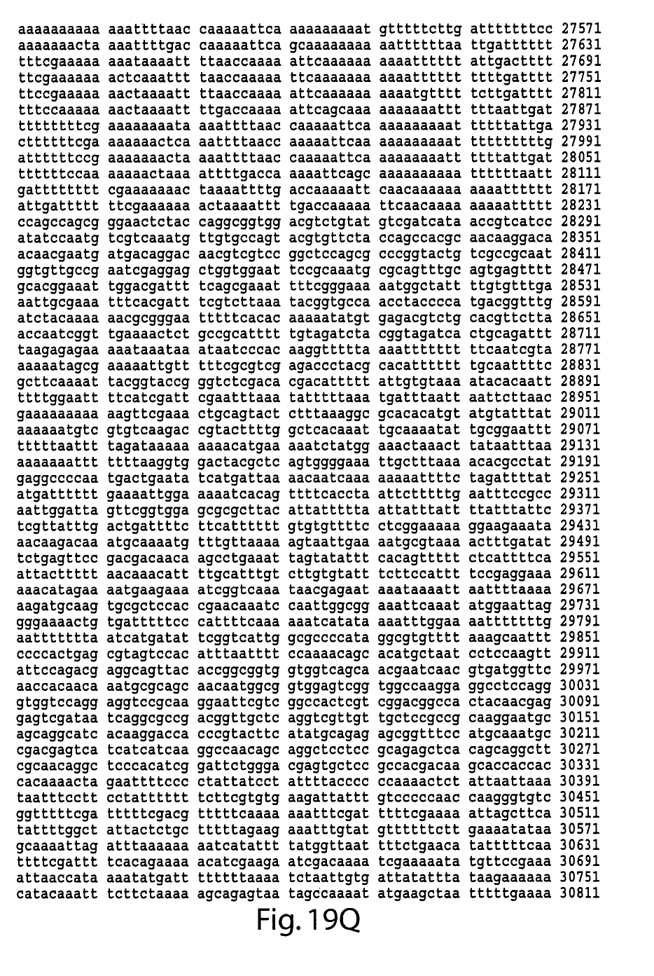

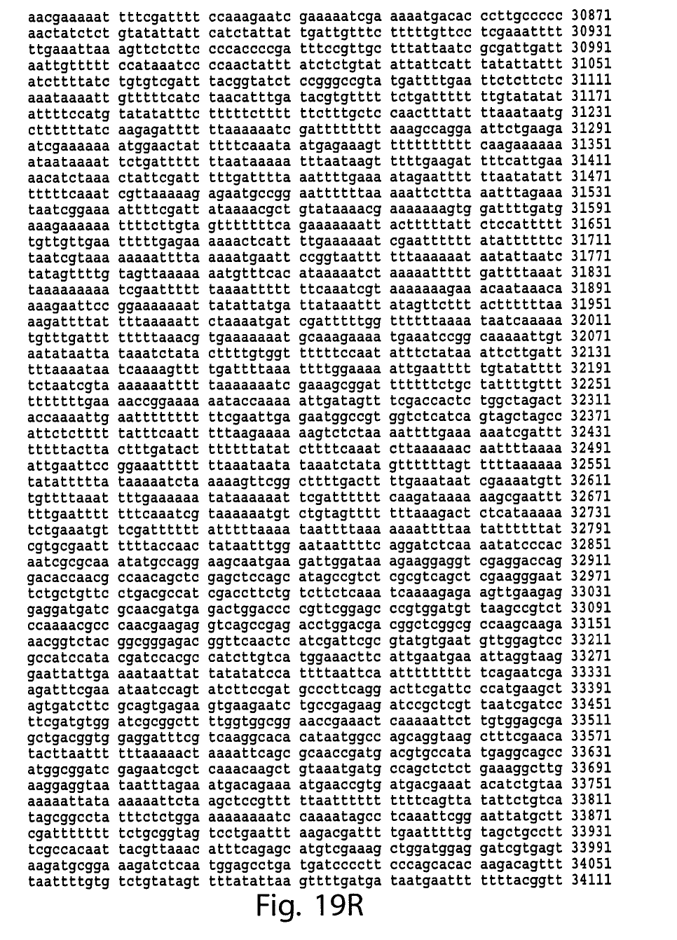

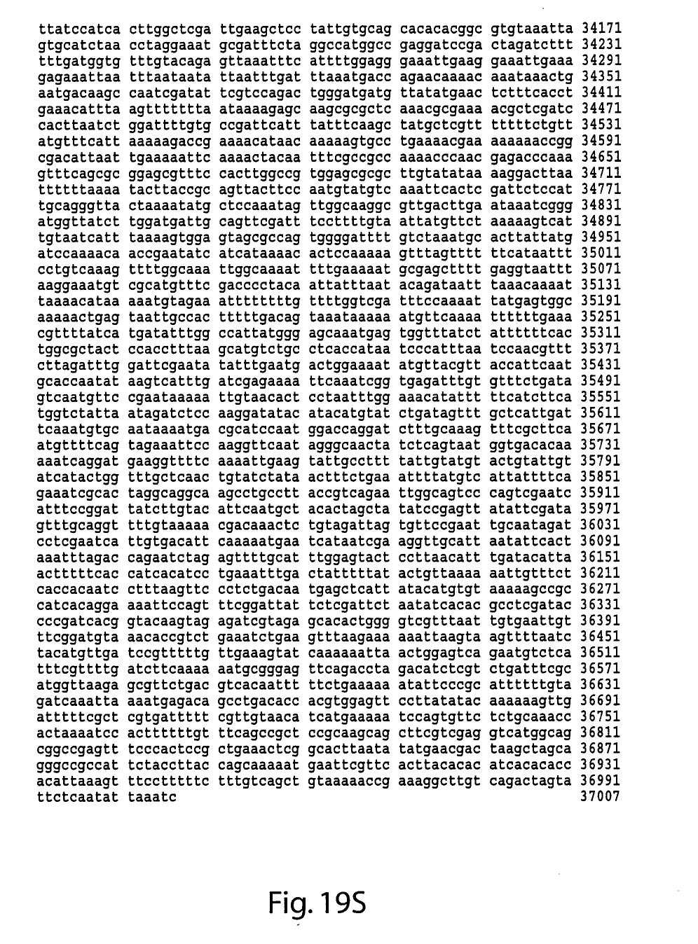

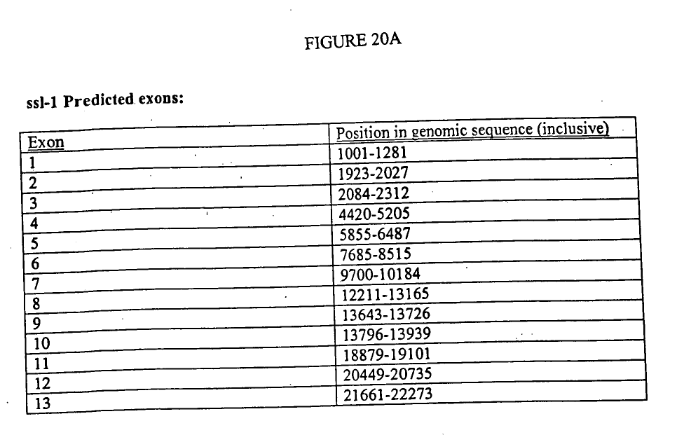

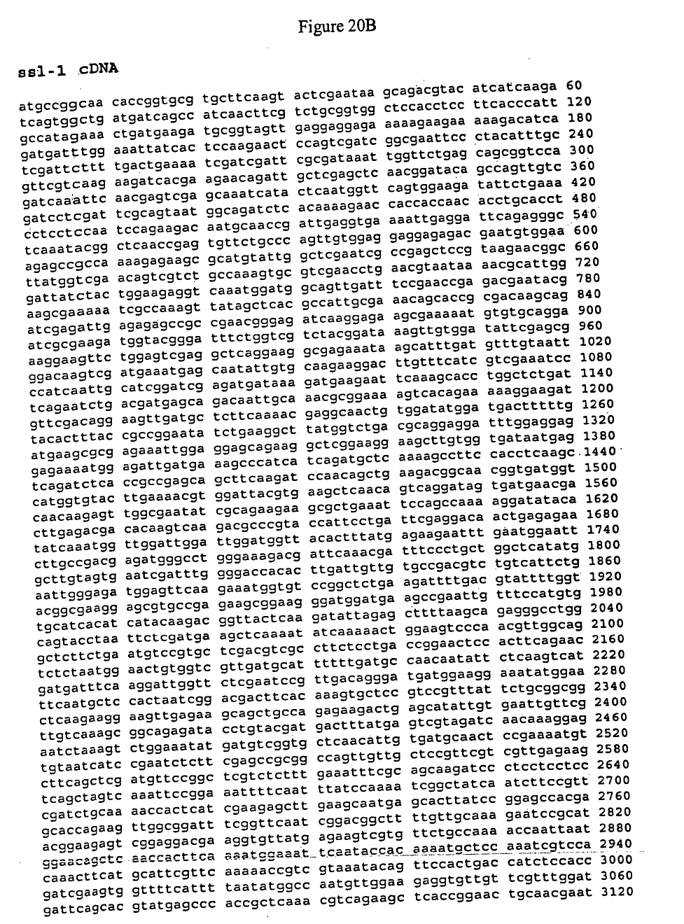

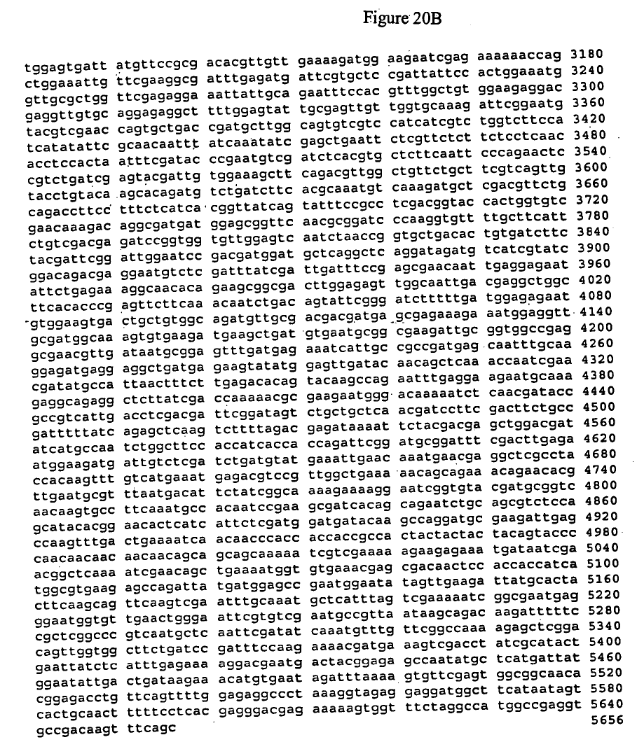

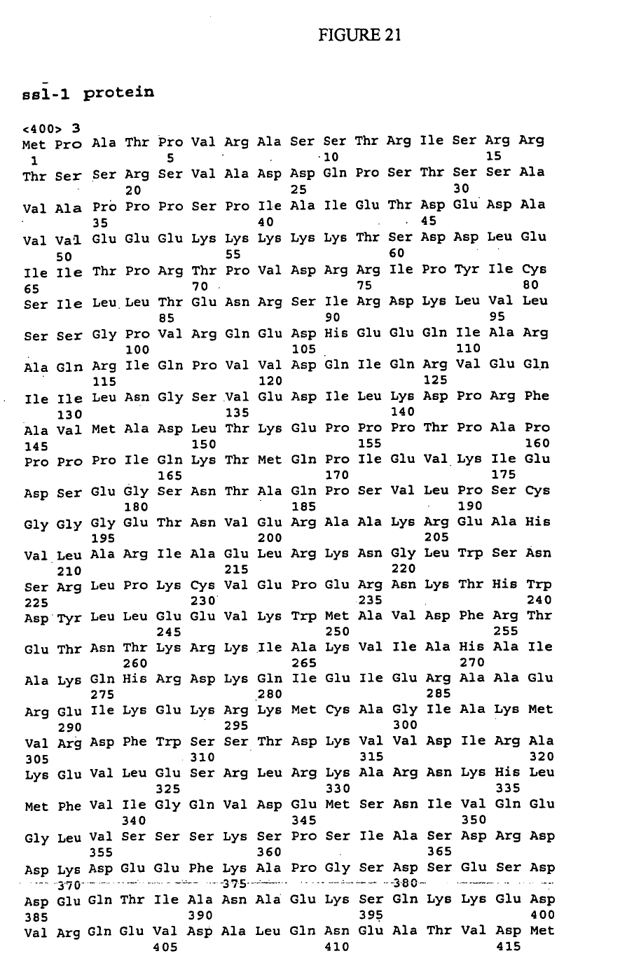

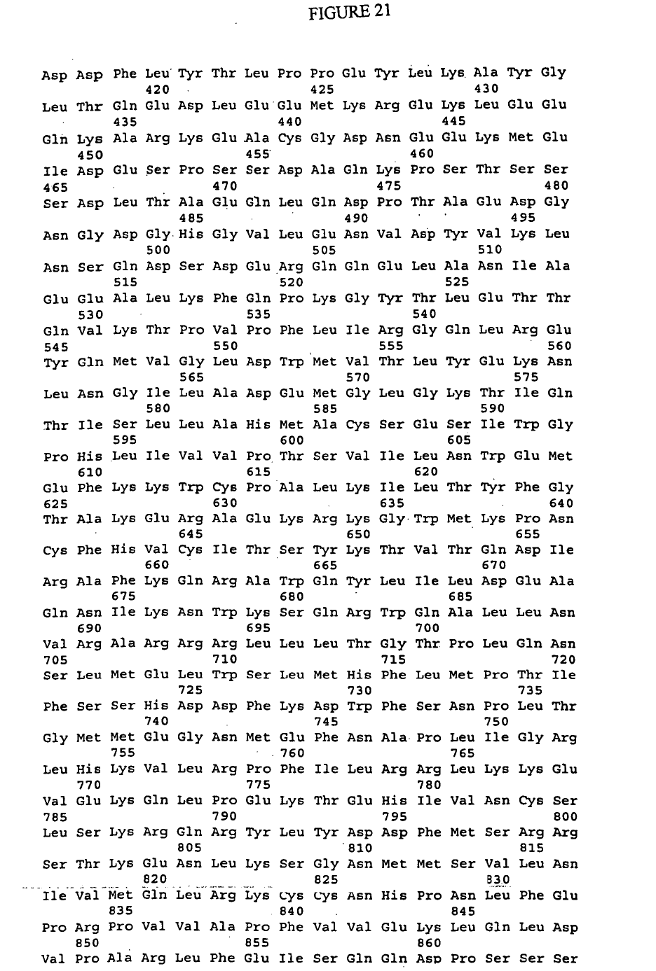

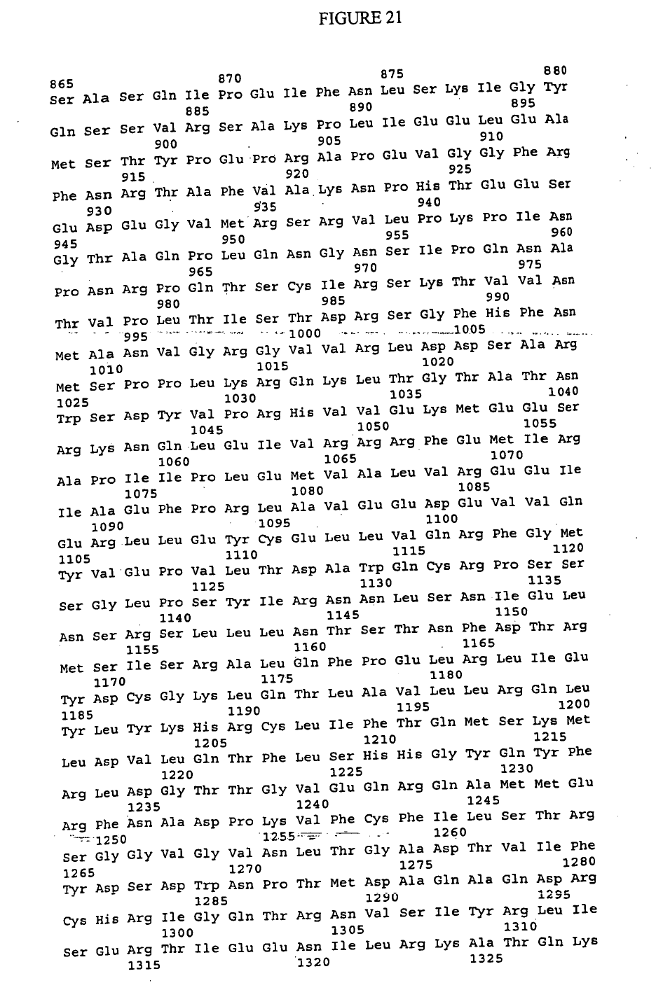

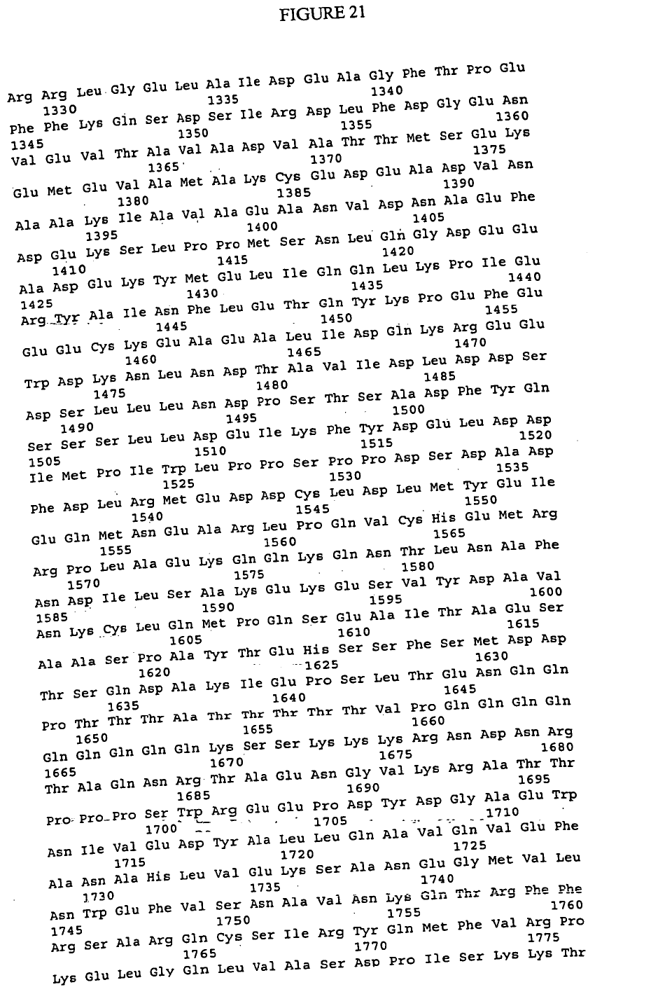

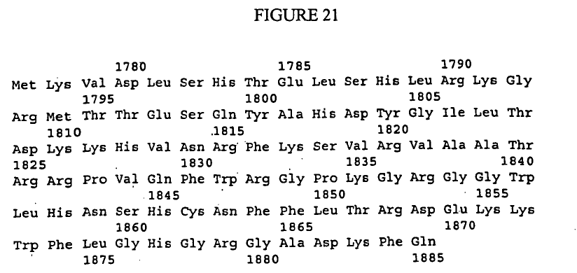

TRRAP copurified with the p400 protein as part of the mammalian TIP60 and p400 complexes (Fuchs et al., Cell 106: 297-307, 2001). The p400 complex was isolated based on its interaction with the adenovirus EIA orcoprotein and was also shown to associate with c-myc. The p400 protein itself is a member of the SWI2/SNF2 family of proteins, and, like many SWI2/SNF2 family members, was shown to possess ATPase activity. We identified a C. elegans homolog of p400, which we named ssl-1 (ssl, SWI2/SNF2-like). ssl-1 genomic sequence and the predicted SSL-1 protein product are shown in FIG. 19. FIG. 16B shows the nucleotide positions of the predicted exons with respect to ssl-1 genomic sequence. The cDNA sequence of ssl-1 is shown in FIG. 20. The deduced protein sequence is shown in FIG. 21. The function of ssl-1 was studied by RNAi. ssl-1 (RNAi) caused an embryonic lethal phenotype reminiscent of that caused by epc-1(RNAi). In both cases, dead embryos generally arrested just prior to morphogenesis and apparently lacked the hypodermal ridge that is a characteristic of enclosed embryos. We are currently characterizing this phenotype further. “Zygotic” RNAi of ssl-1, using the same procedure as described above, caused no vulval defects in wild-type, lin-15A, or lin-15B genetic backgrounds. These results suggest that ssl-1 may act with epc-1 in an essential embryonic process.

trr-1 Acts Redundantly with lin-35 Rb to Antagonize let-60 Ras Signaling

Identifying factors involved in cell fate determination is important for understanding how cells that contain the same genomic information can adopt different cell fates during animal development. As they help to distinguish P3.p, P4.p and P8.p from P(5-7).p, trr-1, hat-1, and epc-1are such cell fate determination genes. Given their molecular identities, trr-1, hat-1, and epc-1 likely act at the level of transcription, either in an instructive or permissive fashion, to create differences in gene expression in P3.p, P4.p and P8.p as compared to P(5-7).p.

Many of the pathways involved in regulating cell fate determination are conserved. In many cases, pathways that control cell fate determination in model organisms has been shown to regulate cellular proliferation in mammals. Pathways that regulate vulval cell fate specification in C. elegans provide clear examples. A conserved let-60 Ras pathway induces vulval cell fates, and this pathway is antagonized by the class B. lin-35 Rb pathway. trr-1, and likely hat-1 and epc-1, act in parallel to lin-35 Rb to negatively regulate let-60 Ras pathway signaling: These comparisons suggest that mammalian counterparts of trr-1, hat-1, and epc-1may similarly act in parallel to Rb and antagonize Ras in the control of cell proliferation.

trr-1, hat-1, and epc-1 Likely Share a Common Function

The vulval phenotypes and genetic interactions of trr-1, hat-1, and epc-1 mutants are strikingly similar. In light of the copurification of their mammalian and yeast counterparts, these data strongly suggest that TRR-1, HAT-1, and EPC-1 proteins function as part of a protein complex. To conclusively demonstrate such an interaction, strains containing mutations in two of these genes will be constructed. If these mutants are acting in the same complex, one would not expect to observe synergism in double mutants. In addition, protein-protein interaction studies will be performed. This complex containing putative complex members, trr-1, hat-1, and epc-1 were the only candidates we identified by RNAi. It is possible that these three genes encode an indispensable core of a putative HAT complex that associates with other proteins whose functions are dispensable for proper vulval development. The large size of TRR-1 may require it to be divided into fragments to perform protein interaction studies.

hat-1 Mutants Likely have Defects in Histone Acetylation

The best studied MYST family acetyltransferases are the yeast Esalp and mammalian TIP60 proteins. EsaIp was found to preferentially acetylate histone H4 (Smith et al., Proc Natl Acad Sci USA 95: 3561-5, 1998; Clark et al., Mol Cell Biol 19: 2515-26, 1999; Suka et al., Mol Cell 8: 476-9, 2001) Furthermore, depletion of Esalp resulted in global reduction of the acetylation of H4 and, to a lesser extent, of other nucleosomal histones (Reid et al., Mol Cell 6, 1297-307, 2000; Suka et al., Mol Cell 8: 476-9, 2001). HAT-I function is assayed using commercially available antisera that specifically recognize acetylated isoforms of histones to determine whether hat-1 mutants have gross defects in histone acetylation. Differences in acetylation between hat-1 mutants and wild-type animals is determined by whole-mount staining of fixed animals or by chromatin immunoprecipitation.

Putative HAT Complex Function

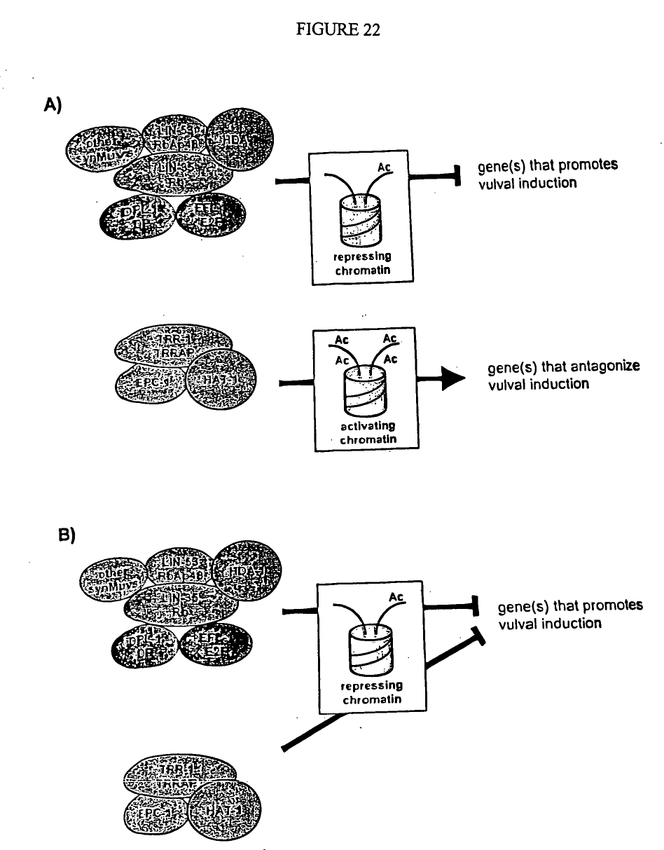

Histone acetyltransferases have been characterized as transcriptional coactivators (reviewed by Roth et al., Biochem 70:81-120, 2001), and TRRAP and its yeast homolog Tralp are proposed to bridge interactions between activation domains of DNA-binding transcription factors and histone acetyltransferases (Brown et al., Science 292, 2333-7, 2001). Therefore, a putative TRR-1/EPC-1/HAT-1 complex may function in transcriptional activation (FIG. 22). If so, one would expect it to activate genes that negatively regulate vulval development.

While most data support the link between acetylation and activation, additional observations suggest that at least some histone acetylation may be important for gene silencing. For example, loss-of-function mutations that affect the MYST family acetyltransferases Sas2p and Sas3p cause defects in silencing of mating type loci and telomeres in yeast (Reifsnyder et al., Nat Genet 14:42-9, 1996; Ehrenhofer-Murray et al., Genetics 145:923-34, 1997). Sas2p and Sas3p are proposed to acetylate newly-deposited nucleosomes, and the modified acetyllysine residues they create are thought to be important for establishing silencing following DNA replication (Meijsing et al., Genes Dev 15: 3169-82, 2001; Osada et al. Genes Dev 15:3155-68, 2001). These residues may include acetyllysine 16 on histone H4, which is implicated in mating type loci and telomeric silencing in yeast (Johnson et al., Embo J 11: 2201-9, 1992; Meijsing et al., Genes Dev 15: 3169-82, 2001). Other acetylated histone isoforms are prevalent in silent chromatin. For instance, Drosophila heterochromatin is enriched in acetyllysine 12 of histone H4 (Turner et al., Cell 69: 375-84, 1992). Just as a MYST family histone acetyltransferase is linked to silencing, loss-of-function studies in Drosophila indicate a role for E(Pc) in transcriptional repression. E(Pc) mutations synergize with polycomb group mutations to strongly derepress homeobox genes and act alone as suppressors of variegation to derepress genes that are juxtaposed to heterochromatin (Sato et al., Genetics 105: 357-70, 1983; Sinclair et al., Genetics 148: 211-20, 1998). These observations allow us to consider the possibility that HAT-1, in association with TRR-1 and EPC-1, may normally downregulate transcription (FIG. 22). By this model, one would expect a putative TRR-1/EPC-1/HAT-1 complex to silence genes that are required for vulval cell fates. Because we do not know the relevant targets of TRR-1/EPC-1/HAT-1, we cannot distinguish between transcriptional activating versus repressing models at this time.

Putative TRR-1/EPC-1/HAT-1 Complex DNA Targeting

Their coimmunoprecipitation and cooperation in reporter gene activation suggest that mammalian TRRAP can be targeted by E2F proteins to DNA (McMahon et al., Cell 94: 363-74, 1998; (Lang et al., J Biol Chem 276: 32627-34, 2001). We investigated the possibility of TRR-1 targeting by DP/E2F heterodimers by studying genetic interactions between-trr-1 and dpl-1. dpl-1 is the only DP family member in C. elegans and therefore loss of dpl-1 activity is expected to effectively reduce all DP/E2F heterodimer function in the organism. dpl-1 synthetically interacted with trr-1 in vulval induction and viability assays. It is especially relevant that we observed synergism in some of these assays when using dpl-1(n3316 RNAi) mutants, which are severely compromised for dpl-1 function. These results combined with the observation that the defects of trr-1 single mutants are stronger than those of dpl-1 single mutants suggest that trr-1 acts only partially or not at all through dpl-1. If not only through DPL-1, how might a putative TRR-1/EPC-1/HAT-1 complex be targeted to DNA? Studies in yeast indicate that the TRRAP homolog Tralp directly interacts with acidic activation domains of transcription factors (Brown et al., Trends Biochem Sci 25: 15-9, 2000). TRR-1 may similarly be targeted to DNA by transcription factors other than DPL-1. The assays we have used to characterize trr-1 provide a means of identifying and evaluating candidate transcription factors and other proteins that may function with TRRAP family members in targeted histone acetylation.

The experiments described in Example II were carried out as described below.

Strains and Genetics

Strains were cultured as described by (Brenner, Genetics 77: 71-94, 1974), and maintained at 20° C. unless otherwise specified. Bristol N2 was used as the wild-type strain. The following mutations were used: LGI: lin-35(n745); LGII: dpy-10(e128), let-23(sy97), rol-6(e187), dpl-1(n2994, n3316) (Chapters 2, 3), unc-4(e120), trr-1(n3630, n3637, n3704, n3708, n3709, n3712) (This study), mex-1(it9), lin-38(n751); LGIII: lon-1(e85), sup-5(e1464), lin-36(n766), lin-37(n758); LGIV: lin-3(n378), let-60(n1876) (Beitel et al., Nature 348: 503-9, 1990); LGV: dpy-11(e224), rde-1(ne219) (Tabara et al., Cell 99: 123-32, 1999); LGX: lin-15B(n744), lin-15A(n767, n433) (Ferguson et al., Genetics 123: 109-21, 1989) and, unless otherwise noted, are described in (Riddle et al., C. elegans II (Cold Spring Harbor, N.Y., Cold Spring Harbor Laboratory-Press, 1997). The deficiencies mnDf90 and mnDf87 (Sigurdson, et al., Genetics 108: 331-45, 1984), translocation nT1 n754 (IV;V) (Ferguson et al., Genetics 110: 17-72, 1985), and chromosomal inversion mIn1[dpy-10(e128) mIs14] (Edgley et al., Mol Genet Genomics 266:385-95, 2001), were also used. mIs14, an integrated transgene linked to the chromosomal inversion mIn1 , consists of a combination of GFP-expressing transgenes that allow mIs14-containing animals to be identified beginning at the 4-cell stage of embryogenesis (Edgley et al., Mol Genet Genomics 266:385-95, 2001).

P(3=8).p Induction Assay

In the wild-type, P(5-7).p adopt vulval fates in which they divide during the L3 larval stage to generate seven or eight descendants. P3.p, P4.p and P8.p adopt non-vulval fates, typically dividing once to generate two descendants that fuse with the hypodermis. Induction was scored in L4 hermaphrodites using Nomarski DIC microscopy by counting the number of descendants produced by individual P(3-8).p cells. Different scores, 1, 0.5 and 0 cells induced, were assigned to cells that were fully, partially or not induced, respectively. Partially induced P(3-8).p cells have one daughter that produces a complement of induced descendants while the other daughter fails to divide.

trr-1 Cloning

We mapped trr-1 to an interval on LGII between the right endpoint of the deficiency mnDf90 and the mex-1 gene. To clone the trr-1 gene, we performed transformation rescue as described by (Mello et al., Embo J 10: 3959-70, 1991), using the pRF4 plasmid (80 ng/μL) as a coinjection marker. We rescued the trr-1 Muv and sterile phenotypes by injecting the cosmid C47D12 (10 ng/μL) into trr-1(n3712)mIn1[dpy-1O(e128) mIs14]; lin-15A(n767) mutants and isolating Rol non-Gfp transgenic lines. trr-1 corresponds to the predicted gene C47D12.1.

RNAI Analyses

Templates for in vitro transcription reactions were made by PCR amplification of either cDNAs and their flanking T3 and T7 promoter sequences or coding exons from genomic DNA using T3 and T7-tagged oligonucleotides. In vitro-transcribed RNA was annealed and injected as described by (Fire et al., Nature 391: 806-11, 1998).

In addition to the genes described above, we injected RNA corresponding to C. elegans genes that encode homologs of the TRRAP complex proteins TIP48/TAP54α (C. elegans predicted gene T22D1.1), TIP49/TAP54 (C27H6.2), Eaf3p (Y37D8A.9), p33ING (Y51H1A.4), and AF-9 (M04B2.3) (Loewith et al., Mol Cell Biol 20: 3807-16, 2000; Eisen et al., J Biol Chem 276: 3484-91, 2001; Fuchs et al., Cell 106: 297-307, 2001; Nourani et al., Mol Cell 21: 7629-40, 2001; Gavin et al., Nature 415: 141-7, 2002; Ho et al, Nature 415: 180-3, 2002). We did not observe vulval lineage defects after injection of these RNAs into either wild-type or synMuv single mutant backgrounds. Lastly, bacteria designed to express double-stranded RNA corresponding to the Gcn5 homolog Y47G6A.6 (Fraser et al., Nature 408: 325-30, 2000) were fed to wild-type and synMuv single mutant hermaphrodites. As described below, we did not observe vulval defects following this treatment.

Deletion Allele Isolation

Genomic DNA pools from mutagenized worms were screened for deletions essentially as described by (Plasterk et al., Nat Genet 17: 119-21, 1997). Deletion mutant animals were isolated from frozen stocks and were backcrossed four times prior to use. hat-1(n4075) removes nucleotides +106 to +1115, epc-1(n4076) nucleotides +2014 to +2899 and ssl-1(n4077) nucleotides +5075 to +5757 of genomic DNA relative to their respective predicted translational start sites.

cDNA Isolation

We used TITAN ONE-TUBE RT-PCR (Roche Diagnostics, Pleasanton, Calif.) to carry out RT-PCR and recovered trr-1 and hat-1 cDNA clones. Existing cDNAs were obtained from the C. elegans EST project to determine gene structures of epc-1, the trr-1 3′ end and the ssl-1 5′ end. We used 5′ RACE (5′ RACE System v2.0, GIBCO) to determine the 5′ ends and SL1 trans-spliced leader sequences of trr-1, hat-1, and epc-1 transcripts.

Allele Sequence

We used PCR-amplified regions of genomic DNA as templates in determining mutant allele sequences. For each allele investigated, we determined the sequences of all exons and splice junctions of the gene in question. All mutations were confirmed by determining the sequence of independently-derived PCR products. All sequences were determined using an automated ABI 373 DNA sequencer (Applied Biosystems).

### EXAMPLE III

ssl-1, a p400 SWI/SNF ATPase Homolog, Acts Redundantly with lin-15B

TRRAP is a component of the mammalian p400 complex, which contains the p400 SWI/SNF family protein and was identified based on its interaction with the adenovirus E1A oncoprotein (Fuchs et al., Cell 106: 297-307, 2001). Although Tip60 was not present in the purified p400 complex, the Tip60 and p400 complexes share many of the same components and more recent analyses have indicated that p400 and Tip60 can copurify as part of a large p400/Tip6O multisubunit complex (Frank et al., EMBO Rep., 4:575-80, 2003).

As discussed in Example II, the ssl-1 (ssl, SWI/SNF-like) gene encodes a homolog of the p400 protein. RNAi of ssl-1 using standard methods caused fully penetrant embryonic lethality like that observed with epc-1(RNAi). zygotic RNAi of ssl-1, performed as described above, did not cause defects in vulval development in either class A or class B synMuv backgrounds. In further studies, we isolated a deletion mutation, n4077, that removes a portion of the fifth ssl-1 exon. ssl-1 (n4077) is predicted to encode a truncated protein containing the first 540 amino acids of the 1671 amino acid SSL-1 protein-and two unrelated amino acids. ssl-1(n4077) homozygotes were partially sterile and produced a few inviable embryos, but were not defective in vulval development. ssl-1(n4077); lin-15A(n767) mutants were likewise not defective in vulval development, however, ssl-1(n4077); lin-15B(n744) mutants often expressed an ectopic vulval cell fate in P8.p. ssl-1 (n4077) likely causes a stronger reduction in gene activity than does ssl-1 zygotic RNAi, and this stronger reduction unmasks a redundancy between ssl-1 and lin-15B.

trr-1; hat-1, trr-1; epc-1 and trr-1; ssl-1 Double Mutants do not Show Synthetic Defects in Vulval Development

Whereas synthetic defects in double mutants imply genetic redundancy, the lack of synthetic defects in double mutants can indicate that two genes act in the same genetic pathway. Based on the similar phenotype and genetic interactions of trr-1, hat-1 and epc-1 mutantsand on the copurification of the proteins encoded by their mammalian and yeast counterparts, we hypothesized that trr-1, hat-1 and epe-1 act together to regulate vulval development. To test this possibility, we constructed double mutants to determine if hat-1 and epc-1 function redundantly with trr-1. We measured the numbers of vulval cell fates in trr-1 (n3712); hat-1(n3681), trr-1 (n3712); hat-1 (n4075), and trr-1(n3712); epc-1(RNAi) mutants and found that the extent of vulval development observed in these double mutants was similar to that observed in single mutant animals. These results suggest that hat-1 and epc-1 act in the same genetic pathway as trr-1, which by analogy to the class A and class B lin-35 Rb synMuv pathways, we have named the class C synMuv pathway.

trr-1, ssl-1 double mutants, and, as described above, ssl-1; lin-15A mutants were not synthetically defective in P(3-8).p cell-fate specification. It is possible that ssl-1 has both class C and class A synMuv activities, however, additional considerations suggest that ssl-1 has properties more like those of a class C gene. For instance, ssl-1; synmuvB mutants have a defect limited to P8.p, whereas synmuvA; synmuvB mutants typically show ectopic vulval cell fates in P3p, P4.p and P8.p. In addition, ssl-1 mutants are sterile, and sterility has not been observed for any class A synMuv gene (Thomas et al., Development 126: 3449-59, 1999). These considerations, along with the copurification of the mammalian SSL-1 and HAT-1 counterparts, p400 and Tip60, suggest that ssl-1 is an atypical class C gene, one that acts redundantly with class B, but not class A synMuv genes.

trr-1, hat-1, epc-1 and ssl-1 Act Redundantly with the lin-35 Rb Pathway to Antagonize let-60 Ras Signaling

Identifying genes involved in cell-fate determination is important for understanding how cells that contain the same genomic information can adopt different fates during animal development. As they help to distinguish P3.p, P4.p and P8.p from P(5-7).p, trr-1, hat-1, epc-1 and ssl-1 are such cell-fate determination genes.

In many cases, pathways that control cell-fate determination and cell division in invertebrates havebeen shown to regulate similar processes in mammals. Pathways that regulate vulval cell-fate specification in C. elegans provide clear examples. A conserved let-60 Ras pathway induces vulval cell fates, and this pathway is antagonized by an at least partially conserved class B lin-35 Rb pathway. trr-1, hat-1, epc-1 and ssl-1 act in parallel to lin-35 Rb and other genes in this pathway to negatively regulate let-60 Ras signaling. We suggest that the mammalian counterparts of trr-1, hat-1, epc-1 and ssl-1 may similarly act in parallel to Rb and antagonize Ras in the control of cell-fate determination and cell division. It is interesting to note that the p400 complex and Rb-containing complexes are targeted by the adenovirus E1A oncoprotein (Whyte et al., Nature 334:124-9, 1988; Fuchs et al., Cell 106: 297-307, 2001). Our finding regarding ssl-1 redundancy with a lin-35 Rb pathway gene suggests that EIA may act in mammals by perturbing the activities of functionally redundant p400 and Rb-containing complexes.

Identification of New Class B synMuv Genes

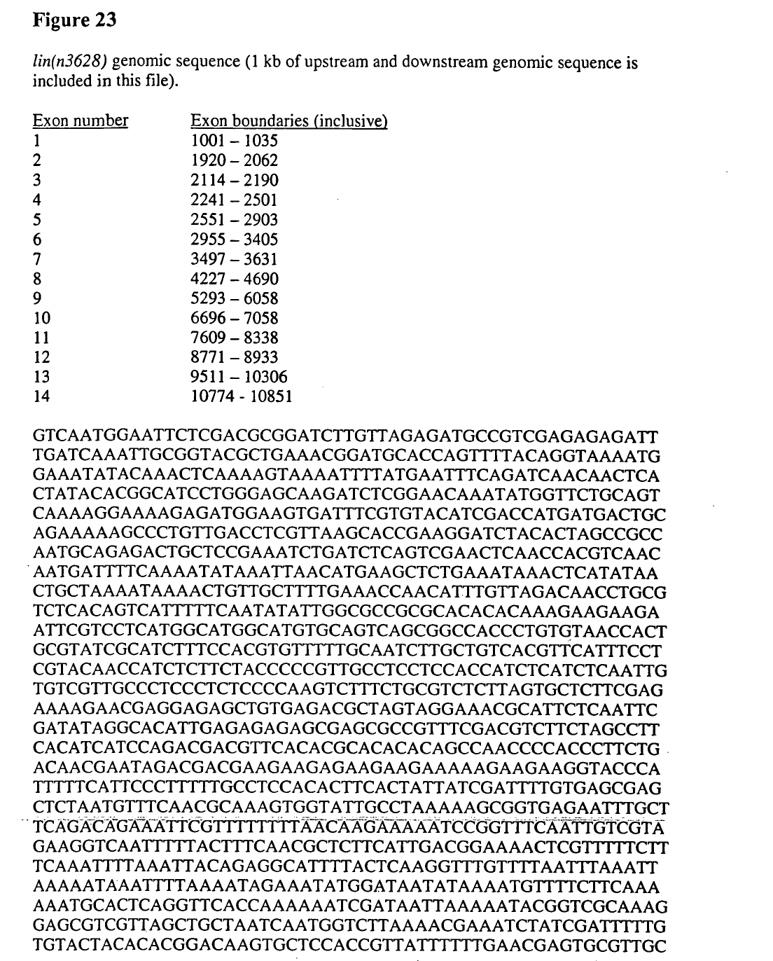

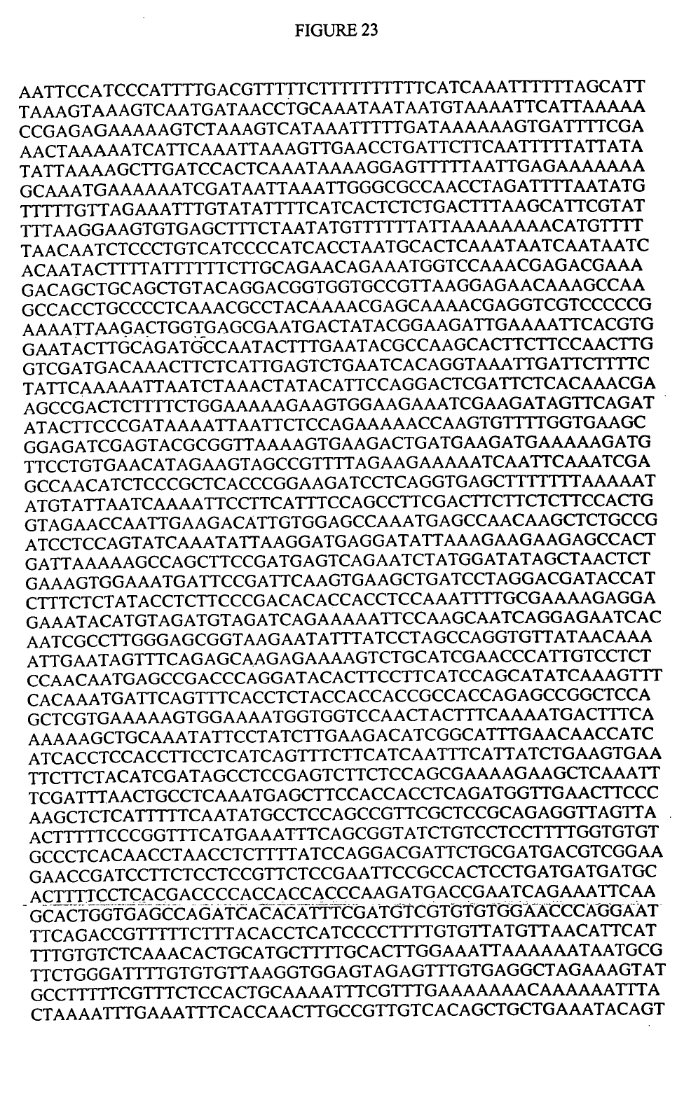

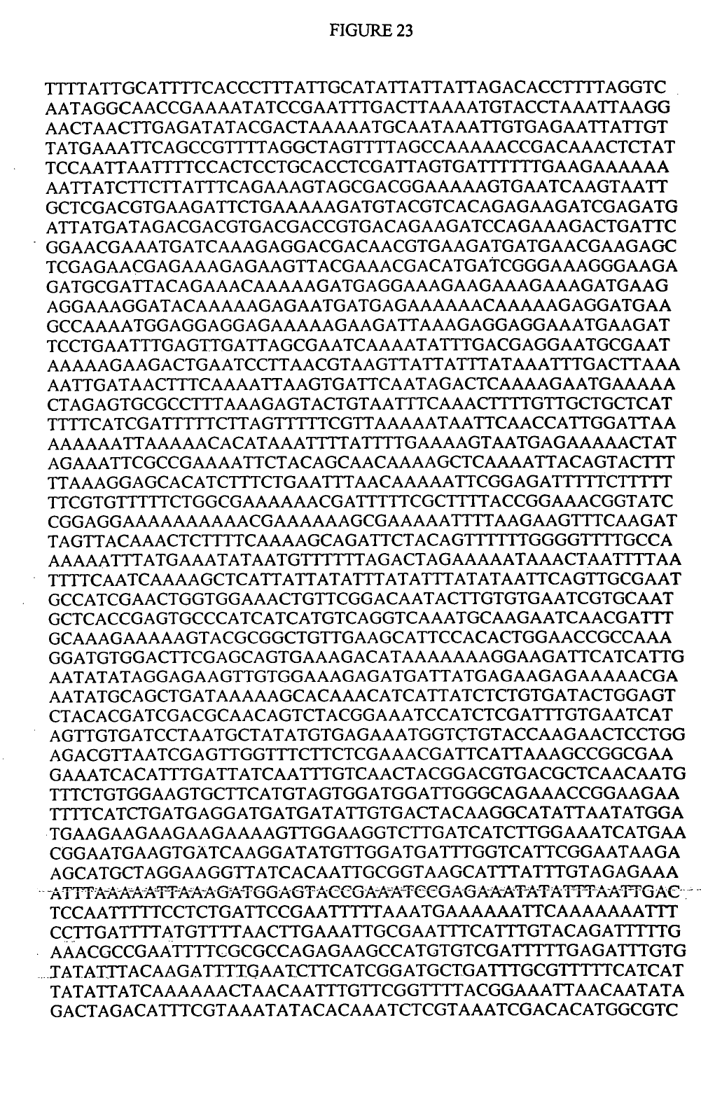

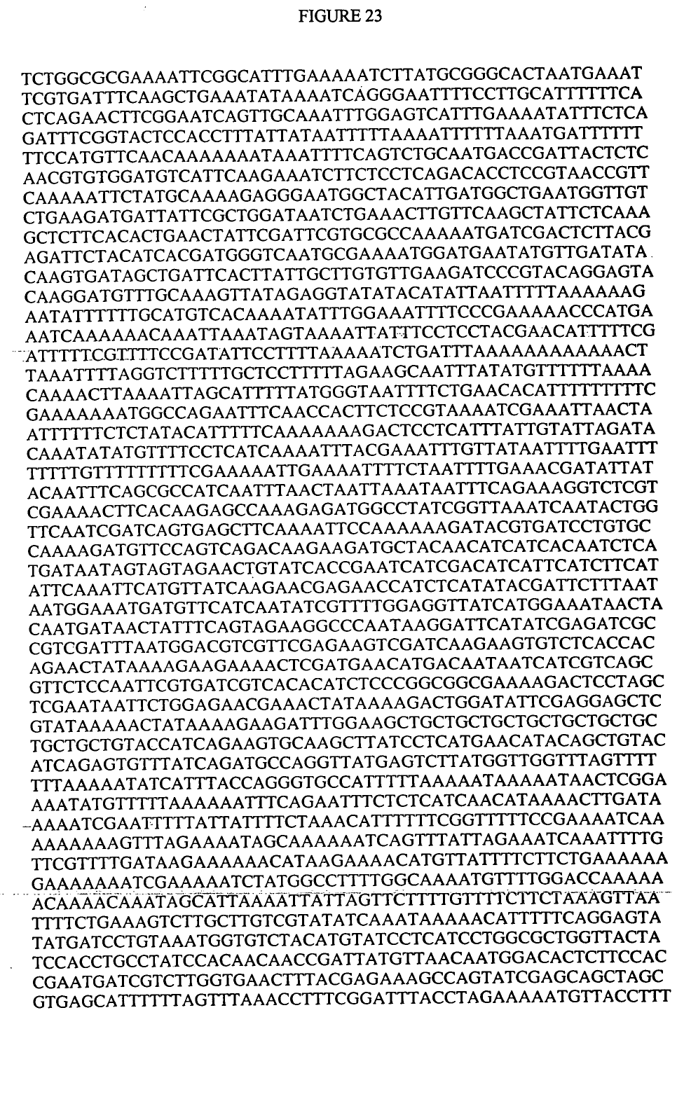

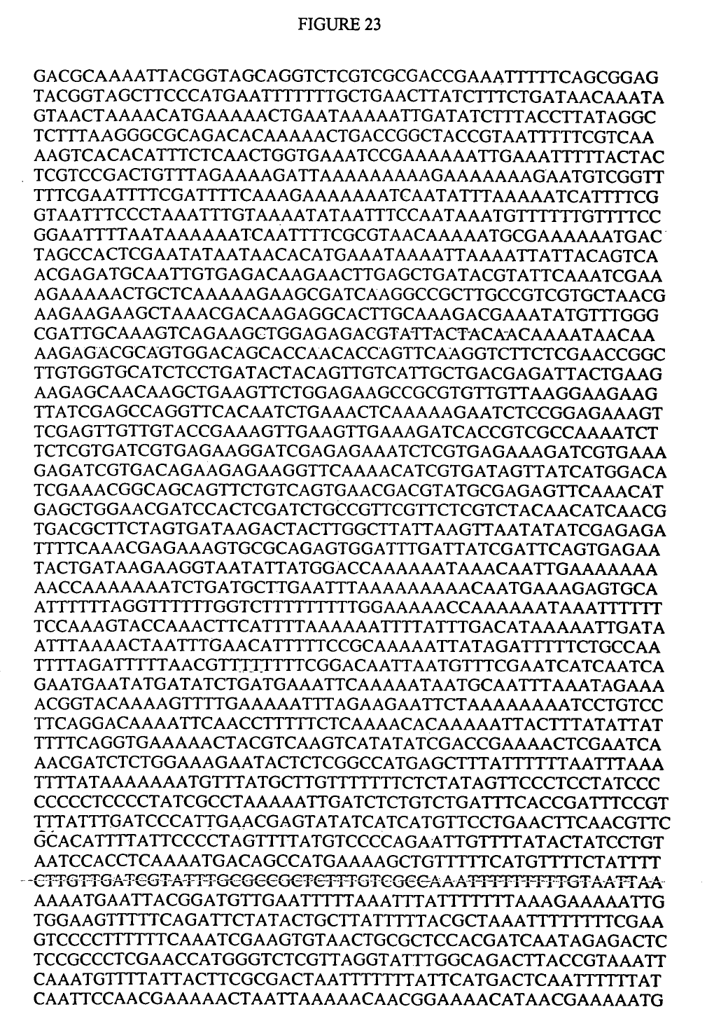

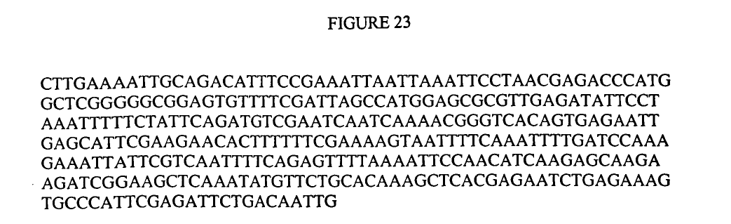

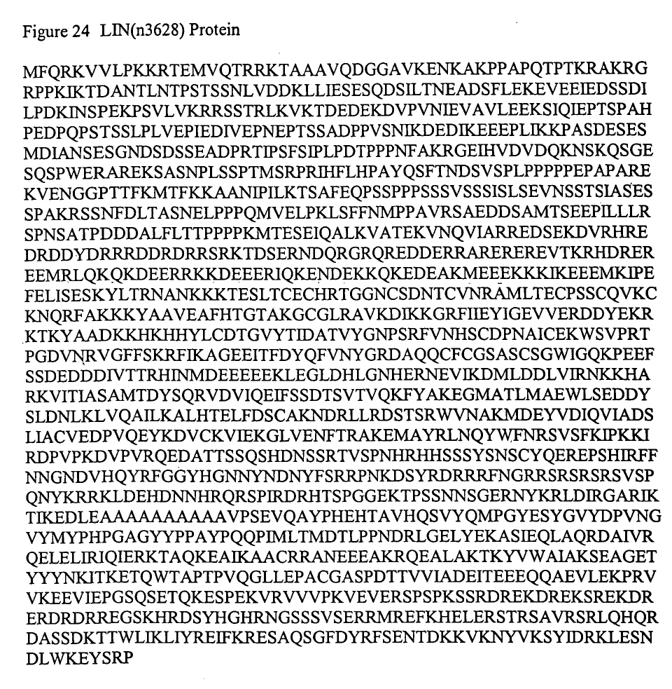

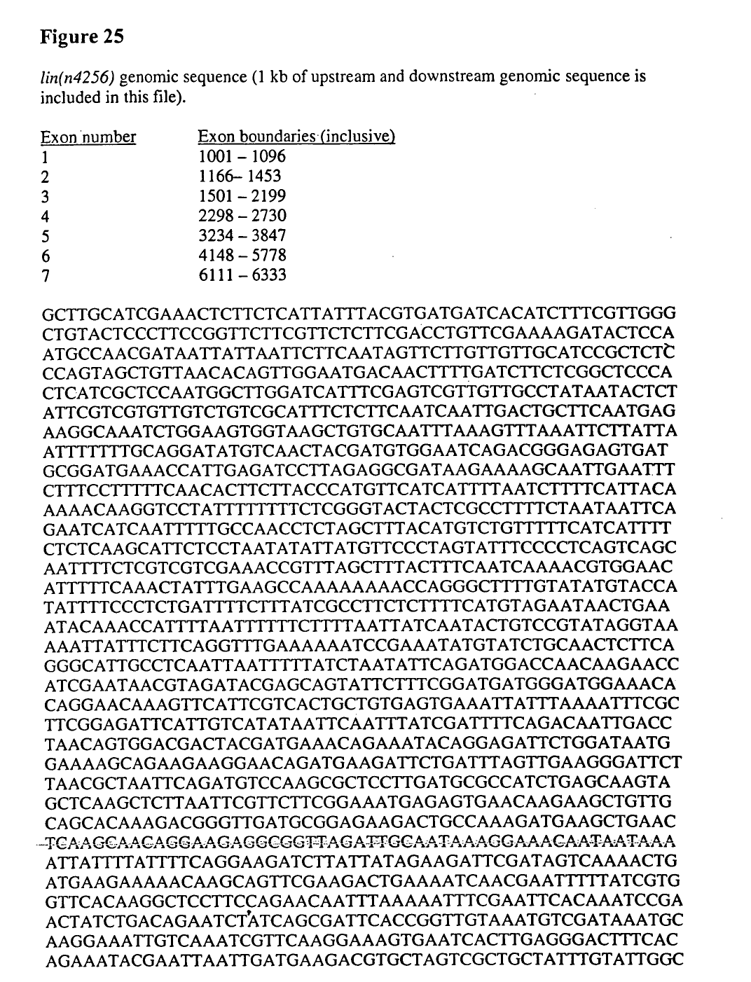

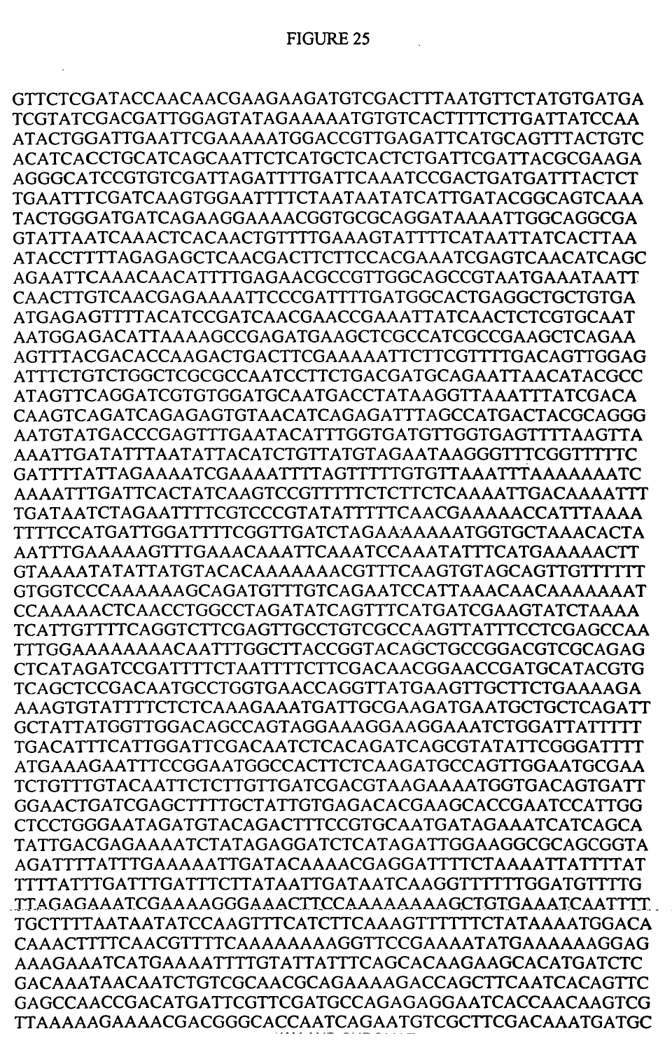

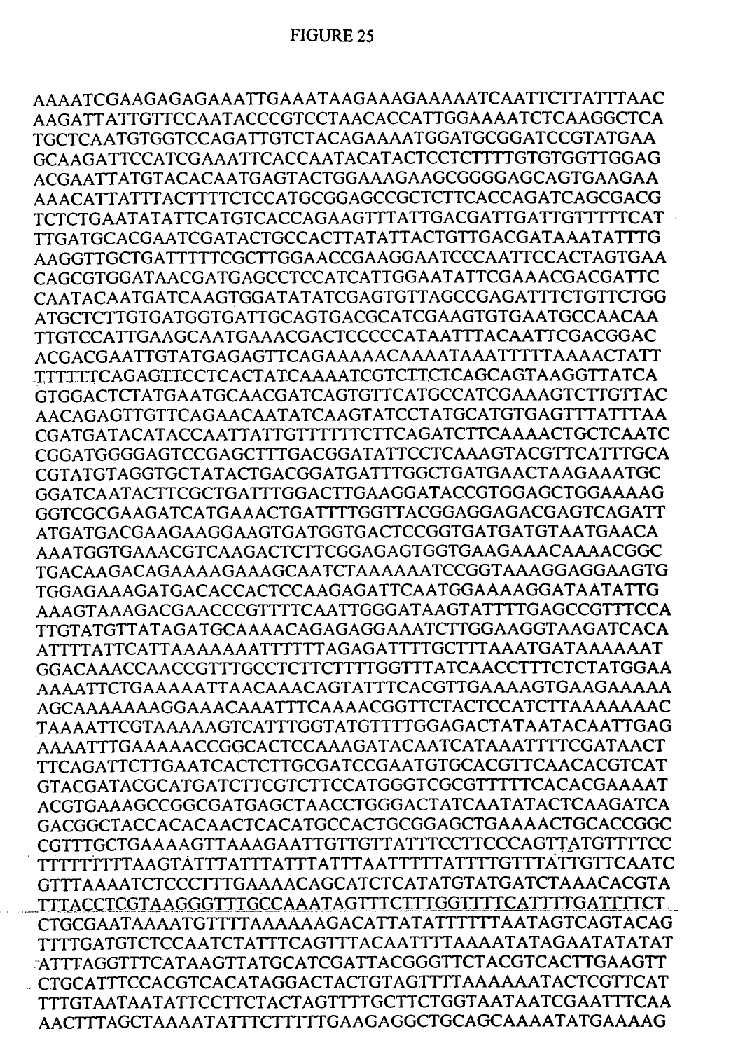

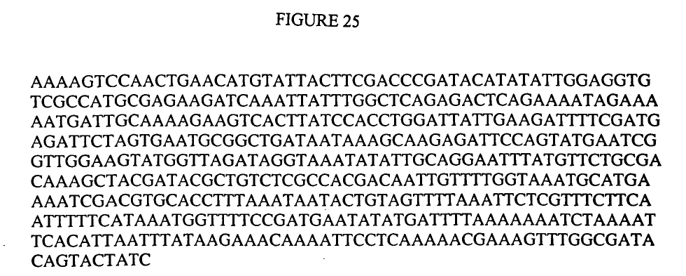

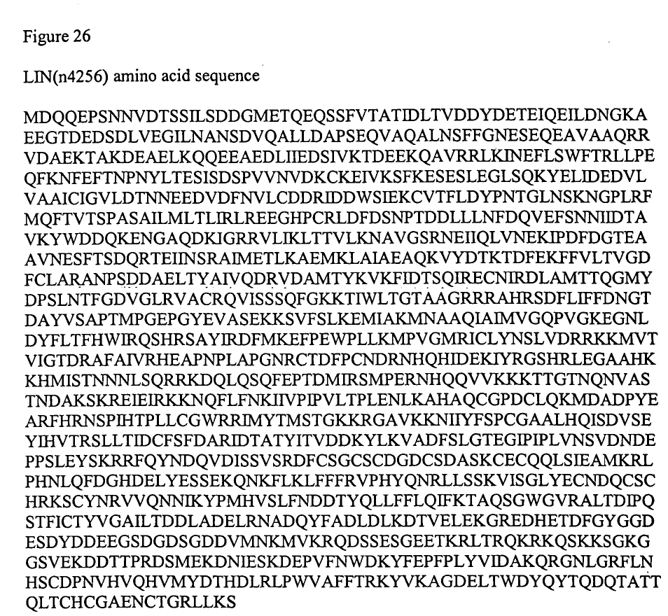

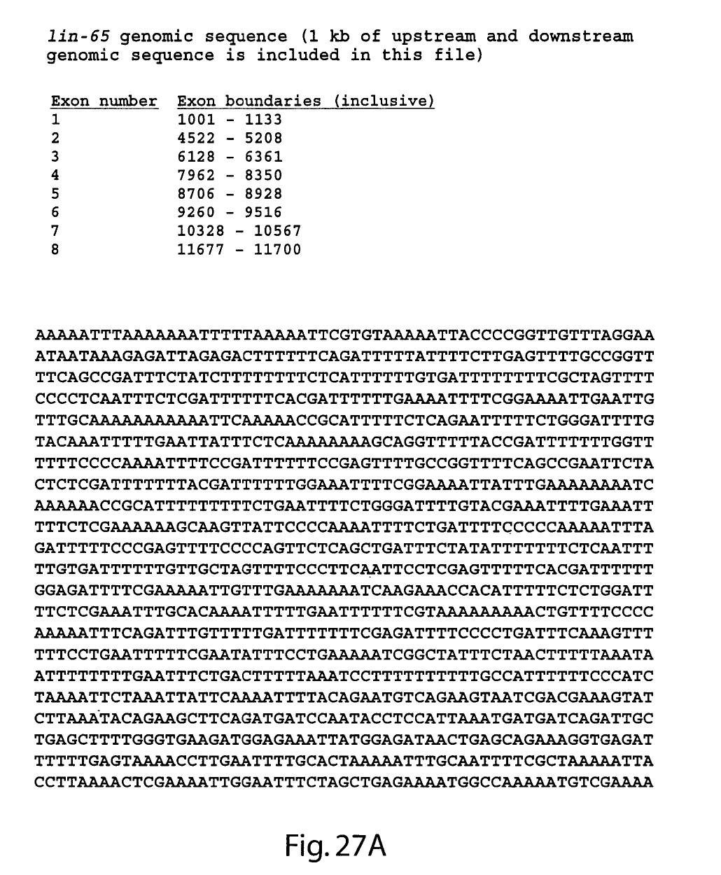

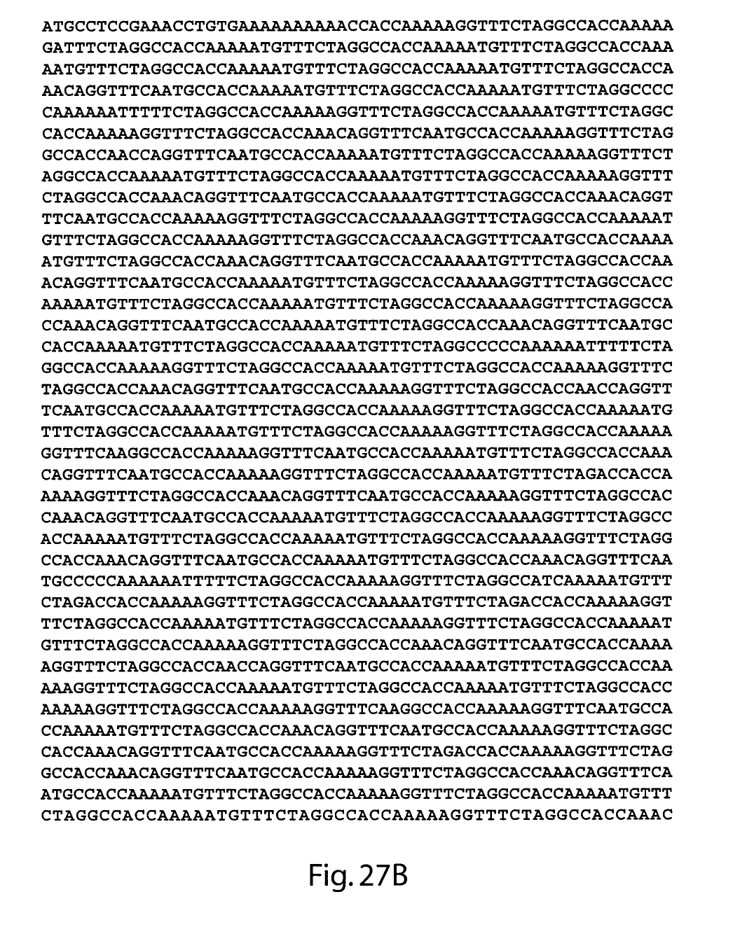

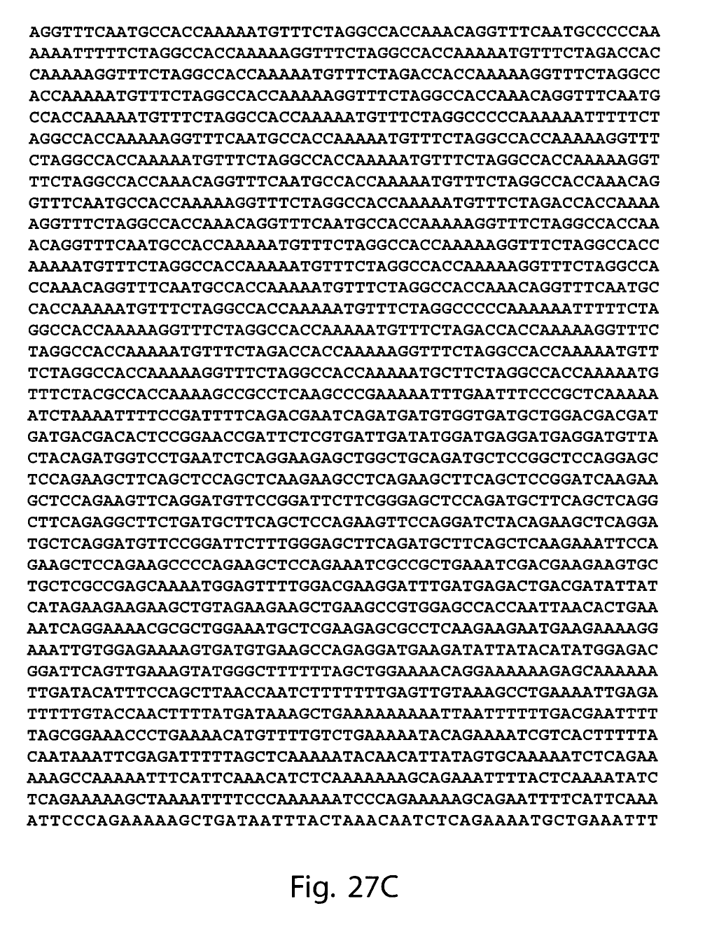

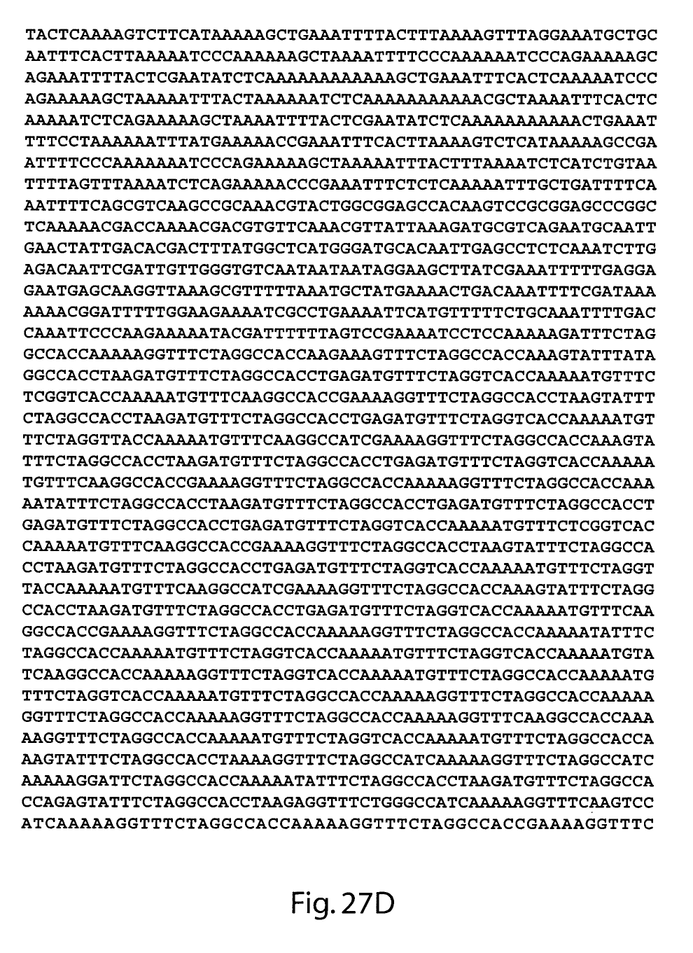

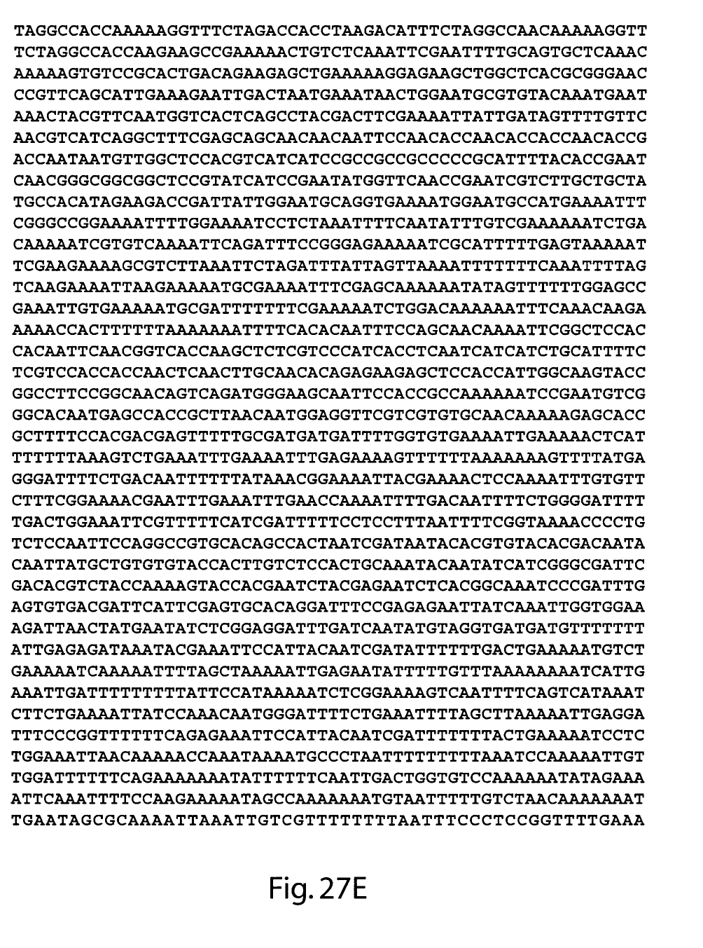

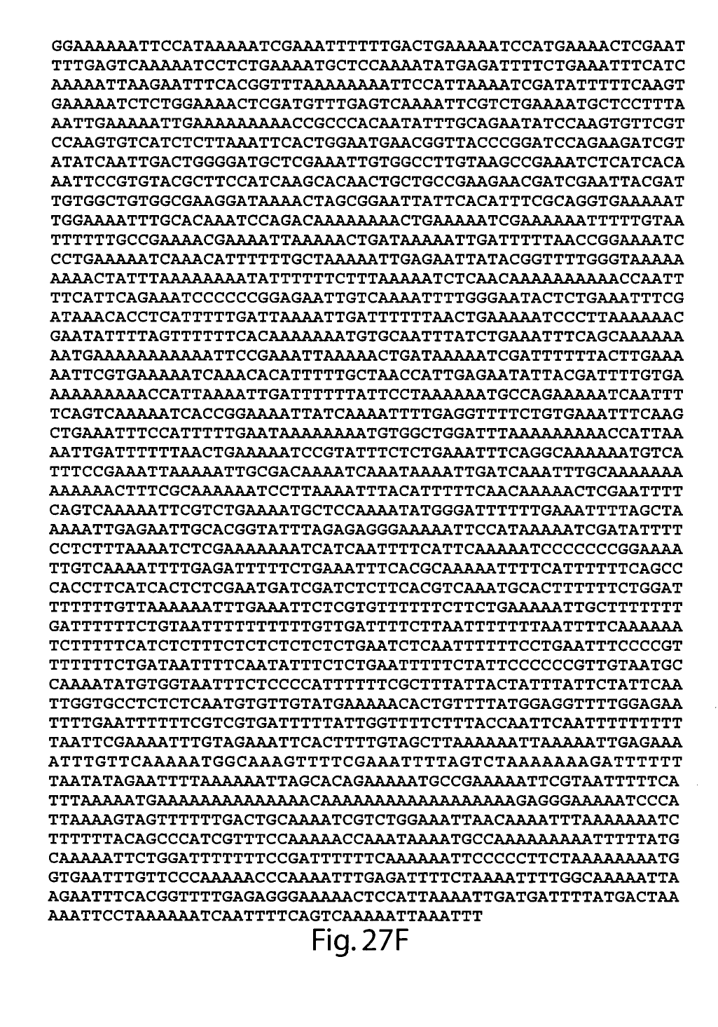

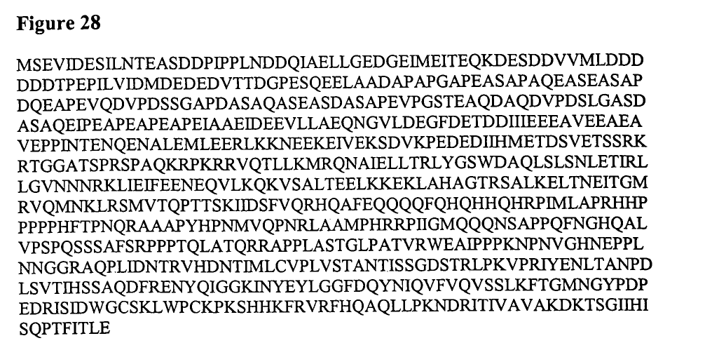

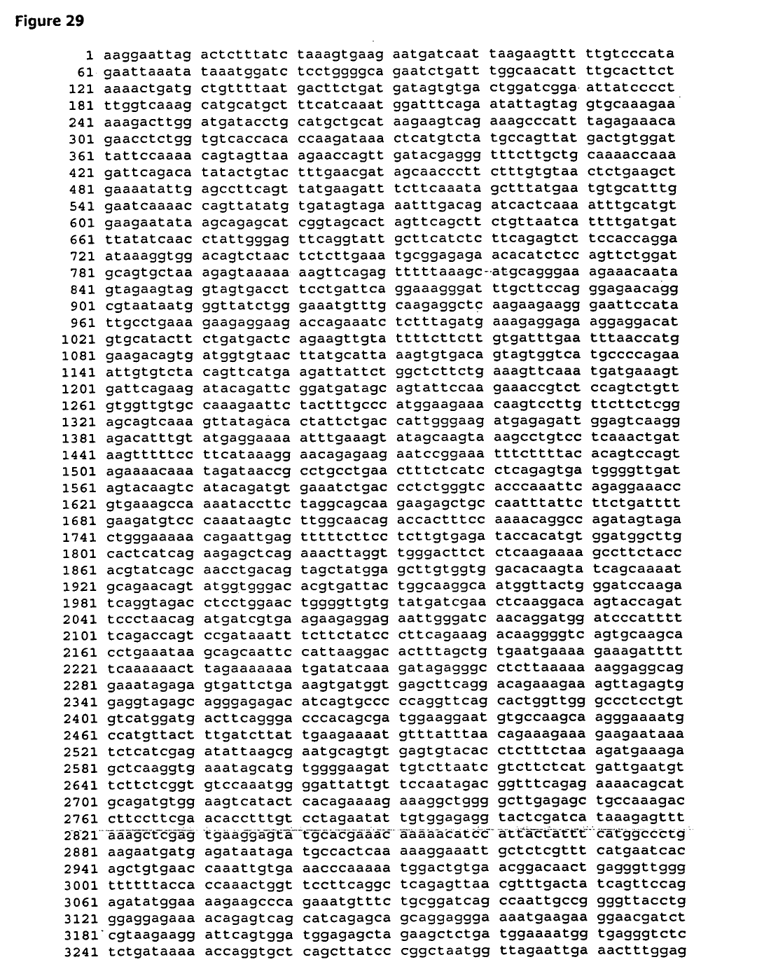

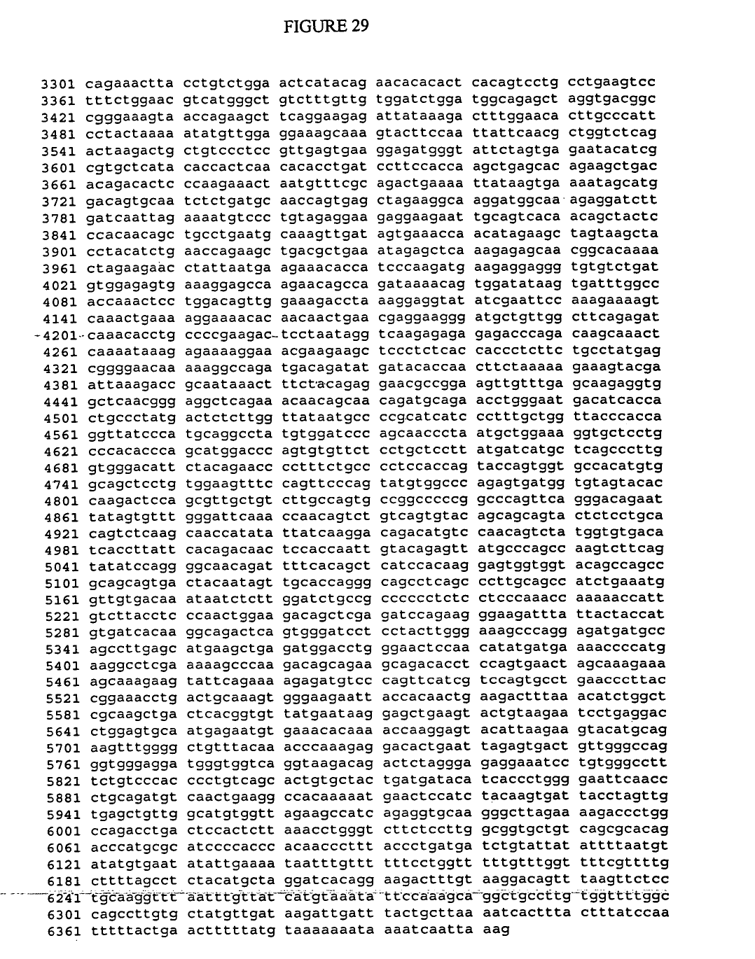

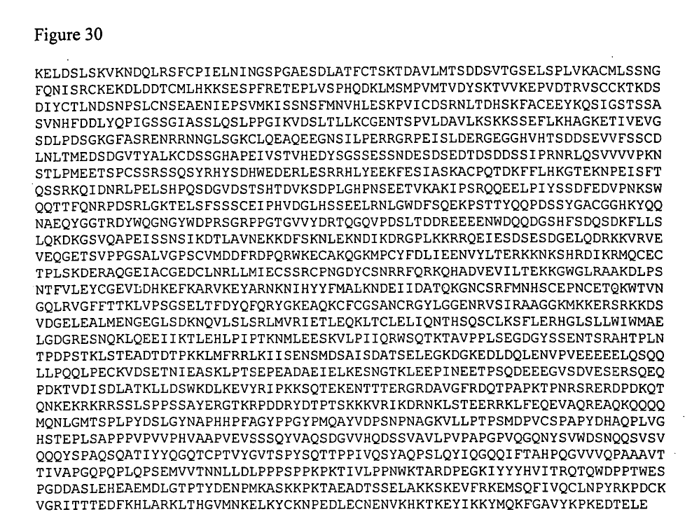

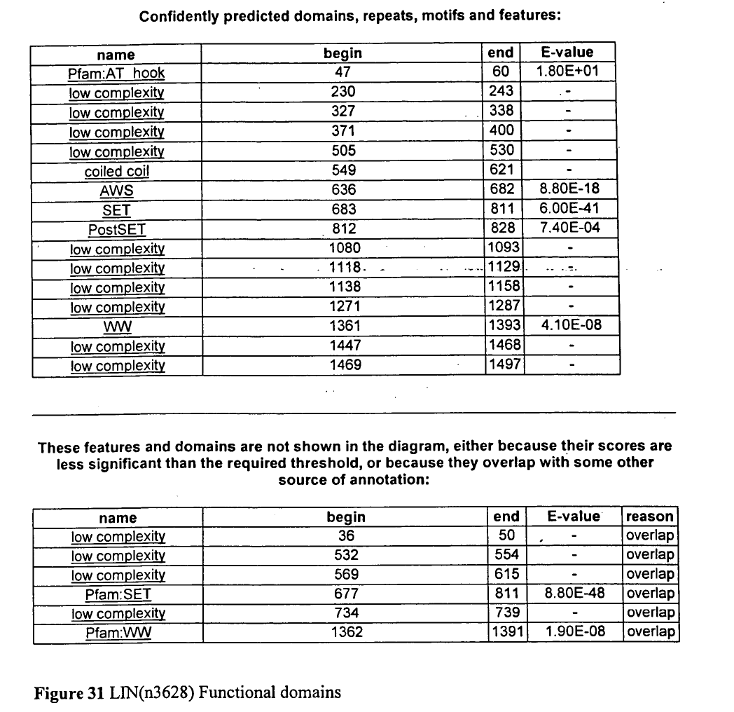

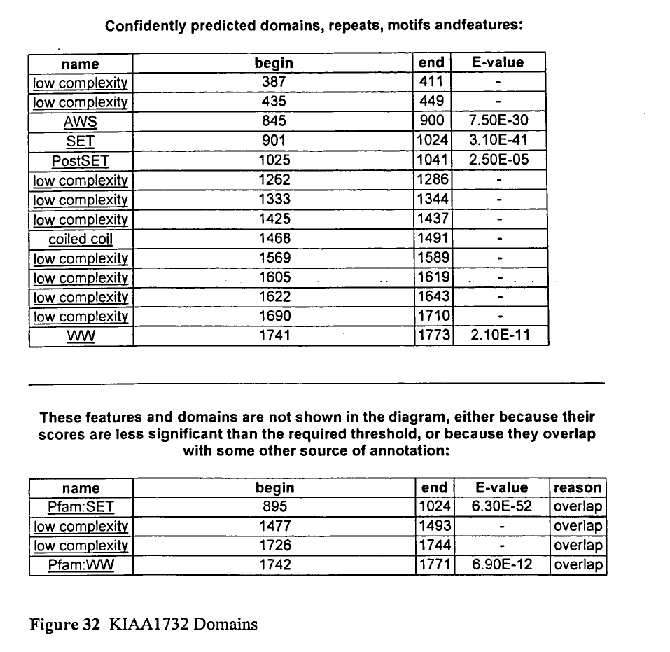

On the basis of genetic interactions, the synMuv genes have been grouped into three classes A, B and C. For an animal to show vulval abnormalities, genes representing two of three classes must be dysfunctional. The class B synMuv genes include genes that encode homologs of the mammalian Rb tumor suppressor protein and other proteins that act with Rb in regulating cell-fate specification and division in mammals. We have recently discovered three new class B synMuv genes: lin(n3628), lin(n4256), and lin-65. lin(n3628) encodes a protein similar to the yeast Set2 histone methyltransferase. The nucleic acid and amino acid sequences of lin(n3628) are shown in FIGS. 23 and 24, respectively. lin(n4256) encodes a protein similar to yeast and mammalian SUV39H1 family histone methyltransferases. The nucleic acid and amino acid sequences of lin(n4256) are provided in FIGS. 25 and 26. lin-65 encodes a protein rich in acidic amino acids. The nucleic acid and amino acid sequences of lin-65 are provided in FIGS. 27 and 28.

The striking parallel between the Rb pathway in mammals and the Rb-related pathway we nave identified in worms suggests that further characterization of the synthetic Multivulva genes will provide insights into how cell proliferation is regulated in humans. Because synMuv genes encode members of a conserved tumor suppressor pathway that antagonizes a conserved Ras oncogene pathway, the class B synMuv genes are likely to be important in understanding cancer progression in mammals. Provided with the human genome sequence, standard methods can be used to identify mammalian orthologs of newly-identified synMuv genes. Such homologs may act as tumor suppressors or oncogenes in mammals. Genetic enhancer or suppressor screens may be perfomed to identify new genes which may function in or interface with this Rb-related pathway. Furthermore, using methods described herein, drug screens can be used to identify compounds that affect cell proliferation. Compounds that block the Muv phenotype of synMuv mutant animals are likely to be useful antitumor agents for the treatment of a mammalian neoplasia.

Compounds that stimulate cell division in animals with a single, silent synMuv mutation are likely to be agonists of cell proliferation and may act in a manner analogous to growth factors. Such compounds are useful in the treatment of a subject in need of increased cell proliferation, for-example, in a subject that has a disordercharacterized by increased cell death, such as Alzheimer's disease, Huntington's disease, stroke, Parkinson's disease, myocardial infarction or congestive heart failure.

Identifying synMuv Targets

The targets of synMuv biological activity, for example, genes that are transcriptionally regulated by a synMuv nucleic acid or polypeptide, are identified using a variety of genetic and molecular approaches. While target identification is discussed below for the class B synMuvs, similar approaches are used to identify the targets of the class C synMuvs or other transcriptional regulatory systems.

At least two genetic screens can be used to identify class B synMuv targets. Both screens are based on the premise that the class B synMuv proteins negatively regulate transcription. Given that class B synMuv proteins are likely to negatively regulate transcription, one would postulate that the Muv phenotype of synMuv mutants is due to the ectopic expression of class B targets. Loss of function mutations in such targets likely suppress the synMuv phenotype. In one example, a simple F₂ suppression screen is used to identify such targets. In fact, such screens have identified Class B suppressor mutations that may affect such genes. Many of the isolates from these screens are as yet uncharacterized.

In a second example, which would likely identify genes whose expression is negatively regulated by the class B synMuvs, mutagenized class A synMuv F₁ animals are screened for a Muv phenotype. Dominant mutations expected from this screen might affect regulatory sequences bound by synMuv proteins and lead to ectopic expression of the target gene in question. Mutations of this type have been shown to affect the expression of egl-1, a gene that promotes programmed cell death in C. elegans. These egl-1 (gf) mutations disrupt a binding site for the TRA-1 transcriptional repressor protein, leading to ectopic egl-1 expression in the hermaphrodite specific neurons and subsequent programmed cell death (Conradt et al. Cell 98:317-27, 1999).

Because transcription factors typically target multiple genes, loss of function of one target may not suppress the phenotype caused by a transcriptional repressor loss of function or, alternatively, recapitulate the phenotype caused by transcriptional activator loss of function. Such challenges are overcome by performing screens in a particularly sensitized genetic background so as to allow the observation of a small effect that may be caused by loss of one target. For example, in one of the screens described above, the Muv phenotype caused by a temperature-sensitive lin-15AB allele was suppressed. A similarly sensitized background may be used for to carry out F₂ suppression and F₁ synMuv screens.

Various molecular approaches involving microarrays are also useful in identifying synMuv targets. In the simplest experiment, expression profiles of synMuv mutants are compared to the wild type. A comparison of synMuv double mutant to the wild type can be problematic because these animals have different amounts of vulval tissue. The generation of vulval tissue likely involves the differential regulation of many genes, only a subset of which might be direct targets of synMuvs. Alternatively, a synMuv single mutant can be compared to a wild-type control. This approach may not succeed if two classes of synMuvs must lose function in order for transcription to be differentially regulated. If mutations in two classes of synMuvs are desired, an appropriate comparison may, for example, be that of a synMuvA; synMuvB; let-60 Ras triple mutant versus a let-60 Ras single mutant. These animals would fulfill the requirements of having the same amount of vulval tissue and disabling two classes of synMuvs. Alternatively, chromatin immunoprecipitation (ChIP) combined with microarray analysis may be used. For example, in a preparation of proteins crosslinked to DNA, DPL-1 or EFL-1 could be immunoprecipitated, the crosslink reversed and the resultant DNA amplified and applied to microarrays. Such microarray experiments described above may identify synMuv targets that could be compared to putative let-60 Ras pathway targets as previously determined by microarray analyses (Romagnolo et al., Dev Biol 247:127-36, 2002). Determining this interface is clearly an important issue as Rb and Ras pathways antagonize each other not only in C. elegans, butalso during cell cycle progression in cultured mammalian cells (Mittnacht et al., Curr Biol. 7:219-21, 1997; Peeper et al., Nature. 386:177-81, 1997).

Do the synMuv Genes Act by Regulating Cell Cycle Pprogression?

Many studies of Rb and E2F in mammals have focused on the roles of these proteins in cell cycle regulation. Might the class B synMuv genes, and possibly other classes of synMuv genes regulate vulval development through direct regulation of P(3-8).p cell cycles? While not being tied to a particular theory, the following observations support this possibility. For example, P3.p, P4.p, and P8.p undergo extra cell divisions in synMuv mutants. Additionally, mutations in a subset of class B synMuv genes that includes dpl-1, efl-1, and lin-35 Rb have been shown to partially suppress the S phase and cell division defects caused by RNA-mediated interference of the C. elegans cyclin D homolog cyd-1 (Boxem et al., Curr Biol. 12:906-11, 2002). There are other aspects of these observations that complicate a strict cell cycle regulation model. First, not only are there extra P3.p, P4.p and P8.p cell divisions in synMuv mutants, but there are also various changes in the differentiation of P3.p, P4.p and P8.p descendants in synMuv mutants. The synMuv genes therefore appear to regulate a cell fate decision, a component of which is the decision to progress through the cell cycle. Studies of Rb in mammals have indicated that Rb may have a role in halting cell cycle progression and stimulating differentiation during myogenesis (reviewed by Kitzmann Cell Mol Life Sci. 58:571-9, 2001). Second, whereas dpl-1, efl-1, and lin-35 Rb mutations can partially suppress defects caused by cyd-1(RNAi), mutations in other class B synMuv genes cannot (Boxem et al., Curr Biol. 12:906-11, 2002). This observation suggests that, if the class B synMuv genes are cell cycle regulators, some of them act in a tissue-specific fashion, for example in P(3-8).p but not in the intestinal cells that were monitored in cyd-1(RNAi) studies. Monitoring cell cycle progression in P3.p, P4.p and P8.p will address these issues.

The identification of synMuv transcriptional targets will enable us to identify their mammalian orthologs. Such targets are promising clinical targets for chemotherapeutics for the treatment of neoplasia. In addition, the identification of synMuv protein-protein interactions is useful in screening for chemotherapeutic drugs that modulate such interactions.

Identification of Additional Mammalian Orthologs

Because the Rb and RAS pathways are conserved between mammals and C. elegans, the powerful genetics and genomics of C. elegans can be exploited, as described herein, for the systematic identification of mammalian genes that correspond to C. elegans genes identified according to methods described herein. Such genes include mammalian orthologs of synMuv class B, and class C genes and their transcriptional targets.

Protein sequences corresponding to genes of interest are retrieved from the repositories of C. elegans sequence information the wormbase web site. The C. elegans protein or nucleic acid sequence is then used for standard [BLASTP] or [tblastn] searching using the NCBI website. The protein sequence corresponding to the top mammalian candidate produced by tblastn is retrieved from Genbank and is used for BLASTp search of C. elegans proteins using the wormbase website. These methods allow us to identify mammalian orthologs of worm genes revealed by our genetic analysis.

An ortholog is a protein that is functionally related to a reference sequence. Such orthologs might be expected to functionally substitute for one another. For example, expression of a mammalian ortholog of a C. elegans gene, when expressed in a worm having a mutation in the C. elegans gene, might be expected to partially or completely rescue the worm phenotype.

RNAi in Mammalian Cell Lines

RNAi has been used extensively to deplete mRNAs in mammalian cell culture (Elbashiret-al.; Nature 411:494-8, 2001). Mammalian orthologs ofeclass C synMuv genes can be identified using RNAi, for example, in mammalian cultured cells. Briefly, an inhibitory nucleic acid is introduced into a mammalian cell having a mutation in a class A or class B synMuv gene, for example, by lipofection. Such cells are then assayed for increased levels of cell proliferation relative to control cells not contacted with an inhibitory nucleic acid. An increased level of proliferation in mammalian cells contacted with the inhibitory nucleic acid identifies the corresponding target gene as a class C synMuv gene.

Microarrays

The class B and class C genes described herein, are useful in identifying their transcriptional regulatory targets. Such targets may be identified using microarrays in combination with chromatin immunoprecipitation (chIP) as described herein. Such methods are described in U.S. Pat. Nos. 6,503,717, 6,410,243, and 6,610,489, hereby incorporated by reference. A nucleic acid target of a class B or class C synMuv polypeptide will likely have a mammalian ortholog. Such an ortholog represents a promising target for the development of novel chemotherapeutics for the treatment of a neoplasia.

The array elements, which are preferably derived from the C. elegans genome, are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28:e3.i-e3.vii, 2000), MacBeath et al., (Science 289:1760-1763, 2000), Zhu et al.(Nature Genet. 26:283-289), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in PCT application W095/251116 (Baldeschweiler et al.), incorporated herein by reference. Alternatively, a gridded array may be used to arrange and link cDNA fragments or oligonucleotides to the surface of a substrate using a vacuum system, thermal, ReV, mechanical or chemical bonding procedure.

A nucleic acid molecule (e.g. RNA or DNA) derived from a biological sample, such as a cultured cell, a tissue specimen, or other source, may be used to produce a hybridization probe as described herein. The mRNA is isolated according to standard methods, and cDNA is produced and used as a template to make complementary RNA suitable for hybridization using standard methods. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% fornamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent,e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% forniamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will De readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

Families of proteins, such as those encoded by the genes described herein, or their orthologs, may be analyzed using protein microarrays. Such arrays are useful in high-throughput low-cost screens to identify peptide or candidate compounds that bind a polypeptide of the invention, or fragment thereof. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides encoded by class B or class C synMuv gene or antibodies against such polypeptides) are spotted on a substrate using any convenient method known to the skilled artisan (e.g., by hand or by inkjet printer). Preferably, such methods retain the biological activity or function of the protein bound to the substrate

The protein microarray is hybridized with a detectable probe. Such probes can be polypeptide nucleic acid, or small molecules. For some applications, polypeptide and nucleic acid probes are derived from a biological sample taken from a patient, such as a a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Screening Assays

As discussed above, C. elegans class B and class C synMuv genes and their encoded proteins function in chromatin remodeling and antagonize the RAS pathway. Given that mechanisms for controlling mammalian cell cycle regulation and C. elegans vulval development are highly conserved, C. elegans and components of the C. elegans synMuv pathway are useful in screening methods for chemotherapeutics and for the identification of novel clinical targets.

Compounds that modulate the function of a Class B, or Class C synMuv nucleic acid or of their encoded proteins are likely to be useful in treating neoplasias. Based on this discovery, screening assays may be carried out to identify compounds that modulate the action of a polypeptide or the expression of a nucleic acid sequence of the invention. Such compounds are useful in treating a neoplasia. The method of screening may involve high-throughput techniques. In addition, these screening techniques may be carried out in cultured mammalian cells or in animals (e.g., nematodes).

Any number of methods are available for carrying out such screening assays. In one working example, candidate compounds are added at varying concentrations to the culture medium of cultured cells expressing one of the nucleic acid sequences described herein. Gene expression is then measured, for example, by standard Northern blot analysis (Ausubel et al., supra) or RT-PCR, using any appropriate fragment prepared from the nucleic acid molecule as a hybridization probe. The level of gene expression in the presence of the candidate compound is compared to the level measured in a control culture medium lacking the candidate molecule. A compound that promotes a decrease in the expression of a nucleic acid sequence disclosed herein or a functional equivalent is considered useful in the invention; such a molecule may be used, for example, as a therapeutic to delay or ameliorate human diseases associated with neoplasia or inappropriate cell cycle regulation. Such cultured cells include nematode cells (for example, C. elegans cells), mammalian, or insect cells.

In another working example, the effect of candidate compounds may be measured at the level of polypeptide production using the same general approach and standard immunological techniques, such as Western blotting or immunoprecipitation with an antibody specific for a polypeptide of the invention. For example, immunoassays may be used to detect or monitor the expression of at least one of the polypeptides of the invention in an organism. Polyclonal or monoclonal antibodies (produced by standard techniques) that are capable of binding to such a polypeptide may be used in any standard immunoassay format (e.g., ELISA, Western blot, or RIA assay) to measure the level of the polypeptide. A compound that promotes a decrease in the expression of the polypeptide is considered particularly useful. Again, such a molecule may be used, for example, as a therapeutic to ameliorate neoplasia.

In one example, candidate compounds are screened for those that specifically bind to and antagonize a synMuv B or synMuv C polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate the cell cycle or decrase cell proliferation may be assayed by any standard technique (e.g., a C. elegans synMuv assay).

In one particular working example, a candidate compound that binds to a polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the polypeptide is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to cause cell death using any assay known to the skilled artisan. Compounds isolated by this approach may also be used, for example, as therapeutics to delay or ameliorate human diseases associated with neoplasia. Compounds that are identified as binding to polypeptides of the invention with an affinity constant less than or equal to 10 mM are considered particularly useful in the invention.

Potential antagonists include organic molecules, peptides, peptide mimetics, polypeptides, nucleic acids, and antibodies that bind to a nucleic acid sequence or polypeptide of the invention and thereby increase or decrease its activity. Potential antagonists also include small molecules that bind to and occupy the binding site of the polypeptide thereby preventing binding to cellular binding molecules, such that normal biological activity is prevented.

Each of the DNA sequences provided herein may also be used in the discovery and development of therapeutic lead compounds. The encoded protein, upon expression, can be used as a target for the screening of therapeutics for the treatment of neoplasia. Additionally, the DNA sequences encoding the amino terminal regions of the encoded protein or Shine-Delgarno or other translation facilitating sequences of the respective mRNA can be used to construct antisense, dsRNAs, or siRNA sequences to control the expression of the coding sequence of interest. Such sequences may be isolated by standard techniques (Ausubel et al., supra). The antagonists of the invention may be employed, for instance, to delay or ameliorate human diseases associated with neoplasia.

Optionally, compounds identified in any of the above-described assays may be confirmed as useful in delaying or ameliorating human diseases associated with neoplasia or inappropriate cell cycle regulation in either standard tissue culture methods or animal models and, if successful, may be used as therapeutics for the treatment of neoplasia.

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of delaying or ameliorating human diseases associated with neoplasia are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, USA. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known to function in neoplasia should be employed whenever possible.

When a crude extract is found to decrease cell proliferation or to suppress a synMuv phenotype, further fractionation of the positive lead extract is necessary to isolate chemical constituents responsible for the observed effect. Thus, the goal of the extraction, fractionation, and purification process is the careful characterization and identification of a chemical entity within the crude extract that inhibits cell proliferation or suppresses a synMuv phenotype. Methods of fractionation and purification of such heterogenous extracts are known in the art. If desired, compounds shown to be useful agents to delay or ameliorate human diseases associated with neoplasia are chemically modified according to methods known in the art.

Pharmaceutical Therapeutics

The invention provides a simple means for identifying compositions (including nucleic acids, peptides, small molecule inhibitors, and mimetics) capable of acting as therapeutics for the treatment of a neoplastic disease. Accordingly, a chemical entity discovered to have medicinal value using the methods described herein is useful as a drug or as information for structural modification of existing compounds, e.g., by rational drug design. Such methods are useful for screening compounds having an effect on a variety of diseases characterized by inappropriate cell cycle regulation.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a neoplastic disease therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplastic disease. Generally, amounts will be in the range of those used for other agents used in the treatment of a neoplastic disease, although in certain instances lower amounts will be needed because of the increased specificity of the compound. A compound is administered at a dosage that controls the clinical or physiological symptoms of a neoplastic disease as determined by, for example, measuring tumor size, cell proliferation, or metastasis.

Formulation of Pharmaceutical Compositions

Administration of a compound may be by any suitable means that is effective for the treatment of a neoplastic disease. Generally, compounds are admixed with a suitable carrier substance, and are generally present in an amount of 1-95% by weight of the total weight of the composition. The composition may be provided in a dosage form that is suitable for oral, parenteral (e.g., intravenous, intramuscular, subcutaneous), rectal, transdermal, nasal, vaginal, inhalant, or ocular administration. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy, (20th ed.) ed. A.R. Gennaro, 2000, Lippincott Williams & Wilkins, Philedelphia, Pa. and Encyclopedia of Pharmaceutical Technology, eds. J. Swarbrick and J. C. Boylan, 1988-2002, Marcel Dekker, New York).

### OTHER EMBODIMENTS

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

1. A method for identifying a compound that treats a neoplasia, said method comprising the steps of:
(a) contacting a cell comprising a mutation in a Class B synMuv gene selected from the group consisting of: mep-1, lin(n3628), lin(n4256), and lin-65 and a second mutation in a synthetic multivulval gene, or an ortholog thereof, with a candidate compound;
(b) detecting a phenotypic alteration in said contacted cell relative to a control cell; wherein a candidate compound that alters the phenotype of said contacted cell relative to said control cell is a compound that treats a neoplasia.

2. The method of claim 1, wherein said cell is in a nematode.

3. The method of claim 1, wherein said cell is an isolated mammalian cell.

4. The method of claim 1, wherein said phenotypic alteration is a decrease in cell proliferation.

5. A method of identifying a nucleic acid target of class B synMuv biological activity, said method comprising:
(a) providing a microarray comprising fragments of nematode nucleic acids;
(b) contacting said microarray with detectably labeled nucleic acids derived from a nematode comprising a mutation in a Class B synMuv gene selected from the group consisting of: mep-1,lin(n3628), lin(n4256), and lin-65 gene;
(c) detecting an alteration in the expression of at least one nucleic acid of a C. elegans comprising a mutation in said Class B synMuv gene relative to the expression of said nucleic acid in a control nematode, wherein an alteration in said expression identifies said nucleic acid as a nucleic acid target of class B synMuv biological activity.

6. The method of claim 5, wherein said C. elegans further comprises a mutation in a second synMuv gene.

7. The method of claim 5, wherein said C. elegans further comprises a mutation in a gene that results in a Vulvaless (Vul) phenotype.

8. A method for identifying a nucleic acid that binds a synMuv class B polypeptide, said method comprising:
(a) providing nucleic acids derived from a nematode cell;
(b) crosslinking said nucleic acids and their associated proteins to form a nucleic acid-protein complex;
(c) contacting said nucleic acid-protein complex with an antibody against a polypeptide selected from the group consisting of MEP-1, LIN(n3628), LIN(n4256), and LIN-65;
(d) purifying said nucleic acid-protein complex using an immunological method; and
(e) isolating said nucleic acid, wherein said isolated nucleic acid is a nucleic acid that binds a synMuv class B polypeptide.

9. The method of claim 30, further comprising the following steps:
(f) detectably labeling the nucleic acid of step (e);
(g) contacting a microarray comprising C. elegans nucleic acid fragments with said detectably labeled nucleic acid; and
(h) detecting binding of said detectably labeled nucleic acid, wherein said binding identifies said nucleic acid as a nucleic acid that binds a synMuv class B polypeptide.

10. A method for identifying a candidate compound that treats a neoplasia, said method comprising:
(a) providing a cell having a mutation in a nucleic acid encoding KIAA1732 and having a second mutation in a synMuv nucleic acid, or ortholog thereof;
(b) contacting said cell with a candidate compound; and
(c) detecting a decrease in proliferation of said cell contacted with said candidate compound relative to a control cell not contacted with said candidate compound, wherein a decrease in proliferation identifies said candidate compound as a candidate compound that treats a neoplasia.

11. The method of claim 10, wherein said cell is an isolated mammalian cell.

12. A method for identifying a nucleic acid that binds KIAA1732, said method comprising:
(a) providing nucleic acids derived from a mammalian cell;
(b) crosslinking said nucleic acids and their associated proteins to form a nucleic acid-protein complex;
(c) contacting said nucleic acid-protein complex with an anti-KIAA1732 antibody;
(d) purifying said nucleic acid-protein complex using an immunological method; and
(e) isolating said nucleic acid, wherein said isolated nucleic acid is a nucleic acid that binds KIAA1732.

13. The method of claim 12, further comprising the following steps:
(f) detectably labeling the nucleic acid of step (e);
(g) contacting a microarray comprising human, nucleic acid fragments with said detectably labeled nucleic acid; and
(h) detecting binding of said detectably labeled nucleic acid, wherein said binding identifies said nucleic acid as a nucleic acid that binds KIAA1732.

14. A vector comprising a nucleic acid having at least 95% identity to (SEQ ID NO:30).

15. An isolated cell comprising the vector of claim 3.

16. A method for identifying a compound that treats a neoplasia, said method comprising the steps of:
(a) contacting a nematode comprising a mutation in a Class C synMuv gene selected from the group consisting of trr-1, hat-1, epc-1, and ssl-1 with a candidate compound; and
(b) detecting an alterated phenotype in said contacted nematode relative to a control nematode; wherein a candidate compound that alters the phenotype of said contacted nematode relative to said control nematode is a compound that treats a neoplasia.

17. A method of identifying a nucleic acid whose expression is modulated by a synMuv class C polypeptide, said method comprising:
(a) providing a microarray comprising fragments of nematode nucleic acids;
(b) contacting said microarray with detectably labeled nucleic acids derived from a nematode comprising a mutation in a Class C synMuv gene selected from the group consisting of trr-1, hat-1, epc-1, and ssl-1 gene;
(c) detecting an alteration in the expression of at least one nucleic acid of a C. elegans comprising a mutation in said synMuv class C gene relative to the expression of said nucleic-acidin a control nematode, wherein an alteration in said expression identifies said nucleic acid as a nucleic acid modulated by a synMuv class C polypeptide.

18. The method of claim 17, wherein said C. elegans further comprises a mutation in a gene that results in a Vulvaless (Vul) phenotype.

19. The method of claim 18, wherein said gene encodes LET-60.

20. A method for identifying a nucleic acid target of a synMuv class C polypeptide, said method comprising:
(a) providing nucleic acids derived from a nematode cell;
(b) crosslinking said nucleic acids and their associated proteins to form a nucleic acid-protein complex;
(c) contacting said nucleic acid-protein complex with an antibody that binds a polypeptide selected from the group consisting of TRR-1, HAT-1, EPC-1, AND SSL-1;
(d) purifying said nucleic acid-protein complex using an immunological method; and
(e) isolating said nucleic acid, wherein said isolated nucleic acid is a nucleic acid that binds a synMuv class C polypeptide.

21. The method of claim 20, further comprising the following steps:
(f) detectably labeling the nucleic acid of step (e);
(g) contacting said detectably labeled nucleic acid with a microarray comprising C. elegans nucleic acid fragments; and
(h) detecting binding of said detectably labeled nucleic acid, wherein said binding identifies said nucleic acid as a nucleic acid target of a synMuv class C polypeptide.
